(19) United States
(12) Patent Application Publication
(10) Pub. No.: US 2026/0242780 A1
(43) Pub. Date: Aug. 20, 2026

(54) COMPONENTS, COMPOSITIONS, AND METHODS FOR CONTROL OF GENE EDITING

(71) Applicant: Acrigen Biosciences, Berkeley, CA (US)

(72) Inventors: David Rabuka, Berkeley, CA (US); Michael Schelle, Berkeley, CA (US); Allison Sharrar, Berkeley, CA (US); Luisa Mayumi Arake De Tacca, Berkeley, CA (US)

(21) Appl. No.: 19/165,338

(22) PCT Filed: Mar. 15, 2024
(86) PCT No.: PCT/US2024/020085

§ 371 Date: Sep. 15, 2025

Related U.S. Application Data
(60) Provisional application No. 63/490,970, filed on Mar. 17, 2023.

(51) Int. Cl. C12N 15/11; C07K 14/195; C12N 9/22; C12N 15/88

(52) U.S. Cl. CPC C12N 15/111; C07K 14/195; C12N 9/226; C12N 15/88; C12N 2310/20; C12N 2840/203

(57) ABSTRACT

The present disclosure provides components, and compositions, methods, and systems thereof for controlling gene editing. More particularly, the present disclosure provides anti-CRISPR (Acr) polypeptides, and compositions, methods, and systems thereof, for controlling Cas protein activity, e.g., Cas12f or Cas, gene editing activity.

### FIELD

The present invention relates to components, and compositions, methods, and systems thereof for controlling gene editing. Particularly, the invention relates to anti-CRISPR (Acr) polypeptides, and compositions, methods, and systems thereof, for controlling CRISPR-associated (Cas) protein activity, e.g., Cas12f or μCas, gene editing activity.

### CROSS-REFERENCE TO RELATED APPLICATIONS

This application claims the benefit of U.S. Provisional Application No. 63/490,970, filed Mar. 17, 2023, the content of which is herein incorporated by reference in its entirety.

### SEQUENCE LISTING STATEMENT

The content of the electronic sequence listing titled ACRIG_41400_601_SequenceListing.xml (Size: 345,480 bytes; and Date of Creation: Mar. 12, 2024) is herein incorporated by reference in its entirety.

### BACKGROUND

Clustered regularly interspaced short palindromic repeats (CRISPR)-associated proteins dominate the nucleic acid-editing landscape because they are versatile, rapid, and easy-to-use editing tools. The most well-characterized CRISPR-Cas nuclease, Cas9, utilizes one or more RNAs to act as a sequence-specific targeting element linking the nuclease to the target nucleic acid. However, presently CRISPR-Cas systems have some limitations for use in eukaryotic organisms including: inefficient delivery to mature cells in large numbers, low efficiency of editing, off-target events, target sequence preferences, and optimal temperatures and conditions for enzymatic activity.

### SUMMARY

Provided herein are system comprising: (i) an anti-CRISPR (Acr) polypeptide comprising an amino acid sequence having about 80% or more (e.g., about 85%, about 90%, about 95%, about 97%, about 98%, about 99%, or more) sequence identity with an amino acid sequence set forth in any one of SEQ ID NOs: 1-6, or a nucleic acid encoding the Acr polypeptide; and (ii) a CRISPR-associated protein or a nucleic acid encoding the CRISPR-associated protein.

In some embodiments, the CRISPR-associated protein is a Cas12f or a μCas. In some embodiments, the CRISPR-associated protein comprises an amino acid sequence set forth in any one of SEQ ID NOs: 7-9 or an amino acid sequence having about 80% or more sequence identity with any one of SEQ ID NOs: 7-9. In some embodiments, the CRISPR-associated protein comprises an amino acid sequence set forth in any one of SEQ ID NOs: 10-259 or an amino acid sequence having about 80% or more sequence identity with any one of SEQ ID NOs: 10-259.

In some embodiments, the Acr polypeptide is capable of modulating an activity of the CRISPR-associated protein. In some embodiments, the Acr polypeptide is capable of specifically modulating an activity of a Cas12f or a μCas CRISPR-associated protein, and the Acr does not modulate the activity of a Cas9 or a Cas12a CRISPR-associated protein. In some embodiments, the activity comprises one or more of: single-strand nucleic acid cleavage, double-strand nucleic acid cleavage, nucleic acid binding, and base editing. In some embodiments, modulating comprises fully or partially inhibiting the activity of the CRISPR-associated protein. In some embodiments, the Acr polypeptide is capable of inhibiting the activity of the CRISPR-associated protein at least 1.5, 2, 2.5, 3, 3.5, 4, 5, 6, 7, 8, 9, 10 or more fold as compared to CRISPR-associated protein activity in the absence of the Acr polypeptide. In some embodiments, the Acr polypeptide decreases or is capable of decreasing the activity of the CRISPR-associated protein by at least 5%, at least 10%, at least 15%, at least 20%, at least 25%, at least 30%, at least 40%, at least 50%, at least 60%, at least 70%, at least 80%, at least 90%, or more compared to CRISPR-associated protein activity in the absence of the Acr polypeptide.

In some embodiments, the CRISPR-associated protein modulates the ON and/or OFF target efficiencies of a CRISPR-associated protein activity. In some embodiments, the ratio of ON target to OFF target activity of the CRISPR-associated protein in the presence of the Acr polypeptide is greater than the ratio of ON target to OFF target activity of the CRISPR-associated protein in the absence of the Acr polypeptide. In some embodiments, the OFF target comprises a nucleic acid sequence having 1, 2, 3 or more amino acid differences compared to the ON target.

In some embodiments, the system comprises the nucleic acid encoding the Acr polypeptide and/or the nucleic acid encoding the CRISPR-associated protein.

In some embodiments, the system comprises a first nucleic acid encoding the Acr polypeptide and a second nucleic acid encoding the CRISPR-associated protein. In some embodiments, the Acr polypeptide and the nucleic CRISPR-associated protein are encoded by a single nucleic acid.

In some embodiments, a translation control element is operably linked to the Acr polypeptide coding sequence and/or to the CRISPR-associated protein coding sequence. In some embodiments, the translation control element is selected from the group consisting of an IRES sequence, a ribosome skip sequence, a noncanonical start codon or any combination thereof. In some embodiments, a first promoter is operably linked to the Acr polypeptide coding sequence and a second promoter is operably linked to the CRISPR-associated protein coding sequence. In some embodiments, the first promoter is a stronger promoter than the second promoter. In some embodiments, the second promoter is a stronger promoter than the first promoter.

In some embodiments, the systems further comprise at least one guide RNA or a nucleic acid encoding the at least one guide RNA.

In some embodiments, the one or more of the nucleic acids encoding the Acr polypeptide, the CRISPR-associated protein or the guide RNA is comprised in a viral vector. In some embodiments, the viral vector is an adeno-associated virus (AAV) vector.

In some embodiments, one or more of the nucleic acids encoding the Acr polypeptide, the CRISPR-associated protein or the guide RNA is comprised in a lipid nanoparticle (LNP).

In some embodiments, the system comprises the Acr polypeptide and/or the CRISPR-associated protein in protein form. In some embodiments, the Acr polypeptide and/or the CRISPR-associated protein is comprised in a lipid nanoparticle (LNP).

Also provided herein are methods for modifying a target nucleic acid comprising: contacting the target nucleic acid with a system as disclosed herein.

In some embodiments, the activity of the CRISPR-associated protein on the target nucleic acid is modulated by the Acr polypeptide. In some embodiments, the activity comprises one or more of single-strand nucleic acid cleavage, double-strand nucleic acid cleavage, nucleic acid binding, and base editing. In some embodiments, modulating comprises inhibiting the activity of the CRISPR-associated protein. In some embodiments, the Acr polypeptide inhibits the activity of the CRISPR-associated protein at least 1.5, 2, 2.5, 3, 3.5, 4, 5, 6, 7, 8, 9, 10 or more fold as compared to CRISPR-associated protein activity in the absence of the Acr polypeptide. In some embodiments, the Acr polypeptide decreases or is capable of decreasing the activity of the CRISPR-associated protein by at least 5%, at least 10%, at least 15%, at least 20%, at least 25%, at least 30%, at least 40%, at least 50%, at least 60%, at least 70%, at least 80%, at least 90%, or more compared to CRISPR-associated protein activity in the absence of the Acr polypeptide.

In some embodiments, the Acr polypeptide preferentially inhibits, fully or partially, the activity of the CRISPR-associated protein on one or more OFF targets as compared to the target nucleic acid sequence. In some embodiments, the OFF target comprises a nucleic acid sequence having 1, 2, 3 or more amino acid differences compared to the target nucleic acid sequence.

In some embodiments, the contacting occurs in vivo. In some embodiments, the contacting occurs in a eukaryotic cell. In some embodiments, the eukaryotic cell is an animal cell (e.g., a human cell). In some embodiments, the eukaryotic cell is a stem cell.

In some embodiments, the target nucleic acid encodes a gene product.

In some embodiments, the method further comprises measuring editing efficiency. In some embodiments, the method further comprises measuring editing efficiency at one or more OFF targets.

Other aspects and embodiments of the disclosure will be apparent in light of the following detailed description.

### BRIEF DESCRIPTION OF THE DRAWINGS

FIG. 1 is a graph of ACRs inhibiting editing by Un1Cas12fl in HEK293T cells (SEQ ID NO: 7).

FIG. 2 is a graph of ACRs inhibiting editing in HEK293T cells by a μCas nuclease having SEQ ID NO: 29.

### DETAILED DESCRIPTION

The disclosed compositions, systems, kits, and methods comprise anti-CRISPR polypeptides, or nucleic acids encoding thereof, useful for nucleic acid modification. The disclosed anti-CRISPR polypeptides allow increased control over one or more CRISPR-associated protein functions or activities (e.g., nuclease functionality, gRNA binding, DNA binding, or localization) thereby facilitating gene editing with improved efficacy, specificity, selectivity, and/or safety for use in in vitro, in vivo, and/or ex vivo applications of therapeutics, diagnostics, and research.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-60 (2005), Altschul et al., Nucleic Acids Res., 25(17): 3389-3402 (1997), and Gusfield, Algorithms on Strings, Trees and Sequences, Cambridge University Press, Cambridge UK (1997)).

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “contacting” as used herein refers to bring or put in contact, to be in or come into contact. The term “contact” as used herein refers to a state or condition of touching or of immediate or local proximity.

As used herein, the terms “providing,” “administering,” and “introducing,” are used interchangeably herein and refer to the placement of the composition or systems of the disclosure into a cell, organism, or subject by a method or route which results in at least partial localization to a desired site. The composition or systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems and Compositions

Advances and developments in CRISPR-Cas genome editing tools, including nucleases and other CRISPR-associated proteins, drive major advances in nucleic acid editing. Nucleic acid editing has many uses including in the diagnostics and therapeutics field. Such breadth is accompanied by a diversity of nucleic acid targets and environments in which to engineer editing activity. As such, there is a need for diverse and additional components and associated methods that provide a toolbox for nucleic acid editing.

Disclosed herein are systems and compositions that comprise polypeptides that have anti-CRISPR (Acr) activity, Acr polypeptides, or nucleic acids encoding the Acr polypeptides. The disclosed Acr polypeptides can modulate any activity of a corresponding CRISPR-associated protein, for example, nucleic acid binding, nucleic acid nicking or cleavage, and gRNA binding. In some embodiments, the Acr polypeptide modulates the target nucleic acid binding. In some embodiments, the Acr polypeptide modulates single-strand nucleic acid cleavage. In some embodiments, the Acr polypeptide modulates target nucleic acid double-strand nucleic acid cleavage. In some embodiments, the Acr polypeptide modulates target nucleic acid modification. In some embodiments, the Acr polypeptide modulates gRNA binding. In some embodiments, the Acr polypeptide modulates base editing.

The Acr polypeptide may modulate the ON (intended) or OFF (non-intended) target activity of the CRISPR-associated protein. For example, the Acr polypeptide may reduce OFF target activity of the CRISPR-associated protein, thereby increasing the ratio of ON target to OFF target activity compared to the CRISPR-associated protein in the absence of the Acr polypeptide. OFF target sequences are characterized as having one or more (e.g., 1, 2, 3, 4, 5, 6, 7, 8, 9, 10 or more) amino acid differences (e.g., substitutions, additions, deletions) compared to the ON target sequences. In some embodiments, the Acr polypeptide may decrease OFF target activity, without significantly affecting ON target efficiency.

“Modulating,” as used herein, refers to decreasing, reducing, inhibiting, increasing, inducing, activating, or otherwise affecting the activity of a protein, e.g., a CRISPR-associated protein. In some embodiments, the modulating means decreasing any of the activities of the corresponding CRISPR-associated protein, from partially decreasing to complete inhibition of the activity.

In some embodiments, the disclosed Acr polypeptides inhibit the activity of the CRISPR-associated protein at least 1.5 fold (e.g., at least 2, at least 2.5, at least 3, at least 3.5, at least 4, at least 5, at least 6, at least 7, at least 8, at least 9, at least 10 or more) fold as compared to the activity in the absence of the Acr polypeptide. In some embodiments, the Acr polypeptide decreases or is capable of decreasing the activity of the CRISPR-associated protein by at least 5%, at least 10%, at least 15%, at least 20%, at least 25%, at least 30%, at least 40%, at least 50%, at least 60%, at least 70%, at least 80%, at least 90%, or more compared to CRISPR-associated protein activity in the absence of the Acr polypeptide.

The disclosed Acr polypeptides comprise a sequence having at least about 70% identity (e.g., at least 75%, at least 80%, at least 85%, at least 90%, at least 93%, at least 95%, at least 98%, or at least 99%) to an amino acid sequence of SEQ ID NOs: 1-6. In some embodiments, the Acr polypeptide comprises a sequence having at least 90% identity an amino acid sequence of SEQ ID NOs: 1-6. In certain embodiments, the Acr polypeptide comprises an amino acid sequence of SEQ ID NOs: 1-6.

The disclosed systems and compositions may further comprise a CRISPR-associated protein or a nucleic acid encoding the CRISPR-associated protein. CRISPR-associated proteins include those that use CRISPR sequences as a guide to recognize specific nucleic acid sequences. Examples of CRISPR-associated protein include, but are not limited to: Cas1, Cas1B, Cas2, Cas3, Cas4, Cas5, Cas6, Cas7, Cas8, Cas9 (also known as Csnl and Csx12), Cas10, Cas12a (formerly Cpf1), Cas12b (formerly C2c1), Cas12c (formerly C2c3), Cas12d (formerly CasY), Cas12e (formerly CasX), Cas 12f, Cas12k (formerly C2c5), Cas13a (formerly known as C2c2), Cas13b, Cas13c, Cas13d, homologs, orthologs, paralogs, modified versions, either engineered or naturally occurring, or active fragments thereof.

In some embodiments, the CRISPR-associated protein comprises one or more nuclease domains capable to cleaving, either a single strand or both strands, of a target nucleic acid. In embodiments in which the CRISPR-associated protein is a nuclease, one or more of the nuclease domains may be mutated to render the nuclease activity reduced or eliminated.

In some embodiments, the CRISPR-associated protein is a Cas12f, or a homolog, ortholog, paralog, modified version, either engineered or naturally occurring, or active fragment thereof. Any known Cas12f is suitable for the disclosed systems and compositions. In some embodiments, the Cas12f comprises an amino acid sequence set forth in any one of SEQ ID NOs: 7-9. In some embodiments, the Cas12f comprises an amino acid sequence having about 70% or more (e.g., 75%, 80%, 85%, 90%, 95%, 96%, 97%, 98%, 99%, or more) sequence identity with any one of SEQ ID NOs: 7-9.

In some embodiments, the CRISPR-associated protein is a μCas and comprises an amino acid sequence set forth in any one of SEQ ID NOs: 10-259, or an active fragment thereof. In some embodiments, the CRISPR-associated protein comprises an amino acid sequence having about 70% or more (e.g., 75%, 80%, 85%, 90%, 95%, 96%, 97%, 98%, 99%, or more) sequence identity with any one of SEQ ID NOs: 10-259, or an active fragment thereof.

In some embodiments of the systems and methods herein, the Acr polypeptide preferentially modulates one CRISPR-associated protein as compared to another CRISPR-associated protein. In some embodiments, the Acr polypeptide preferentially modulates a Cas12f and/or a μCas as compared to a Cas9 or Cas12a. In some embodiments, the Acr polypeptide preferentially modulates a Cas12f and/or a μCas and does not affect (e.g., inhibit or modulate) the activity of a Cas9 or a Cas12a. In some embodiments, the Acr polypeptide preferentially modulates a Cas12f as compared to its modulation of a μCas. In some embodiments, the Acr polypeptide preferentially modulates a μCas as compared to its modulation of a Cas12f.

Any of the Acr polypeptides or CRISPR-associated proteins described herein may comprise one or more (e.g., 1, 2, 3, 4, 5, 6, 7, 8, 9, 10, 15, 20, 25, 50, 100, 150, 200, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, one or more amino acid substitutions in the amino acid Acr polypeptide results in an increase in inhibition of the CRISPR-associated protein relative to the polypeptide without the one or more amino acid substitutions. In some cases, one or more amino acid substitutions in the amino acid sequence of an Acr polypeptide results in detectable inhibition where the Acr polypeptide without the one or more amino acid substitutions has no detectable or minimally detectable inhibition of the same activity. In some embodiments, one or more amino acid substitutions in the amino acid sequence of a Acr polypeptide results in an alteration of the inhibited activity.

In some embodiments, the CRISPR-associated protein and/or Acr polypeptide comprises one or more nuclear localization sequences (NLSs). The nuclear localization sequence may be appended, for example, to the N-terminus, a C-terminus, or a combination thereof. In such cases with the CRISPR-associated protein and/or Acr polypeptide comprises two or more NLSs, the NLSs may be in tandem, separated by a linker, at either end terminus of the protein or polypeptide, or one or more may be embedded in the protein or polypeptide.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine. In some embodiments, the NLS is a monopartite sequence. A monopartite NLS comprise a single cluster of positively charged or basic amino acids. In some embodiments, the monopartite NLS comprises a sequence of K-K/R—X-K/R, wherein X can be any amino acid. Exemplary monopartite NLS sequences include those from the SV40 large T-antigen, c-Myc, and TUS-proteins. In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40.

The NLS may be appended by a linker. The linker may be a polypeptide of any amino acid sequence and length. The linker may act as a spacer peptide. In some embodiments, the linker is flexible. In some embodiments, the linker comprises at least one glycine and at least one serine. In some embodiments, the linker comprises an amino acid sequence consisting of (Gly2Ser)n, where n is the number of repeats comprising an integer from 2-20.

The CRISPR-associated protein and/or Acr polypeptide may comprise an epitope tag (e.g., 3xFLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein or polypeptide.

The CRISPR-associated protein may be part of a fusion protein comprising another protein or protein domain. The CRISPR-associated protein may be fused to a protein or protein domain that has another functionality or activity useful to target to certain DNA sequences (e.g., nuclease activity such as that provide by FokI nuclease, protein modification activity such as histone modification activity including acetylation or deacetylation or demethylation or methyltransferase activity, transcription modulation activity such as activity of a transcriptional activator or repressor, base editing activity such as deaminase activity, DNA modifying activity such as DNA methylation activity, and the like). In such situations, the activity which the Acr polypeptide modulates may result in or include modulation of the activity of the protein or protein domain that has another functionality or activity useful to target to certain DNA sequences. For example, if the Acr polypeptide modulates DNA binding of the CRISPR-associated protein which comprises a protein or protein domain with functionality to act on a given DNA sequence, the modulation of DNA binding would result in modulation of the corresponding activity of the protein or protein domain.

In some embodiments, the CRISPR-associated protein and/or Acr polypeptide may be fused with one or more (e.g., two, three, four, or more) protein transduction domains or PTDs, also known as a CPP—cell penetrating peptide. A protein transduction domain is a polypeptide, polynucleotide, carbohydrate, or organic or inorganic compound that facilitates traversing a lipid bilayer, micelle, cell membrane, organelle membrane, or vesicle membrane. A PTD attached to another molecule, facilitates the molecule traversing a membrane, for example going from extracellular space to intracellular space, or cytosol to within an organelle. In some embodiments, a PTD is covalently linked to a terminus of the CRISPR-associated protein and/or Acr polypeptide (e.g., N-terminus, C-terminus, or both). In some embodiments, the PTD is inserted internally at a suitable insertion site. Examples of PTDs include but are not limited to a minimal undecapeptide protein transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natd. Acad. Sci. USA 97:13003-13008); Transportan, and the like.

The CRISPR-associated protein and/or Acr polypeptide may be fused via a linker polypeptide. The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use, including but not limited to, glycine-serine polymers, glycine-alanine polymers, and alanine-serine polymers.

In some embodiments, the CRISPR-associated protein and/or Acr polypeptide is provided as a split-protein or split-polypeptide (e.g., the CRISPR-associated protein and/or Acr polypeptide can be delivered as a split-protein or split-polypeptide, or a nucleic acid(s) encoding a split-protein or split-polypeptide) such that two separate proteins or polypeptide together form a functional entity. In some cases, the sequences that encode the two parts of the split-protein or split-polypeptide are present on the nucleic acid. In some cases, the sequences that encode the two parts of the split-protein or split-polypeptide are present on different nucleic acids.

In some embodiments, the compositions or systems further comprise at least one guide RNA (gRNA) or one or more nucleic acids comprising a sequence encoding the least one gRNA. In instances when the composition or system comprises more than one gRNA, each may be encoded on the same or different nucleic acid as the other gRNA.

The gRNA may be a crRNA, crRNA/tracrRNA (or single guide RNA, sgRNA). The terms “gRNA,” “guide RNA,” and “CRISPR guide sequence” may be used interchangeably throughout and refer to a nucleic acid comprising a sequence that determines the sequence specificity of the CRISPR-associated protein. A gRNA hybridizes to (complementary to, partially or completely) a target nucleic acid sequence (e.g., the genome in a host cell).

In some embodiments, the at least one gRNA is encoded in a CRISPR RNA (crRNA) array. CRISPR arrays contain a series of direct repeats separated by short sequences called spacers. The CRISPR-associated protein described herein may have a preference for direct repeat sequences. These can be determined by methods known in the art including those described in the Examples herein. For example, the CRISPR RNA (crRNA) may contain multiple gRNAs or may contain more than one different sequence each configured to hybridize a distinct target nucleic acid sequence.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

The gRNA may be a non-naturally occurring gRNA.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. Sequence requirements for PAMs for any given nuclease can be determined using known methods, for example, the protocol of Walton et al. (Walton R T, et al., Science. 2020 Apr. 17; 368(6488):290-296, incorporated herein by reference in its entirety).

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

The systems may comprise one or more nucleic acids encoding the Acr polypeptide, the CRISPR-associated protein, and/or the gRNA. In some embodiments, individual nucleic acids encode each of the Acr polypeptide, the CRISPR-associated protein, and the gRNA. In some embodiments, the Acr polypeptide and the CRISPR-associated protein are encoded by separate nucleic acids. For example, the system may comprise a first nucleic acid encoding the Acr polypeptide and a second nucleic acid encoding the CRISPR-associated protein. Alternatively, in some embodiments, the Acr polypeptide and the CRISPR-associated protein are encoded by a single nucleic acid. In some embodiments, the gRNA is encoded by a separate nucleic acid than either or both of the Acr polypeptide and the CRISPR-associated protein

In some embodiments, the one or more nucleic acids comprise one or more messenger RNAs, one or more vectors, or any combination thereof.

In certain embodiments, engineering the Acr polypeptide and/or the CRISPR-associated protein for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

The present disclosure also provides vectors containing the nucleic acids encoding the Acr polypeptide, the CRISPR-associated protein and/or the gRNA, and cells containing the vectors. The vectors may be used to propagate the Acr polypeptide, the CRISPR-associated protein and/or the gRNA in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system (e.g., the Acr polypeptide, the CRISPR-associated protein and/or the gRNA). The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the composition or system may be removed from the cells under certain conditions. For example. this may allow for DNA integration by transforming bacteria of interest, but then being left with engineered strains that have no memory of the plasmids or vectors used for the integration.

A variety of viral constructs may be used to deliver the present composition or system (such as an Acr polypeptide, a CRISPR-associated protein, and one or more gRNA(s)) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a nucleic acid encoding the Acr polypeptide and/or the CRISPR-associated protein is contained in a plasmid vector that allows expression and subsequent isolation and purification of the protein produced by the recombinant vector. Accordingly, the Acr polypeptide and/or the CRISPR-associated protein disclosed herein can be purified following expression, obtained by chemical synthesis, or obtained by recombinant methods.

To construct cells that express the present system, expression vectors for stable or transient expression of the system, or any of its components, may be constructed via methods as described herein or known in the art and introduced into cells. For example, nucleic acids encoding the components of the present system (the Acr polypeptide, the CRISPR-associated protein, and/or the gRNA) may be cloned into a suitable expression vector, such as a plasmid or a viral vector, with each component in operable linkage to a suitable promoter or translation control element. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The composition or system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufiman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Different promoters, regulatory elements, or translation control elements may be used to achieve proper balance (expression level ratio) between the components of the systems (e.g., the Acr polypeptide, the CRISPR-associated protein and/or the gRNA). For example, in some cases a nucleic acid includes a promoters, regulatory elements, or translation control elements that is operably linked to (and therefore regulates/modulates translation of) a sequence encoding the Acr polypeptide, the CRISPR-associated protein and/or the gRNA. In some cases, the sequence encoding the Acr polypeptide, the CRISPR-associated protein, and/or the gRNA are operably linked to the same promoters, regulatory elements, or translation control elements.

A variety of promoter types are suitable for use. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein.), it may be a spatially restricted promoter (e.g., tissue specific promoter, cell type specific promoter, etc.), and it may be a temporally restricted promoter (e.g., the promoter is in the “ON” state or “OFF” state during specific stages of embryonic development or during specific stages of a biological process, e.g., hair follicle cycle in mice).

Inducible and tissue specific expression of RNA or proteins can be accomplished by placing the nucleic acid encoding such a molecule under the control of an inducible or tissue specific promoter/regulatory sequence. The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired nuclease or RNA operably linked thereto.

Examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter; a serotonin receptor promoter (see, e.g., GenBank S62283); a tyrosine hydroxylase promoter (TH); a GnRH promoter; an L7 promoter; a DNMT promoter; an enkephalin; a myelin basic protein (MBP) promoter; a Ca2+-calmodulin-dependent protein kinase II-alpha (CamKIIa) promoter; a CMV enhancer/platelet-derived growth factor-β promoter; and the like. Suitable liver-specific promoters can in some cases include, but are not limited to: TTR, Albumin, and AAT promoters. Suitable CNS-specific promoters can in some cases include, but are not limited to: Synapsin 1, BM88, CHNRB2, GFAP, and CAMK2a promoters. Suitable muscle-specific promoters can in some cases include, but are not limited to: MYOD1, MYLK2, SPc5-12 (synthetic), α-MHC, MLC-2, MCK, MHCK7, human cardiac troponin C (cTnC) and desmin promoters. Adipocyte-specific spatially restricted promoters include, but are not limited to, aP2 gene promoter/enhancer, e.g., a region from −5.4 kb to +21 bp of a human aP2; a glucose transporter-4 (GLUT4); a fatty acid translocase (FAT/CD36) promoter; a stearoyl-CoA desaturase-1 (SCD1) promoter; a leptin promoter; an adiponectin promoter; an adipsin promoter; a resistin promoter; and the like. Cardiomyocyte-specific spatially restricted promoters include, but are not limited to, control sequences derived from the following genes: myosin light chain-2, α-myosin heavy chain, AE3, cardiac troponin C, cardiac actin, and the like. Smooth muscle-specific spatially restricted promoters include, but are not limited to, an SM22a promoter; a smoothelin promoter; an α-smooth muscle actin promoter; and the like. For example, a 0.4 kb region of the SM22a promoter, within which lie two CArG elements, has been shown to mediate vascular smooth muscle cell-specific. Photoreceptor-specific spatially restricted promoters include, but are not limited to, a rhodopsin promoter; a rhodopsin kinase promoter; a beta phosphodiesterase gene; a retinitis pigmentosa gene promoter; an interphotoreceptor retinoid-binding protein (IRBP) gene enhancer; an IRBP gene promoter; and the like.

Examples of inducible promoters include, but are not limited to, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; an estrogen receptor; an estrogen receptor fusion; an estrogen analog; IPTG; and the like. Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Inducible promoters include sugar-inducible promoters (e.g., lactose-inducible promoters; arabinose-inducible promoters); amino acid-inducible promoters; alcohol-inducible promoters; and the like. Suitable promoters include, e.g., lactose-regulated systems (e.g., lactose operon systems, sugar-regulated systems, isopropyl-beta-D-thiogalactopyranoside (IPTG) inducible systems, arabinose regulated systems (e.g., arabinose operon systems, e.g., an ARA operon promoter, pBAD, pARA, portions thereof, combinations thereof and the like), synthetic amino acid regulated systems, fructose repressors, a tac promoter/operator (pTac), tryptophan promoters, PhoA promoters, recA promoters, proU promoters, cst-1 promoters, tetA promoters, cadA promoters, nar promoters, P_{L} promoters, cspA promoters, and the like, or combinations thereof. In certain cases, a promoter comprises a Lac-Z, or portions thereof. In some cases, a promoter comprises a Lac operon, or portions thereof. In some cases, an inducible promoter comprises an ARA operon promoter, or portions thereof. In certain embodiments an inducible promoter comprises an arabinose promoter or portions thereof. An arabinose promoter can be obtained from any suitable bacteria. In some cases, an inducible promoter comprises an arabinose operon of E. coli or B. subtilis. In some cases, an inducible promoter is activated by the presence of a sugar or an analog thereof. Non-limiting examples of sugars and sugar analogs include lactose, arabinose (e.g., L-arabinose), glucose, sucrose, fructose, IPTG, and the like. Suitable promoters include a T7 promoter; a pBAD promoter; a lacIQ promoter; and the like. In some cases, the promoter is a J23119 promoter. Many bacterial promoters are known in the art; bacterial promoters can be found on the internet at parts.igem.org/promoters.

In some cases, the promoter is a reversible promoter. Suitable reversible promoters, including reversible inducible promoters are known in the art. Such reversible promoters may be isolated and derived from many organisms. Such reversible promoters may be isolated and derived from many organisms, e.g., eukaryotes and prokaryotes. Modification of reversible promoters derived from a first organism for use in a second organism is well known in the art. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR)), tetracycline regulated promoters, (e.g., promoter systems including TetActivators, TetON, TetOFF), steroid regulated promoters (e.g., rat glucocorticoid receptor promoter systems, human estrogen receptor promoter systems, retinoid promoter systems, thyroid promoter systems, ecdysone promoter systems, mifepristone promoter systems), metal regulated promoters (e.g., metallothionein promoter systems), pathogenesis-related regulated promoters (e.g., salicylic acid regulated promoters, ethylene regulated promoters, benzothiadiazole regulated promoters), temperature regulated promoters (e.g., heat shock inducible promoters (e.g., HSP-70, HSP-90, soybean heat shock promoter), light regulated promoters, synthetic inducible promoters, and the like.

Thus, it will be appreciated that the present disclosure includes the use of any promoter/regulatory elements/translation control elements capable of driving expression of the desired Acr polypeptide, CRISPR-associated protein, and/or the gRNA operably linked thereto.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and ColE1 for proper episomal replication; internal ribosome binding sites (IRESes), a 2A peptide encoding sequence, a noncanonical start codon, versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

In some embodiments, the translation control element capable of driving expression of the desired Acr polypeptide, CRISPR-associated protein, and/or the gRNA comprises an IRES sequence. And internal ribosome entry site (IRES) is any nucleotide sequence that allows for translation initiation in the middle of a messenger RNA (mRNA) sequence as part of the greater process of protein synthesis.

In some embodiments, the translation control element capable of driving expression of the desired Acr polypeptide, CRISPR-associated protein, and/or the gRNA comprises a ribosome skip sequence. A “ribosome skip sequence” refers to a sequence that during translation, forces the ribosome to skip the sequence and translate the region after the ribosome skip sequence without formation of a peptide bond. Several viruses, for example, have ribosome skip sequences that allow sequential translation of several proteins on a single nucleic acid without having the proteins linked via a peptide bond. In some embodiments, the ribosome skip sequence is 2A peptide encoding sequence, for example, a P2A, T2A, E2A or F2A sequence. In some embodiments, the ribosome skip sequence comprises a sequence motif of DXEXNPG (SEQ ID NO: 267).

In some embodiments, the translation control element capable of driving expression of the desired Acr polypeptide, CRISPR-associated protein, and/or the gRNA comprises a noncanonical start codon.

When introduced into the cell, the vectors may be maintained as an autonomously replicating sequence or extrachromosomal element or may be integrated into host DNA.

The present composition and systems (e.g., proteins, polynucleotides encoding these proteins, or compositions comprising the proteins and/or polynucleotides described herein) may be delivered by any suitable means. In certain embodiments, the composition or system is delivered in vivo. In other embodiments, the composition or system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present compositions and system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation, or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, biolistics, and the like.

In some embodiments, the vector is a viral construct, e.g., a recombinant adeno-associated virus construct, a recombinant adenoviral construct, a recombinant lentiviral construct, a recombinant retroviral construct, etc. Suitable viral vectors include, but are not limited to, viral vectors based on vaccinia virus; poliovirus; adenovirus; adeno-associated virus; SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, the vector is an AAV vector. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector” or “rAAV vector” it is meant an AAV virus or AAV viral chromosomal material comprising a polynucleotide sequence not of AAV origin (e.g., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs). In some instances, the recombinant viral vector also comprises viral genes important for the packaging of the recombinant viral vector material. Packaging refers to the series of intracellular events that result in the assembly and encapsulation of a viral particle, e.g., an AAV viral particle. Examples of nucleic acid sequences important for AAV packaging include the AAV “rep” and “cap” genes, which encode for replication and encapsulation proteins of adeno-associated virus, respectively. The term rAAV vector encompasses both rAAV vector particles and rAAV vector plasmids.

A “viral particle” refers to a single unit of virus comprising a capsid encapsulating a virus-based polynucleotide, e.g., the viral genome (as in a wild-type virus), or, e.g., the subject targeting vector (as in a recombinant virus). An AAV viral particle refers to a viral particle composed of at least one AAV capsid protein (typically by all of the capsid proteins of a wild-type AAV) and an encapsulated polynucleotide AAV vector. If the particle comprises a heterologous polynucleotide (e.g., a polynucleotide other than a wild-type AAV genome, such as a transgene to be delivered to a mammalian cell), it is typically referred to as an “rAAV vector particle” or simply an “rAAV vector.” Thus, production of rAAV particle necessarily includes production of rAAV vector, as such a vector is contained within an rAAV particle.

A rAAV virion can be constructed a variety of methods. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. In order to produce rAAV virions, an AAV expression vector can be introduced into a suitable host cell using known techniques, such as by transfection. Particularly suitable transfection methods include calcium phosphate co-, direct micro-injection into cultured cells, electroporation, liposome mediated gene transfer, lipid-mediated transduction, and nucleic acid delivery using high-velocity microprojectiles. Suitable cells for producing rAAV virions include microorganisms, yeast cells, insect cells, and mammalian cells, that can be, or have been, used as recipients of a heterologous DNA molecule.

An AAV virus that is produced may be replication competent or replication-incompetent. A “replication-competent” virus (e.g., a replication-competent AAV) refers to a phenotypically wild-type virus that is infectious and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector.

Retroviruses, for example, lentiviruses, are suitable for use in methods of the present disclosure. Commonly used retroviral vectors are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog, and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

As noted elsewhere herein, proteins may instead be provided to cells as RNA (e.g., an RNA comprising the translational control element as discussed elsewhere herein). Methods of introducing RNA into cells may include, for example, direct injection, transfection, or any other method used for the introduction of DNA.

The Acr polypeptide and/or CRISPR-associated protein may also be introduced or provided as protein. In such instances, the Acr polypeptide and/or CRISPR-associated protein may be delivered as an RNP (ribonucleoprotein complex) in which it is already complexed with an appropriate guide RNA.

The disclosed nucleic acids (e.g., vectors) and proteins can be delivered to cells using any convenient method. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like.

In some cases, the Acr polypeptide, the CRISPR-associated protein, and the gRNA, or the nucleic acid(s) encoding thereof are delivered to a cell in a particle, or associated with a particle. In some cases, the Acr polypeptide, the CRISPR-associated protein, and the gRNA, or the nucleic acid(s) encoding thereof are delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol.

The Acr polypeptide, the CRISPR-associated protein, and the gRNA, or the nucleic acid(s) encoding thereof may be delivered using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the desired cell, can be used.

Lipidoid compounds (e.g., as described in U.S. Patent Application Publication No. 2011/0293703) are also useful in the delivery of polynucleotides, and can be used to deliver the disclosed Acr polypeptide, CRISPR-associated protein, and gRNA, or the nucleic acid(s) encoding thereof. In one aspect, the aminoalcohol lipidoid compounds are combined with an agent to be delivered to a cell to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver the Acr polypeptide, the CRISPR-associated protein, and the gRNA, or the nucleic acid(s) encoding thereof to a target cell. U.S. Patent Application Publication No. 2013/0302401 relates to a class of poly(beta-amino alcohols) (PBAAs) that has been prepared using combinatorial polymerization.

Sugar-based particles, for example GalNAc, as described in International Patent Publication No. WO2014118272 (incorporated herein by reference in its entirety and Nair, J K et al., 2014, Journal of the American Chemical Society 136 (49), 16958-16961) can be used to deliver the Acr polypeptide, the CRISPR-associated protein, and the gRNA, or the nucleic acid(s) encoding thereof to a target cell.

In some cases, lipid nanoparticles (LNPs) are used to deliver the Acr polypeptide, the CRISPR-associated protein, and the gRNA, or the nucleic acid(s) encoding thereof to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol)2000) carbamoyl]-1,2-dimyristyloxlpropyl-3-amine (PEG-C-DOMG) may be used. A nucleic acid may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to the Acr polypeptide, the CRISPR-associated protein, and the gRNA, or the nucleic acid(s) encoding thereof to a target cell.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

Nanoparticles suitable for use in delivering the Acr polypeptide, the CRISPR-associated protein, and the gRNA, or the nucleic acid(s) encoding thereof to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure. In general, a “nanoparticle” refers to any particle having a diameter of less than 1000 nm. In some cases, nanoparticles suitable for use in delivering a nuclease or nucleic acid to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a nuclease or nucleic acid to a target cell have a diameter of from 25 nm to 200 nm.

In some cases, an exosome is used to deliver the Acr polypeptide, the CRISPR-associated protein, and the gRNA, or the nucleic acid(s) encoding thereof to a target cell. Exosomes are endogenous nano-vesicles that transport RNAs and proteins, and which can deliver RNA to the brain and other target organs.

In some cases, a liposome is used to deliver the Acr polypeptide, the CRISPR-associated protein, and the gRNA, or the nucleic acid(s) encoding thereof to a target cell. Liposomes are spherical vesicle structures composed of a uni- or multi-lamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver the Acr polypeptide, the CRISPR-associated protein, and the gRNA, or the nucleic acid(s) encoding thereof to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver the Acr polypeptide, the CRISPR-associated protein, and the gRNA, or the nucleic acid(s) encoding thereof are to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−0.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with the Acr polypeptide, the CRISPR-associated protein, and the gRNA, or the nucleic acid(s) encoding thereof, to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a protein and/or nucleic acid using a spontaneous vesicle formation procedure.

The Acr polypeptide, the CRISPR-associated protein, and the gRNA, or the nucleic acid(s) encoding thereof, may be delivered encapsulated in PLGA microspheres such as those further described in US published applications 20130252281, 20130245107, and 20130244279.

Supercharged proteins can be used to deliver the Acr polypeptide, the CRISPR-associated protein, and the gRNA, or the nucleic acid(s) encoding thereof to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver the Acr polypeptide, the CRISPR-associated protein, and the gRNA, or the nucleic acid(s) encoding thereof to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

In some embodiments, the components of the present compositions or systems may be mixed, individually or in any combination, with a carrier which are also within the scope of the present disclosure. Exemplary carriers include buffers, antioxidants, preservatives, carbohydrates, surfactants, and the like.

Also disclosed is a cell comprising the compositions or systems described herein. In some embodiments, the cell is a prokaryotic cell. In some embodiments, the cell is a eukaryotic cell. In some embodiments, the cell is a mammalian cell. In some embodiments, the cell is a human cell.

### Methods

The disclosure also provides methods of modifying a target nucleic acid sequence (e.g., DNA or RNA). The phrase “modifying a nucleic acid sequence,” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence. The modifications may comprise one or more of nucleic acid binding, base editing, transcription modulation, nucleic acid modification, protein modification, and histone modification.

The methods comprise contacting a target nucleic acid sequence with a composition or a system disclosed herein. The Acr polypeptides may modulate any activity of a corresponding CRISPR-associated protein used in the methods disclosed herein. For example, the Acr polypeptide can modulate nucleic acid binding, nucleic acid nicking or cleavage, and/or gRNA binding. In some embodiments, the Acr polypeptide modulates the target nucleic acid binding. In some embodiments, the Acr polypeptide modulates single-strand nucleic acid cleavage. In some embodiments, the Acr polypeptide modulates target nucleic acid double-strand nucleic acid cleavage. In some embodiments, the Acr polypeptide modulates target nucleic acid modification. In some embodiments, the Acr polypeptide modulates gRNA binding. In some embodiments, the Acr polypeptide modulates base editing.

The Acr polypeptide may modulate the ON or OFF target activity of the CRISPR-associated protein. For example, the Acr polypeptide may reduce OFF target activity of the CRISPR-associated protein, thereby increasing the ratio of ON target to OFF target activity compared to the CRISPR-associated protein in the absence of the Acr polypeptide. In some embodiments, the Acr polypeptide may decrease OFF target activity, without significantly affecting ON target efficiency. As such, disclosed herein are methods of decreasing OFF target activity of a CRISPR-associated protein comprising contacting the CRISPR-associated protein with an Acr polypeptide as disclosed herein. In some embodiments, the methods increase the ratio of ON target to OFF target activity compared to the CRISPR-associated protein in the absence of the Acr polypeptide.

In some embodiments, modifying a DNA sequence comprises a deletion. The deletion may be upstream or downstream of the PAM binding side, so called unidirectional deletions. The deletion may encompass sequences on either side of the PAM binding site, a bidirectional deletion. The deletion of the DNA sequence may be of any size.

In some embodiments, contacting a target nucleic acid sequence comprises introducing the composition or system into the cell. As described above the composition or system may be introduced into eukaryotic or prokaryotic cells by methods known in the art.

In some embodiments, the methods further comprise measuring the editing efficiency. Any method for measuring editing efficiency may be suitable for use with the disclosed methods. See for example, Germini et al., Trends in Biotechnology Vol. 36, Iss. 2, P147-159, Feb. 1, 2018. For examples, methods for evaluation of editing efficiency can include DNA sequencing or use of mismatch-sensitive endonucleases. In some embodiments, the method comprises measuring editing efficiency at one or more intended targets. In some embodiments, the method comprises measuring editing efficiency at one or more OFF, or non-intended, targets.

The cell may be a prokaryotic cell, a plant cell, an insect cell, a vertebrate cell, an invertebrate cell, an animal cell, a mammalian cell, or a human cell. In some embodiments, the cell is a plant cell. In some embodiments, the cell is an insect cell. In some embodiments, the cell is a vertebrate cell. In some embodiments, the cell is an invertebrate cell. In some embodiments, the cell is a mammalian cell. In some embodiments, the cell is a human cell. In some embodiments, the cell is a stem cell. In some cases, the cell is ex vivo (e.g., fresh isolate—early passage). In some cases, the cell is in vivo. In some cases, the cell is in culture in vitro (e.g., immortalized cell line).

Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

Suitable cells include, but are not limited to: bacterial cell; an archaeal cell; a eukaryotic cell; a cell of a single-cell eukaryotic organism; a plant cell; a protozoa cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). In some cases, the cell is a cell that does not originate from a natural organism (e.g., the cell can be a synthetically made cell; also referred to as an artificial cell).

Non-limiting examples of plant cell include cells from: plant crops, fruits, vegetables, grains, soybean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, seaweeds (e.g., kelp), and the like.

Suitable cells include a stem cell (e.g., an embryonic stem (ES) cell, an induced pluripotent stem (iPS) cell; a germ cell (e.g., an oocyte, a sperm, an oogonia, a spermatogonia, etc.), a somatic cell, e.g., a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

In some embodiments, the stem cell is a hematopoietic stem cell (HSC). HSCs are mesoderm-derived cells that can be isolated from bone marrow, blood, cord blood, fetal liver, and yolk sac. HSCs are characterized as CD34+ and CD3-. HSCs can repopulate the erythroid, neutrophil-macrophage, megakaryocyte, and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells, respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some embodiments, the cell is a T cell. The invention is not limited by the type of T cell. The T cells may be selected from, for example, CD3+ T cells, CD8+ T cells, CD4+ T cells, natural killer (NK) T cells, alpha beta T cells, gamma delta T cells, or any combination thereof (e.g., a combination of CD4+ and CD8+ T cells).

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

A cell is in some cases a plant cell. A plant cell can be a cell of a monocotyledon. A plant cell can be a cell of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

A plant cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

In some embodiments, introducing the system into a cell comprises administering the system to a subject. In some embodiments, the subject is human. The administering may comprise in vivo administration. In alternative embodiments, a vector is contacted with a cell in vitro or ex vivo and the treated cell, containing the system, is transplanted into a subject.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The disclosed method may modify a target DNA sequence in a cell so as to modulate expression of the target DNA sequence, e.g., expression of the target DNA sequence is increased, decreased, or completely eliminated (e.g., via deletion of a gene). In one embodiment, the disclosed system cleaves a target DNA sequence of the host cell to produce double strand DNA breaks. The double strand breaks can be repaired by the host cell by either non-homologous end joining (NHEJ) or homologous recombination. In NHEJ, the double-strand breaks are repaired by direct ligation of the break ends to one another. In homologous recombination repair, a donor nucleic acid molecule comprising a second DNA sequence with homology to the cleaved target DNA sequence is used as a template for repair of the cleaved target DNA sequence, resulting in the transfer of genetic information from the donor nucleic acid molecule to the target DNA. As a result, new nucleic acid material is inserted/copied into the DNA break site. The modifications of the target sequence due to NHEJ and/or homologous recombination repair may lead to, for example, gene correction, gene replacement, gene tagging, transgene insertion, nucleotide deletion, gene disruption, gene mutation, gene knock-down, etc.

In some embodiments, the systems and methods described herein may be used to correct one or more defects or mutations in a gene (referred to as “gene correction”). In such cases, the target sequence encodes a defective version of a gene, and the disclosed compositions and systems further comprise a donor nucleic acid molecule which encodes a wild-type or corrected version of the gene.

In another embodiment, the method of modifying a target sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the systems and methods described herein may be used to insert a gene or fragment thereof into a cell. In particular embodiments, the disclosed systems may be used to generate a cell that expresses a recombinant receptor. In some embodiments, the recombinant receptor is a T cell receptor (TCR) or a chimeric antigen receptor (CAR). Also provided herein are cells, e.g., a T cell, comprising a recombinant receptor and/or a nucleic acid encoding thereof and a system (e.g., nuclease and at least one gRNA) as described herein.

In some embodiments, the system and methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present systems and methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present systems and methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present system and method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present system and method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present systems and methods may be adapted to use in plants. In one embodiment, a series of plant-specific RNA-guided Genome Editing vectors (pRGE plasmids) are provided for expression of the present system in plants. The vectors may be optimized for transient expression of the present system in plant protoplasts, or for stable integration and expression in intact plants via the Agrobacterium-mediated transformation. In one aspect, the vector constructs include a nucleotide sequence comprising a DNA-dependent RNA polymerase III promoter, wherein the promoter is operably linked to a gRNA molecule and a Pol III terminator sequence, and a nucleotide sequence comprising a DNA-dependent RNA polymerase II promoter operably linked to a nucleic acid sequence encoding the nuclease.

In certain embodiments, the present systems and methods use a monocot promoter to drive the expression of one or more components of the present systems (e.g., gRNA) in a monocot plant. In certain embodiments, the present systems and methods use a dicot promoter to drive the expression of one or more components of the present systems (e.g., gRNA) in a dicot plant. In some embodiments, the present system is transiently expressed in plant protoplasts. Vectors for transient transformation of plants include, but are not limited to, pRGE3, pRGE6, pRGE31, and pRGE32. In some embodiment, the vector may be optimized for use in a particular plant type or species, such as pStGE3.

In one embodiment, the present system may be stably integrated into the plant genome, for example via Agrobacterium-mediated transformation. Thereafter, one or more components of the present system (e.g., the transgene) may be removed by genetic cross and segregation, which may lead to the production of non-transgenic, but genetically modified plants or crops. In one embodiment, the vector is optimized for Agrobacterium-mediated transformation. In one embodiment, the vector for stable integration is pRGEB3, pRGEB6, pRGEB31, pRGEB32, or pStGEB3.

The present system may be used in various bacterial hosts, including human pathogens that are medically important, and bacterial pests that are key targets within the agricultural industry, as well as antibiotic resistant versions thereof.

The system and method may be designed to target any gene or any set of genes, such as virulence or metabolic genes, for clinical and industrial applications in other embodiments. For example, the present systems and methods may be used to target and eliminate virulence genes from the population, to perform in situ gene knockouts, or to stably introduce new genetic elements to the metagenomic pool of a microbiome. The present systems and methods may be used to treat a multi-drug resistance bacterial infection in a subject. The present systems and methods may be used for genomic engineering within complex bacterial consortia.

The present systems and methods may be used to inactivate microbial genes. In some embodiments, the gene is an antibiotic resistance gene. For example, the coding sequence of bacterial resistance genes may be disrupted in vivo by insertion of a DNA sequence, leading to non-selective re-sensitization to drug treatment.

In some embodiments, introducing the system into a cell comprises administering the system to a subject. In some embodiments, the subject is human. The administering may comprise in vivo administration. In alternative embodiments, a vector is contacted with a cell in vitro or ex vivo and the treated cell, containing the system, is transplanted into a subject.

The components of the composition, system or ex vivo treated cells may be administered to a cell or subject with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

The methods described here also provide for treating a disease or condition in a subject. The method may comprise administering to the subject, in vivo, or by transplantation of ex vivo treated cells (e.g., disclosed T cells), a therapeutically effective amount of the present system, or components thereof. A “subject” or “patient” may be human or non-human and may include, for example, animal strains or species used as “model systems” for research purposes, such a mouse model as described herein.

In some embodiments, the systems and methods are used to treat a pathogen or parasite on or in a subject by altering the pathogen or parasite. In some embodiments, the systems and methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene.

The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

In some embodiments, an effective amount of the components of the present system or compositions as described herein can be administered. Within the context of the present disclosure, the term “effective amount” refers to that quantity of the components of the system such that modification of the target nucleic acid is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

A wide range of additional therapies may be used in conjunction with the methods of the present disclosure. The additional therapy may be administration of an additional therapeutic agent or may be an additional therapy not connected to administration of another agent. Such additional therapies include, but are not limited to, surgery, immunotherapy, radiotherapy. The additional therapy may be administered at the same time as the above methods. In some embodiments, the additional therapy may precede or follow the treatment of the disclosed methods by time intervals ranging from hours to months.

In some embodiments, a therapeutically effective amount of a system (e.g., nuclease and/or gRNA) or compositions described herein, is administered alone or in combination with a therapeutically effective amount of at least one additional therapeutic agent. In some embodiments, effective combination therapy is achieved with a single composition or pharmacological formulation or with two distinct compositions or formulations, administered at the same time or separated by a time interval. The at least one additional therapeutic agent may comprise any manner of therapeutic, including protein, small molecule, nucleic acids, and the like. For example, exemplary additional therapeutic agents include, but are not limited to, immune modulators, chemotherapeutic agents, a nucleic acid (e.g., mRNA, aptamers, antisense oligonucleotides, ribozyme nucleic acids, interfering RNAs, antigene nucleic acids), decongestants, steroids, analgesics, antimicrobial agents, immunotherapies, or any combination thereof.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

In some cases, desirable delivery systems provide for roughly uniform distribution and have controllable rates of release of their components (e.g., vectors, proteins, nucleic acids) in vivo. A variety of different media are described below that are useful in creating composition delivery systems. It is not intended that any one medium is limiting to the present invention. Note that any medium may be combined with another medium or carrier; for example, in one embodiment a polymer microparticle attached to a compound may be combined with a gel medium. An implantable device can be used to deliver a nuclease, or a nucleic acid encoding thereof, and gRNA, or a nucleic acid encoding thereof, to, for example, a target cell in vivo.

Carriers or mediums contemplated include materials such as gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In some cases, a carrier/medium can include a microparticle. Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids—in any combination thereof.

In some cases, a carrier/medium can include a liposome that is capable of attaching and releasing therapeutic agents (e.g., the subject nucleic acids and/or proteins). Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life.

In some embodiments, a cationic or anionic liposome is used as part of a subject composition or method, or liposomes having neutral lipids can also be used. Cationic liposomes can include negatively-charged materials by mixing the materials and fatty acid liposomal components and allowing them to charge-associate. The choice of a cationic or anionic liposome depends upon the desired pH of the final liposome mixture.

### Kits

Also within the scope of the present disclosure are kits that include the compositions, systems, or components thereof as disclosed herein.

For example the kits may contain one or more reagents or other components useful, necessary, or sufficient for practicing any of the methods described herein, such as, editing reagents (Acr polypeptides, nucleases, guide RNAs, vectors, compositions, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The kit may include instructions for use in any of the methods described herein. Kits optionally may provide additional components such as buffers and interpretive information. Normally, the kit comprises a container and a label or package insert(s) on or associated with the container. Instructions supplied in the kits of the disclosure are typically written instructions on a label or package insert. In some embodiment, the disclosure provides articles of manufacture comprising contents of the kits described above.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses.

The kit will typically be provided with its various components in one or more packages, e.g., a fiber-based, a cardboard, polymeric, or a Styrofoam box. The enclosure(s) can be configured so as to maintain a temperature differential between the interior and the exterior, for example, to provide insulating properties to keep the reagents at a preselected temperature for a preselected time. The packaging can be air-tight, waterproof (e.g., impermeable to changes in moisture or evaporation), and/or light-tight.

### EXAMPLES

The following are examples of the present invention and are not to be construed as limiting.

### Example 1

### Vector Construction

Nuclease expression vectors Codon-optimized (human) genes encoding two nucleases (UnCas12f (SEQ ID NO: 7) and SEQ ID NO: 29) were synthesized and cloned into a mammalian expression vector under the CMV promoter, pTwist_CMV (Twist Biosciences). The vector included an in-frame SV40 nuclear localization signal (NLS) (PKKKRKV) (SEQ ID NO: 260) followed by a linker (GIHGVPAA) (SEQ ID NO: 261) fused to the N-termini of the nucleases. A nucleoplasmin NLS (KRPAATKKAGQAKKKK) (SEQ ID NO: 262) was fused to the C-termini of the nucleases followed by a 3x HA tag.

Single guide RNA (sgRNA) vectors An sgRNA designed by Kim et al., 2021 having the DNA sequence ACCGCTTCACCAAAAGCTGTCCCTTAGGGGATTAGAACTTGAGTGAAGGTGGGC TGCTTGCATCAGCCTAATGTCGAGAAGTGCTTTCTTCGGAAAGTAACCCTCGAAA CAAAgaaaGGAATGCAAC (SEQ ID NO: 263) was utilized for UnCas12f. An sgRNA was designed for the nuclease having SEQ ID NO: 29 based on predicted crRNA and tracrRNA binding and folding patterns. The sgRNAs were placed downstream of the U6 promoter with a starting G, and then placed upstream of the spacer sequence. The spacer sequence (as DNA) used for Kim-T1 (GSp162) is CACACACACAGTGGGCTACC (SEQ ID NO: 264) for which the PAM is TTTA.

Acr vectors Potential Anti-CRISPR (Acr) sequences for use with nucleases were identified in the NCBI database. Codon-optimized (human) genes encoding the Acrs were synthesized and cloned into the same CMV expression vector as the nucleases.

### Example 2

### Acr Activity

Editing efficiency of UnCas12f (SEQ ID NO: 7) and the nuclease having SEQ ID NO: 29 in the presence of Acrs was tested in HEK293T cells. The vectors constructed in Example 1 were transfected into cells using Mirus Transit 2020 reagent. 50,000 cells were plated per well of a 96 well plate and immediately transfected with 67 ng of nuclease expression vector, 67 ng of sgRNA vector, and 67 ng of Acr vector. In conditions with no Acr, 67 ng of pmax GFP plasmid (Lonza) was transfected instead of Acr in order to keep the total amount of transfected DNA the same. Some wells were left to grow without transfection as a negative control for which no editing is expected. Samples were incubated for 72 h and harvested with Quick Extract (Lucigen). Genomic DNA was amplified using genomic region-specific primers for TCRA amplification: FWD=CTGAGTCCCAGTCCATCACG, REV=AGGGTTTTGGTGGCAATGGA, (SEQ ID NOs: 265 and 266, respectively).

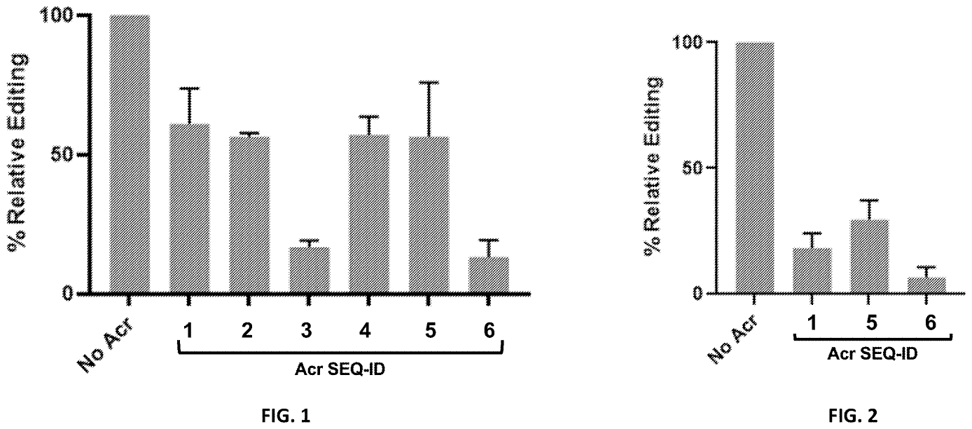

Samples were sequenced by Sanger sequencing. TIDE analysis was performed following the method of Brinkman et al., 2014 as well as TIDE's website (tide.nki.nl) recommendations. Non-transfected cells were also harvested, amplified, and sequenced via the same methods to set a limit of detection (L.O.D.), under which editing levels cannot be determined. TIDE output data on editing efficiency was plotted using Prism software. FIG. 1 shows the ability of a variety of Acrs (SEQ ID NOs: 1, 2, 3, 4, 5, and 6) to inhibit the editing efficiency of the nuclease having SEQ ID NO: 7. FIG. 2 shows the ability of a variety of Acrs (SEQ ID NOs: 1, 5, and 6) to inhibit the editing efficiency of the nuclease having SEQ ID NO: 29. The relative inhibitory activity of the Acrs differed as between UnCas12f and the novel nuclease SEQ ID NO: 29.

### Example 3

### Measured Off Target Inhibition of Acrs

The two nucleases (UnCas12f (SEQ ID NO: 7) and SEQ ID NO: 29) were transfected into HEK293T cells with the Acrs used in Example 2 and either a guide matching the target genomic sequence or a guide with a single mismatch for the same target. The mismatched guide acts as an artificial off-target (OT). Editing efficiency was measured for the matched guide (ON) and OT. The pattern of inhibition for ON and OT is compared between the Acrs when they are paired with UnCas12f or the nuclease SEQ ID NO: 29 to identify Acrs, and Acr-nuclease pairs with an improved ON/OT ratio.

Sequences

| SEQ | |
|---|---|
| ID NO | Acr Polypeptide Sequence |
| 1 | MTDNNYISEIQLKYKPKPISDSIKGSQDIYNLLISKVYDEDTIGYKETFKILLLNNANKV |
| | IGYTTIAEGGLTSTVVDLRVILQTALVANATSIIATHNHPSGVKGPSIQDDMLTAKIKE |
| | ACKAVELRLLDHIIVTPEDGYYSYCDDGKL |
| 2 | MSKVFLLGANKEIDRAKQVVEINQIIQMEGYSYDRYVVYEIRKNDWGIAYKLINQRT |
| | KEFYTTDIIRPLKEKFGIGYYYDSENPQFMDSFEVAILLQQAKDKKKSEDKKAEQEKIR |
| | VEQVKEIGRKRFAEIFPEDAQAVIVARLRQNESDSYTDYYSYSTQRTVIIGFSKHKRDL |
| | FSEMRKHASNFEETAYLAEFNEDYEHREKYSMGDGYYLGKSKYSGWIIEKEPVYNRE |
| | RTIEDFAYTAGNEDNIHLSKTDTMPQSKPTEENTGGCTLVEYSAKAVAVFGDTKSIKD |
| | ELKAMGGRFNSRLTFNGKKLAGWIFPKSQEQRLAYYFGLN |
| 3 | MRMILSDIGKVLWKALSAVVTATSNIAFCAGGLVVGIIGEILFGMLSIMFGILISLFSVM |
| | AAIGFFIWLLTI |
| 4 | MFWLGGLTGYVAGTLVTLLVTYFGYRIGEMNKVDEEWLAESVDKEIEQLRAIRDDN |
| | RDHNRESLKNVRSKFSEYMEKKDKERGNE |
| 5 | MSKEKNLNDIITKELAETFDRYQLEAYVLECVKARGYCQLRARNSQVRFFNFTDEDP |
| | EGRLPGSFHACYLGVVMGLLHEWGFHTERIVATGTNAQIGECDVYVTLQGMVEPRE |
| | LHRLFSSASPQEIGAATNPIHTVLRISQPVKMERMEVTNSIANAYNDPLLLTQTNPVGL |
| | RSLIIAVTLTAALVALVAMAVLQSIT |
| SEQ | |
| ID NO | Cas Polypeptide Sequence |
| 6 | MAEKGLSTGEAYKLMLRSALEKFESLIEWNKSNYDEYKILLRKLKISRGDIKATSNDE |
| | TTDNTKDVGDALENLVNFIINKSFFFKVTSNIRTGSNEIDEVITLTKDGKAALEYFNIPR |
| | SLLVIEDNLFLGECKNYKTPLSVTYVGKFYSLMKQCDCNLGIMFTYKGLSGKETSWN |
| | DSHGLTKVLRLIEKHSSNNPNFYILEFKLEDYEAILEGKTFFNLISSKMIALQISANHNK |
| | FLEEPLEDDLQVLINYCKENK |
| 7 | MAKNTITKTLKLRIVRPYNSAEVEKIVADEKNNREKIALEKNKDKVKEACSKHLKVA |
| | AYCTTQVERNACLFCKARKLDDKFYQKLRGQFPDAVFWQEISEIFRQLQKQAAEIYN |
| | QSLIELYYEIFIKGKGIANASSVEHYLSDVCYTRAAELFKNAAIASGLRSKIKSNFRLKE |
| | LKNMKSGLPTTKSDNFPIPLVKQKGGQYTGFEISNHNSDFIIKIPFGRWQVKKEIDKYR |
| | PWEKFDFEQVQKSPKPISLLLSTQRRKRNKGWSKDEGTEAEIKKVMNGDYQTSYIEV |
| | KRGSKIGEKSAWMLNLSIDVPKIDKGVDPSIIGGIDVGVKSPLVCAINNAFSRYSISDND |
| | LFHFNKKMFARRRILLKKNRHKRAGHGAKNKLKPITILTEKSERFRKKLIERWACEIA |
| | DFFIKNKVGTVQMENLESMKRKEDSYFNIRLRGFWPYAEMQNKIEFKLKQYGIEIRKV |
| | APNNTSKTCSKCGHLNNYFNFEYRKKNKFPHFKCEKCNFKENADYNAALNISNPKLK |
| | STKEEP (Un1Cas12f1) |
| 8 | MIKVYRYEIVKPLDLDWKEFGTILRQLQQETRFALNKATQLAWEWMGFSSDYKDNH |
| | GEYPKSKDILGYTNVHGYAYHTIKTKAYRLNSGNLSQTIKRATDRFKAYQKEILRGD |
| | MSIPSYKRDIPLDLIKENISVNRMNHGDYIASLSLLSNPAKQEMNVKRKISVIIIVRGAG |
| | KTIMDRILSGEYQVSASQIIHDDRKNKWYLNISYDFEPQTRVLDLNKIMGIDLGVAVA |
| | VYMAFQHTPARYKLEGGEIENFRRQVESRRISMLRQGKYAGGARGGHGRDKRIKPIE |
| | QLRDKIANFRDTTNHRYSRYIVDMAIKEGCGTIQMEDLTNIRDIGSRFLQNWTYYDLQ |
| | QKIIYKAEEAGIKVIKIDPQYTSQRCSECGNIDSGNRIGQAIFKCRACGYEANADYNAA |
| | RNIAIPNIDKIIAESIK (AsCas12f1) |
| 9 | MGESVKAIKLKILDMFLDPECTKQDDNWRKDLSTMSRFCAEAGNMCLRDLYNYFSM |
| | PKEDRISSKDLYNAMYHKTKLLHPELPGKVANQIVNHAKDVWKRNAKLIYRNQISMP |
| | TYKITTAPIRLQNNIYKLIKNKNKYIIDVQLYSKEYSKDSGKGTHRYFLVAVRDSSTRM |
| | IFDRIMSKDHIDSSKSYTQGQLQIKKDHQGKWYCIIPYTFPTHETVLDPDKVMGVDLG |
| | VAKAVYWAFNSSYKRGCIDGGEIEHFRKMIRARRVSIQNQIKHSGDARKGHGRKRAL |
| | KPIETLSEKEKNFRDTINHRYANRIVEAAIKQGCGTIQIENLEGIADTTGSKFLKNWPY |
| | YDLQTKIVNKAKEHGITVVAINPQYTSQRCSMCGYIEKTNRSSQAVFECKQCGYGSRT |
| | ICINCRHVQVSGDVCEECGGIVKKENVNADYNAAKNISTPYIDQIIMEKCLELGIPYRSI |
| | TCKECGHIQASGNTCEVCGSTNILKPKKIRKAK (SpaCas12f1) |
| 10 | MTKVIKLALICQQSDSNGMPVDYKEVNKILWELQRQTREIKNKSIQYCWEYHNFSSD |
| | YYKRNGEYPKEKDVLLFTLGGYVNDKFKTGNDLYSANCSTTVRGVCGEFKNSKKDFI |
| | SGKRSIISYKENQPLDLHNKSIRLEYSDHEFYVYLKLLNRQGFKKFNFADTQIMFKILV |
| | RDNSTKTILERCLDEVYSVSASKLIYDKKKKCWVLNLSYSFSGEITHNLDENRILGVDL |
| | GIHYPICASVYGEWKRFTIDGGEIEEYRRRVEARKKTLLKQGKNCGDGRIGHGVKTR |
| | NKPVYSIEDRISRFRDTANHKYSRALINYAIKNNCGVIQMENLEGVTAHSDKFLKNWS |
| | YYDLQTKIEYKAKEAGIKVVYINPRYTSQRCSKCGYTDTDNRPEQAKFICKKCGFSEN |
| | ADFNASQNIGIKNIEQIIKEEIQI |
| 11 | MQKVLKVYLICEQLDPDGSPVDYKDIFKLLWDLQKQTREIKNKSIQYCWEFSNFSSDY |
| | YKEHHEYPKDKEILNYTLGGFVNDKFKTGNDLYSANCSTTVRTACAEFKNAKADFM |
| | RGEKSIISYKANQPLDLHNKSIRLEYQKGTFLFYLKLLNRSAVKKHGFQSSEIRFKAIV |
| | KDNSSQTILERCTAGVYDMAASKLLYDQKKKCWVLNLVYAFEPETPEALDPEKILGV |
| | DLGVHYPICASVYGDLKRFIIDGGEIESFRKRVEARKISMLKQGKNCGGGRIGHGIQTR |
| | NKPVYAIADKIARFRDTVNHKYSRALIDYAIKNNCGVIQMEKLTGVTADANRFMKN |
| | WTYFDLQTKIEYKAQEAGIQIVYIEPKYTSQRCSKCGYIDRENRPEQSKFICRKCGFSE |
| | NADYNASQNIGIRNIEKLIEKQLQTKCESETDTT |
| 12 | MKKTVRLQIVKPMDEDWEILGRVLHDIRYQTRQVLNKTIQLCWEYSNFSSEYKALHG |
| | DYPKNKEILHYTSMHGYAYNQLKEQYYYIQSGNCSQTVKRAVDKWKSDLKEILRGD |
| | RSIPSYKKDIPIDIVKDAVSLEHDEKNGNYIATLSLMSTAYRKEMERKSGQFRVLLHSG |
| | DNSKKTILKRLVQGEYQHTASQIVKKGKKWFLNLSYKFDVLQTPFQTERVMGVDMG |
| | VIYPIYMAFNYHDHLRYKIQGGEIERFRRQVESRKKALQDQGKYAGSGRVGHGTKTR |
| | IDPLEVIRDKIANFRETKNHHYSRYVVDMAEKHECATIQLEELKGIHQDDAFLKRWSY |
| | HDLQEKITYKAEEKGIQVIKVDPQKTSQRCHHCGNIDSNNRKEQASFLCTSCGMETNA |
| | DFNAAKNISIPGIEQIIQTEMKS |
| 13 | MCALTKIMKYELRYLDGFPDFSAMQNAVWPLQRQTREILNRTIQEAYRWDYFSATK |
| | KKETGEYPDLQKETGYKRLDGYIYHVLSPDYPDFSSSGVNATIQKAWKKYKSSKADV |
| | WKGEMSLPSYKSDQPIVLHAKQIKLSGDTRAAAATLSLFSNKFKKEHEISGNVQFAIT |
| | LHDNTQRTIYQKLRNGEYKLSESQLVYDKKKWFLYLAYSFNPAEHALDPEKILGVDM |
| | GEKFALYASSFGEYGHFKIEGSEVTEYAKALERRRRSLQQQARYCGEGRIGHGTKTR |
| | VGVVYREEDRIANFRSTINHRYSKALIEYAVKNGYGTIQMENLTGIKENLQFPRRLQH |
| | WTYYDLQSKIEAKAKEHGIAVVKVNPKHTSQRCSRCGHIAAENRPKQEVFQCVKCG |
| | YACNADFNASQNISIKDIEKLIQETIGANPK |
| 14 | MVLTRKLQLVPCAEGMSKEEAKKEVDRVYKILRDGIYAQNKAYNIFISRRYTAILLGA |
| | SKEELAKLTLIGERNPKKDDPSYSLYEYGKINFIKGIPQASALGQHAISDLSKQKKDGL |
| | FKGKVALACRRLDAPMWIKQKYEFYHNYADNKELSENLYSEDLKIYMKLANICVFE |
| | VVLGNPHKSAAIRAELERVFDENYKKLDSSIQIVNNKIMLYLAINIPEKQIELKEDVVV |
| | GVDLGIAIPAVCALNNSRYIKKSIGSANEFLRIRTQLQSEKKRLQRKLRDTNGGHGRK |
| | KKLAPLDKLSKRERNFVQTYNHMISRRVVDFAVKNNAKYINVEDLSDYKNNGSEYIL |
| | RNWSYYELQQQIEYKAEMYGIVVRKVNPYHTSQICSQCGHWEEGQRKSQSEFECKA |
| | CGYTANADFNAARNIALSTDFVKK |
| 15 | MSKGVVTKVMKYTLRYIGGCGDFHKMQEAVWKLQRQTREVLNKTVQLAFDWDHR |
| | SREAYRTSGEYLDIVKETGYKRLDGYIYNRLKTDYADFASSNLNATIQVAWKKYMAS |
| | KSDILTGKMSFPSYKSNQPIVLHNSSIRFSTEKYGVPAAELTVFSNALKKENGLSTNPA |
| | FEILLMDGTQRSIFQRVISGEYKHGQCQINYEKRKWFLYLTYTFEAGKTPLDPDKILGV |
| | DIGETLAICASSTSEWGRFVIQGGEVTRYSKQIEERRRSQQRQATYCGEGRIGHGTKAR |
| | VAPIYATEDRIANFRDTINHRYSKALIEYAVKHGFGTIQMEDLSGIKGEKDFPKFLRHW |
| | TYYDLQNKIEAKAKEQGINVVKVQPAYTSQRCSKCGCIDKENRKNQELFCCVKCGFK |
| | ANADFNASQNISIKGIDKIIEKEYNANIE |
| 16 | MNKVVKLALISKVKDKDGNDVKYGDVCKILWELQRQTREIKNKAVQFCWEWNGFS |
| | SEYNNLFGEYPKDKDYLKNKSEGKPIVLRSFVYDRLKSDYYLNSSNLSTTTSLAFKEF |
| | KQYLTDIRKGERSVLNYKNNQPLELHNECIWLESNNGKFITRFSFLNKAGKDFYSIDN |
| | FTFEVIVKDNSTKTILERCIDSIYGIRASKLIYNQKKKQWFLNLSYSFEAKEIATLDKDK |
| | ILGVDLGIALPICASVYGDLDRFTIKGGEIEHFRKSIESRKRSLLQQGKVCGDGRIGHGI |
| | HTRNKPAYNIEDKIARFRDTANHKYSRALIDYAVRKGCGTIQMEMLKGITEEKDPFLK |
| | NWTYFDLQQKIEYKAKEKGIKVVYIAPKYTSRRCSKCGHIDKDNRLTQANFLCLNCG |
| | YKENADYNASQNIAIKDIDKIIIEETKGGES |
| 17 | MNKVVKLSLICEQTDKDGNKIEHGEVYKILWELQRQTREIKNKTIQYCWEYSNFSSD |
| | YYKINHEYPNEKEILSFTLKGFVNDKFKKGNDLYSGNCSTTTGNVCSEFKNSKSDFLK |
| | GEKSIINYKAYQPLDIHNKCILIEHTNNEFYVRLKLLNRPAIKKYNFANSEFNFKIIVKD |
| | NSTRTILERCIDKIYDVAASKLIYDKKKKMWVLNLVYAFDNKSEYVLDKNRILGVDL |
| | GIHYPICASVYGEWNRFTIDGGEIEKFRKTVEARKKSMLRQGKNCGDGRIGHGISARN |
| | KPVYKIEDKIAKFRDTANHKYSHALIQYAIKNNCGVIQMEDLTGITNEADRFLKNWSY |
| | YDLQTKIKYKAKEVGIDIVYIKPKYTSQRCSKCGYIDKENRNKQASFVCLKCGFKANA |
| | DYNASQNISIKDIDKLIEEMYNSSANTE |
| 18 | MSKGSLAKVMKYELRYLDGAGSFEQMQERLWVLQRQTREILNRSTQISFHWDYTSR |
| | EHFEQTGQYLDVFSETGYKRLDGYIYSRVKDSCGDMASGNINATLQKAWNKYGTSK |
| | LDVLRGQMSLPSYKKDQPLVIEKHNIRLSMDGQQALAEITLFSNKFKKENSLSSNVRF |
| | AFQLHDGTQRRILNSVLSGEYGLGQCQLVYDRPKWFLLLTYTFTPQNRQLDPDRILGV |
| | DLGECYALCASVFGEYGSLRIEGGEVTAYAKKLEARKRSLQKQAAVCGEGRKGHGT |
| | KTRVADAYQMQDRIANFRDTVNHRYSKALIDYALKNQCGTIQMEDLSGIRQDTGFPK |
| | FLQHWTYYDLQSKIENKAKEHDIRIVKINPRYTSQRCSKCGAIDSGSRTSQARFCCTKC |
| | GFTANADYNASQNISIKGIDLLIEKELGAKAE |
| 19 | MGKGEISKVMKYELRYLDGSGSFEEMQQRVWALQRKTREIQNRTVQIAFHWDYINR |
| | EHFIQTGNNLNVLQETGYKRLDGYIYDRLKGQSAEMSGANLNATIQTAWKKYNSAK |
| | PKVLSGTMSVPSFKRDQPLIINSNCVKFSRSESECLAELTLFSREYKKEHDLSSNVRFAI |
| | RLHDSTQRSILERVLSGAYRKGQCQLVYQRPKWFLFLTYSFFPMQHDLDPEKYLGVD |
| | LGECCALYASSVGEYGSLKLEGGEITAFAKQLEARKRSMQKQAAYCGEGRIGHGTKT |
| | RVADVYKMENRIANFRDTVNHRYSKALIDYAVKHQYGTIQMEDLSGIKNDTGFPKFL |
| | RHWTYFDLQEKIDAKAREHGIHVVKVNPQYTSQRCSKCGSIDSRNRKSQKEFCCLNC |
| | GYKVNADFNASQNLSIKGIDVIIQKYIGAKSKQTENNG |
| 20 | MKEIAKVVKLELGWVFTDDGERAFPYSDLFEIQRQVALVKNKTIQLCWEWNNFGAD |
| | FHAMSGAYPKTGDVTGYKTLDGYVYQRLKDDFSRMFKKNLNASVRSAQKAFETAK |
| | KSLHKGERSILSYRKDAPIELHNSAVSFSQEGRKYKAAVKVFSLSYAKEKGYAGTGVE |
| | FELSHLQGSPKEIVQRCMSGEYKIGESKLIWNEKKKKWFLYLTYKFTPAAVALDPEKI |
| | MGVDLGIACVAYMGFRFCEDRHVIPGHEVEHFRRRVEARKVELQRQGKFCGEGRIGH |
| | GRATRTKPVDQIGHAIARFRDTANHKYSRFIVDMAVKHGCGVIQMEDLHVHAEDKL |
| | LKDWTYFDLRTKLEYKAKEKGIEVRFVNPRYTSQRCSCCGYIEKENRKTQKEFICLEC |
| | GFAANADYNAALNLATAGIEQIIDEYVSANHK |
| 21 | MSKGTLAKVMKYELRYLDGFPSFYEMQEAVWGLQRQTREILNKTLQMAFHWDYTS |
| | REHFKETGSYLDVRTETGYKRFDGYVYECLKSDYSDIASKNLNATLQKAWKKYRNT |
| | RLDVLKGTMSLPSYRSDQPLTLDKNTVKLHSDGVDDWVELTLFSKAYKTQHGLSAN |
| | VRFAIPMHDRTQRSIFQNLIDGVYALGECQLVYDKKKWFLLVTYVFTPEQHTLDPEKI |
| | LGVDMGEAYAIYASSVYGYGTLKIEGGEVTDYAKKLERRKWSYQKQARYCGDGRIG |
| | HGTKTRIAEVYKAEDRIANYRDTINHRYSKAVVDYAVKNGYGTIQMEDLSGIKEDTG |
| | FPRRLQHWTYYDLQTKIENKAKEHGIRVVKIDPRCTSQRCSRCGHIDPKNRPSQSQFC |
| | CTACDFRANADFNASQNISTKGIDKIIAKTLRAKPE |
| 22 | MGTVTKVMKYELRYLDGSGSFHDMQNYVWQLQRQTREILNRTIQEATLWDYRSRE |
| | HFLEAEEYLDVYAETGYKTLDGYIYNRLKGSYGDFAGANLNATLRKAWKKYKTSKT |
| | EVLRGTMSLPSYKGDQPLVLHNGSVKLQGDSRDAVVELTLFSNTFKKREGIKGNPSFS |
| | LLVRDNTQRSIYQSLIDGVYKLGECQLVYQKKKWFLLLTYTFEARQHEVDPEKILGV |
| | DLGEAYAIYASSKDNFGSLKIEGGEVTDYAKGLERRKRALQQQARYCGEGRVGHGT |
| | KTRVTEAYKAEDRLANFRKTINHRYSKALIDYAVKNGYGTIQMEDLSGIKADTGFPK |
| | RLQHWTYYDLQSKIEAKAKEYGIHVVKVDPSYTSQRCSKCGHIDSQNRKTQERFLCV |
| | SCGFSCNADFNASQNLSIKGIEKIIKKTKGAKVE |
| 23 | MAKKGNSQKKQIVKVMKYELKYEKGCADFNEMQNELWKLQRQTREVMNRTIQLCY |
| | HWSYVQAEYCKQHGCARRDVKPCDVYETNATSLDGYIYQLLKVEYPDFFMKNLNA |
| | TLRKAHQKYDALLFDIQEGNSSIPSFKKDQPLIFEKKAICISKCLPDKRQITLSCFSDSYI |
| | DAHPTLDKITFTVRARSASEKSIFDHIISGKYALGTSQLVYEKKKWFFLLSYKFTPESV |
| | DVNPEKVLGVDLGVVNALCAGSVENPHDSLFIKGTEAIEQIRRLEARKRDLQKQARY |
| | PGDGRIGHGTKTRVSPVYQTRDAIARMQDTLNHRWSRALIDFACKKGYGTIQMEDLS |
| | GIKAMESEKPYLKHWTYFDLQSKIIYKAEEKGIRVVKVNPKCTSRRCSACGYISKENR |
| | KNQAEFLCVNCGYHHNADYNAAQNLSIPQIDRLIEKQLKEQESEESEAGTNPK |
| 24 | MDLRNWLRRVRIHMAKGTVTKVMKYELRYLSGFSDFHAMQQAVWGLQRQSREILN |
| | KTIQMAFHWDYISRENFNANGVYLDVKAETGYKTYDGYIYNSLKSAYADMAAANL |
| | NAAIQKAWKKYKDAKMEVLRGTMSTPSYRSDQPVLINKNCVKLFDGGVRLTLFSDR |
| | FKRENNLNGNLEFAVQLHDGTQRSIFANLLNGTYALGQCQLVYDKRKWFLLVTYIFT |
| | PEKHELDPEKILGVDLGQTYALYASSVCARGTFRIEGGEAAECAHRLEQRKRSLQQQ |
| | ARFCGEGRVGHGTKTRVAAVYSAGDKIASYRDSINHRYSKALVEYAVKNGYGTIQM |
| | EDLTGIQNDLDHPKRLQHWTYYDLQTKIENKAKEHGVGVVKVNPRYTSQRCSRCGHI |
| | ERENRPTQKVFCCKACGFEGNADYNASQNLSMRNIDKIIEKELSAKGE |
| 25 | MSGSTIAKVMKFELFYREGGGEFHEMQKLLWELQRQTREVLNKSVQIYYQWKWKK |
| | QQHFEETGQSLDIYTELHYHRISSYAYNVLKEKYSSFYKANLSSTIKTACDKCESSEKD |
| | ILCGTMSVPSYKRDQPLLLHDTSLSIRRNGTQWFADCKLFSAELVKNLGLKRGQSLVF |
| | SIKALDKTQINILERIEDGNYAIRQSQLTYEKKKWFLYLTYRFDKPKSELDPNRILGVD |
| | LGVSNAFCASVYGELDKLMIPGDEAIETIRRLEQIKYSKLRQARYCGEGRIGHGTSTRI |
| | APAYSTRDKISNLQKTLNHRWSHAIVRYALRQGCGVIQMEDLSGIKEANDFPLRLQH |
| | WTYYDLQTKIKNKANEYGIEVRQVDPQFTSQRCSKCGCIQKENRPAQAKFCCIKCGY |
| | RTNADYNASQNLALPDIDRIIQEELKQIGANRK |
| 26 | MQQVVRFEILKPVDNDWKILGRVFRELQYESRLVLNKTIQYNWEFSNCVIGFKEKFGI |
| | APKISDISKYKDGKRGLEGYIYDKLKDIYTKNYSKNLGCLISKATSQWYAIKNDVYKG |
| | EKIPPEYKKSNTPIIVDKQAMTLFKENNLYYAKVALVSTNHIKEYGLNSCKFTILLNTK |
| | NNGNKVILDKVINGEFDYSQSQIIEKNKKWFLYLSYKIKEKTLPDDYNRVMGIDLGKN |
| | KAVVIAVHGTEIRDYILGGEIIDYKKKMYNMVWHRQKQSRYCGEGRIGHGRKTRLK |
| | DVYNIKEKIANFSDLTNHKYSKYIVELAKRHKCGIIQMEDLSGLSTDNKFLKQYPIYDL |
| | QQKIIYKAEREGIKVVKIKPHFTSQMCSNCHYISTQNRPKDDRGWEYFKCVNCGLEID |
| | ADLNAARNIANPQIELIIEEQLKIQEIDDKI |
| 27 | MAEKTIVKVMKFELRYIDGAGEFSEMQKHLWELQKQTREVLNKTIQMGYALECKRF |
| | AHHDKTGQWLDDKELTGSKYKAVADYINAELKEDYNIFYSDCRNSTVRKAYKKFKD |
| | AKNKIFSGEMSLPSYRSNQPIIIHNRNVIIRGNAESALVGLKVFSDGFKALHGFPAAVNF |
| | KLCVKDGTQRAIIENVISEIYKISESQLIYDNKKWFLILAYRFTQKKNDLNPDKILGVDL |
| | GVKFAVYASSIGEYGSFRIKGGEVTEFIKRLEKRKKSLQNQATVCGDGRIGHGTKTRV |
| | ADVYKARDKISNFQDTINHRYSRAIVDYARKNGYGTIQLEKLDNSIEKKGDYSPVLVH |
| | WTYYDLRTKMEYKAAEYGIKVIAVEPKYTSQRCSKCGYISSENRKTQESFECIKCGYK |
| | CNADFNASQNLSVRDIDRIIDEYLGANPELT |
| 28 | MANEFTCITRKIEVHLHKHGDSDEAIQRYKEEYRMWDDINNNLYKAANRIVSHCFFN |
| | DTYEYRLKLHSPRFQEIEKLLSNPKRNKLSDDDIKELKAERKLLFSDFKSQRQTFLRGG |
| | IETGTNPEQNSTYKVISNEFIDCIPSEVLTNLNQNISSTYREYTLDVERGIRTIPNFKKGIP |
| | VPFSIKQHGEIALKKRDDGTIYVRFPKGLEWDLNFGRDRSNNREIVERVLSGQYGVGN |
| | SSIQESKNKKQFLLLVVKIPKENRVLDKERIVGVDLGVNTPLYAALNDNEYGGMGIGS |
| | REQFLKVRERMNAQKRELQRNLRHSTNGGHGRSQKLQALDRLEGKERNWVHLQNHI |
| | FSKSIIEYALKNDAGVIQMERLTGFGRDNNEEVQNEYKYILRYWSYFELQTMIEYKAK |
| | AAGIEVRYINPYHTSQTCSFCGHYEKGQRINQPTFICKNPDCTKGKGKQKSNGAYEGI |
| | NADWNAARNIARSNEFVEKKKK |
| 29 | MATEYTCITRKIEVHLHRHGDSEEDTQRLKDEYHIWDVINDNLYKAANRIVSHCFFND |
| | AYEYRLKLHSPRFQEIEKLLRYSKRNKLTDEDIKQLKAERKELFSIFKKQRLEFLQGGS |
| | GKGSEQNSTYKVVSNEFGEIIPSHVLTCLNQNITSTYSAYSKEVEYGNRTIPNFKRGIPV |
| | PFPIKQQGTLQLKRREDGSIYIRFPLGLEWDLSFGRDRSNNREIVERVLNGQYDVGNSS |
| | IQETKNKKRFLLLIVKIPKQAVTLNPDRIVGVDLGINIPLYAALNDNEYGGMGIGSREQ |
| | FLKMRMRMAAQKRELQRNLRHTTHGGHGRTQKLQALERLEGKERNWVHLQNHIFS |
| | KSIIEYAQRNDAGVIQMERLTGFGRDKHDEIDSDFKFILRYWSFFELQTMIEYKAKAA |
| | GIEVRYIDPYHTSQTCSFCGHYEKGQRISQSTFVCKNPDCEKGKGKKHSDGTYEGINA |
| | DWNAARNIALSTKIVDRKKK |
| 30 | MATEYTCITRKIEVPLHRHGEDEEAKQRLIDDYRVWDTINDNLYKAANRIVSHCFFND |
| | AYEYRLKIHSLRFQEIEKLLKYSKRNKLTDEDIKQLKAERKQLFADFKKQRHTFLRGG |
| | VAEGANPEQNSTYKVISNEFLEVIPSEILTNLNQNISSTYKNYSLDVERGIRTIPNYKRGI |
| | PVPFSIKQRGELMLKRRDDGSIFIRFPMGLEWDLSFGRDRSNNREIVERVLSGQYDVG |
| | NSSIQESKNRKRFLLLVVKIPKENHNLNPDRIVGVDLGINIPLYAALNDNEYGGMGIGS |
| | REQFLNMRMRMDAKKRELQRNLRQSTNGGHGRKQKLQALERLEGKERNWVHLQN |
| | HIFSKSIIEYAVKNNAGAIQMERLTGFGRDKNDEVDSDFKFILRYWSFFELQTMIEYKA |
| | NAAGIEVRYIDPYHTSQTCSFCGHYEKGQRLNQSTFVCKNPDCEKGKGKKLSNGTYQ |
| | GINADWNAARNIALSDKIVDRKKK |
| 31 | MATEYTCITRKIEVHLHKHGDSEEAAQRFKEEYRIWDDINNNLYKAANRIISHCFFND |
| | TYEYRLKLHSPRLQEIEKLLSNPKRNKLSDEEVKQLKAERKQLFADFKKQRHVFLRG |
| | GVEEGANPEQNSTYKVVSNEFIDFIPSEVLTNLNQNISSTYREYSLDVERGVRTIPNYK |
| | KGIPVPFSIKQKGEIVLKKREDGSMYVRFPKGLEWDLNFGRDRSNNREIVERVLSGQY |
| | DVGNSSIQETKNKKRFLLLVVKIPKQVASFDPSRIVGVDLGINVPLYVAINDNEYGGM |
| | GIGSREQFLKMRMRMAAQKRELQRNLRHTTNGGHGRTQKLQALNRLEGKERNWVH |
| | LQNHIFSKSIIEYAVRNNAGVIQMERLTGFGRDKNDEVGADFKFLLRYWSFFELQSMI |
| | EYKAKATGIEVRYINPYHTSQTCSFCGHYEKGQRINQATFVCKNPECTKGKGKQRTD |
| | GTFEGINADWNAARNISFSTDFVDKKKK |
| 32 | MATEYTCITRKIEVHLHRHGDSEEAAQRLKEEYRIWDEINDNLYKAANRIISHCFFND |
| | AYEYRLKLHSPRFKEIERLLKYAKRNKLTDDDIKALKAERKELFAEFKRQRQSFLGGS |
| | EQNSTDRVVSHEFLDVIPSEVLTCLNQNIASTYKEYARDVERGVRTISNFKKGIPVPVR |
| | VKRNGALLLRKREDGSIYLSFPKGLEWDLNFGRDRSNNREIVERVLSGQYDVGGSSIQ |
| | EAKNGKRFLLLVVKIPKESRALNPDRVVGVDLGVNIPLYAALNDNTYGGLSIGSRDQF |
| | LKVRMRMAAQKRELQRNLRVATNGGHGRKQKLQALDRLEGKERNWVHLQNHIFSK |
| | SIIEYALRNEAGAIQMERLTGFGHDRNDEVDEGFKFILRYWSFFELQTMIEYKAKAAGI |
| | EVRYVDPYHTSQTCSFCGHYEKGQRVNQATFICKNPDCTKGKGKERSDGTFEGINAD |
| | WNAARNIALSDKIVERKKK |
| 33 | MATEYTCITRKIEVHLHKHGDSEEATQRLKNEYHIWDEINNNLYKAANRIVSHCFFND |
| | TYEYRLKLHSPRFQEIEKLLNNSKRNNLSAEEIRQIKIERKLLLSEFKKQRYAFLRGGIE |
| | EGANPEQNSTYKVVSNEYIDKIPSDVLTNLNQNISSTYKEFSLDVEKGVRTIPNYKKGL |
| | PIPFSIKKNGDLLLKKRDDGTIYIRFPKGLEWDLSFGRDRSNNREIVERILSGQYDVGNS |
| | TIQETGNKKRFLLLVVKVPKKNIVSNPNRVVGVDLGINYPLYAALNDNEHGGISIGSR |
| | DQFLKMRMRMAAQKRELQRNLRHTTNGGHGRTQKLQALERLEGKERNWVHLQNHI |
| | FSKSIIEYAIKNNAGTIQMERLTGFGRNENNEVGSEYKFLLRYWSFFELQTMIEYKAKA |
| | SGIDVRYINPYHTSQTCSFCGHYEKGQRLNQATFVCKNSACTKGKGKQKSDGTYEGI |
| | NADWNAARNIALSTDFVDKKKK |
| 34 | MATEYTCITRKIEVHLHRHGDSEEAAQRLKEEFRIWDEINDNLYKAANRIISHCFFNDA |
| | YEYRLKLHSPRFQEIEKLLKYAKRNKLTDDDIKALKAERKELFAEFKRQRQSFLGGSE |
| | QNSTYKVVTDEFLEVIPSHVLTCLNQNISSTYREYALDVEHGRRTIPNFKKGIPVPFPIK |
| | ATGELLLRKREDGSIYIRFPKGLEWDLNFGRDRSNNREIVERVLSGQYDVGNSSIQETK |
| | NRKRFLLLVVKIPKESRALNPDRVVGVDLGVNIPLYAALNDNTYGGMSIGSRDQFLK |
| | VRMRMAAQKRELQRNLRVATNGGHGRKQKLQALDRLEGKERRWVHLQNHIFSKSII |
| | EYALRNEAGAIQMERLTGFGHDRNDEVDEGFKFILRYWSFFELQTMIEYKAKAAGIE |
| | VRYVDPYHTSQTCSFCGHYEKGQRVNQATFICKNPDCTKGKGKERSDGTFEGINADW |
| | NAARNIALSDKIVERKKK |
| 35 | MATDVTCITRKIEVHLHKHGDSEEGAQRLKEEYRIWDDINNNLYKAANRIISHCFFND |
| | TYEYRLKLHSPRFQEIEKLLSKPKCNKLSADDIKQLKAERKVLFADFKKQRQVFLRGG |
| | LEEGTNREQTSTYRVASKEFIDTIPSEVLTNLNQSISSTYKKYALEVERGVRTIPNYKK |
| | GIPVPFAIKHKEELALKKRDDGSIYVRFPKGLEWDLSFGRDRSNNREIVERVLSGQYD |
| | VGNSSIQETKNKKRFLLLVVKIPKENRVLNKERVVGVDLGINTPLYAALNDNKYGGL |
| | SIGSRDQFLKVRMRMTAQKRELQRNLRHTTNGGHGRTQKLQALDRLEGKERNWVH |
| | LQNHIFSKSIIEYALQNDAGVIQMERLTGFGHDNNDEVDEKFKFILRYWSFFELQTMIE |
| | YKAKAAGIEVRYINPYHTSQTCNFCGHYEKGQRINQATFVCKNPDCIKGKGKQHSDG |
| | SFAGINADWNAARNIALSNDVVDKKKK |
| 36 | MTAEYTCITRKIEVHLHKHGESEEATQRFKDEYRIWDDINNNLYKAANRIISHCFFND |
| | AYEYRLKLQSPRFQEIEKLLGNTKRNKLSAEDIKVLKAERKLLFSDFKKQRQIFLRGG |
| | VEEGPNPEQNSTYKVVSQEFIDVIPSEVLTNLNQNISSIYREYALDVERGIRAIPNYKKG |
| | IPVPFSIKQKGEIVLKKREDGSIYVRFPKGLEWDLNFGRDRSNNREIVERVLNGQYDA |
| | GNSSIQETKNKKRFLLLVVKIPKESRSLNKERIVGVDLGINVPLCAALNDNEYGGISIGS |
| | RDQFLKVRMRMAAQKRELQHNLRHTTTGGHGRTQKLQALDRLEGRERNWVHLQN |
| | HIFSKTIIEYALKNNAGVIQMERLTGFGRDGKEEVQNEYKFILRYWSFFELQTMIEYKA |
| | KAVGIEVRYINPYHTSQTCSFCGHYEKEQRISQTTFVCKNPKCTKGKGKLKSDGTFEGI |
| | NADWNASRNIAKSTEFVDKKKK |
| 37 | MATEYTCITRKIEVHLHKHGDSEEATQRFKDEFRIWDDINNNLYKAANRIITHCFFND |
| | AYEYRLKLHSPRLQEIEKLLSNSKRNKLSDEEVKQLKAERKQLFADFKKQRQVFLRG |
| | GVEAGANPEQNSTYKVVSNEFIDTIPSEVLTNLNQNISSTYREYSLDVERGIRTIPNYKK |
| | GIPVPFSIKQKGEIVLKKREDGSIYVRFPKGLEWDLSFGRDRSNNREIVERVLSGQYDV |
| | GNSSIQETNNKKFLLLVVKIPKQMASVDPNRIVGVDLGINVPLYAALNDNEYGGMGI |
| | GSRDQFLKVRMRMAAQRRELQRNLRYTTNGGHGRTQKLQALDRLEGKERNWVHLQ |
| | NHIFSKSIIEYAVRNNAGIIQMERLTGFGRDENDEVGTDFKFLLRYWSFFELQSMIEYK |
| | AKAANIDVRYINPYHTSQTCSFCGHYEKGQRINQSTFVCKNPECAKGKGKQRADGTF |
| | EGINADWNAARNIAFSTEVVDKKKK |
| 38 | MATEYTCITRKIEVHLHRHGDSDEAIQRYKDEFHIWDEINNNLYKVANRIISHCFENDT |
| | YDYRLKLHSPRFQEIEKLLRNPKRNKLSGEDVKRLKAERKALDADFKKQRQAFLRGG |
| | VEEGTNKEQTSTYIVVSHEFIDIIPSEILTNLNKNIFSTYKKYRLDVEKGARTIPNYKKGI |
| | PVPISIKRSGELMLKKREDGSIYVRFPKGLEWDLFFGRDRSNNREIVERVLNGQYDVGI |
| | STIQETKNKKRFLLLVVKIPKESKNLNPNRVVGVDLGINIPLYAALNDNEYGGLGIGSR |
| | EQFLKVRMRMAAQKRELQRNLRHTTNGGHGRAQKLQALDRLEGKERNWVHLQNHI |
| | FSKSIIEYALRNGAGVIQMERLAGFGRDKNEEVENEFKFILRYWSFFELQTMIEYKAN |
| | AAGIEVRYIDPYHTSQTCSFCGHYEKGQRINQSTFVCKNPDCVKGKGKQHADGSYDG |
| | INADWNAARNIALSTTVVDKKKK |
| 39 | MATEYITKTRKIEVYLHRHGDSDEAKQRYQQEWQIWHDINDNLYKVANRIMTHHFL |
| | NDEFVSRLRSTNPRYVEIEKILKHCKRNKLSQEEINSLQQENRALDALFTEKKNEFLGT |
| | TQEHNTILRIVRKEFGDVIPNDVYDCVIAERVKYTDKQKHLQIINGESSVPNYRKGMP |
| | VPFRIKIGANHNTLGILRRNDNPNHIYVKFPKGLEWDLVFGKDPSNNRKIVERILSGQY |
| | DAGNSSIQQAKNGKCFLLLVVKIPKSNIQLNKDRVVGVDLGINIPLYAALNDNIHSRLS |
| | IGSREQFLKMRMRMYAQKRELQRNLRHSTNGGHGRKQKLQALERLEGKERNWVHL |
| | QNHIFSKSVIEFAQKHNAGVIQMERLTGYGKDANGEMREEAKFLTRYWSYFELQTMI |
| | EYKANAAGIEIRYIDPYHTSQTCSFCGHYEKGQRVSQSTFICQNPECKQGKGKQKSDG |
| | TFEGINADWNAARNIALSTQYVDKKKK |
| 40 | MATEYTCITRKIEVHLHRHGEDEDAVQRYKNEFQIWNEINNNLYKVANFISSHLFFND |
| | AFVDRLRVQSNEYRDLLDLISKTTDAKEIKALENRKKALDAEFKRQQKIFLKGGSEDE |
| | KGSEKTAIRRIAVETFPNIPYSIINSLNDQISKTYNSSRFDVSIGKRTVPNYKKGIPVPFL |
| | MANGSGKIALREREDGSPYVLFPRGLEWDLHFGKDSSNNREIVKRVFNGEYKACDSS |
| | LQQAKNKKIFLSLVVKIPKKNHNLNPDRIVGVDLGINIPLYAALNDNDYGGMGIGSRE |
| | QFLKVRMRMSAQKRELQRNLRQSTNGGHGRAQKLQALERLEGKERNWVHLQNHIFS |
| | KSIIEYALKNNAGAIQMERLTGFGRDKNDEVDSNFKFILRYWSFYELQTMIEYKANAA |
| | GIEVRYVDPYHTSQTCSFCGHYEKGQRLNQSTFVCKNPDCEKGKGKKLSDGTYQGIN |
| | ADWNAARNIALSDKIVDRKKK |
| 41 | MNDSPVIKNKRHVKVLRLRILKPVSGTWQDLAKLLRDTRYRVYRLANLAVSEAYLG |
| | FHMWRTGRAETYELDTPGALNRRLRRMLDEEGVRADELDRFSKTGALPDTVVGALS |
| | QYKIRAATGKSKWQEVIRGKSSLPTYRLDMAIPLRCDKRNHARLARVENGDVTLDL |
| | MLCLRPYPRVVIQTGNIGGGAQAVLDRLLANPSQNPDGYRQRLFEIKHDDRDNKWW |
| | LYITYDFPAADPPRSSADRIVGVDIGVSCPIYVAINDGHARLGRRQFSSLGARIRSLQNQ |
| | IVARRRSMQAGGKVALSGQTSRSGHGRKRKLRPIQKLEGRISHAYTTLNHQLSSSVID |
| | FALSHGARVIQMEDLASLKDALRGTFIGARWRYHQLQQFLEYKAKESGLTLRKINPQ |
| | FTSRRCSRCGFIHVEFDRARRDASRRDGYVARFVCPAPKCGFEADPDYNAARNIATPD |
| | IEKLISDQCKIQSIPTRSLTDQSEAADKDTLAQGQSRSGG |
| 42 | MSEKRHNKVAKFQILKPAAGTTWPELANLLFAVRYRVFRLANLCISEHYLHYHLWR |
| | MGKTEEIPKLKISELNKKLREMIIEENDKKEKQNKINQDAINKKGALTSYVVDTLSQN |
| | KLGAVTSKSKWKEVILGKASLPTFRLNMAIPVRCDKPEQCRLKINANGDVELELMICE |
| | RPRPRIILKTGGLSGSMKSVLDRLLENSAQSMEGYRQRNYEIIQDRNDGKKWYLHVS |
| | YDFPATERKPNSEIIVGVDVGFALPLYAALGNGHARLGWKQFHSLAKRIRSLQNQVV |
| | SRRRKMLRGGKDSLTQDTARSGHGRNRLLQPIEKLAGRIEKAYTTLNHQLSRSVVDF |
| | AKNHGAGIIQMEDLEGMKDAINGTFLGERWRYFELRQFIEYKAKEAGIEVRLANPKY |
| | TSRRCSACGYINMAFTREYRDSHRKNGKSAEFFCPECDKLPADDEQPQKPYPTDADY |
| | NAAKNLAALDIEKIIRRQCEKQGISYDKSPENNDL |
| 43 | MANTEFTCITRKIEVHLHRHGDSDEAALRLKNEYHFWDEINDNLYKAANRIISHCFFN |
| | DTYEYRLKLHSPRFQEIEKLLKNAKRNKLTDEEIKELKAERKLLFSDFKKQRYTFLRG |
| | GIEDGANPEQNSTYRVVSNEFIDTIPSEVLTNLNQNISSTYKNYTIDVERGLRTIPNYKR |
| | GMPVPFSIKKHGVLALRKREDGSIYVAFPKGLEWDLSFGRDRSNNREIVERVLSGTYD |
| | VGNSSIQEAKNGKRFLLLVVKIPKEVKILDTSRVVGVDLGVAVPLYAAISDNEYGGMS |
| | IGSYDQFIKVRMRMNAQKREMQHNLRHTTNGGHGRKQKLHALERLEGKERNWVHL |
| | QNHIFSKSIIEYALQNNAGAIQMERLTGFGHDKNNEVDEGYKFILRYWSFFELQTMIE |
| | YKAKAAGIEVRYIDPYHTSQTCSFCGHYEKGQRLNQSKFVCKNPDCVKGKGKQRSD |
| | GSFEGINADWNAARNIALSTKIVDRKKK |
| 44 | MENNITITRKYALIPEFSDRKEWKKRVYDFMINDLEQKIDYRNKKKQDTSELESQLEY |
| | IKNGGDFTRSMVNNYTYSLVRKAMEEETRRKNYILSWIFSEMRANRIDQMESLKDKF |
| | KFVSDTINYAYRKAGSNKGSLFDETEIHCILKSYGIAFSQELTKEIKELVKNGVLEGKV |
| | VIPTYKLDSPFTIAKSHFSFEHDYDSFEELCEHINDSDCKMYMNYGGDNRKDGINPASI |
| | ARFRISLGHGKNKDELKSTLLKVYSGEYQYCGSSIQITKNKIILNLTMKIPKIETKLDEN |
| | TVVGVDLGIAVPAMCALNNNMYERLAIGSADDFLRTRTKLQSQRRRLQKSLKNSNG |
| | GHGRNKKLKVLERLGKSETHFAETYCHMVSKRIVDFALKNNAKYINIENLNGYNTSS |
| | FILRNWSFYKLQQYITYKAERYGIVVRKINPCYTSQVCSVCGNWEDGQRKTQASFEC |
| | ANPKCESHKKYKYGFNADFNAARNIAMSTLFMEDGEVTEKKKEEAREYYGIKKENS |
| | EAV |
| 45 | MATEYTCITRKIEVHLHKHGDGEEAEKRRAEEFRMWNEINDNLYKAANRIVSHCFFN |
| | DAYEYRLKIQSPRYKEIQRKLRYSKSNKLTDDEIKSLKAERKELDNEFRKQYRAFMLG |
| | GSSEGFKSTTEQNSTERIVNNEFGDIIPSNVLSCLNQNVFQTYKQYRTDVEFGKRTISNF |
| | KKGMPVPFSIKAHKSLMLKKREDGSIFVYFPKGLEWDLSFGRDRSNNREIVERILSGQ |
| | YDAGTSSLQEGKNGKIFLLLVVKIPKQSNALDPNRVVGVDLGINIPLYAALNDNEYGG |
| | MSIGSREQFLKMRMRMVAQKRELQRNLRHSTNGGRGRSHKLQALERLEGKERNWV |
| | HLQNHIFSKNIIEFAVKNNAGVIQMERLTGFGHDRNDEVDDGFKFILRYWSFFELQSMI |
| | EYKAEAAGIEVRYIDPYHTSQTCSFCGHYEKGQRIDQATFVCKNPECEKGKGKKRSD |
| | GTYTGINADWNAARNIALSDKFVDKKKK |
| 46 | MANDEICITRKIEVHLHLHGDDEEGRARRKKDFETWNTINDNLFKVANLIATHQFFND |
| | AYEFRAKIHSPQYTKIEKDLNNSQKLKLTQEQVRELEKEKSKLDKEIEEQRKTFLQCSR |
| | QNSTFRVASKLFLDVIPSNVLTCLNQKICSTYTSYKSEVESGKRTLPNFKKGLPVPFQM |
| | NLNKKLQLRRRTDGSIFVLFPKGLEWDLFFGKDKSNNREIVNRVLSGEYGVGESSIQQ |
| | NKKGKTFLLIVVKIPKKAICLDSKRVVGVDLGINVPLCAALNDNESAKMYIGSREQFM |
| | KVREQLYVRKRELQRSLTTSTRGGRGRKQKLQALERHEGKERNWTHLQNHIFSKSVI |
| | EFAQKQNAGVIQMENLSGFGRDKNDEVDEGYKYILRYWSYFELQQMIEYKAKASNIE |
| | VRYVDPKYTSQTCSYCGHYEKGQRISQSTFVCKNPECEKGKGKKTKDGKYEGINAD |
| | WNAARNIALSGKVEERKKKKKRKIQSNQ |
| 47 | MNTINNTYIRTLKFNLNLTPKFETEEDNKKYINDVYSYLRDAMWAQNRAMNIVLDRT |
| | KEAYTLGRGMNRVKEIYYSYSHQKPISNDKKESFLESLLQYAPIDDQFVKNEVKKLRK |
| | FYESKKKPPKEETVSKNCEALKNKYIKYVGKSKDDIKRELDLLENYCAYPEDIYEKFA |
| | NGLSTPAYIKQKVESYWKQDGIKTKVIYSMDENLRRIKDAPLFIPPNIFYNKKDELIGL |
| | VYDYTDYISFLEDLENKRNVNIYLSIPYKKGEDKLKFKLVLGNPHKSRDSRLSIKRIFE |
| | EEYRIKGSSIGFTKNKETGKNTNLTLYLTVEVPQNKDNTLDENVVVGVDVGIAIPCVC |
| | ALNNDKYTRENIGSYDTLFAKRTQFKMQRSRLNSQLKLSKGGHGRKRKLKKLELLSG |
| | KEKNYADTECRKYASDVIKFCLKNHAKYINLEHLKGYRENPKVLAGWSFYKIQTYIE |
| | QAAEKHGIIVRKINPCYTSQICSVCGNWHPENRPKGKLGQAYFNCHNIDCKTHNTDLY |
| | KYGINADFNAARNIAMSTLFITDSDEITKKHWKEAREYYGIDESDDKEEKLNKVA |
| 48 | MIIARKLKITVIGSDEERKEKYRWIRDEQYNQYRGLNMGMTYLATGEILRMNESGLEI |
| | RLEKQKTELESKVEKAKLNIEKIKIKIEKIKTSKKINEEKILENQNKIDDEKANIIKYKNN |
| | IIKIEQALKVAKIKRMDIQMEFKEKYIDDLYQVLDKVPFQHLDNKSLITQRVKNDIKA |
| | DKTSGLLKGERSIRNYKRTFPLLTRGRDLKFYYDDKDIKIKWIEGIEFKVVLGNRIKNS |
| | LELRHTLNKVVNEEYKICDSSLQFDKNNNLILNLTLDIPENNKNEKVEGRVVGVDLG |
| | MKIPAYVVLNDVEYIKKSIGSIDDFLKVRTQMQSRRRKFQKQLQSANGGRGRNKKLQ |
| | SLSRFEEKEKKFAKTYNHFISSNIIKFAVDNKASQINLEFLSLKETQEKSVLRNWSYYQ |
| | LQQFIEYKAKREGIGIKYVDPYHTSQTCSVCGNYEEGQREIQEKFICKNPKCKCELNA |
| | DYNAARNIAKSTKYIKSKEESEFYKLNKKE |
| 49 | MTKVVKLALICEQNDKDNNPIDYKEIYKILFELQRQTREIKNKSIQYCWEYSNFSSDY |
| | YKLNHEYPKEKEILSYTLDGFVNDKFKNGNDLYSGNCSTTVRSACGEFKNSRSDFLK |
| | GTKSVINYKGNQPLDLHNKAIRFDCIGKEYYVYLKLLNRPAFKKHNYANTEIRFKVLL |
| | YDNSSKTIVERCIDKIYKISASKLIYNEKKKCWMLNLSYSFSNDSETELDKDKILGVDL |
| | GIHYPICASINGERKFFKIDGGEIEHTRRKIEARKKSLLKQGISCGEGRIGHGIKTRNKPV |
| | YDIEDKISRFRDTANHKYSRALINYAVNNNCGIIQMEDLTGITSDSNRFLKNWSYFDL |
| | QTKIEYKAQEVGIKVVYIDPHYTSQRCSKCGYISKDNRTEQALFWCQKCGYKTNADY |
| | NASQNIAIKDIDKIINAKK |
| 50 | MLKIKIECLNREVKNIITTRALKLTIVGDEETRNKQYKYIRNEQYEQYKALNLCMSLL |
| | NTHYVLNSYNTGAENKLKNQLEKLQNKIDKNNLELEKEDIKNSKKEKLTKQNMQFK |
| | GELIKLQEEYNKASKYRSDVDLAMKDMYIDDLYMAIQNQVTFKNKDFMSLVTQRAK |
| | KDFKNCLINGLARGERSLTNYKRNFPLMTRGERWLKFRYKEESDDILIDWIQGITFKVI |
| | LGSRKNENTTELRHTLHKVITGEYKICDSEMKFDKSNNLMLNLTMDIPVKENTNYIDG |
| | RVLGVDLGIKYPAYVCLSDDTYKRMAIGSAQDFIRVREQIRTRRFRLQEQLKMVKGG |
| | KGREKKLGALERIKDKERGFVKTYNHMVSKNIVEFAYKNKCEYIHVEDLNKNGFDN |
| | AILSKWSYYELKTMIEYKAERKGIKVRYVNPEYTSQKCSKCGHTDKENRQSQEKFKC |
| | LNCGFELNADHNASINIARSNDIKK |
| 51 | MADFVITRRIEVHLHHDPVADPEKVEYNRQWEYWRTINNNLYLAANRISSHLFFTDE |
| | YEHRLRIQHPRYRDIERTLASVSKVKRMSKEEIAALRVERRTIEQELRAQTAAFLQTSR |
| | QNVTYRIASDEFGDLIPSDVLTNLNQNITSTYNEFKKQVVRGERTLSNYKKGIPVPFSM |
| | KKGEGLRRRDDGSYYVLFPGGLEWDLAFGRDRSNNRAIVERAFNGDYEVGNSSLQE |
| | KNRKVFLLLVVKIPAQELALDPNRIVGVDLGLNIPLYAALNDNEYGGLAIGSREQFMK |
| | VRERMSARRRELQRALRHSTQGGRGREHKLQALERFQAKERNWVHTQNHIFSRAVV |
| | EYAKQNSAGVIQMESLKSFGRDKEDHIEAGFRYVVRYWSYFELQTLIAEKAKREGIEV |
| | RMIDPYHTSQTCSFCGHYEKGQRISQGVFICKNPECAKGKGRQLKDGTYSGINADWN |
| | AARNIALSTEVVKK |
| 52 | MITARKIQVTIVSNNRDEDYRFIRNEMREQNKALNVGMNHLYFNYIARQKLRLADLA |
| | YQEKESKLVSQIDKIYDDIKKAKTDEKREQLKEKLEKQKKKLEKMRKQKNNDLFQQ |
| | YQQIIGTSEQTSVRDAISEQFNLMSDTKDRLSQKVTQDFKNDIKAGLLTGERVLRTYK |
| | KDNPLYIRGRSLNLCKEEDTFYFKWIKGIVFQCVLGIKGQNKTELYKTLERVLDGNYK |
| | ICDSALQFNKNNKLILILTLDIPDAVRESKIEGRVVGVDLGLKIPAYCSLNDDKYPRLAI |
| | GDIKDFLKVRTSLQRQYRSLQRALKSSKGGKGRYKKLKALDRFREKEKNYVTTYNHF |
| | LSREIVKFARKYRAEQINLELLSMAESTNKSVLRNWSYYQLQQFIEYKAAREGIKVKY |
| | VDPYRTSKTCSECGHFEEGQRTDQAHFTCKQCGFEANADYNAARNVAKSTKFITTKE |
| | QSEYYQKDSVS |
| 53 | MNKVIKIYASLDKAQQCALPIYGKDSLSVQLLKVQSEVRSLKNRAMRMSYDYDQAQ |
| | YEYFKYMERCKQEYGLNSYPSAQPSDFSKYKTFDGYLYDALAKDYPLMNKRISATVT |
| | RKVWGEYKKDKGEILSGKKSLRTYREGQPIPIRAKDTKLLYEDNFDYTMTVSVFSKD |
| | AAKTLGMKPGGCRFILHEQTDSEKAILDRLLSGEYKLCETLLAYDDRKNHDTKMARG |
| | WYFCIGYSFEKDTSNPSLDKDKILGVDIGVANVIYLGWSKDDHFKKYIPGSEIRKFQAT |
| | EERRKKDILRCSVARGDGNVGHGRKCATRKAEKHEHHIHNFKETKNWHYAHFVVDT |
| | AVENGFGTIQMEDLSGINKSETFDRTWTFYSLQQKIEQLAAENGIVVKKVKPQYTSQ |
| | MCSKCGYISSRSRRSQSEFRCVSCDYRRNADHNAAMNISKSKIEALVEEQLLRQGGEA |
| | D |
| 54 | MYVEFIRIKGGEILITARKIKLTIAENREEGYSFIRKELQEQNKALNMAINHLYFNYVA |
| | REKIKLADETYKVKLEEGECYLERKYIELKEAKTDKQKENIKKSIEATKKKLETLRKV |
| | ENKEVSNNFKEIIATSEQINLRDLISNNFNLKSDTKDKLTQKVVQDFYNDIVGVLRGER |
| | TLRRYKKDNPLYIRGRSLTLYREGEDYYIKWMNGIVFKCVLGVKKQNSLELQKTLDK |
| | VIFEEYKLCDSSIGFKDNKLILNLTLDIPVSNTNKFEKVIGRIVGVDLGMKIPAYCALND |
| | SEDVRKAIGSIDDFLKVRTQMRSRRRKLQRALKSTNGGQGKNKKLSALNSFEAKEKN |
| | FAKTYNHFLSSNIIKFATDNKAEQINMEFLSLSETQNKSVLSNWSYYQLQQMIEYKAE |
| | RIGIKVKYVDPYLTSQTCSECGHYEDGQREVQSEFQCKKCGCKTNADYNASRNIAKS |
| | DKYITKKEESEYYKNKI |
| 55 | MRDKLKASFAGEKLKKIKNAQISSPSATSDRFPIPMWQQTGFRVETNNEDLVIDIPFPL |
| | YKYREEVDKFKPWEKLEFIDTSKKKHIQLILSTNSRKRNVGWVKDWSTEAEIKRVMS |
| | KKLVINKIEITRGKRINERNYWFVNFVIAYEKPVRKLDSKITGGIDVGVSNPVVCAINS |
| | GLNRLTIRDNDVVDFTRAELARLRSQRRGDRFRRGGHGIKHKFKPSETIQKKYEQRRK |
| | KKMEEWASRITRFFLNNGVGLVYMENIDKKSISDGEDYFKVQLRVTWPVKEMQKLF |
| | ERKLKENGIEVKSIDPKYSSQLCSKCGKWNTHFNFRYRQLNGYPPFECKYCDYGKDK |
| | EKEIIYADYNAARNLANPNFDKRRKVAIPSEVLKGNIKEEVVAEN |
| 56 | MITVRKLKLTIVGDEETRNQQYKLIRDEQYQQYRALNLCMSLLSTYNILNNWNSGAE |
| | NKLNSQIEKLNKKVEKNKNDLKKDNLKENRIKKINESIQTLTKEKEKLQQEYLSSSEY |
| | RSDIDKKVKEMYIDDLYTVVQSQVNFKSKDMMSLVTQRAKKDFTTALKNGMAKGE |
| | RSLTNYKRDFPLMTRGERWLKFEYDEDSDDIYINWLHGIRFKVVLGYKKNENSIELRH |
| | TLHKVINKEYKICDSSMQFDRNNNLILNLTLDIPLNIKNEHIEGRTLGVDLGIKYPAYV |
| | CLSDDTYKRKSIGCAEDFIKFREQIRARRYRLQKQLSMVKGGKGRNKKLQALDRIKD |
| | KERNFVKTYNHMISKNVVEFAKNHKCESINLEKLTKYGFPNMILSKWSYYELQNMIE |
| | YKAEREGISVKYVDPAYTSQTCSKCGHVDKENRTSQEKFKCIECGFELNADHNAAINI |
| | ARSNNYVK |
| 57 | MPQGKKLVTAQIKLYLHRPVEPDNISWAQAGQILRDVSYETVLGLNHAITEWHLYER |
| | ERARHYRETKENLPAEERKKTWNRIYSEVRGLMKTASSGMASMVTRTAITRYQNSLK |
| | DIRSLKQSVPSYRLGHPILLREGGGETKLWRDNGNYMFRATLRNRSNEPTRLTFLLDT |
| | FKLEKSKKAVLDRVISGEYKLGACQVAQDRRKRWFTRIAYSFPRPELKKDTSICVGVD |
| | LGLACPFYCAVNNGHDRLSCNEALIVERFRWQIRRRRRAFQNSLKFSNRGGHGRNKA |
| | LAPLEKLAEKEINFRDTKYHQYTSRIIEFALKQNAGVIQIEGLEGFRASQQGILRDWAI |
| | ADFHSKLKYKAEHAGIEVREIDARYTSQRCSECGNINEANRQSQSDFLCTVCGYKTHA |
| | DYNAARNISIVGIEKIIAEEIARKGLAEQEAQDVPQG |
| 58 | MVKRGEHMNTVRKIKIIINNENNELRKKQYKFIRDSQYAQYQGLNRCMGYLMSGFY |
| | VNNMEIKSEEFKTWQKGVTNSANFFQEISFGKGIDSKSSITQKVKKDFSTALKNGLAK |
| | GERNINNYKRTFPLMTRGRDLKFKYDDNELDILINWVNKIQFKCVLGEHKNSLELQH |
| | TLHKVINNEYKIGQSSLYFNKKNELILILTIDIPTAKSSYEPIKDRILGVDLGMAVPVYM |
| | SINDNSYIKKSLGSYSEFAKVRKQFKERRNRLYKQLEACKGGRGRKDKLKAMNQFKE |
| | KEKNFAKTYNHFLSKNVVEFALKNKCEFIHLEKIESKGLENSVLENWTYYDLQEKIIY |
| | KAKREGIEIKFVNSSYTSQTCSKCNYVDKENRKTQVKFICKNCGFKANADYNASQNIS |
| | KSKEFIK |
| 59 | MITVRKLKLTIINDDETKRNEQYKFIRDSQYAQYQGLNLAMSVLTNAYLSANRDIKSD |
| | LFKETQKNLKNSSSIFNDIPFGKGIDSKSSITQKVKQDFSIAIKNGLAGGERNITNYKRT |
| | FPLMTRGRDLKFSYKDDCSDEIIIKWVNKIVFKVVIGRKDKNYLELMHTLNKVINGEY |
| | KVGQSSIYFDKSNKLILNLTLYIPEKKDDNSIKGRTLGVDLGIKYPAYVCLNDDTFIRQ |
| | HIGESLELSKQREQFRNRRKRLQQQLKNVKGGKGREKKLAALDKVAVCERNFVKTY |
| | NHTISKRIIDFAKKNKCEFINLEQLTKDGFDNIILSNWSYYELQNMIKYKADREGIKVR |
| | YVNPAYTSQKCSKCGYIDKENRPTQEKFKCIKCGFELNADHNAAINISRLEE |
| 60 | MNTVRKIKIIINNGDDEIRKSQYQFIRNAMYAQYQGLNRCMGYLMSGYYANNMDIKS |
| | QGFKDHQKTITNSLYIFNDIEFGKGIDSKSSITQKVKKDFSTALKNGLAKGERTVTNYK |
| | RSFPLMTRGRDLKFSYGDNDEILINWVNKIQFKAITGNSKNSIELEHTLHKIINGDYKV |
| | GQSSLTFNKKNELILILTIAIPEVKGDEYKPVANRTLGVDLGLAFPVYMALNDITYIRK |
| | SLGSYNEFAKQKLQYKARRERLYKQLDSVKGGKGRKDKLKALDQFKEKEKNFAKTY |
| | NHFLSKKIVLFAIKNQCEYINLEKIDSSGLENRVLGLWTYYDLQKDIEYKAKLAGIKV |
| | RYVKAAYTSQRCSRCGDIEKENRQEQSKFVCKKCGLDINADYNASINIAQSTEFIK |
| 61 | MGNINIGGENMKEFRTVKKLKLTIVADTKEEREEKYKFIRDSQYAQYQGLNLAMGIL |
| | VSGFLKGNRKLDSEAFKQAQKEMMAIREQTFEDINFGKGITSSSLITQKVKADFKTAL |
| | KNGLAKGERNVTNYKRTFPLMIKGNADSCYDQGKRKPLDFYYDNDDIYIRWCNGIIF |
| | KVVLGTRINENTTELKHTLHKIIKGEYRVSQSSLQFDKNNNLIMNVNIRFKKELNTDFI |
| | EGRTLGVDLGLKYPAYVCLSDNTYIRKGLGSAEEFLKTRQQMKKRRTTLQHQLKLV |
| | KGGKGRNKKLKALEQFQNKERNFAKTYNHQLSCNIVKFAKENKCEFINLEKLTKEGF |
| | DNNILSSWSYYELQNMIEYKAERENIKVRYINPAYTSQKCSKCGYIDKENRKTQSEFN |
| | CLECGLKLNADHNAAINIANSTEYIK |
| 62 | MITVRKVKLIVNSEEAEEINRTYKFIRDSMYAQYQGLNRCMGYLLSGYYANGMDIKS |
| | DGFKNHMKTIKNSLNIFDDINFGIGIDSKSAITQKVKKDFSTSLKNGLAKGERGATNYK |
| | RNFPLMTRGRDVKISYLEDTNTFVIKWVNKIEFKVILGQKDNIELSHTLHKIINKEYTL |
| | GQCTFEFDKNNKLLLALNINIPDNLISKNKEIIPGRVLGVDLGVKVPAMICLNDNTFIK |
| | KSIGSYNEFFKVRSQFKARRERLYKQLESSNGGKGRKHKLKATMQFRDKEKNFARTY |
| | NHFLSKNIIEFAQKYTCETINLEELNKKGFDNNLLGKWGYYQLQSMIEYKAERVGIKV |
| | KYVDPAFTSQTCSKCGYVDEENRITQDKFECQKCGFTLNADHNAAINIARK |
| 63 | MPNITRKYQLKVVGDKEEIDRVYKYIREGTEAQNKALNEAMSALYAANLLDMSKDD |
| | KKELSKLFSRVTNGKNESGFTDDICFATGLGTTSSIKQKVKQDFNNACKKGLMYGRV |
| | SLPSYKADNPLLVSKSYVQLLSESDKNFGIYNTYETPMDLVDALEKETNPEVYLKFAN |
| | NILFKFVFGNPWKGREQRKVFERIFSGEYKICGSSIGIDGKKIILNLCMDIPKQKHNLDE |
| | SIIVGVDLGLAIPAMCALNNDDYKRLSIGSIDDLLRVRIQLQNERRRIQGNLKNSKGGH |
| | GRQKKLKALENLKDRERNFVQTYNHMVSKRVVDFAVKNNARYINIEDLSGFWKTRY |
| | GKSKSEDEKVLRNWSYYELQNYITYKAQLHGITVRKVRAEYTSQTCSYCGNKGIRKE |
| | QKKFVCVNPDCKCHKIYDGYINADFNAARNIAMSNDFAE |
| 64 | MKLVKTMRYQIIKPLSCDWDTLGTVLRELQRDTHSVLNKTIQLCWEWQGYSSEYKA |
| | ANGTYPTPKDTLGRSLEGYVYDRLKVQFPKMYTPNLSQTIQRAMLKWSADSKAIFKG |
| | EVSIPSYKKDVPLDLRKDSIHIERRGHDYILSLGLVSRAYKKELGLPECQIEVLIGTPDK |
| | TQRVILARLLTGEYTVSGSQIVWDKRNRKWFVNLAYHFEARPEQLDKTKILGVDLGV |
| | VFPVYMAVADGHFRAGIPGGEIEEFRRRVEARRRQLLRQGKYCGDGRIGHGRATRTR |
| | PLDKIADKIARFRDTINHKYSRYVVETARKLGCGVIQMEDLTGIREENLFLANWPYHD |
| | LQRKIEYKAREYGIEVRYVRPQYTSQRCSDCGYIHPDNRPEQAKFRCLACGFETNADY |
| | NAARNIATEGIEELIAAALNKASVV |
| 65 | MVKTMKFQIIKPINMLWKDFENILRQLQQDTRNIKNKTIQLCWEYQGFSSEYKEKHGE |
| | YPKHSDILNYKTITGYIYDRLNNEYYRLNTGNLSDTIKSATDKWRNDIVDILKGEVSIP |
| | SYKRDAIIHVKNTNYKIVPEDGRYYLRLSLLSNVYRKELDLEKGQIDVAIRVADKNQK |
| | VTLERIFSGEYKQSSSQLMKKKNKWFFYMAFKFDPKTAKDLDPNNVMGIDMGITHPI |
| | YFAFNNSLKRGKIEGGEVESFRRRVEQRRRELLAQGKYCGKGRRGRGYETRVKPIKRI |
| | GDKIERFKDTANHKYSRYIVDVAVKNNCGIIQMEDLKGISKNNIFLKNWPYFDLQTKI |
| | EYKAKEKGIVVKKIAPRYTSQRCSKCGYINKENRVSQDTFKCVKCDFGHKFYVNADY |
| | NAARNIATPGIEEIIKKQIEKQKEMDEWENETIKLPLVGSDK |
| 66 | MFMTKVTKVYLISEQIDKDGNKIDFKKISELLWNLQRQTRDIKNKCVQLCWEWLNFS |
| | SDYYKKSEEYPKEKDTLGYTLSGFVYDRIKNGSDLYSSNLSTSSRDTCTAFSNYKKEM |
| | LNGERSVLSFKANQPLDIHNKAIKLSYENGNFFVALKMLNRAGKEKYGINDDLRFRM |
| | QVRDKSVRTILERLMNDEYKVSASKLMYDKKKKLWKLNLCYSFDNHVISTLDPEKIM |
| | GVDLGVVYPIMASVNGDYARFSIKGGEIEAFRNRVEARRRSLLNQSRYCGDGRIGHG |
| | RKKRTEPAAQIADKIARFRDTTNHKYSRALIDYAIKNGCGTIQMEKLTGITSNAEHFLK |
| | EWSYFDLQTKIESKAKEAGIKVVYINPKFTSQRCNKCGYIHTDNRPVQARFCCQKCGY |
| | EENADYNASQNIGTKHIDVIIEETLKMQCEPEVPTE |
| 67 | MKPMNKVVRLALICEHSDKDGNPVDYSDVYKLLWQLQAQTREIKNKTIQYCWEYSN |
| | FSSDYYKENHEYPKEKDVLHYALDGFVNDKFKVGNDLYSSNCSYMTRKVCAEFKKS |
| | KSDFLKGTRSIISYKSNQPLDLHNKSIRIEYKDNDFFAFLKLLKRPAFNRLGYKNSEIGF |
| | KVIVRDKSTRTILERCVDQIYGISASKLIYNKKKKQWFLNLVYAFEPDNANNLDPSKIL |
| | GVDLGIHYPICASVYGDLQRFTIHGGEIEEFRRRVESRKLSLLKQGKNCGDGRIGHGG |
| | KTRNKPVYSIEDRIARFRDTVNHKYSRALIDYAVKKECGTIQMEDLSGITAESDRFLK |
| | NWSYYDLQSKIEYKAKEKGIKIVYIDPKYSSQRCSKCGHIDKENRKTQSSFVCLKCGF |
| | EENADYNASQNIGIKDIDKIIENDLSSKCETDVN |
| 68 | MKITKTMKYEIDKSIDVPWKTFLSVLRDVQYAVWKTGNIAVKMTWDFQQEAWSYR |
| | QRFGEQLKFSDLGTGNKSQSTDIYQRACSEYPNVASSVLDATIRMAQDRYKTDAPDIY |
| | DGLKTIPYFKRELPIPIRAQQTKLTRKGTKRYVSFALLSKEGAKKSELPTRYNVQIRTG |
| | KGAREIFDRLVDGEYKLCDSKILRKKGKWYLALSYSFEAENPQELDPKRVMGVDLGI |
| | VKTAYMAFNFDEYLRYEIEGGEISAFRGRIESRRKSLLKQARYCGEGRRGHGRKTRM |
| | KPLEKLRDKVANFRRTKNHHYSKYIVEMAAKHGCGTIQMEDLSGINKRDKFLANWS |
| | YYELQSFVKYKAKERGIKVVLVDPNYTSQRCSCCGYIAEGNRKTQETFKCVICGYKT |
| | NADFNAARNIAEPRIKALIDAELKRQEKERKEAM |
| 69 | MSKVMKYELKYLGEEDFYEMQKMLWSLQEDTREILNKTIQIFFHWDYTNKESLETTG |
| | KALNLVEETGYKDISGYVYDKLKSRYPDMSRGNLSATIRAASKKYRSSKVDILKGTM |
| | SIPSYKKDQPIILRPDGIRLHEREMIYTVELSLFSGDFKKKKAWKSNVLFQIKACDKAQ |
| | KAIMQRLLSREYKLGESKLVYKKKKWFIYITYSFKKTDAKLDKNKILGVDLGVTYAI |
| | YACSIGEYGSFSIKGEEALEYAKRLEARTISKQKQARYCGEGRIGHGIKTRLSTVYSTR |
| | NKLANHRDTLNHRYSKAVVDYAVKNRYGTIQMENLSCIKKNTGFPKRLQHWTYYDL |
| | QSKIEYKAAEQGIQVIKINPKFTSLRCSQCGCIHKDNRKTQESFQCVECGYKDNADHN |
| | AALNISIPQIDLIIKEEMTSAKEK |
| 70 | MPIKALRVQIIKPFNTDYDSQPITWDELGRTLRDLRYAASKMANYVIQQNYMWEFFR |
| | QQYKQEHGSYPSVSEHKDKLYCYPRLTAMFPLAAGQMVNQIERHAKTVWSARKSEV |
| | LKLHQSVPSFKLNFPIIVHHDSYRISEVPEDGKSSTHVFLLQANLLSREAATRTRYSFLI |
| | NAGEKSKQTIVERIISGEYRQGALQIVGDRKNKWYCIIPYEFKTEENNTLDPQCIMGID |
| | LGISKAVYWAIIGSHKRGWIDGHEIEEFRRRVQARRKSIQEQGKYCGDGRIGHGRKRR |
| | LLPIEVLENRESNFKNTTNHRYSRFIIEAAIKNQCGVIQMEDLSGINERSTFLRNWTYY |
| | DLQMKIKAKAEEVGIEVRIVNPQYTSQRCSQCGHIDRDNRSNQATFVCTHCGYGGLY |
| | HCFACGKSQVEAGVCHLCGGETKIMKINADYNAARNLAICGIDQIIVQTLEGEGVR |
| 71 | MTQHIRVMQYQIVKPCNGDWSTLAKVLSYLQNATRQVLNKTIQLCWEWQGFGSDY |
| | KRKFGENAVDRELLGYSLFGFCYHQLRQEFPLIHSSNISQSIQRAVLRWNSDAPEILNG |
| | VKSVPCYKRGVGVDLHKDTVRLKRGKNNEFVVSMNLLSLIGRKEFGFKSAALDTVIK |
| | VADGRQRQIFSRVLSGEYGLGACQIVCHKRKWFLQMRYKLAVKERQLNSNHILAVR |
| | MGIERPLYIAFSHTSSRVTLNGDNVTAFRKKFMARYAGMIRQKTMQGGGNIGRGKAK |
| | RFERIESMRTKISGFQKSLNHKYSRIIVEQAVLNRCGTIQIESPNVIRRQTAFLGNWAYF |
| | DLKRKVEYKAQQLGIKVVCLKTRDYEQRCSQCGHLNAVRETSKVLSGAYARSFFCES |
| | CGLLTTVDENAVQNLTLPNKPVMSQD |
| 72 | MALVKSVRIQISRCEELDYKRMSSIFRDIRYKTCRASNEAMRLFLLNAFQSIRYKELHD |
| | IYPDIKSLTGKSLNTYIYNSMKEIMDICQTGNVAQTQQFVKNRFNTDIKDLLVNEVSYT |
| | NFKKDMPLFLHNKSYTICKDEDSGKYKVECSLFNRQYQKENNIKRVTFVLGKMDGN |
| | QKATLDKIIAGEYKQGAGHIKQDKKKKWYFTISYSFEPQIRKINTNRILGVDLGMVNT |
| | AVLQIWDIDKQKWDWLEWKECMLDGGKVYNFNQRVEGMKRSLQRSRKVSSVTSPY |
| | SKGKKGRGVQARIKALNRLNHKISNFKDTINHQYSKYIIDFALKHNCGIIQMEDLSKIK |
| | DKAEEKFLAHWTYYDLQSKIGYKAKAHGIKVVKIKPAYTSLRCSKCGHIDKENRKEQ |
| | AKFKCVKCKYKLHADVNAARNIAIPEIERLIEIEKEEVNAI |
| 73 | MTKNKMTTKTMKYEIRFEKALYNLLSDIQFEVFMLKNKATSIAYDWQNFSFSYHSRF |
| | GEYPKIKELSGVTLTNDILGELREVQAKFVSSATVASSVKEAVEKFTSDKSKILKGEISI |
| | MRYKRDGSFPIRSQQINHLTKINSKTYTCKLSLLSREGAKEREMKNGQMDVELRTGK |
| | GAYEILDRIIEGSYKLCDSRITKNKNKFYLLITYSFESDKVETLDENRIMGIDLGINIPAM |
| | LAISDNKYYREAVGDVTEISNFQKQVESRKRKLQKQRKWCGEGSIGHGTKTRIKPLEV |
| | LSGKIKRFKDTKNHNWSRYIVDQAVKHNCGIIQMEDLSGIAEENTFLKTWTYYDLQQ |
| | KIKYKAEEKGINVVFIKPNYTSQRCSCCGHISKENRDVEKNGQDKFICVNCGYGSKFY |
| | VNADWNAAKNIATKDIENIIKEQLESQEKELKHNMKYAM |
| 74 | MDVGTVVKMTRCRIEQDDELYKVLDDLRYMIYRIKNKATSMAWDWEQFSFGYHER |
| | FGEYPVAKEVVGKNVSRDAYQHVKGLGEEFSSSFVDTAVEEASKHFKNHRSAILRGK |
| | EGIPVYRSDTSFTIRHTQIKGLKKEQRDKYSGLFTLLSPKGSKVREQASTRYLFKIRSGG |
| | SSSAILDRILEGTYSLCDSKIVYEKKKRHYFLLVTYKFEAQKIEMNPERVMGVDVGFA |
| | VPATIAITDNPYACHFVGDAREVLAFEQRVLGTRRALYRSRSRAGDGSRGHGRKTLM |
| | KPTEVINSKVANFKATKNHEWSKFIVDYAVRHGVSTIQLENLEKIAEESPFLKRWTYY |
| | DLQQKIAYKAKEYGIKVIRVSPDYTSARCNKCGAIHRKQERTLWRPKQSQFNCLHCH |
| | HRDHADRNAARNLAIPNIDQIIKAEKVEWTSYWTNVYKNPPA |
| 75 | MESEKEKYVVQSYGIKLIKPVGVDEGDWDFAGKVLRDLDYACFRVKNKAATRTYM |
| | NVIEKLEYENIHGKGTYDKYFKTRYGKTFSAYNSECAREDKELNKGDLYREYFNLMA |
| | REGEKVVKYNMKKILNGNASNITFKRNQPVPITSRMIFITKESGKYYAELTLLSPEKAK |
| | ELGRKGKNGTRIKFLLSSKGQEKVILDRLTSGEYDLRDSHIHVKKKGNKLTNYLIIAYR |
| | HKVKDDNDLIPNKVLGVDLGVSKAAYMAVSESPVSEYINGGEIEQFRNGVEARRNG |
| | MRNQLKYHSSNRSGHGRATKLKPLEKLREKVSNFRKLTNHRYAKFIVDTALKNNCSII |
| | QMEELKGISKNDTFLKRWSYFDLQEKIENKAIAVGIEVKKVSPKFTSQRCNKCGYIDK |
| | ESRKSQEKFECVNCGHKTNADLNAARNLSMLNVEKEIKAQCKAQKIKY |
| 76 | MLQVTKAVRFQIIKPLNFSWDEFGRILNDLSYHTTLMCNAAVQMYWEHNVMRNRYK |
| | AEHGRYPQDKEIYGQSFRNVVYHRLREMYPLMASSNVSQTNQFALKRWQTDLREVM |
| | RLQKSVPSFRLGTPVQVANQNYSLYIAKGEPPEYCAEITLLGKDAACRRFTVLLDAGD |
| | APKKAVFRRIVEGKYKQGVMQIIKHPRKKKWFCIVSYTITKDPAPGLDQERVMGVNL |
| | ATGEAVYWAFSFSPKRGSIPAGEIEAAEKKIRAITARRREMQRTAGVTGHGRKRRLKA |
| | TRVLAGKTANIRDAINHKYSRRIVRIAAANRCGKIRLADMSALGMSGALKAWPWSDL |
| | VQKIGYKAAEQGIDVEIVEKPGDRAKAWHTCSECGYSAPENVGDNTEFLACKECGAR |
| | ISLEYNAALNIAVLARDSIPEQQTSAS |
| 77 | MEITKATRYQLIKPLDVSWEDFGQILRDLSYHTTKMCNAAVQLYWEYHNQRLAYKQ |
| | EHGKYPEDKVMYGMSFRNVVYHRLRGIYPLMASSNTSQTNQFALNRWKNDVPDVM |
| | RLQKSIPSFRLGAPIQVANANYRLYVAEGEKPEFRADVTLLGKDAAQGRFSLLLDGGD |
| | APKKAIFRHIVDGTYKQGVMQIVRHPRKKKWFCIISFTFTSEEKSLDESRAMGVNFGS |
| | GEALCWAFNFGPKRGTIPAAEIEAAEAKIAAITARRRGMLRTAEARGHGRTRRLKPTE |
| | SLQGQAANIRDAINHKYSRKIVSVAVGNRCGIIRLADTSGLELDGAFKHWPWAGLAE |
| | KIRYKAEEAGIVVETADAKKAFYTCSKCGYSHPENTDGNKEFLTCKNPDCEAQVNLQ |
| | YNTAKNIAVNVPGPEEKKSPKKEKRKKEIAKQ |
| 78 | MTKAKDRELSKVFRVEVLKPLNLTWDEFGGLLRRTQYHAAHLANEVITTQYLLAKG |
| | KLERNGSFCALVAHACHDCSLHADVKCTVCKWARDKFKADARRILRADISLPSYKN |
| | NLCMIKNRSVKLRETPDGWAARLAILPKADGKNQVQPEVLLRTEEMKRRSPGAYQV |
| | LERIASKEYKQGTTQVKRDTRTGKIYLLISYSFRPERESGLEASRVMGVDLGVSTPAY |
| | CAFNDSLKRKSLLIEGRKLLKTKWQIEGRRRDIRRHNDQRDLRRGHGKEAKFRPMEA |
| | VEQHWVDFRQSWNHVLARRIIEYALSNHAGAIHLEDLSPGTNSKFLGRNWPVAELLD |
| | YIEYKAKERGIAVKRVNPFKTSQTCSDCGAIKESFTFGDRKKAGFPDFVCDACGFRTH |
| | ADYNAARNIAKAP |
| 79 | MNKCIKVILNKCINIDIKEAKKIIKNMSYLSCKASNKAIDMWKQHSLNIMELKSNDKN |
| | FNQKEYEQATYGKNYKNVIEGYMKEIMNICNTSNVSTLHQQQVQNDWKRLRKDVL |
| | NYRANLPTYKLDTPCYLKNNNYKLRNHNGYFVDISLFSMKGLEQIGQKKGYQLQFEI |
| | DKMDGNKKSTINKLINNGYKQGSAQLKISDKGKIELIMSFSFEAKESNLDENRILGIDL |
| | GIVNVATMAIWDGNTQEWDWVNYKHNILNGQELIRFRQKLFNMGMSEFEMQNEVY |
| | KQNQKIHQKQLNKHNIGAIDGLELVKYRDTIDKKKREMSIASKWVGEGRVGHGYKN |
| | RMKPLEKIRNKASNFADTFNHKYSKYIVEFAIRGNCGVIQMEDLSGATKNTHGKFLK |
| | DWSYYDLQTKIEYKAREVGIDVIYVKPQYTSKRCSKCGNIHTDNRDCKTNQAKFKCM |
| | NVTCGHEENADINASKNISIPYIDKIIEEYIKENDIYKNKK |
| 80 | MIQNRRQNRLILTRKIQIIPLGEKEEIDRVYKYLRDGIFYQNKAMNQYMSALYIAAIKD |
| | ISKEDRKELNRLYSRVSNSKKGSAYDKSIEFAKNMNLGYVVKQVKQDFANSCKNGLL |
| | CGKVSLPTYRKNNPLLVHVNFVRLRSTNYHQDNGMYHNYESHTDFLDHLYSKDLEV |
| | FIKFANNITFKMIFGNPHKSAYLRSEIQQIFEENYKVCGSSIQIDGKKIILNLSMDIPKQE |
| | LELDENIVVGVDLGLAIPAMCGLNTNDYIRQSIGSKDDFLRIRTQLQSQRRRLQKSLAS |
| | TSGGHGRQKKLKPLEKLKDRERNFVKTYNHYVSKNVVDFAVKNKAKYINVEDLSGF |
| | DSNQFILRNWSFYELQQFITYKAAKYGIEVRKINPYHTSQICSCCGHWEEGQRIDQAHF |
| | KCKSCGAELNADFNASRNIAMSTDFV |
| 81 | MATKEKRIVKVMRLRILKPAGEMSWSQLGKLLRDTRYRVFRLANLAVSEAYLNFHL |
| | WRTGRSQEFKADDMGKLSRRLRQMLADEGVAADELDRFSPTGAVPDAVSGPLFQYK |
| | IRAITNKNKWREVIRGTASLPTFRLDMAIPVRCDKARMRRLERMENGDVQVELTICRK |
| | PYPRVVLQTGDIGGGQEAILARLLDNTGNDLTGYRQRVFEIKQERQTSKWWLYITYD |
| | LPAPQTGKADPDVVVGVDVGYAVPLYVAINNGHARLGWRQFDALGRRIRKLQTQVL |
| | ARRRSIQRGGRVNISHATARSGHGVKRKLLPTEILQRRIDKAYQTLNHQLSASVIDFAR |
| | DHGAGVIQVEDLEGLKEELTGTYIGARWRYHQLHQFLKYKAEENGIEFRAVNPRFTS |
| | RRCSKCGHINVAFDRAYRDAHRENGKTARFICPQCGFEADADYNAARNLATLDIEALI |
| | EAQCARQGLTKDAL |
| 82 | MITVRKLKLSIRESDEESRKVKYQFIRDSQYAQYRALNLAVGILSSAYLKSNKDTKQE |
| | SYKNAIKSLTNSNPIFSEIEFGRGVDTLSLVTQRAKKDFKNSIKNGLARGERNLTSYKR |
| | TYPLMTRGRDLKFRYEGDDIVIRWVNKIEFNVITGSNKIKENTVELKHTLHKVINKEY |
| | NVKESSLMFDKNNNLILNLTLDIPNELSYQPIEGRTLGVDLGIATPAYVCLSDDTYVRK |
| | GIGSIEDFLKVRTQFQKRRRVLNQSLVMAKGGKGRKKKLKALESFKEKERNFAKTYN |
| | HQLSHEIVKFAKNHKCESINLEKLTKEGFNNRILRNWSYYELQSMIEYKAEREGIKVR |
| | YVNPAYTSQKCSKCGHVDKANRQTQAQFKCVECDFELHADHNASINIARSEEFVQGA |
| | GS |
| 83 | MVKRGEHMNTVRKIKIIINNENNELRKEQYKFIRDSQYAQYQGLNRCMGYLMSGFYV |
| | NNMDIKSEEFKTWQKGVTNSANFFQEISFGKGIDSKSSITQKVKKDFSIALKNGLAKG |
| | ERNINNYKRIAPLMTRGRNLKFKYDDNELDILINWVNKIQFKCVLGEHKNSLELQHTL |
| | HKVINNEYKIGQSSLYFNKKNELILILTIDIPTAKSSYEPIKDRILGVDLGMAVPVYMSI |
| | NDNSYIKKSLGSYSEFAKVRKQFKERRNRLYKQLEACKGGRGRKDKLKAMNQFKEK |
| | EKNFAKTYNHFLSKNIVEFALKNKCEFIHLEKIESKGLENSVLANWTYYDLQEKIIYKA |
| | KREGIGIKFVNSSYTSQTCSKCNYVDKENRKTQAKFICKNCGFKANADYNASQNISKS |
| | KEFIK |
| 84 | MITVRKIKLTIMGDEETRNRQYKWIKDEQYNQYKALNIGMSYLATHLFLKMSESGLE |
| | QKTEKNIKTIEKQISKIENNITKEESKKKVNEEKLNNLINELDTLNASLSKLKEELEEISN |
| | NRSNVDDTFKRMYVDDLYNALSKVPFQHSDMKSLVSRKVKLDFNTDMKDLMSGNR |
| | SVRNYKRNHPLLVRGRDLRFRYDGSNIKIKWIQGIEFKAILGKISKTIELRHILNKVIDG |
| | EYKVCDSSLEFNKNNHLILNLAIDFPYTNKIEFIEGRVVGVDLGIAVPAYVALNDIAYV |
| | KKSIGDIDDFLRVKTQMKKRRNLQINLTSVKGGKGRSKKLKALDRLSEKESNFVRTY |
| | NHFLSKSIVKFAIDNKAGQINLELLSENALSDKIIKNWSYYQLQQFIKYKAERYGIKVK |
| | YVDPYRTSQTCSVCGHYEEGQRSKQDIFTCKNEKCKMFEKEVNADYNAARNIAISTK |
| | YIDDIKESEYYYKKSF |
| 85 | MSIPLTRKIKLSVYVPNTITEEKERHEYKTFVWQLLRKVNQQNYEFHNLLVKKLIELD |
| | SVLEGRLTEDDNFIEIRNRYYSDRKDKKNQREFFDYIRMKKDELIKEFGGKNLHGYIY |
| | TYLKNYIKSLPEEQQFMASYTYGSISKNVVDKYTNDQFDVFRGVKTIATYKNTQPIPIN |
| | IIGHVKKNEKGELYKNTGKEWFNKYDDIYTFKFKSIKKHEIELQLNFGKDRSNNRIIVD |
| | RIYNNDPNYKICDSKIQVVGTEIFLLLVFKQFVTKRELGLDVRKVIGVDLGVKNVATIA |
| | TNFSDHIEIIGEGEFVLMRTKLQIEKQKRSIQRNSKYSRGGHGRNRKLQKLNEYRNYE |
| | RNFRSTFNHKISKDVIEIAIKHGAGQINIEDLSSIPLKEKNNRILRFWSYYDLIQKIIYKA |
| | KREGIIVNLINPSYTSQKCHQCGQIGNRPKQDTFLCTNPTCKAFNEPINADVNAAKNIA |
| | KISL |
| 86 | MQRSITLKILRPRDEKISWEEMGYLLGGLSMKVCRMSNFCMTHHLLHALKLETELLN |
| | PRGDLYCYPVLAEEYPEVPSGIICAAETRARKLFKRSAAKVLRSETSLPSFRKDSSIPIP |
| | VAGYRILQDGDGNYCAEIQLISRQGAKTQKLPGRICLVLADNWRDKSAKSALQKVAA |
| | GKVRRGVATLFRAKKDWYLCIPYVTEPADIGENFEPGLVMGVAFGMFDVLAYGENT |
| | LLKRGAISGEEVLSHQDKFMARRKKIQEQYAWSGRKGQGREDALKPLRHLYEVEKN |
| | YRDLVNNRYAKWVVDIAVKNRCGEIHLDSGNSTSKGNKEILLSHWSLYDLKDKICRK |
| | AEEKGIRVTECNVPNLRTRCSHCGTEQAVENRKRMFLCKNCGYGTTDKNKSNGYISA |
| | DYNAARNLAVYDTGDTEPV |
| 87 | MIITRKIAITIVSEEAQESYNYLRQQIYYYYKALNFGMNHIYFNYVAKEKIKLADSAYK |
| | EREEKYINAIHIAKEKLQKDLSVSQRAQAEKSLEVNTNNLDKLRKAISKDAKETFQKV |
| | MGAVERTNVTDAIKKEFPMLQRDSIDFAASKVASDENNDLKLGLMTGSRTLRIYKRN |
| | QAYPFRSRRLKFYKENGDFFINSSKSLLFKCLLGVKRQNSKELIQILEKILDNQYKICDS |
| | SLEFNKKKLILNLCIEVDENTHSENMKVPGVVKGRIVGVDLGIQIPAYCTLNDSPFKKK |
| | AIGSVDDLLRIRTQMQARRRRLSKNLISARGGKGRGKKLKALDRFEEYERNYVRTYN |
| | HFISKQIIQFTLQNQAEQINLELLQMEHTKSKSILRNWSYYQLQQMIEYKAKREGIVIK |
| | YVDPYHTSQVCSKCGHFEENQRMDQNTFRCKKCKYRTNADYNAAKNIANSTRYISSI |
| | QESEYYNIKNRNIAK |
| 88 | MIITRKIAITIVSEEAQESYNYLRQQIYYYYKALNFGMNHIYFNYVAKEKIKLADSAYK |
| | EREEKYINAIHIAKEKLQKDLSVSQRAQAEKSLEVNTNNLDKLRKAISKDAKETFQKV |
| | MGAVERTNVTDAIKKEFPMLQRDSIDFAASKVASDENNDLKLGLMTGSRTLRIYKRN |
| | QAYPFRSRRLKFYKENGDFFINSSKSLLFKCLLGVKRQNSKELIQILEKILDNQYKICDS |
| | SLEFNKKKLILNLCIEVDENTHSENMKVPGVVKGRIVGVDLGIKIPAYCTLNDSPFKKK |
| | AIGSVDDLLRIRTQMQARRRRLSKNLISARGGKGRGKKLKALDRFEEYERNYVRTYN |
| | HFISKQIIQFTLQNQAEQINLELLQMEHTKSKSILRNWSYYQLQQMIEYKAKREGIVIK |
| | YVDPYHTSQVCSKCGHFEENQRMDQNTFRCKKCKYRTNADYNAAKNIANSTRYISSI |
| | QESEYYNIKNRNIAK |
| 89 | MNDRSIITRKLTLIPAFSDRPKWEEKVMSYTEQFYIDKIAYYKNKLTKTKGKEEKQKI |
| | KDKLASLEEQENEFEESGILTQANVTDYTYDLVRNAMASEANRKNAIISYIILELLHNG |
| | GQTMDFNARNKLINDLVNYGLRVKGSSKGSLFDELDIENPLNAYGFAFKQDLKKKIR |
| | DMVNSKRVLDGKSSVITYKADSPFSINKENMSFTHDYSSFEELSDHIRDNDTNLYFNF |
| | GSSGNPTIARFKINLGAGRHKKNKDELIATLLKLYSGEYQFCGSRIGIEKNKIILNLVLSI |
| | PKKVRALDENTVVGVNLGVAVPAMCALNNNEYERLAIGSADEFLRVRTKLQAQRRR |
| | LQKSLKDASGGHGRTKKLKALERVAKAESHFANTYCHMISKRIVDFALKNNAKYINL |
| | ENLTGYDTNDFILRNWSYYKMQQYTTYKAEKYGIIVRKVNPCYNAQACSVCGNYAP |
| | GQRKSRAVFICANPACKSHKKNHGKLDAEFNNARNVAMSTLYMNDGQVTEKSFKEA |
| | RDYFGIEEEIETI |
| 90 | MITARKVKLTITENREDGYNFIHNELREQNQALNMAMNHLYFNYVAREKIKLADETH |
| | KIKLAEDQGYLDQKYTELKEVKTDKKKQNIRKSIQAAKKRLETLRKAENKQVAEKFK |
| | EIIAASEKTNLRDFITDNFNLTSDTKDRLTQKVSADFKNDIVDVLRGERTLRRYKKGNP |
| | LYIRGRNLTFYIKDEEYYIKWMKSIVFKCVLGVKKQNSLELQKTLDKVIEGKYKVCDS |
| | SIEFKQNSLILNLTLNIPVCNSFDKVEGRVVGVDLGMKIPAYVTLNDSDYIRRAIGSIDD |
| | FLKVRTQMQSRRRNLQRALKSTKGGKGREKKLKALNQFEVKEKNFAKTYNNFISSNI |
| | VKFASDNKAKQINMEFLSLSETQNKSVLRNWSYYQLQQMIEYKANRVGIKVKYVDP |
| | YHTSQICSKCGHYEEGQREKQEVFICKNPECKNFNIEVNADYNASRNIAKSNKYITKK |
| | EESEYYKIN |
| 91 | MILTRKIKLVIVSENREEGYNLIRTEIREQHKALNLAYNHLYFEHNAIQKLKQNDEDY |
| | KQKRNKLQELINKKYEEHQKAKNLEKKEALREAYNNKKQELYNFEKEYNEKARQTY |
| | QQVVGFTQQTRVRNLINRECNLMSDTKDGITSKVTQDYKNDCKAGLLIGKRSLRNYK |
| | KDNPLLVRGRSLKFYKEDGDYFIKWNKGTIFKCILHIRKKNVVELQSVLENVLLGAYK |
| | VCDSSIGFNNKDMILNLSLNIPDKETQGYIPGRVVGVDLGLKIPAYLSLSDKVYVRKGI |
| | GSIDDFLRVRTQMQKRRRRLQKSLAAVKGGKGREKKLKALDHLKGKEANFAKTYN |
| | HFLSTQIVTFAVKNQAGQINMEFLEFDKMKNKSLLRNWSYYQLQIMVEYKAKREGIII |
| | KYVDAYLTSQTCSKCDHYEDGQREKQENFMCKNCGLEVNADYNASQNIAKSTSYIS |
| | DSTESEYHKKKQQVLKEILGENDIMNEQLSLFNNCDDIA |
| 92 | MSKITRKIKIIPDIDGITHEESNKKCYNTFYKFDRKLYKVANLLVSQLYGLDSLLSLMR |
| | LQNDEYVKCLSKLSFKSITDATKEEIKKRMKEIDAELISIKNDIAPKHPQTYSYRAVTSS |
| | EYAKDIPSDILNNLKQDVYQHFNENKKEQIRGERSLATYKKGMPIPFNLKKKHGIISVG |
| | DNYYLPWFEDTRFRLNFGRDRSNNRAIIDNCIKTKKYKLCAAAKIQLKERKLFLLITV |
| | DIPKAESVPVKGKVMGVDLGVVNPAYVAVNDGPERSRIGNGEAFQKQRDVFRRRFR |
| | ELQRSQLTQGGHGRKHKTKATEILRGKERNWVQTENHRISREIVNLASRWKVETIQM |
| | ESLKGFGKNQEGEVEYNHKRLLGRWSYFELQKDIEYKAAMAGIAVQYVNPAYTSQT |
| | CHVCGQRGNRIERDTFICTNPECTCYNQAQDADMNAAINIAKSKDVIK |
| 93 | MITVRKLKILIDGESRNESYKFIRDSMYAQYLALNKAMSYLGTAYLSRDKEIFKEAIKS |
| | LNNSNPIFDNINFGKGIDTKSSVNQTVKKHIQADIKNGLAKGERSIRNYKRDYPLMTR |
| | GRDLKFFYCDTNSTKVKVKWVNGIIFDVMLGKEYNKNDLELRSFLNRVINKEYKISQ |
| | SSICFDKHNRLILNLSVNITDNIPNEVVKGRIVGVDLGMKIPAYVTLNDSEYIGKPIGDI |
| | NDFLKVRKQFKERKERLQKQLAINKGGRGITNKMQLMDAFTNKEKNFANTYNHGVS |
| | KAIINFAKKYKAEQINVEFLALAGSEKEILSSTIRYWSYYQLQQMIEYKANREGIAVKY |
| | VDPYLTSQTCCKCGNYEVGQRINQELFECKLCGNKMNADRNASFNIARSTKYISSKEE |
| | SDFYKQLK |
| 94 | MQRSVTLKIIRPEDETISWEELGYLLRGLSFKVCRMCNFCMTHQLLHALKLETELLNP |
| | QGNLYCYPRLAEEYPDVPTGIICAAETRARKVFRRSAEAVLHSETSLPRFRKDSSIPVP |
| | VAGYKILQDSDHNVYADVQLLSRQGAKTQKLPGRIRLVLADNWRDQSAKAALRQIA |
| | DGKVKRGVASLFRVKNDWYFQIPYVTEAVNTGEGFEPDLVMGVAFGLQDALVYAFN |
| | TSLKRGAVSGEEVLAHQEKYAARRKKIQEQYNWSGRKGHGREDALKPLRHLYETER |
| | NYRSLVNSRYAKWVVDIAMKNRCGMIHLDSANYVSSGKKILLSRWPLYDLKEKIRR |
| | KAEEKGIQVTECSIPNLRTRCSLCGKEQEPEGEKHTFVCKDCGYGKADKNRRSGSITV |
| | DYNAARNLAAYKSEDTKL |
| 95 | MITVKLQLYKPTKCKEQRLFSIIREFTSCANWYLDKLQESRTTSRVKIHNGYYETARK |
| | SFRLLSANVQLALDKAIETQRAFLNKKGKKSVPKFKKQFACFRQDTFKIFDRYVQFN |
| | MPGRERVNIPFKVCNPNHKQFIKQQPKRSQLINKKGKWFLYVSYETDKPAIDGNINIIG |
| | VDLGVKKIVTVSNPEASVNVFFSGNKAIYTRNKYQRYRKQIQRAKDTGKAKRGYRAL |
| | KRISGKEKNWIKDTNHKISKQIVNIAKQNKADIAIENLKGIRERIKATKKVRRMLHSWS |
| | FRQLISFLQYKSAMAGVRIVSVDPRHTSQRCPRCGHISKDNRKSQSAFKCSQCHYSVN |
| | ADLVGSRNIALTALNLYGEGKRPSERAVMLLPMAEGKTLMASCLEAPSVRAG |
| 96 | MPTITLRLELHNPTKVKQDMYERMTEVNTAFTNWLLNHPKLNQATSKIFKEFSPQRFP |
| | SAVVNQTIREVKSQKKNQKTKKFQTLWCCFNNQNVKVEKKGSFYTVSFPTLEKRIGV |
| | PVVTRPYQEAWLNRLLDGTAKQGAAKLYKKRKKWYLAIAITFDVKPRHETKVMGV |
| | DVGLRYIAVASVGTKSLFFKGSQCAFIRRRYAALRRTLGKAKKLQMIRKIGRKESRW |
| | MKDQNHKISRQIVNVALANGVGVIRMEALTGIRKRAKSAKEAGRSLHAWAFHQLQT |
| | MIAYKAEMAGIRVEWVDPTYTSQTCKCGHREKANRNGIRFRCQRCGYTLHADLNGA |
| | INIAKAISGFAAEPSALVTGAPPIGVHRNPMGRGDDTPLKLLRCPNQKWMRTQTTQES |
| | HAL |
| 97 | MWHVELKRTARVKLAIPDDRRDDLKRTMLTFREVAQRFADRGWERDEDGYVITSRT |
| | RLQSLVYKQVREDTGLHSDLCIGAVNLAADSLRSAVERMKAGKNVGKPTFTVPTAT |
| | YNTGAVSYFTDGDGTGYCTLAAYGGRVRAEFVYPPDEDCPQRQYLGGDEWEPKGAT |
| | LHYERDDGEYYLHVTVERDEPETELGEAENGTVLGVDLGVENIAVTSAGAFYSGGLF |
| | NHRRDEYERIRGSLQQTGTESAHRTIEKMGDRERRWNTDVLHRISKAIVQEAITHDCS |
| | HIAFEDLTDIRDRMPGAKKFHGWAFRQLYEYVEYKAAEFGIATTQVDPAYTSQRCSK |
| | CGTTLRENRTSQAAFCCQKCGYEVHADYNAAKNVATKLLRSGQKSPAGGATNQLAL |
| | KSGTLNGNGDFTPASS |
| 98 | MPTITLRLELYNPTKVKQDMYERMTEVNTAFANWLLNHPELNQATSKIFKEFSPQRFP |
| | SAVVNQTIREVKSQKKNQKTKKFQTLWCCFNNQNVKVEKKGSFYTVSFPTLEKRIGV |
| | PVVTRPYQEAWLNRLLDGTAKQGAAKLYKKRKKWYLAIAITFDVKPRHETKVMGV |
| | DVGLRYLAVASVGTKSLFFKGSQCAFIRRRYAALRRTLGKAKKLQMIRKIGRKESRW |
| | MKEQNHKISRQIVNVALANGVGVIRMEALTGIRKRAKSAKEAGRSLHAWAFHQLQT |
| | MIAYKAEMAGIRVEWVDPTYTSQTCKCGHREKANRNGIRFRCQRCGYTLHADLNGA |
| | INIAKAISGFAAEPSALVTGAPPIGVHRNPMGRGDDTPLKLLRCPNQKWMRTQTTQES |
| | HAFRRAECQIISSQTRVPRPDGSAGSTMSRRGRRWRISCT |
| 99 | MKASRFLILNDYEKNGILSSPQGGESMPTITLRLELYNPTKVKQDMYKRMTEVNTAF |
| | ANWLLNHPELNQATSKLFKEFSSQRFPSAVVNQTIREVKSQKKNQKTKKFRTLWCCF |
| | NNQNVKVEKKGEFYTVSFPTLEKRIGVPVVTRSYQEAWLNRLINGTAKQGAAKLYK |
| | KRKKWYFAIAITVEVQQREETKVMGIDLGLRYIAVASVGTKSLFFKGSQCAFIRRRYA |
| | ALRRTLGKAKKLHMIRKIGRKESRWMKDRNHKISRQIVRFALANGVGVIRMEELTGI |
| | RKRETSAKEAGRSLHSWSFHQLQTMIAYKAEMAGIRVEWVKPTYTSQTCRCGHREK |
| | ANRNGIHFQCKKCGYTIHADLNGAINIAKAISGFAAEPSALVTGAPPIGVHLNPMGRG |
| | DDTPLNLGVVRTRNG |
| 100 | MANKNVNDSKIKTHILTKKVQLIVDTDRFDTDEEKKAEVDRVYKYLRDSMRCQSRE |
| | MNQYYMHLWMMSVARNLNDDRYSMKKYMNNIIDAVHPYLDKNNKEFNNKQKKIT |
| | RDFEKKIKNLCEEYQAETEILNNDTLRELNKCFNRKKDGAYDTNFVNEMPEGLGIVM |
| | GRTVEQDFQNDCKAGLLSGIRNPRSYKINYPLIIPKSFVAYGVGSGKAMQGRGIIFPDM |
| | EYSDFHNMLFSTKNPNITYNFVHDIDFKLVFGSMKRSHELRVIFDRIKMGEYSICGSTIE |
| | INNKKKIMLNLSYEAPIYEKPNLDENTVVGVDLGMAIPAVCSLNNDDRTYKYIGDSHE |
| | LEFIKKGIQAQRRSYQRNAVYNKGGHGRNRKLENLDRLKKRERNTTRTHNQRYAKQI |
| | VDFALANNAKYINLEKLKGFSNNEKNKLVLRNWCYYELQQYIEIDASKYGIKVRYIY |
| | PMNTSRTCSVCGTLTTDEDVKNGVGRVSQDEFICKDPNCPSHTLYTRGPKGHKVPYF |
| | NADRNASRNIAMSEDFVKKNAKDNAFKKIDDLYEINNDIDAA |
| 101 | MQLNTITLTVKLQIRPLGDKEAQNEVWRNLRNINRDVFKAANLLMSHLHFVEAFEHQ |
| | FMQTDRILEKEMLDRENKKKNKKLNAQQIETIAQEIAEIKQKRKELKIEVAEKVKIFY |
| | NGKNESFAYNFIRHEYPQIPSYSVSILVKTVQNKFKEEWKEVKKGEKSISNFKKTIPIPI |
| | DDKQIANTDKGKKLCYIKKEGEKFVWELNSLEAKFEIVTGKYKQSKYMGEDKNKRT |
| | TLERVMNFEYKVADSSLKIEENKIFLLLVCKIPQQQATSLPNVSIGVDLGIRTPACIACN |
| | DGLRVISLGSKQDFLAIRQRFYEHRKRLQKSLAMTKGGKGREKKLKALEKLRKAERN |
| | WVRTYNHTLSKKIIHFAIQCRASRIQIELLEGFGRNENKVDEKGNYLLGKWSYYELQN |
| | MIKQKAEQYKLVVTTIDPYHTSKTCHVCGELGYRKGADFFCQNERCKEYQKRQNAD |
| | HNAAFNIAKSTTFVAKKEDCTYFKLEQEKKSKKEEE |
| 102 | MRRTVKIKLSLNQNQQKLLQETIKQFKQACQRVVDYGWNRNGLKTYQKNKLHKAT |
| | YSEIREKTDLPANLVIRARDRASETIKACVKKIKNGEKASKPTFKSDSIVYDKRTLTVW |
| | LEEERCSIATTNGRIKADFVLPNEYNDYYDKYLNNGWKITQSTIEKHSYEDDEPFYLH |
| | LGLEKEIQKNQSVNPTIMGIDLGIENLAVTSTGRFYSGTELFSRRERYEEVRGKLQAKG |
| | TRSAHLTIKQMSGRENRFACDTLHRISKKIVEEAISKDVDVIAMEELEKIRQKISNNKK |
| | FQTWAFKKLQEYIEYKANERGIEVKFVDPKYTSQRCSRCGTTLKQNRINQHFECKDC |
| | GYRVDSDYNAAKNIGFKTILDGQMSQSRMGNGQLALKSGVLKPNGNYYSYPN |
| 103 | MIVTRKIQIRPINKEHYKILEDYLRTCRLIANKSQTLFYVYWQEIMANNLKGKNIDAYF |
| | KERYQHSFKQTIYHILRPKHLEIPSRITDDTLNIAYKDFCNDLKNGLLKGERSLRTYNN |
| | GWIPVRSQTTKITKQNNKYILEWLKGIKLEVYFGRDKSGNQVVVDRILNGQSKFCDSK |
| | FIKKEKKWFLLLCVNEPEKENNLFDDISIGVDCGINIPAVCAVNKGYGRAYIGSSVLLT |
| | RFRMQKDQRQRQKNYISSSGMHGRQKVDRAIFKAQDFEHRYMHTFHHKISKEVIKFA |
| | LKNRASKIIMEDLKRFGQNQEESEEKKKIRRFWGYQQLQSMIEYKAKLENIAVKYIPP |
| | AYTSQTCSQCGYTDANNRKQSKFVCLNPKKKCGFEANADYNAALNIAKGGIKKVNN |
| | KDV |
| 104 | MPTITLRLELHNPTKVKQGMYERMTEVNTAFANWLLXLELHNPTKVKQGMYERMT |
| | EVNTAFANWLLNHPELNQATSKIFKEFSSQRFPSAVVNQTIREVKAQKKKQKAKKFR |
| | TFWCCFNNQNVKVEKKGVFYTVSFPTLEKRIGVPVVTRSYQEAWLNRLLNGTVKQG |
| | AAKLYKKRKKWYLAVAITVEVQQREETKVMGVDLGLRYIAVASVGTKSLXAKLYK |
| | KRKKWYLAVAITVEVQQREETKVMGVDLGLRYIAVASVGTKSLFFKGNQCAFVRRR |
| | YAALRRRLGKAKKLHMIRKIGRKESRWMKDQNHKISRQIVAKKLHMIRKIGRKESR |
| | WMKDQNHKISRQIVRFAVANGVGVIRMEALTGIRKRATSAKEAGRSLHAWAFHQLQ |
| | TMIAYKAEMAGIRVEWVNPTYTSQTCKCGHREKANRNGIRFRCQRCGYTLHADLNG |
| | AINIAKAISGFAS |
| 105 | MILTRKIKLVIVSENREEGYNLIRTEIREQHKALNLAYNHLYFEHNAIQKLKQNDEDY |
| | KQKRNKLQELINKKYEEHQKAKNLEKKEALREAYNNKKQELYNFEKEYNEKARQTY |
| | QQVVGFTQQTRVRNLINRECNLMSDTKDGITSKVTQDYKNDCKAGLLIGKRSLRNYK |
| | KDNPLLVRGRSLKFYKEDGDYFIKWNKGTIFKCILHIRKKNVVELQSVLENVLLGAYK |
| | VCDSSIGFNNKDMILNLSLNIPDKETQDYIPGRVVGVDLGLKIPAYLSLSDKVYVRKGI |
| | GSIDDFLRVRTQMQKRRRRLQKSLAAVKGGKGREKKLKALDHLKGKEANFAKTYN |
| | HFLSTQIVTFAVKNQAGQINMEFLEFDKMKNKSLLRNWSYYQLQIMVEYKAKREGIII |
| | KYVDAYLTSQTCSKCDHYEDGQREKQENFMCKNCGLEVNADYNASQNIAKSTSYIS |
| | DSTESEYHKKKQQVLKEILGENDIMNEQLSLFNNCDDIA |
| 106 | VITARKVKLTITENREDGYNFIHNELREQNQALNMAMNHLYFNYVAREKIKLADETH |
| | KIKLAEDQGYLDQKYTELKEVKTDKKKQNIRKSIQAAKKRLETLRKAENKQVAEKFK |
| | EIIAASEKTNLRDFITDNFNLTSDTKDRLTQKVSADFKNDIVDVLRGERTLRRYKKGNP |
| | LYIRGRNLTFYIKDEEYYIKWMKSIVFKCVLGVKKQNSLELQKTLDKVIEGKYKVCDS |
| | SIEFKQNSLILNLTLNIPVCNSFDKVEGRVVGVDLGMKIPAYVTLNDSDYIRRAIGSIDD |
| | FLKVRTQMQSRRRNLQRALKSTKGGKGREKKLKALNQFEVKEKNFAKTYNNFISSNI |
| | VKFASDNKAKQINMEFLSLSETQNKSVLRNWSYYQLQQMIEYKANRVGIKVKYVDP |
| | YHTSQICSKCGHYEEGQREKQEVFICKNPECKNFNIEVNADYNASRNIAKSNKYITKK |
| | EESEYYKIN |
| 107 | MMTKSVKIKLGTPIDSNWNTVRKILADLRYNSSKMLNFSIQQCYQWMIYRNEYREKY |
| | GKYPRAKDIYGYSHRNHIYRQAAEIFPIFNRGNISQTVGMATSRWSSDQKDVMSLRKS |
| | IPSYRLQAPIYIANQSYTISRTENGLVVDCSLVSKKYAKEETNDKTRYRISLVVKDNST |
| | ETILDRIVSGEYSQGYGQIVKDRRKEKWYLLVAYRFEPKKTKETGRILGIDLGIVYPLY |
| | MALNDSHHRYRIDGGEIEHFRRNIEKRKNQLLDQGKYCGDGRRGHGIRTRIAPIEFAR |
| | EKIKNFRNTTNHKYSKFVVDIAQKHDVETIQLEKLDGISEDSTFLKNWSYYDLQQKIQ |
| | YKAQEKGIKVAYIDPRYTSQRCSRCGNISRENRQDQQRFRCTKCGFSANADYNAAKN |
| | ISTQNIEKIIEEELKK |
| 108 | MTTKVMRYQLIEMVDNEKRFMYKMLDDLRYEVFKISNRAIQMFWDIDNTSYAFKQK |
| | FQENLDLKELTGVKSLAYISRALKEEYQKLNNTSVEQVSRKVEKEWKKNKSNMITAD |
| | TSMIRYKRKNANIKLKNTQFKIEPLDNNFYRISARLLSKSYAKDLHENGFEFAQKFKE |
| | KGKKKEKTIKEFIKKNDDNMWVHFKIKAHDGSQKSIIERVVNKEYKVGGSDIIYCDRK |
| | RKYYLNLSYTFEAEQAKVDENKILGIDVGVNTPATLAISDDKWYKEFIGDKQEIENYR |
| | NQVESRRRRLQKNAALYSGEGSTGHGRKTRLKSVDKIRDKIARFKDYKNHIWSRAIV |
| | NEAIKHGCGTIQMEDLTGIAANTNEKFLKTWSYFDLQTKIQYKAEEVGIKVVKVKPA |
| | HTSARCNNCGHIHSKENKDKWRPKEFHHEKFICQNCNHTAHADLNAAKNIAMKDIE |
| | KIIKDQLESQEKYYKNQMKYILD |
| 109 | MITTRKFKLAIVSDNRNEAYNFIRSEIRNQNKALNVAYNHLYFEHIATEKLKHSDEEY |
| | QQHLTKYQEVASNKYQDYLKVKEKAKASKDDEKLQKRVDKAREGYNKAQEKVYKI |
| | EKEFNKKSKETYQKVVGLSKQTRIGKLVKSQFTLHYDTEDRITSTVISHFNNDMKTGL |
| | LRGDRSLRTYKNTHPLLVRARSMKFYEENGDYYIKWIKGIVFKIIISAGSKQKANIGEL |
| | KSVLINILDGHYKVCDSSISLNRDLILNLSLNIPVSKENVFVPGRVVGVDLGLKIPAYVS |
| | VNDTPYIKRGIGNIDDFLKVRTQLQSQRKRLQKALKSTSGGKGRSKKLKGLDRLKAK |
| | EKNFVNTYNHFLSKNIIQFAVKNNASVIHMEELHFDKLKHKSLLRNWSYYQLQTMIE |
| | YKAEREGIEVKYVDASYTSQTCSKCGHCEEGQRVLQNAFICKNKECKGYGHKVNAD |
| | FNASQNIAKSTDIIRGTEIAKTNDTAKNTKSIKGNQQSDDEVERKQLELELN |
| 110 | MSDTSPYKHQTKNIGLLVHPEVSKATSKIFKEFSHGKFPSAVVNQTIREVKSKKKKQN |
| | AKSFKKLWCCFNNQNLKMEKVGDFYTVSFPTLEKRIGVPVVARPYQQAWLERILNGT |
| | VKQGASELYRKKKEWYIAIPITFEVEQRETKVMGVDLGLRYIAVASVGTKSLFFKGN |
| | QVAFVRRLFAARRRKLGKLKKLSAIKKSKDKESRWMKDQNHKISRQIVDFALTNGV |
| | GIIRMEDLTEI*NRAKSKKEAGRNLHSWAFYQLQKMIKYKAEMVGICFELVKPDYTSQ |
| | TCKCGHREKANRIGIQFRCKKCGYTCHADLNGAINIAKAHSGLVAPSVLVTGTPPMW |
| | VHSNHMGRGDDTPLNLGVVQNGNGLRTLTTQESHGFSRVECQYGRIICWTWTKTVY |
| 111 | VITVRKIKLTIMGDEETRNRQYKWIKDEQYNQYKALNIGMSYLATHLFLKMSESGLE |
| | QKTEKNIKTIEKQISKIENNITKEESKKKVNEEKLNNLINELDTLNASLSKLKEELEEISN |
| | NRSNVDDTFKRMYVDDLYNALSKVPFQHSDMKSLVSRKVKLDFNTDMKDLMSGNR |
| | SVRNYKRNHPLLVRGRDLRFRYDGSNIKIKWIQGIEFKAILGKISKTIELRHILNKVIDG |
| | EYKVCDSSLEFNKNNHLILNLAIDFPYTNKIEFIEGRVVGVDLGIAVPAYVALNDIAYV |
| | KKSIGDIDDFLRVKTQMKKRRNLQINLTSVKGGKGRSKKLKALDRLSEKESNFVRTY |
| | NHFLSKSIVKFAIDNKAGQINLELLSENALSDKIIKNWSYYQLQQFIKYKAERYGIKVK |
| | YVDPYRTSQTCSVCGHYEEGQRSKQDIFTCKNEKCKMFEKEVNADYNAARNIAISTK |
| | YIDDIKESEYYYKKSF |
| 112 | VKLVKTMRYQIIKPLSCDWDTLGTVLRELQRDTHSVLNKTIQLCWEWQGYSSEYKA |
| | ANGTYPTPKDTLGRSLEGYVYDRLKVQFPKMYTPNLSQTIQRAMLKWSADSKAIFKG |
| | EVSIPSYKKDVPLDLRKDSIHIERRGHDYILSLGLVSRAYKKELGLPECQIEVLIGTPDK |
| | TQRVILARLLTGEYTVSGSQIVWDKRNRKWFVNLAYHFEARPEQLDKTKILGVDLGV |
| | VFPVYMAVADGHFRAGIPGGEIEEFRRRVEARRRQLLRQGKYCGDGRIGHGRATRTR |
| | PLDKIADKIARFRDTINHKYSRYVVETARKLGCGVIQMEDLTGIREENLFLANWPYHD |
| | LQRKIEYKAREYGIEVRYVRPQYTSQRCSDCGYIHPDNRPEQAKFRCLACGFETNADY |
| | NAARNIATEGIEELIAAALNKASVV |
| 113 | VEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKRGSKLNNYLIVAYR |
| | LKVKDDKDLIPNKILGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 114 | VPIKALRVQIIKPFNTDYDSQPITWDELGRTLRDLRYAASKMANYVIQQNYMWEFFR |
| | QQYKQEHGSYPSVSEHKDKLYCYPRLTAMFPLAAGQMVNQIERHAKTVWSARKSEV |
| | LKLHQSVPSFKLNFPIIVHHDSYRISEVPEDGKSSTHVFLLQANLLSREAATRTRYSFLI |
| | NAGEKSKQTIVERIISGEYRQGALQIVGDRKNKWYCIIPYEFKTEENNTLDPQCIMGID |
| | LGISKAVYWAIIGSHKRGWIDGHEIEEFRRRVQARRKSIQEQGKYCGDGRIGHGRKRR |
| | LLPIEVLENRESNFKNTTNHRYSRFIIEAAIKNQCGVIQMEDLSGINERSTFLRNWTYY |
| | DLQMKIKAKAEEVGIEVRIVNPQYTSQRCSQCGHIDRDNRSNQATFVCTHCGYGGLY |
| | HCFACGKSQVEAGVCHLCGGETKIMKINADYNAARNLAICGIDQIIVQTLEGEGVR |
| 115 | MQKVLKVQLICEHFDQEGNAVDYKTICKLLWELQKQTREIKNKSIQYCWEYSNFSSD |
| | YYKEHHEYPKEKDILSYTLGGYVNDKLKAGNDLYSANCSTTIRTACAEFKNAKSDFL |
| | RGDKSIISYKANQPLDLHNKSIRLEYQNGTFYFWLKLLNKSAVKANGFRNTEIRFKAL |
| | VKDNSTRTILERCAASVYDIAASKLLYDRKKKCWYLNLVYAFEPQLAKALDPEKILG |
| | VDLGIHYPICASVFGDLKRFTIDGGEIEAFRRRVEARKISMLKQGKNCGEGRIGHGIRA |
| | RNKPVYAISDKIARFRDTMNHKYSRALIDYAVKNGCGVIQMEKLTGVTADANRFMK |
| | NWTYFDLQTKIEYKAKEAGIQIIFIDPHYTSQRCSKCGYIDRENRPVQSRFSCQKCGFT |
| | ENADYNASQNISIRNIEKLIEAQLQTKCESQADNS |
| 116 | LATKCIKLAGEYAKENSLEKDKFFKELRDIQYKTWLACNRAITYYYSNDMQNFIQKD |
| | VGIPKEDDKLLYGKAFKSWVANRISEILGDGISSYATDCISQFVSNRYKNDKKAGLLK |
| | GNVALSQFKRDIPVMLRERAYSIIDTPKGLGIEISFFSKTKQQELGIKRILFTFPKIDGSS |
| | KSILTRIMDKTYKQGSIQITYNKRKKKWMFAISYTFENKLEKVLNDNLVMGIDLGITK |
| | VATMSIYDIEKHQYKNMCFKEQTIDGTELIHYRQKIEARRKSLSISSKWASDNATGHG |
| | YKRRMKKANNIGDKYNRFKDTYNHKVSRYIVDLAYKHGVKTIQMEDLSGFSEHQSE |
| | SLLKNWSYYDLQNKIKYKAEEKGINTIFINPQYTSKRCSKCGNIHEENRDCKNNQAKF |
| | ECIICGHKENADINASKNIAIPYIDKIIKEYIKDTI |
| 117 | MKITKQTKIRFNFQSPNAETNRAVYEKLNGCIYLTWKAFNRAYNEFIFQHINKQKKGE |
| | NYTAEENKKFRNSGYRSILDIDIHSSIKASISQKAYKQFLNDTKKGGVLSGSRTWSSYK |
| | LPSPIPFQIRMLSIFKDTDGYFMRIPYFSKDFPFNLMIERNEQKVIVDRILSKEYELNDSSI |
| | QKDKIHNKWYINLCYSFDKEPDSSIDKSIVVGVDLGIAIPVMCAINSSEYIRGSFGNRQE |
| | IDNFRARIKSKRWQILKQNNSFYDLRTGHGKSGKLKPLIPLEDRIKKFMNTYNHKLSH |
| | AIVAFALQNKAGIINFENLENLSEVKQKNMYLRDWNNADIITKTEYKAKEQGIEVHFI |
| | NPAYTSQRCSKCGHIEKENRESQSEFKCTKCGYEANADFNAARNISQMNDKSSLKIDT |
| 118 | MQRSVTLKIIRPEDEKISWEELGYLLRGLSFKVCRMCNFCMTHQLLHALKLETELLNP |
| | QGNLYCYPRLAEEYPDVPTGIICAAETRARKLFRRSAEAVLHSETSLPRFRKDSSIPVP |
| | VAGYKILQDADHNVYADVQLLSRQGAKTQKRPGRIRLVLADNWRDQSAKAALQQIA |
| | AGKVKRGVASLFRVKNDWYFQIPYVTEVVNTGEGFEPDLVMGVAFGLQNALVYAFN |
| | TSLKRGAISGEEVLAHQEKYAVRRKKIQEQYNWSGRKGHGREDALKPLRHLYETERN |
| | YRSLVNSRYAKWVVDIAVKNRCGMIHLDSANYVSSGKKILLSRWPLYDLKEKIRRKA |
| | EEKGIQVTECSIPNLRTRCSRCGKEQEPEGEKRTFVCKDCGYGKADKNRRGGFISVDY |
| | NAARNLAVYKSEEKEL |
| 119 | MAKGTLSKVMKYELSYLDGCGDFQNMQKELWALQRQTREILNRTIQIAYHWDYTDR |
| | EKFKKTGQHLDVKSETGYKRLDGYIYDELKEDVKNFASVNVNATIQKAWSKYKSSK |
| | TDVLRGDMSLPSYKSDQPLVLHGQSMKLSEGEDGVVMQATLFSNTYKKEQEYSNVR |
| | FAVRLHDTTQRTIMKNILSGDYGLGQSQIVYKRPKWFLYLTYNFSPKQHEADPDKILG |
| | VDLGETIAIYASSIGEYGGLRIEGGEVRAFAKQLEARKRALQKQATYCGEGRVGHGT |
| | KTRVADVYKAEDKIANFRNTVNHRYSKKLIDYAIQHQYGTIQMEDLTGVKKDTGFPK |
| | FLQHWTYYDLQQKIEAKAKEHGIRIIKVNPAFTSQRCSKCGNIDSGNRPSQAVFCCTK |
| | CGFKANADFNASQNISIPGIDKIIKESYGANME |
| 120 | MNKVVRIYLISEHTDKKGDPVNYQDINKLLWELQKQTRTIKNKTIQYCWEYQNFSSD |
| | YYKEHHAYPSEKEILSYTLDGYVNDKLKNSSDLYSVNRSSTIRNAIKEFKNAKADMIK |
| | GVKSVISYKSDQPLSLHNQSVRIEYIGGQYFANIKLVNSPYAKEHDFASTMIRFKFWIR |
| | DKSAETIIQRCLSNEYKISESEMFYDRKKKQWYINLCYSFSASKNDSLDIHKILGVDLGI |
| | AYPMCASVYGDYARFTIHGGEIEKFRRTVEARKLSMLKQGKNCGEGRKGHGIKCRN |
| | KPAYNISDKIARFRDTINHKYSKALIDYALKNNCGVIQMEELTGITADADRFLKNWTY |
| | YDLQTKIKYKAEENGIKFKLIKPKYTSQRCSKCGYIDKENRKTQAHFLCLKCGFECNA |
| | DYNASQNISIENIDMIIEQELKSDANIGCT |
| 121 | MTKVVRVYLIIEQKDKNGDIVEYTKINKLLWDLQKQTRIIKNKAIQYYWEYKNFSRD |
| | YCEKNGNYPSVEEILTYKTVDGYINNRLKIDNDLCAINRSSTIKHAIAEYKNAESDIKD |
| | GTRSIINYKSDQPLDLHNTSIHIERVKEEYYLFANMVSREYAKANNFANARICFKLML |
| | KNNKSAQTIIDRCLNGDYKISESKIIYDRKKKQWCINLAYSFSPSNVQQLDYNKILGVD |
| | LGITYPLCASVYGEYDRLAIHKGEIENFRNKVEARRYSMLRQGKNCGDGRIGHGIKCR |
| | NKPAYNIGDKIARFRDTTNHKYSRALIEYAIKNNCGTIQMENLAGITDKAERFLKNWT |
| | YYDLQTKIQYKAKECGIKIQIINPQYTSQRCSRCGYIHSDNRKTQENFLCLKCGFAANA |
| | DYNASQNISIKDIDKIIKKNWKICEPDIYRIT |
| 122 | MPTITRKIELTLCTEGLSEEQRKEQWGLLYHINDNLYKAANNISSKLYLDDHVSSMVR |
| | MKHAEYLSLLKELARAEKQKTPDADAIAELRKKVAAAETEMTDQEHAICKYATEMS |
| | TETLAYKFATEIETHVFGQILTCLKQAAQSNFNSDAKDVKRGERAIRNYKKGMPIPFP |
| | WNRSLKIEADGGNFYLRWFNGLRFLLNFGKDRSNNRLIVKRCMKMDADYEGEYKLC |
| | NSSIQIAKREGKTKLFLLLVVSIPQEHVELNKKIVVGVDLGINVPAYVATNITEERKAIG |
| | DREHFLNSRMAFQRRYKSLQRLKGTAGGKGRTKKLEPLERLRKAEHNWVHTQNHLF |
| | SREVVDFAVKAHAATIHMEDLSGFGKDNDGNADEKKEFVLRNWSYYELQNMIAYK |
| | AAKYGIKVEKIRPAYTSKTCSWCGQQGFREGVTFICENPACKQCGEKVHADYNAARN |
| | IANSKAIIKKNE |
| 123 | MPTITRKIELTLCTEGLSEEQRKEQWGLLYHINDNLYKAANNISSKLYLDDHVSSMVR |
| | MKHAEYLSLLKELARAEKQKTPDADAIAELRKNVAAAEKEMTDQERAICKYATEMS |
| | TQSLSYRFATELETNIFAKILDCLKQGVFATFNSDARDVKRGERAIRNYKKGMPIPFA |
| | WNDSLRIEKDNKDFYLRWYNGLRFLFNFGKDRSNNRLIVERCLKMDADYDGEYKLC |
| | NSSIQIAKREGKVKLFLLLVVSIPQEHVELNKKIVVGVDLGINVPAYVATNITEERKAI |
| | GDREHFLNTRMAFQRRYKSLQRLKGTAGGKGRTKKLEPLERLRKAEHNWVHTQNH |
| | LFSREVVDFAVKTHAATIHMEDLSGFGKDNDGNADERKEFVLRNWSYYELQNMIAY |
| | KAAKYGIKVEKIRPAYTSKTCSCCGQQGFREGVTFICENPECKQYGEKVHADYNAAR |
| | NIANSKEIIKKNE |
| 124 | MPTKCIKVALEFIKKDNNISEKQINKELKDIQFKTHLACNRAMTYMYSNDSETIIQRDI |
| | GIPKEDDKFLYGKSFGSWIENRMNEIMDGVLSNNVAQTRAFVINTYNQDKKNGLFKG |
| | NVTLSQFKRDMPIILHNKAFKIIETSKGLGVEIGLFNLKKQKELGIKRIVFLTPRLGESE |
| | KSIFKRLMDKSYKLGTAQISYNQRKRKWMIAISYTFNKEKDTIYLDNNKVMGIDLGIV |
| | NVVAMSIYDNAKEQYIKMSWKDRLISGTELISYRQKLESRRKNLAIASKWASSNRCG |
| | HGYKNRMRAVNKQGDKFNRFKDTFNHKISRYIVNMALKYHAGIIQMEDLSGFSNEQS |
| | ESLLKNWSYYDLQEKIIYKAKENGIEVILIDPKYTSKRCSECGNIDDKNRDCKKDQEHF |
| | KCTACNYTDNADINASKNIAILKIDSIIENYLKNKNKG |
| 125 | MIKIVKTMKYEIKYDKELYNLLSDIQHAVWLIKNRATTAAYDWQQFSFAYNERFGEY |
| | PKEKDVIGKTLAPDVYGFLKEIGSFVSSSIVDSAVQEAITKFKNDKVKILKGEQSIQIYR |
| | RNGSFPIRASQLKGLTKLDNKTYNAKLSLLSNEGAKERDCKGQFLVTLVTGNGAYEIL |
| | DRVINGEYKMCDSRIYKRKNKFYLLLTYKFEKETDKVLDENRIMGVDIGVAVPAVLA |
| | INEDKFYRQYVGDAKEVSDFVAQINDRKKRLQRSRKWAGEGSRGSGRKKLMKPVDA |
| | ISNKIHNYRETKNHTWSRFIVNEALKNECGTIQIEDLSGISKDNAFLKEWTFYSLQQKII |
| | DKAKEHGIQVVKVKPNYTSQRCNKCGFIHKDVNKEIWRPTQSSFKCLNCGHETNADL |
| | NAARNIAMKDIEKIIVDQLNVQEQHQKHAEKYLV |
| 126 | MGAKIVKLQLIYKSDEVMPYKDYCKELFALMREMSIIKNKIATYLYLDKRFSVALPIE |
| | KDTKVKSASGLASAFASELLVNYKTGEVEVLKNHSSNRSAAGQDVQAKFKKFLKES |
| | MGAYAVNPPSFKDNGTLCLHDRCIQIYYDEDNKDYGAKLFLLSHSYAKELGIKSSNG |
| | FDYKLLVGDDSSRAIIERIIAGEYKISASNLKWDKRKKKWYLLLCYSFTEKRTTSYDPK |
| | EYENNVMGINFGVICPMYMSFNHCKNRYNIEANEIEAFRKQVEARRIALRRQRKYCG |
| | DGSIGHGKNKRNSPAMKIDDKIARFRDTCNHKYARYAVDMAIKHQCGIIVIEDLTSIA |
| | EKEERLFLKTWSYYDLQNKIEYKAKEAGIKVIKINPQYVSRRCSKCGYISFEKEENIKA |
| | PDYRKFHCVECGFQSHVDYNASQNNATIGIEAIIAEQIKEHNSENVKNQPAAKAAKSK |
| | SKIKETA |
| 127 | MITSRKIKLAIVSDNKDTAYSFIREETRNQNRALNVAYTHLYFEYVAQEKLKQSDKEY |
| | QQHLEKYKNAAAKKYQEFLTIKEKSKSDENLQPKMDKVRETYNKAMEKVYKIEKDY |
| | SKKAREIYQQSVGLAKQTRLGKLIKSEFDLHYDTVDRIGSNAMSDFSNDRKSGILSGE |
| | RSLRNYKKTNPLMVRARSMKLYEEDNNFYIKWINDIVFKIIISAGSKQRMNIAELKSVF |
| | IKLLSGQCKMCDSSISLDKGLILNLSIDMPITKENVFIPNRVLGVDLGLKIPAYVSLNDT |
| | HYIKGAIGNIDDFLKVRTGLQSQRRRLQKSLQSTGGGKGRRKKLQALERLKTKEKNF |
| | VNTYNHFLSKNIVQFAVKNNAGAIHMEELKFDKMKNKSLLRNWSYYQLQTMVEYK |
| | AKSEGIEVYYVDASYTSQTCSKCGNLEEGQREARDTFVCKKCGYNVHADYNASQNI |
| | AKSTKAINKTIEITNIV |
| 128 | MTKVTKVYLISEQIDKDGNKIDFKKISELLWNLQRQTRDIKNKCVQLCWEWLNFSSD |
| | YYKKSEEYPKEKDTLGYTLSGFVYDRIKNGSDLYSSNLSTSSRDTCTAFSNYKKEMLN |
| | GERSVLSFKANQPLDIHNKAIKLSYENGNFFVALKMLNRAGKEKYGINDDLRFRMQV |
| | RDKSVRTILERLMNDEYKVSASKLMYDKKKKLWKLNLCYSFDNHVISTLDPEKIMGV |
| | DLGVVYPIMASVNGDYARFSIKGGEIEAFRNRVEARRRSLLNQSRYCGDGRIGHGRK |
| | KRTEPAAQIADKIARFRDTTNHKYSRALIDYAIKNGCGTIQMEKLTGITSNAEHFLKE |
| | WSYFDLQTKIESKAKEAGIKVVYINPKFTSQRCNKCGYIHTDNRPVQARFCCQKCGYE |
| | ENADYNASQNIGTKHIDVIIEETLKMQCEPEVPTE |
| 129 | MIKVVKTMKYEIMYDKELYDLLSEIQYAIWLIKNRATPAVYDWQQFSFSYNERFGEY |
| | PKEKDVLGKTLAPDIYGFLKELGSFVSSQIIDTAVQEAIKKFKNDKMKILKGEQSIQIYR |
| | RNGSFPIRASQLKDLTKINNKTYEAKLSLLSNAGAKERDCKGQFLVKLVTGNGAYEIL |
| | DRIINGEYKMSDSRIYKKKNKFYLLLTYKFEKETEKVLDENRIMGVDIGVAVPAVLAI |
| | NEDKYYRQYVGDAKEVSDFVAQINDRKKRLQRSRKWAGEGSRGNGRKKLMKPVDA |
| | ISNKIHNYRETKNHTWSRFIVNEALKNECGTIQIEDLSGITTDNAFLKDWTFFSLQQKIT |
| | DKAKEHGIKVVKVKPNYTSQRCNKCGFIHKNVGKEIWRPTQSSFKCLNCNHVTNADL |
| | NAARNIAMKDIEKIIEVQLKAQEQNAKHEEKYLV |
| 130 | MPKQISKVMKYELKYLGEEDFYEMQKMLWSLQEDTREILNKTIQIFFHWDYTNKESL |
| | ETTGKALNLVEETGYKDISGYVYDKLKSRYPDMSRGNLSATIRAASKKYRSSKVDILK |
| | GTMSIPSYKKDQPIILRPDGIRLHEREMIYTVELSLFSGDFKKKKAWKSNVLFQIKACD |
| | KAQKAIMQRLLSREYKLGESKLVYKKKKWFIYITYSFKKTDAKLDKNKILGVDLGVT |
| | YAIYACSIGEYGSFSIKGEEALEYAKRLEARTISKQKQARYCGEGRIGHGIKTRLSTVY |
| | STRNKLANHRDTLNHRYSKAVVDYAVKNRYGTIQMENLSCIKKNTGFPKRLQHWTY |
| | YDLQSKIEYKAAEQGIQVIKINPKFTSLRCSQCGCIHKDNRKTQESFQCVECGYKDNA |
| | DHNAALNISIPQIDLIIKEEMTSAKEK |
| 131 | MAEHTVITRKIEVHLHRSGDSEEAKELLREKYHMWDTINDNLYKAANLIISHCFFNDA |
| | YEDRLRIQSPRFKQIQDSLRNAKRNKLGESEIKELKSEREQLFDEFKKQRYTFLRGGVA |
| | EGPNPEQNSTYRVASDNFLDTIPSDILTCLNKNITTTYKSYRKEIEFGNRTIPNFKKGIPV |
| | PFPIKKDKKLRISKRDDGSIFIKFPGRLEWDLDFGRDRSNNREIVERVLNGMYDVGDSS |
| | IQETRSGKRFLLLVVKIPKVKNVSLDSNRVVGIDLGINTPLFAALNDNDYERVSIGSRD |
| | QFLNVRNRMNAQKREMQKNLRSSTTGGRGRRHKLQALERLEGKERNWVHLQNHIFS |
| | KGAIEFAIKNNAGVIQMERLTGFGRNANDEVENDRKFLLRNWSYFELQQLIEYKADA |
| | AGIEVRYIDPYHTSQTCSFCGHYEAGQRVDQAHFICKNPECEKGKGKKNDDGTYAGI |
| | NADWNAARNIARSNKIVDRKKK |
| 132 | LILTRKIQIIPLGEKEEIDRVYKYLRDGIFYQNKAMNQYMSALYIAAIKDISKEDRKELN |
| | RLYSRVSNSKKGSAYDKSIEFAKNMNLGYVVKQVKQDFANSCKNGLLCGKVSLPTY |
| | RKNNPLLVHVNFVRLRSTNYHQDNGMYHNYESHTDFLDHLYSKDLEVFIKFANNITF |
| | KMIFGNPHKSAYLRSEIQQIFEENYKVCGSSIQIDGKKIILNLSMDIPKQELELDENIVV |
| | GVDLGLAIPAMCGLNTNDYIRQSIGSKDDFLRIRTQLQSQRRRLQKSLASTSGGHGRQ |
| | KKLKPLEKLKDRERNFVKTYNHYVSKNVVDFAVKNKAKYINVEDLSGFDSNQFILRN |
| | WSFYELQQFITYKAAKYGIEVRKINPYHTSQICSCCGHWEEGQRIDQAHFKCKSCGAE |
| | LNADFNASRNIAMSTDFV |
| 133 | MKTKKHTKVMRYEIIKPLDSTWEVFGQVLRQVQYETRHALNKSIQLSWEWQGFSAE |
| | YKRQSDHYPNLKEITNYTNLQGYAYNTLKHEFPSLYRGNFSQTVKRATDKWNSDRG |
| | EILRGERSIPNFKKDIPIDVVAAAFKEGKEQNLRIRKILSSEEYNNETGKRIQGYTVKVN |
| | LISDSYKKELGRNSTAFEMLIKVGDNTQKAIIERLIEGKYNIAASQILYQKGKGSGKKG |
| | KWFLNLSYSFEKEDVTLNPDLIMGIDMGIVHPIYIAFSDSYARYHINKGEINRFRNQIEK |
| | RKKELLHQGTYCGEGRKGHGIQARIKPIEVISDKIANFRKLCNHRYSKFVVDIAVKHG |
| | CGTIQMEDLQGISKDDVFYKNWSYFDLQEKIAYKAKESGIKVIKVNPRYTSQRCSQCG |
| | NIDSENRVNQADFLCTACGFKALADYNAARNISTRNIEKIIDKAVGKEIIDVVDLEYGN |
| | IS |
| 134 | MVKVVKIYLISEQVDEQGKDVDYNTICGVLWDLQWETREIKNKTVQLCWEWSGFSS |
| | DYYKKYGEYPKEKNLLDYTMGGFVYDKLKSKYHLYTANLSTTSQNTCGIFRTYKVD |
| | FVKGNRSVLSFKADQPLDVHKKSISIDRIDDNYFVKLKLLNKSGIQKYGIRDDFHFRM |
| | LVKDNSTKTILERCVGGDYKAAASKIIYDKKKKMWCLNLSYEFDVNTAKDLNKNRIL |
| | GIDIGIVYPVVASVNGELDRFVIQGGEIETFRRRVENRKKSLLKQTKYCGDGRIGHGRN |
| | KRTEPVDIISDQIARFRNTANHKYSRAVIDYAVRKQCGTIQMENLKGITDKSDRFLKN |
| | WSYYDLQQKIEYKAKEKGINVVFINPKYTSQRCSRCGYIDSANRPKLPNQSKFLCIKC |
| | GFTENADYNASQNIALYNIEKLIDAEA |
| 135 | MPTRVMKYQIVKPMNCDWKLLNRHLLDLQQEAHQILNKTIQLCWEWQGFCSEYKA |
| | KYDIYPVDKDIFHLTLAGYVNRMLRLQFPKSNSNNLGTSRIKAINRWHTDLKDVKSG |
| | QKSIASFKADVPIDLHYLSIKLHKEKDCYYADLSLISNIYKKELGRESGKLLVLLKSGD |
| | EVSRDILRNCLDGIYKIRASDISHKKNKWFLNLHYNYEETNKSLDKDRILGIDMGIAYP |
| | VYMSVYNTQITSYIEGGEIERFHKQVEKRSAEFQRQGKYCGNGRIGHGVKTRLKPLSF |
| | ATDKIANFRETTNHKYSKYIVDFAVKNNCGNIQMEDLTGIKNERIFLRNWTYFDLQK |
| | KIRYKAEENGINVLLIKPHYTSQRCSQCGDIDKKSRITQENFTCISCGYQTNADHNASI |
| | NISTPNIENIINDFMSLKID |
| 136 | MFMTKVTKVYLISEQIDKDGNKIDFKKISELLWNLQMQTRDIKNKCVQLCWEWLNFS |
| | SDYYKKSEEYPKEKDTLGYTLSGFVYDRIKNGSDLYSSNLSTSSRDTCTAFSNYKKEM |
| | LKGERSVLSFKANQPLDIHNKAIKLSYENGNFFVALKMLNRAGKEKYGIKDDLRFRM |
| | QVRDKSVRTILERLMNDEYKVSASKLMYDKKKKLWKLNLCYSFDNHVISTLDTEKI |
| | MGVDLGVVYPIMASVNGDYARFSIKGGEIEAFRSRVEARRRSLLNQSRYCGDGRIGH |
| | GRKKRTEPATQIADKIARFRDTTNHKYSRALIDYAIKNGCGTIQMEKLTGITSNAEHFL |
| | KEWSYFDLQTKIESKAKEAGIKVVYINPKFTSQRCNKCGYIHTDNRPVQARFCCQKCG |
| | YEENADYNASQNIGTKHIDVIIEETLKMQCEPETPTE |
| 137 | MNKVMRYQIIKPIDIDWKTFGDILNKMRQEVRFTKNKTIALYNDWLTYCFQYKNEHN |
| | EYPKLINYCGYKVFSGYAYDKFKTDVVFSNTANYTTSVREACSAYDTHKTDILKGNC |
| | SIPSMGANQPIDLHNKSLSVDVNESGDYIATISLLSNRGKKEFGLKSGQIKVVLKAGDK |
| | SSKDILQRCVSKEYKICGSKIIYKGKKTFINLCYGFEPEASDLDKSKVMGIDLGVSVPA |
| | YMAFNFDKYKRDSIKDNRIMTTKWMMDQQLSIAKQSCKYLSDGNSGHGRKKKMIC |
| | YDKYSNKSRNLSQTINHGWSKYIVDVAFRNGCGTIQMEDLSGVTSEKDKFLKNWTFY |
| | DLQQKIEYKAKEKGIDVVKINPRYTSQRCSECGCICKRNRPDQKTFKCISCGNSSNADF |
| | NAAKNIATIGIEDIIANTEVIE |
| 138 | MSIAVKVMKYQIVCPVNVEWKVFETYLRTLSYQSRTIGNRTIQKIWEFDNLSLNHFKE |
| | TGEYPSAQHLYGCTQKTISGYIYDQLKEEYQDINKANMSTTIQKTLKNWNSRKKEIW |
| | RGEMSIPSFRNNLPIDIHGNSIQIIKEKSGDYIASVSLFSSKFIKENDLPNGKILVKLSTRK |
| | QNSMKVILDKIVNSIYAKGACMLHKHKNKWYLSITYKATIKEEHKFDEELIMGIDMG |
| | KINVLYFAYNKGLVRGAISGEEIEAFRKKIEYRRISLLRQGKYCSENRIGKGRKKRIKPI |
| | DVLNDKVAKFRNATNHKYANYIVQQCLKYNCGTIQLEDLQGISKEQTFLKNWTYFD |
| | LQEKIKNQANQYGIKVVKIDPSYTSQRCSECGYIHKKNRQDQSTFECQQCSFKIHADY |
| | NAAKNISVYNIEKVIQKQLELQEKLNLTKYKEQYIEQMENIN |
| 139 | MTYLSIAVKVMKYQIVCPVNVEWKVFETYLRTLSYQSRTIGNRTIQKIWEFDNLSLNH |
| | FKETGEYPSAQHLYGCTQKTISGYIYDQLKEEYQDINKANMSTTIQKTLKNWNSRKK |
| | EIWRGEMSIPSFRNNLPIDIHGNSIQIIKEKSGDYIASVSLFSSKFIKENDLPNGKILVKLS |
| | TRKQNSMKVILDKIVNSIYAKGACMLHKHKNKWYLSITYKATIKEEHKFDEELIMGID |
| | MGKINVLYFAYNKGLVRGAISGEEIEAFRKKIEYRRISLLRQGKYCSENRIGKGRKKRI |
| | KPIDVLNDKVAKFRNATNHKYANYIVQQCLKYNCGTIQLEDLQGISKEQTFLKNWTY |
| | FDLQEKIKNQANQYGIKVVKIDPSYTSQRCSECGYIHKKNRQDQSTFECQQCSFKIHA |
| | DYNAAKNISVYNIEKVIQKQLELQEKLNLTKYKEQYIEQMENIN |
| 140 | MNKVVRLALICEHSDKDGNPVDYSDVYKLLWQLQAQTREIKNKTIQYCWEYSNFSS |
| | DYYKENHEYPKEKDVLHYALDGFVNDKFKVGNDLYSSNCSYMTRKVCAEFKKSKS |
| | DFLKGTRSIISYKSNQPLDLHNKSIRIEYKDNDFFAFLKLLKRPAFNRLGYKNSEIGFKV |
| | IVRDKSTRTILERCVDQIYGISASKLIYNKKKKQWFLNLVYAFEPDNANNLDPSKILGV |
| | DLGIHYPICASVYGDLQRFTIHGGEIEEFRRRVESRKLSLLKQGKNCGDGRIGHGGKTR |
| | NKPVYSIEDRIARFRDTVNHKYSRALIDYAVKKECGTIQMEDLSGITAESDRFLKNWS |
| | YYDLQSKIEYKAKEKGIKIVYIDPKYSSQRCSKCGHIDKENRKTQSSFVCLKCGFEENA |
| | DYNASQNIGIKDIDKIIENDLSSKCETDVN |
| 141 | MGKGVLAKVMKYELRYLDGCGDFSNMQEQVWALQRQTREILNRSIQIAFQWDCAN |
| | SEHHRKTGEYLDLKTETGYKRLDGHIYNCLKGQYEDMATSNLNATIQKAWKKYNSS |
| | KKEILRGSMSIPSYKMNQPLTLDKNTVKLSEGERNPIVTLTLFSDKFKRAQGVSNVKFS |
| | MPLHDGTQRAIFANLMNGTYQLGECQLVYKRPKWFLFVTYKFPPVEHPLDPDKILGV |
| | DMGEACALYASTFGEHGYLKIDGGEITKYAKKMEARIRSMQKQAAHCGEGRIGHGT |
| | KTRVSVVYQAKDKVARFRDTINHRYSKALIDYALKNQCGTIQMEDLTGIKEDTGFPK |
| | FLRHWTYYDLQSKIEAKAAEHGIQVVKINPRHTSQRCSRCGHIDKANRTSQADFCCTK |
| | CGFSANADFNASQNISIRNIDKIIAKAIGANRKQT |
| 142 | MRIKIIAKKKGINMNKIMKYQILKPTNISWEDFGNILYNLRSEVRKIKNRTIALYHEWT |
| | NYTLECHDRTGEWPKPKEVYNYGTMGGYIYDRLKGEVKYSNSVNFNSSVRDAMSK |
| | YDTHKKDILAGKASVPSMGDGQPIDIYNKNIVLHHLDNEKKDYAATLSLLNNGAKAE |
| | LGLPSGRVDVILTIKNETQTAILDRCLSGEYRICGSQLIYEAAGKEKKGKKDKPKVWL |
| | YLCYGFEPEAPELDDSRIMGIDLGMKLPAVMAFNFNDKKYEVIDDNRILDRKIRLDK |
| | MLSMSKHQCQWRCDGNSGHGRKKKVGVYENYANKSHNMSMTINHQWSKYIVDTA |
| | VKNKCGVIQMEDLSGIKASRQNFLGNWTYYDLQQKITYKAEEKGIKVIKVDPQYTSQ |
| | MCPICGYINKRNRATQADFECLECGHIANADYNAARNIATPDIANIIKNRLTQQKKEG |
| | KSID |
| 143 | MNENMCALTKIMKYELRYLDGFPDFSAMQNAVWPLQRQTREILNRTIQEAYHWDYF |
| | SATKKKETGEYPDLLKETGYKRLDGYIYHVLAPDYPDFSSSGVNATIQKAWKKYKSS |
| | RADVWKGEMSLPSYKSDQPIVLHAKQIKLSGDNRAAAVTLSLFSNKFKKEHAISGNV |
| | QFAITLHDNTQRTIYQKLRNGEYKLSESQLVYDKKKWFLYLAYSFNPAEHALDPEKIL |
| | GVDMGEKFALYASSFGEYGHFKIEGSEVTEYAKALERRRRSLQQQARYCGEGRIGHG |
| | TKTRVGAVYREEDRIANFRSTINHRYSKALIEYAVKNGYGTIQMENLTGIKENLQFPR |
| | RLQHWTYYDLQSKIEAKAKEHGIAVVKVNPKHTSQRCSRCGHIAAENRPKQEVFQCV |
| | KCGYACNADFNASQNISIKDIEKLIQETIGANPK |
| 144 | MQQVVRFEIIKPIDNDWKVLGKVLRDLQYESRQVLNKTIQYNWEFSNCAIGFKEKFGI |
| | NPKISDISKYKGSHALQNYIYNELKNVYTKISTSNLTCLIEKATSQWKTFQKDVYKGE |
| | RNPPEYKKSNTPIIIHKQCINILKENGKYYADIALVSKKHLEEYKLNSCRFTLLLNTKN |
| | NGNKAILDMILNGELDYSQSQIIEKNKKWFLYLSYKVAEKVLPDDYNRIMGIDLGVN |
| | KAVVIAIHGTEIRDYIPGGEIIAYKNKMYNMVWHRQKQARYCGEGRIGHGRKTRLKN |
| | IYKIKEKIANFSDLTNHRYSKYIVELAKKHKCGVIQMEDLSGLSTNNKFLKKYPIYDLQ |
| | QKIIYKAEREGIKVVKIKPNYTSQMCSNCHFISEENRPKDERGWEYFKCVNCGLEIDA |
| | DLNAARNMANSQIENIIKEQLKIQGIKNKNNGTDEESRKAVNS |
| 145 | MKYELTYLDGCGDFHNMQNELWALQRQTRELLNRTVQIAYHWDYKGSEHYRETGQ |
| | PMDVYTETGYKRLDGYIYSCLKDDADSFSAANVNATIQKAWKKYKSSKSDIMQGKM |
| | SLPSYKRDQPLILYARNVKISNEQRNPAVQFTLFSKSYKEEKGYSDVQFVMHLNDATQ |
| | KAIFQKIASKEYGLGECQLVYSKPKWFLLLTYNFTPEDRRLDPDRILGVDMGETFALY |
| | ASSKDEYGSLKIEGGEVREFAKRMEARKRSMQRQSAHCGEGRIGHGTKTRVSDVYK |
| | AEDKIANFRSTVNHRYSKKLIEYAVQHQYGTIQMEDLSGIRESTGFPKFLRHWTFYDL |
| | QQKIEAKAREQGIRVVKVDPSYTSQRCSKCGNIDKANRPTQAQFCCSKCGYKTNADF |
| | NASQNLSIRGIDRIIKETLGANPK |
| 146 | MSIKAIRLEIVKPYNESDAENVVTWNELGEALREVRYACSKAQNYVITERYLWERFKI |
| | DYKNQNGVYPDPKAFKERTNLYSQLTKMFPHVASNIINQTDRLATKKWSNEKKDVL |
| | SLKRSLTSFKLDVPIPVHYEGYKIFKVNDSEREKYIIRVTLLSKKSDKQMAYNLLLKVK |
| | DNSSKTILDRLISEELDSKIIQIVSTNKKKWFCIIPYDFTERNTEVVDGRIMGIDLGIAKA |
| | VYYAFNDSYKRGSIDGGEIERFRKSVRARRITIQNQGKYCGDGRIGHGVKRRLKPVEI |
| | LREKEKNFRNLINHRYSRHLVEIAVKNKCAVIQMEDLTGITKDNAFLKDWPYYDLQQ |
| | KIKEKAAEYGILFKTINPYKTSQRCSRCGYIDRENRPEQSVFVCQNCGYGSLYLCENC |
| | NKEQNHAGICDTCGGKTNLITVNADYNAAKNIATENIEEIIKKEMGKEYNPPK |
| 147 | MSVKIASFSLIYKEDYNELNYDDLYNVLIQLQNESAKIANRAVQIYWENSNYCRQVKS |
| | MTGRFPTKDELLKHYGCSEQNYVYRLLTKEFYKNSTGNISTIIQFVGKRYKRYYPDYL |
| | SGKRSIESYKSSFPIYLCKNNIRVFKEDGKYYIKLGLISALYKSELSIHSGSVIFELGFGK |
| | SSSYKTLLDNIINQLFSLTSSKIIFLKKKIIIQLGYNTNSNIILDNSSCRVMGIDIGVAKPFV |
| | YAFNDITDFNFVDGNEIKNFQKQMLSRRQSLGRQTKNCADSKIGHGIHKRIEGIEKLG |
| | QKESNFRNRINHQYSRMIVDAAIKYKCTTIQIEDLSGISSENKFLKSWPYYDLQSKIEY |
| | KAKECGIDVVKINPKFTSQRCSKCGYISKENRKTQAGFKCKNCGFEENADLNAARNIA |
| | IPKIDSIIQESLRATT |
| 148 | MVKTTKIYLCMDSKDTYKLLWKLQDNTRMLKNKAVQMLWEWNNFSQEYKKEHEE |
| | YPKPKDILNYTVGGYMYDKLKSESLLASSNLSSTLQLVEKQFKTNAKDFLRGDKSIIC |
| | FKKDQPLDIHNKSIRLSHENGTFYADIVLMNKATANEHNGGGCALPFRLWIKDKSTRT |
| | IVERCYDGVYSVSGSKIKYDEKKKMWYINLSYGFDKYSTAELDKEQVLGVYLAQETP |
| | FTASVIGDHDRLQVSVNEIEHYRNAVESRRRSILKQSAVCGEGRIGHGYKKRVEPMEK |
| | LSHKVADTRDTINHKYSKAIVEYAVKKGCGTIRMEKLTGISETDRYLRNWPYFDLQT |
| | KIEYKAKERGIDIVYVDSADIQRCCRCGHVNEEETTGSRFKCTECGFEHDVAYNASQN |
| | LSAGGKDIKVNGRK |
| 149 | MRLYSIKCNKEKKLNEVKRMESEKEKYVVQSYGIKLIKPVGVDEGDWDFAGKVLRD |
| | LDYACFRVKNKAATRTYMNVIEKLEYENIHGKGTYDKYFKTRYGKTFSAYNSECAR |
| | EDKELNKGDLYREYFNLMAREGEKVVKYNMKKILNGNASNITFKRNQPVPITSRMIFI |
| | TKESGKYYAELTLLSPEKAKELGRKGKNGTRIKFLLSSKGQEKVILDRLTSGEYDLRD |
| | SHIHVKKKGNKLTNYLIIAYRHKVKDDNDLIPNKVLGVDLGVSKAAYMAVSESPVSE |
| | YINGGEIEQFRNGVEARRNGMRNQLKYHSSNRSGHGRATKLKPLEKLREKVSNFRKL |
| | TNHRYAKFIVDTALKNNCSIIQMEELKGISKNDTFLKRWSYFDLQEKIENKAIAVGIEV |
| | KKVSPKFTSQRCNKCGYIDKESRKSQEKFECVNCGHKTNADLNAARNLSMLNVEKEI |
| | KAQCKAQKIKY |
| 150 | MDKTLRDIQYVTCKASNKAMQMYYMWEYEKLEYKNKYGEYPNEKEMYGKTYRNV |
| | VEAEVKAIMNTINTSNTGQTNAFVMKKWNTDKKDIMNYRKSVASYKLNMPIYLKNS |
| | SYKILQGESGYEIDCAIFNKSQGLKHLTFTIDKLDGTKKATLNKLIESKNLSELINGAYK |
| | QGAMQIVKKKNKWCMLISFGFEAVKRELDTNRIVGVDVGIVNALTFQVWDNTTQK |
| | WDRLSWRDCLLDGKELIHYRQKIMARRIALLKNSKLSDENKGKAGHGRVKRIGPINTI |
| | SDKVKAFRDTLNHKYSKYVIDFAIKNNCGCIQMEDLSGYSESVSETFLKNWSYFDLQS |
| | KIKYKAEENGLAINFIKPYHTSLRCSLCGNIQKENRDCRNNQSRFKCTVCGYEENADI |
| | NAAKNISLPNIEQLIKEQLKIK |
| 151 | MEGEKEKYVVQSYGIKIIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKKGNKLNNYLIVAYR |
| | LKVKDDKDLIPNKVLGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 152 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKRGSKLNNYLIVAYR |
| | LKVKDDKDLIPNKILGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 153 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKRGSKLNNYLIVAYR |
| | LKVKDDKDLIPNKVLGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECMNCGHKTNADLNAARNLSMLDIEKVIKAQCKAQKIKH |
| 154 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKRGSKLNNYLIVAYR |
| | LKVKDDKDLIPNKVLGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 155 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVEKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKKGNKLNNYLIVAYR |
| | LKVKDDKDLIPNKVLGVDLGISKTAYMAVSDSPVSEYINGGEIEQFRNGIEARRNSMR |
| | NQLKYHSSNRSGHGRSTKLIPLEKLRAKVNNFKELTNHRYAKFIVDTALKNRCSIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 156 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKKGNKLNNYLIVAYR |
| | LKVKDDKDLIPNKVLGVDLGISKTAYMAVSDSPVSEYINGGEIEQFRNGIEARRNSMR |
| | NQLKYHSSNRSGHGRSTKLIPLEKLRAKVNNFKELTNHRYAKFIVDTALKNRCSIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKTQCKAQKIKH |
| 157 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKKGNKLNNYLIVAYR |
| | LKVKDDKDLIPNKVLGVDLGISKTAYMAVSDSPVSEYINGGEIEQFRNGIEARRNSMR |
| | NQLKYHSSNRSGHGRSTKLIPLEKLRAKVNNFKELTNHRYAKFIVDTALKNRCSIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 158 | MEGEKEKYVVQSYGIKLIKPVGVDEGDWDFAGKVLRDLDYICFRVKNKAATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKKGNKLNNYLIVAYR |
| | LKVKDDKDLIPNKVLGVDLGISKAAYMAVSDSPVSEYINGGEIEQFRNGIEARRNSMR |
| | NQLKYHSSNRSGHGRSTKLIPLEKLRAKVNNFKELTNHRYANFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 159 | MEGEKEKYVVQSYGIKLIKPVGVDEGDWDFAGKVLRDLDYICFRVKNKTATRTYMN |
| | VIEKLEYENVHGKGTYDKYFKTRYGKTFSAYNMEQAKEDKELNNGSFLREHFDSMA |
| | REGEKVVKNNMKKILNGSASNITFKRNQPIPIRSRMIFITKESGKYYAELTLLSPEKAKE |
| | LDRKGRKGTRIKFLLSSKGQEKVILDRLTSGEYDLRDSHIHVKKRGSKLNNYLIVAYR |
| | LKVKDDKDLIPNKVLGVDLGISKAAYMAVSDSPVSEYINGGEIEQFRNGIEARRNSMR |
| | NQLKYHSSNRSGHGRSTKLIPLEKLRAKVNNFKELTNHRYAKFIVDTALKNRCTIIQM |
| | EDLSGISKTDTFLKRWSYSNLQEKIENKAKTKGIEVKKVSPKFTSQRCNKCGYIDKESR |
| | KSQEKFECVNCGHKTNADLNAARNLSMLDIEKVIKAQCKAQKIKH |
| 160 | MATKCIKLAGEYVKENSLEKDKFFKELRDIQYKTWLACNRAITYYYSNDMQNFIQKD |
| | VGIPKEDDKLLYGKAFKSWVANRISEILGDGISSYATDCISQFVSNRYKNDKKAGLLK |
| | GNVALSQFKRDIPVMLRERAYSIIDTPKGLGIEISFFSKTKQQELGIKRILFTFPKIDGSS |
| | KSILTRIMDKTYKQGSIQITYNKRKKKWMFAISYTFENKLEKVLNDNLVMGIDLGITK |
| | VATMSIYDIEKHQYKNMCFKEQTIDGTELIHYRQKIEARRKSLSISSKWASDNATGHG |
| | YKRRMKKANNIGDKYNRFKDTYNHKVSRYIVDLAYKHGVKTIQMEDLSGFSEHQSE |
| | SLLKNWSYYDLQNKIKYKAEEKGINTIFINPQYTSKRCSKCGNIHEENRDCKNNQAKF |
| | ECIICGHKENADINASKNIAIPYIDKIIKEYIKDTI |
| 161 | MATKCIKLAGEYAKENSLEKDKFFKELRDIQYKTWLACNRAITYYYSNDMQNFIQKD |
| | VGIPKEDDKLLYGKAFKSWVANRISEILGDGISSYATDCISQFVSNRYKNDKKAGLLK |
| | GNVALSQFKRDIPVMLRERAYSIIDTPKGLGIEISFFSKTKQQELGIKRILFTFPKIDGSS |
| | KSILTRIMDKTYKQGSIQITYNKRKKKWMFAISYTFENKLEKVLNDNLVMGIDLGITK |
| | VATMSIYDIEKHQYKNMCFKEQTIDGTELIHYRQKIEARRKSLSISSKWASDNATGHG |
| | YKRRMKKANNIGDKYNRFKDTYNHKVSRYIVDLAYKHGVKTIQMEDLSGFSEHQSE |
| | SLLKNWSYYDLQNKIKYKAEEKGINTIFINPQYTSKRCSKCGNIHEENRDCKNNQAKF |
| | ECIICGHKENADINASKNIAIPYIDKIIKEYIKDTI |
| 162 | MATKCIKLAGEYAKENSLEKDKFFKELRDIQYKTWLACNRAITYFYSNDMQNLIQKD |
| | VGIPKEDDKTIFGKSFVAWVENRMNEIMEGISSANVAQTRQFVNNRYSQDKKNGLLK |
| | GSVSLSQFKRDLPIIIHNKAYNVIETSKGLGVEISFFNKEKQKELNVKRIKLLFPKLDNS |
| | SKQILIRLMDKTYKQGSIQVTYNRRKKKWMFAISYTFENKLEKVLDDNLVMGIDLGIT |
| | KVATMSIYDIEKHEYKKMYFKEQTIDGTELIHYRQRIEARRKSLSIASKWASDNATGH |
| | GYKRRMKKANNIGDKYNRFKDTYNHKVSRYIVDLAYKHGVKTIQMEDLSGFSEHQS |
| | ESLLKNWSYYDLQNKIKYKAEEKGINTIFINPQYTSKRCSKCGNIHEENRDCKNNQSK |
| | FECVVCGHKENADINASKNIAIPYIDKIIKEYIKDTK |
| 163 | MATKCIKLAGEYAKENSLEKDKFFKELRDIQYKTWLACNRAITYFYSNDMQNLIQKD |
| | VGIPKEDDKLLYGKAFKSWVANRISEILGDGISSYATDCITQFVSNRYKNDKKAGLLK |
| | GNVALSQFKRDIPVMLRERAYSIIDTPKGLGVEISFFSKTKQQELGIKRILFTFPKIDGSS |
| | KSILTRIMDKTYKQGSIQITYNKRKKKWMFAISYTFENKLEKVLDDNLVMGIDLGITK |
| | VATMSIYDIEKHEYKKMYFKNQTIDGTELIHYRQRIEARRKSLSIASKWASDNATGHG |
| | YKRRMKKVNNIGDKYNRFKDTYNHKVSRYIVDLAYKYGVKTIQMEELRGFSEHQSV |
| | SLLKNWSYYDLQNKIKYKAEEKGINTIFINPQYTSKRCSKCGNIHEENRDCKNNQAKF |
| | ECVICGHKENADINASKNIAIPYIDEIIKEYIKDTI |
| 164 | MKINKCIKVTLIKCLNYDYKEIKQIIRDFNYTACKASNKAMRMWLFHTQDMIDKRNE |
| | DKLFNQIQYEKDTYGKSYRNVIEGEMKKLMPLANTSNVGTLHQQLVQNDWGRLKK |
| | DILSCKANIPNYKINTPYFIKNDNFKLRNHNGYFVGIAFFNKEGLQQYGYKIGHKFEFQ |
| | IDKLDGNKKATINKIINGEYKQGSAQISISKKGKIELIISYGFEKEEIPVLDNNRILGIDLG |
| | ITNVATMSAYDSIKDEYDYFSWKTNVISGKELIAFRQKYYNLRRDLSIASKTAGKGRC |
| | GHGYKTKMKPVDKIRNRIANFADTYNHKISKYIVEFAVKNRCGVIQMEDLSGATSEV |
| | HNKMLKDWSYYDLQQKIEYKAREQGIEVKKVNPKYTSKRCNKCGCIHEDNRDCKNN |
| | QAKFECKVCGHSENADINASRNIAIPEIDKIIDKTEILHSENRQAS |
| 165 | MKFNKCIKVTLIRCLNYDYRETKQIVRDFQYKYSKAYNMATNYLYLWDTNSMNLKN |
| | LYDTKIVDKELLGKSKCAWIENRMNEIIQDSNTSNVAQARQDVVNKYNKCKKDGLF |
| | KGKVSLPTYKLDSKVIIHNNSYKLRNHNGYFIDIGLLNKDKQKELNVGRFEFQIDKLD |
| | GNKKATINKIINGEYKQGSAQISISKKGKIELIISYSFDKEEVPVLDKNRVLGIDLGITNV |
| | ATMSVYDSIKDEYDYFSWKTNVISGKELIAFRQKYYNLRRDISIASKVAGKGRCGHG |
| | YKTKMKPVDKIRNKIANFSDTYNHKISKYIVEFAVKNNCGVIQMEDLSGTTADTNNK |
| | MLKDWSYYGLQQKIEYKAKEQGIEVKKVNPKYTSKRCNKCGCIHEDNRDCKNSQAK |
| | FECKVCGHKDNADINASKNIAIPDIDIIIKETEIL |
| 166 | MNSIKINKCIKVTLKKCLNLDIKEVKNIIKDMNYLACKASNKAIKMWQLHTEEMMER |
| | KFEDKNFNISQYEKDTYKKSYRNVIEGKMKDIMDICNTSNVGTLHQQLVQNDWGRL |
| | KKDVLNYRANVPTYKLDTPYFIKNHNYKLYNNGGWFVDIAFFNKQGLIQYGYKVGH |
| | KFEFEIDKIDNNKKSTITKIINGEYKQGSAQISLSKKGKIELIISFSFEKELKNNLDKNRIL |
| | GIDLGIVNTATMSIWDNNKQAWDWVDYKENRIDGKELIKFRQKLFSMGMSNSEIEKE |
| | IFKANSKIKENQLRKRELGVIDGLELAKYRDTVYKKRREMSIASKYAGKGRSGHGRK |
| | TKMKPVDKIRNKVYNFADTYNHKYSKYIVDFAVKHNCGIIQMEDLSNATANKKEKF |
| | LKDWSYFDLQTKIEYKAKEYGIEVIKINPKYTSKRCSKCGGIHIDNRDCQHNQANFEC |
| | KICGHKENADINASRNISIPYIDKIIAKTKVQAD |
| 167 | MVTKVVECKIIGFDSLLFEKEDVTENAKKDNKKAKEAWKRNIEMIDQAVRDFVAAK |
| | NRAITYLFTEYIKVRDAMATSGESSFSSVWKKMHDEKGRSKAMYHYLVKALPGYLT |
| | GNVSEGSRRLEKRFDQAVKDGLLYGRVSLPSFRDNAALDFRGDSVKIKQEIIKGEEKL |
| | YVELNLKRGIKLHCNIGFSGNKGAKAVAERIGKGEYKAGTASIVKRKSRYFIQICYSFD |
| | SDIYMRSNGMVGLAEGLDPKRTLGVDLGLARPVAMQPYDSDSRKYLSGGKNDEFVN |
| | TERYTNRFTISGDRIAAFRKQVEKKKSVLQRDLRDVSPGHTGKGRIKRIKPVEKFKDRI |
| | ANFRKTTNFVYAKRIVDTAIKYHCGTIRIEDLTIDVSSRKSERFLKTDWTYYDLQQKIE |
| | SRAANFGIVVQKVAPQYTSQKCSKCGHVSADNRKDQSHFKCVSCGFEMNADLNAAR |
| | NIAALSPNDKIKKEKNKEAKDGQMQLEFILN |
| 168 | MVTKVVKCKVVGFDSLPFEKKDAAGNAKINNKKAKEAWNRNIEMIDQAVKDFVAA |
| | KNRGITYLFTESIKIQDAMDASGESSFYDAWKKMHGGTQLRTTMDHFLVKELPGYLA |
| | ENVCESSGRLVTRFNQAKKDGLFKGCVSLPSFRDNAALDFRGSSVKIKQETINGEEKL |
| | YVELRFKRGIKLHCNIDFSGNNGAKAIAKRVCNGEYKSGTASIVKRKGKYFIYICYSFE |
| | PETYMRLNGMDGLAEKLDTKRILGVDLGLAKPVAMQPYDSISQKYLSGGKNDEFVN |
| | TEEYTNRFTISGDQITAFRKQVEKRKRALQRDLRDVSPGHTGKGRIKRIKPVETFKDRI |
| | ANFRNTTNFIYAKRIVDTAIKYHCGIIRIEALTIDVSSRKSERFLKTDWTYYDLQQKIES |
| | RAANFGIVVQKVKPNYTSQKCSKCGHVSADNRKDQSHFKCVSCGFEMNADLNAARN |
| | IATLSPTDKINKKKKREVKDDQV |
| 169 | MTKTKMITKATRFQIIKPLDTGWDELGQVLRDLSYHTTKLCNMAVQLYWQHHNYRL |
| | AHKEETGKYPAAAEDKERYGCSFRNHVYRKMREMYPDMASSNTSQTNQFAMSRWQ |
| | NDVREIMRLQKSIPSFRLGTPAQVANANYTLSVEEQDNGRSSFIATITLLSLAAGKQTR |
| | YSILLDGGDRSKKAIFRRIMEGEYKQGAMQITYNKRKKKWFCIVSFSFIPEKQNRELDP |
| | DRAMGLMFGTGNYTVFAAYSVGQKRYTLPAGEVIATEKKIHAIAERRKEIQRHAGYR |
| | GHGIKRKLQGTEVLAGQASAIRDTLNHKYSRRVVDVAMANRCGTIKILDKLVTPGAS |
| | VSELVNYPWAGVVEKIKYKAEEKGIAVKVVSLDGTRCHKCDAEIEIDSDNESPLTCPS |
| | CGAKIDKDYNTAKRIAKS |
| 170 | MITNTCKLEVSFIDTKFSDLFAMINATTQVKRYATDRITEWKSFSSRYHEENGEYPKLE |
| | DIYGFKSIEGLIYHELSKKKTGLSSRSLIATIHSCFLSYKQASKKHSHPNWSDSQPIIMQ |
| | GKYLNLHHESGNYIVDYPLFSKQYRVEHEITHILRMRLNTKENHHKNILDRILSGEYH |
| | LCQSQLKYVKKQNKSHWYFLLAYSHKSEGNPNIDPTRVLGVFMSENVALYASSKRLP |
| | MVFKIDGGEIPTFASRQEAIIRSKQRQAACCGDGRIGHGYKTRTKSIYPNKQTLAHFRE |
| | TINSRYAKALIDFAKTNGFGEVRLEDLKGIKADREFPRFLIHWTYFDLQEKIKNAARK |
| | CGIKVTLVSSETIGITCSSCGHIDPNNRDRGVKNRFVCTKCNIVKQTDWNASQVLSSLE |
| | I |
| 171 | MLTKCIKIPIEYSKDNILKSEEFYKELRDIQYKSWRACNRALTYFYMHDMENIMLKES |
| | GKDIKSDKELYGKTYGSWIENRMNEIMKGVLSNNVAQTRQYISNRYGQDRKNGLLK |
| | GNVSLSEIKRNMPIIIHAKAFKIIDIIKGLGVEVSFFNLNKQRELGVKKIKFLFPKIHSSEK |
| | SILKRLVDKSYKQGIAQLSFNERKKKWLLTISYSFEKRIEELDEDLVMGIDLGISKITTF |
| | SILNAKTKEYIQMNFKDKFVDGKELIHYRQKLEARRRELSIASKYTSKNNLGHGYKTK |
| | MQSVNMAGDKYNRFKETYNHKVSRFIIEIALRYRVKNIQMEDLSGFSEHQTESLLGN |
| | WSYYDLQNKIAYKAEELGINIIFIDPKYTSQRCNKCGNINSKNRNCKENQEKFECIRCG |
| | YKENADINASMNIAIPNIENIINNTIKIK |
| 172 | MKEKYFVKVAKFEILKPANGMTWKEFRQLLMSVRYRVFRLANLCVSENFLQFHLWR |
| | KKEIDKIPTLKISELNRQLREMLLEEKKTSDAEQTRICKRGALPASIVDALSQYKIRALT |
| | AKSKWRDVIRGNASLPSFRNDAAIPICCHKPSHRRLEKTSNGNVELELMICMKPYPRII |
| | LKTEKISGNMKATLERLLANRGNSDTGYQQRFFEVVQDRQEGRRWYLHVTYKFPAS |
| | LLRLNPQVIVGIDLGFSCPLYAAINNGLARLGWRHYESLGKRIRNLQNQVFARRRSMQ |
| | SGGNASLTMDTARGGHGRKRILRPIEKLAGRVNNAYSTLNHQLSRSVIEFAKNHGAG |
| | VIQMENLEGLKEQLTGTFLGSRWRYHELQQFIEYKAKEVGIEVRKINPQYTSRRCSEC |
| | GYINIKFDRAFRDANRKDGKTAKFICPKCKWEGDPDYNAARNLATLDIENLIRQQLIN |
| | QGIPSEREETPVL |
| 173 | MQECKYVQRSIKLKIVCPLDENITWEEFGFLLRGLSFKVARASNFCMLHHLLYAMKL |
| | ETVHLNRKGGLYCYPYLAEEYPEVPAGILCAAETRARKLFKQHAVEILRSDRALPNFR |
| | KDVGIPVPAASYKIMQDGKGDFLMEIQLLSRQAAKTGKLSGRICLALATNWRDKTAV |
| | AALQKIAEGSLKTGVGTLFREKKNWYFVVPYQREKGLSKEATENDECVMGVKLGVQ |
| | NALVYAFDRSLKRGSLSGEEVKARQEQFYARKQNILEQYKWSGRKGHGRERALKPIQ |
| | ELYEKERNFRNTANLRYANWIVEIAVKNRCGEIHLESGKGVGFGQNSIMLHFWPVWD |
| | LKNRIKQKAEEHGIKVVECNVPDIWGACSECGAKSGNSVEKQSFSCPSCGYGKQEEK |
| | YGVGYVSAEYNAARNLALWRKEE |
| 174 | MGKVHTRTIKLKLIVSTEDKNVAWKRIRQISNDAWRAANWIASGQLFNDQLVRRIYA |
| | RRKINPREDSEAVEQIEKEFEAFFGTKRQATTEWDIKEAFPDLPPYVTNPLNHVVVAS |
| | YKKEKPDMLSGNRSLRTYKRGMPIQTAKAAINFSKNETGQHFVKWTLGRKEFIDFEIY |
| | YGQDRANNRLTIDRIINGQIDYSAPMIQLKDKNLFLLLPVKEPEIAADLDPDIALGVDL |
| | GVATPAYMALSKGPARRAVGDKEDFLKVRLQMQSRKRRLQRSIKSAHGGKGRQKKL |
| | KALNQIGEKERQFARTYNHYISREIVNFAIRHNAGTIKLEMLEGFGQEEQQAFVLRNW |
| | SYFELQTFIEYKAKKVGIKILKIDPYRTSQTCSSCGHFEEGQRKDQSTFECKNCGEKLH |
| | ADYNAALNIARSKKIVTKKEQCEYYLNRNQNTI |
| 175 | MADQGKTLFKVMPLRILKPVGDTRWDALGQMLRDTRYRVYRLANLAISEAYLGFHL |
| | FRTGKAEQFKTDTIGQLSRRLRQMLLDEKVPRDNLDRSSMTGAVPDTVASGLHQYKI |
| | RSVTNVNKWQQVVRGKSSLPTFRADMAVPIRCDKAGQRRLERNTQGDVELDLMICR |
| | RPYPRVVLATGTLGPGQQTILDRLLENEDNAKSGYRQRLFEVKEDSQTHQWWLYVT |
| | YEFPAPVLPVAHGDIVVGIDLGVSVPLYAAINNGHARLGRRQFQALGYRIRTLQNQVI |
| | MRRRAIQRGGRVGVSQPTARSGHGVHRKLLPTEKLRRRIDKSYTTLNHQLSAAVIDF |
| | AKNHGAGVIQIEDLSTLKEQLVGTFLGGRWRYHQFQQFLAYKAKENGITLREVNPKY |
| | TSRRCSECGFIHVDFDRAFRDRHHTEGMVTKFVCPQCKYEADPDYNAARNIATLDIEQ |
| | RIKVQCQVQKLM |
| 176 | MAKKQDDNTMTIARKITLIPVASERKEWKKRIDAFLEKDFPMQIEIKKKQIKNTSKPER |
| | VDGYKQQLAELEKQYEEFKENGIKEYTHKMASDYTYDIVRRAMESEARRKNYILSYI |
| | YTKMIQDEVANLPTLTEKNKWVSANVKECYRKAGNKNGSIFTNVDIDNPLAGYGSDF |
| | GQAFTRKIKKLIKDGILEGNVSVPNYKLDSPFALSNQNFGIFTDCENITELKKNIGKPTY |
| | PVYVCLGKHGLPTIAKFKINFGHKQNKNKAELISTIIKILTGEYSVGGSTFGIDDDKIEM |
| | NLSITMQKQKMDLDENTVVGVDLGLAVPAVCALNNNEYDKQYIGSGNDLVTRRTKF |
| | QNEYTQLQKALKLAKGGHGRKRKLLALERLKEKEKNFVDTYCHRVSKKVVDYAIKH |
| | RAKYINIENLKGYDSSEFVLRNWSFYKLQQYITYKAEQCGIEVRKINPSFTSQVCSFCG |
| | HWEEGQRKDQATFKCKNPNCKSHKLYTVNADYNAARNIAMSTQFTDDKFKCSKKTI |
| | QDAADYYGIVLEDDKNNDNKKAA |
| 177 | MLLIQIEEVNQMITSRKIKLAIVSDNKDTAYSFIREETRNQNRALNVAYTHLYFEYVAQ |
| | EKLKQSDKEYQQHLEKYKNAAAKKYQEFLTIKEKSKSDENLQPKMDKVRETYNKA |
| | MEKVYKIEKDYSKKAREIYQQSVGLAKQTRLGKLIKSEFDLHYDTVDRIGSNAMSDF |
| | SNDRKSGILSGERSLRNYKKTNPLMVRARSMKLYEEDNNFYIKWINDIVFKIIISAGSK |
| | QRMNIAELKSVFIKLLSGQCKMCDSSISLDKGLILNLSIDMPITKENVFIPNRVLGVDLG |
| | LKIPAYVSLNDTHYIKGAIGNIDDFLKVRTGLQSQRRRLQKSLQSTGGGKGRRKKLQA |
| | LERLKTKEKNFVNTYNHFLSKNIVQFAVKNNAGAIHMEELKFDKMKNKSLLRNWSY |
| | YQLQTMVEYKAKSEGIEVYYVDASYTSQTCSKCGNLEEGQREARDTFVCKKCGYNV |
| | HADYNASQNIAKSTKAINKTIEITNIV |
| 178 | MPKQEEEGMIITRKIEIACRNKDQYPLLQDYLKQCRLMANKAMTNYYILWQEMLETK |
| | PKNQTQKDYFLKKHGCSFQNTGYRYLRRCFGGMPSYLRRDVANRVYQDFRADLKN |
| | GLLKGERSLRNYRTGHLPLHNKDIQLCKEETSSDYLLRWRMGIEFVLLFGRDRSGNRII |
| | ADRLIEGTYRVSGSQIYKDWRKKKWFVLLCVKIPQQDNNLDETAVVGVDLGLSTPAV |
| | CALSNGMARAYIGSSNLLKRFEMQKKQRSRQREFIPATNKHGRQKVVKAIYAMKDK |
| | ERRFVHSFNHAISKKIIQFALKYKAAVIRMEDLSGYNGDETVLRNWSYYELQSMIEYK |
| | AEKEKIRVEKIPACYTSRACSQCGFIDKENRKTQAQFECVKCGFKENADYNAALNIAR |
| | GGVKIPEENQEDFGENE |
| 179 | MILTRKIQIIPLGEKEEIDRVYKYLRDGIFYQNKAMNQYMSALYIAAIKDISKEDRKEL |
| | NRLYSRVSNSKKGSAYDKSIEFAKNMNLGYVVKQVKQDFANSCKNGLLCGKVSLPT |
| | YRKNNPLLVHVNFVRLRSTNYHQDNGMYHNYESHTDFLDHLYSKDLEVFIKFANNIT |
| | FKMIFGNPHKSAYLRSEIQQIFEENYKVCGSSIQIDGKKIILNLSMDIPKQELELDENIVV |
| | GVDLGLAIPAMCGLNTNDYIRQSIGSKDDFLRIRTQLQSQRRRLQKSLASTSGGHGRQ |
| | KKLKPLEKLKDRERNFVKTYNHYVSKNVVDFAVKNKAKYINVEDLSGFDSNQFILRN |
| | WSFYELQQFITYKAAKYGIEVRKINPYHTSQICSCCGHWEEGQRIDQAHFKCKSCGAE |
| | LNADFNASRNIAMSTDFV |
| 180 | MQRSIILKILRPYDESIKWEELGYLLRGLSYKVCKISNYCMTHHLLRALNLETENLNPQ |
| | GHLYCYPRLAQDYPDVPAGILCAAEGRARKVFLKCGKEVLRSETALPNFRKDCSIPIP |
| | VAGYSLLKAGEDTYVANVQLLSRQAAKTQKLPGRIQLVLAKNWRDRSAGPVLQQLT |
| | EGTLKRGIASLFRRKRDWYISIPYEAEAQKTEDGSFAPGLVMGVILGTQCALAYAFNR |
| | SPKRGAIGGEEILAHKEKFRARKKHIREQYNWSGRKGHGRGSALKPLQALYEKERNY |
| | RNLTNERYAKWVIEIAKKNRCGKIHLDGGSAGGTGTPHVMLAYWPQGELRKKIRYK |
| | AEACGIEVVECDGRDVWNRCSKCGAVQEVSGERRWFTCSHCGYGKDDKKTSSSFVT |
| | VDYNAARNMAIMESKP |
| 181 | MATEYTCITRKIEVHLHKHGDTEEAAQRLKDEYHIWDNINDNLYKAANRIVSHCFFN |
| | DAYEYRLKIHSPEFRQIEKSLQHAKKNKLSEDDIKKLKAERKSLCADFREQRLAFLRG |
| | GATEGANPEQNSTYQVVSHEFLDVIPSDILTCLNQNIASVYKQYALEVEMGKRTIPNF |
| | KKGIPVPFAIKQNKQLRLRKRNDGSVYVLLPRELEWDLSFGRDRSNNREIVERVLSGQ |
| | YDVGNSSIQETRSGKRFLLLVVKIPKASRTLDTKKVVGVDLGIATPLYAALNDNEYGG |
| | LSIGSQEQFLKIRMRMTAQKRELQRNLRYTTNGGHGRTQKLQALERLEGKERNWVH |
| | LQNHIFSKSIIEYALKNNAGVIQMERLTGFGHDKNDEVGTEYKFILRYWSFFELQSMIE |
| | YKAKAAGIEVRYINPYHTSQTCSFCGKYEKGQRINQATFVCKNPDCVKGKGRQKSDG |
| | TYEGINADWNAARNIALSEEFVDKKKK |
| 182 | MNTVRKIKLTILGDIETRNKQYKWIRDEQYKQYRALNLCMTYMVTNLMLRNSESGL |
| | ENRKEKEILKIQNKIKKDEEKLEKELKKEKSKEEKIQDIKFNIEELKLQKEKLENELRNI |
| | KEYRSNIDEEFKKMYVDDLYNVLNKVPFQHEDMKSLVTQRIKKDFNNDVKEIMRGD |
| | RSVRNYKRNFPILTRGRDLKFQYIEKSEDIEIRWIEGIKFKCILGKTSKSLELKHTLHKVI |
| | NKEYKVCDSSLQFDKNNNLILNLTLDIPQDNKYEKITNRVVGVDLGLKIPAYVALNDT |
| | KYIRKAIGSIDDFLKVRTQMQSRVRKLQKSLQTARGGKGRNKKMKALDRFREKERNF |
| | ARNYNHFLSYNIVKFALDNKAEQINLELLEMKETQNKSILRNWSYYQLQSFIKYKAER |
| | IGIKVEYIDPYHTSQICSECGNYEEGQRVEQATFVCKRCGHKINADYNAARNIAMSKE |
| | YISKKEESKYYKNNKNMV |
| 183 | MSKNTYTITRKIQLIPVGDKEEVNRVYTYLRDGMKAQNMALNQYISALYFAMQNDA |
| | TKDSRKELRNLFSRISTSKKGSAYDDTIEFAKGLPSCMMTRKVESDFKNAMKKGLKY |
| | GKLSLPTYRDTNPLLIHIDYVRLRSTNPHLDNGLYHNYKNHTEFLEHLYDENLELFIKF |
| | ANKITFKIILGNPHKSSELRSVFKNIFEDCYHIQGSSIEIEKNKIILNMSMSIPVKKIELDE |
| | NIVVGVDLGMAIPAVCALNTNEYIHKSLGNYNDFIRERTKIQAQKKRMQKSLTYTNG |
| | GHGRKKKLKPLNRFKERERNWVKTYNHKISKQIIDFAIKNKAKYINLEDLSGIAKEDK |
| | DKFVLRNWSYFELQQFITYKAEKYGIIVRKIKPEYTSQKCSCCGHLEENQRINQSEFIC |
| | KNPNCKNFGKIVNADYNGARNIAMSTNFVEEKETKSKPKKVA |
| 184 | MQRIIILKILRPYDESIKWEDLGYLLHGLSYKVCKISNYCMTHHLLRALNLETENLNPQ |
| | GHLYCYPRLAQDYPDVPAGILCTAEGRARKVFQKCGKEVLRSETVLPNFRKDCSIPIP |
| | VAGYSLLKAGEDTYLANIQFLSRQAAKTQKLPGRIQLVLANNWRDRSAGKVLQQLTE |
| | GTLKRGIASLFRRKRDWYISIPYEVEAQKTEDGSFAPGLVMGVILGTQCALAYAFNRS |
| | PKRGAIGGEEVLAHKEKFRARKKHIREQYNWSGRKGHGRGSALKPLQALYEKERNY |
| | RNLTNERYAKWVIEIAKKNRCGKIHLDGGSAGGTGTPHVMLAYWPQGELRKKIRYK |
| | AEACGIEVVECDGRDVWNRCSKCGAVQEVSGERRWFTCSHCGYGKDDKKTSSSFVT |
| | VDYNAARNMAIMESKP |
| 185 | MATITRKIELYIDKSNLTDDEYKAQWQYIRQIDNTLFLAANRISSHCLLNDELEIRLKL |
| | QMPEYCEIEKSLRNSKKNKLSKEEISELKLRRKELDAVVKRQKEEFLKTSQNSTYQLV |
| | AYEFTNIPTEILTNLNNDIVGKYGKARLDIIKGIKSPSTYKKGIPIPFSVNKKSPFVFIDG |
| | DLEWFKGKTKETTGKILRFKLHFGKDKSNNRAIVERLVESAKLGKKKGEAYIVNNSSI |
| | QLVEKENTTKIFLLLSLDIPTTKRALDSNLVMGIDLGINYPIYYATNGNAYIKGHIGDR |
| | DSFLNERMVFQRRFRELQRLQCTQGGRGRLKKLAPLEKLREKERNWVRTKNHIFSREI |
| | IKCALKIGAGTIHLEKLKNIGKDKDGNIEDSKKYILRNWSYHELQEMIEYKAAMEGIT |
| | VKYVNPAYTSQTCSCCGNRGERISQSVFKCLCPECSEYGKEVNADYNAARNIAKSEIF |
| | VKK |
| 186 | MDNTITITRKYTLIPTFSDTKEWTKKVMEYTKTSYIEKIKYYEEKIKKTKKKDKEEREK |
| | YENRLSQLKEQQLDFEENGTLLQTNVNDYTYDLVREAMASESDRKNMIISYVCGELI |
| | NRDAKDMDFKERNKLISELCNYGYRVKGSKKGSLFDHLDIDNPLGGYGVSFCQDLTK |
| | KIKELVNNKRWLDGKVSTLHYKDDSPFSIAKATMGFAHDYDSFEELCEHIREKDCNL |
| | YFNFGNNGKPTIARFKINLGANRKNKDELISTIIRVYSGEYQYCGSSIGIEGTKIILNLSM |
| | KIPKQEKELDENTVVGVDLGIAVPAVCALNNNVYARKFVGNKDDFFKVRKQLNAQY |
| | KRVQSALKRASGGHGRKKKLKALERLRKKEAHFVETYCHMVSKAVVDFALKYNAK |
| | YINLENLTGYDTDDIVLRNWSYYKLQQYITYKASKYGIEVRKINPCYTSQICSECGNY |
| | HPENRPKGDKGQAYFNCHNKECITHGKKSPYQYGINADFNAARNIAKSTLWMGKGK |
| | VTEESKKKAREYYGIEEEYEELNKEVA |
| 187 | MITARKIKLTIAENREEGYSFIRKELQEQNKALNMAINHLYFNYVAREKIKLADETYK |
| | VKLEEGECYLERKYIELKEAKTDKQKENIKKSIEATKKKLETLRKVENKEVSNNFKEII |
| | ATSEQINLRDLISNNFNLKSDTKDKLTQKVVQDFYNDIVGVLRGERTLRRYKKDNPLY |
| | IRGRSLTLYREGEDYYIKWMNGIVFKCVLGVKKQNSLELQKTLDKVIFEEYKLCDSSI |
| | GFKDNKLILNLTLDIPVSNTNKFEKVIGRIVGVDLGMKIPAYCALNDSEDVRKAIGSID |
| | DFLKVRTQMRSRRRKLQRALKSTNGGQGKNKKLSALNSFEAKEKNFAKTYNHFLSS |
| | NIIKFATDNKAEQINMEFLSLSETQNKSVLSNWSYYQLQQMIEYKAERIGIKVKYVDP |
| | YLTSQTCSECGHYEDGQREVQSEFQCKKCGCKTNADYNASRNIAKSDKYITKKEESE |
| | YYKNKI |
| 188 | MPILTRTIELIPIGDKEERDRCYKWIRDFMEEQSKMMNQYMSALYIAAVEEVSKDDRK |
| | ELNNLYNRIATSKKGSAFSKEECNLPKGLGANYGQRVRSDFDTACENGLLHGRVSLP |
| | TYKKNFPIILAPIYVNLQKNNIEEKGKSAGFYHNYASYNELYDALKEENKPEIIWNFVQ |
| | KMQYQIKFGNPYKSAFLRDEILHFLEGEYKAVGSQLSINSRGKIILNLSLDVPQKKVKL |
| | DENIVVGIDIGLAVPVMCAINNDYYKRLAVGDFEAFTRMREKLYSQKCKLQRQLKYT |
| | SGGHGRKKKLASLNAIRDREHRFVHTMNHKYSSEVINFALKNNAKYINMEDLTGFGK |
| | DNKGNAIDDYQFVLRNWSYFELQKMIQDKAQKYGIVVRKVESAYTSQLCSCCGEMG |
| | ERVSQSVFRCLNPNCISHNKYEKQRKSGVGNYHFNADFNAARNISMSTNYTKKKRKT |
| | KAEKVEERKKNAIEKTAG |
| 189 | MVKMKKETKQKRNTLCKKITLYPLGSKEEVTRVYNFIRDGQYMQYKGLNLIMGQLA |
| | SQFYKCNSELDDPQYKEWVEQEIRNTNSLLQAMEFPKGIDSRSLIIQRVKQDFSSALKN |
| | GLARGERSITNYKRTLPLMTRGRDLKFSCNYSELEELEYKCFSKDFKVYVSWVNKIKF |
| | KVVLGSVKRSLALRKELCQILRSVYSIQGSSIEIKDKKIILNLSMGIPLMEKVLDENTVV |
| | GVDLGIAVPAVCGLNTNKNTRRFIGSKDDLLKIRTKVQSQRKSLNKKLRECTGGHGR |
| | SKKLQALDRLSESETNYVKTYLHMVSKEIIRFAVAHNAKYINIEKLEGYDASSFILRN |
| | WSYYQLQSFIEYKAKIKGIIVRKVNPYHTSQICSYCGHWEEGQRLSQDKFKCKCCGVE |
| | LNADFNASRNIALSTEFVE |
| 190 | MAKDTFTITRKIQLVPVGDKTEVNRVYNYIREGMKAQNLAMNQYISALYLGMQNDV |
| | SKDDRKELNNLFSRISTSKKGSAYDESIQFAKGLPIGSMTRKVKSDFDTAMKKGLKYG |
| | KLSLPTYKDSNPLLVHVDYVRLRSTNPHQDSGLYHNYTNHTEFLEHLYKSDFELFIKF |
| | ANYITFKIILGNPHKSAEIRDVFKNIFEECYAIQGSSIGIYNNKIICNLSISIPKKQLCLDEN |
| | IVVGVDLGLAVPAVCALNTVPYIHKSLGNYDDFVRERTKMQSQRKRLQKSLNYANG |
| | GHGRKKKLQSLERLKKRERNWVQTYNHKISKQIVDFAIKNKAQYINIEDLSGFDSSQF |
| | VLRNWSYFELQQFIEYKANKYGIIVRKINPYHTSQTCSFCGHWEEGQRISQSEFICKNP |
| | ECVNHNKSINADYNAARNIAMSTDFVKNN |
| 191 | MDNTITITRKYALIPEFSDRKEWKKRVYDFTINDLEQKIDYRNKKKQDASELESQLEYI |
| | KNGGDFTRNMVNNYTYSLVRTAMEEEARRKNYILSWIFSEMRANRVDQMESLKDKF |
| | KFVSDTINYAYRKAGSNKGSLFDETEIHCILKSYGIAFSQELTKEIKELVKNGVLEGKV |
| | VIPTYKLDSPFTIAKSHFSFEHDYDSFEELCEHISDSDCKMYMNYGGDNRKDGINPASI |
| | AKFKISIGHGKNKDELKSTLLKVYSGEYQYCGSSIQITKNKIILNLTMKIPKIETKLDEN |
| | TVVGVDLGIAIPAMCALNNNMYERLAIGSADDFLRTRTKLQSQRRRLQKSLKNSNGG |
| | HGRNKKLKVLERLGKSETHFVETYCHMVSKRVVEFAVKNRAKYINIENLNGYDTSQF |
| | ILRNWSYYKLQQYITYKAERYGILVRKINPCYTSQVCSVCGNWEEGQRKTQSSFECA |
| | NPECKSHEKYKYGFNADFNAARNIAMSTLFMETGNVTEKSKEEARKYYGIEKD |
| 192 | MDNTITITRKYALIPEFSDRKEWKKRVYDFTINDLEQKIDYRNKKKQDASELESQLEYI |
| | KNGGDFTRNMVNNYTYSLVRTAMEEEARRKNYILSWIFSEMRANRVDQMESLKDKF |
| | KFVSDTINYAYRKAGSNKGSLFDETEIHCILKSYGIAFSQELTKEIKELVKNGVLEGKV |
| | VIPTYKLDSPFTIAKSHFSFEHDYDSFEELCEHISDSDCKMYMNYGGDNRKDGINPASI |
| | AKFKISIGHGKNKDELKSTLLKVYSGEYQYCGSSIQIAKNKIILNLTMKIPKIETKLDEN |
| | TVVGVDLGIAIPAMCALNNNMYERLAIGSADDFLRTRTKLQSQRRRLQKSLKNSNGG |
| | HGRNKKLKVLERLGKSETHFVETYCHMVSKRVVEFAVKNRAKYINIENLNGYDTSQF |
| | ILRNWSYYKLQQYITYKAERYGILVRKINPCYTSQVCSVCGNWEEGQRKTQSSFECA |
| | NPECKSHEKYKYGFNADFNAARNIAMSTLFMETGNVTEKSKEEARKYYGIEKD |
| 193 | MNLHGYKTDAKGFSSFLQSGNPVNLHGCKTRKEIKMESTTVITRKYTLIPEFSECKEW |
| | KKRVYDFTVKDIEQRIEYKKKKKQDISDLEIQLECIKNNSEFTRSMVNNYTYNLVRTA |
| | MEEEARRKNYILSWIFSEMTANRVDQMESLKDKFKFVSSTINYAYRKAGSNKGSLFD |
| | ETEIHCMLKSYGIAFSQELTKKIKELVKNGVLEGKVVIPTYKLDSPFTIAKAHFSFDHD |
| | YDSFEELCEHINDSDCKMYMNYGGNNTKTGTNPASIARFRINLGHGKNRDELKATLL |
| | KVYSGEYRYCGSSIQISKNKITLNLSMKIPKVEMKLDENTVVGVDLGIAIPAMCALNN |
| | NLYERESIGKVNDFTRVRKKHQAQVKRLQKSLKSATGSHGRSKKMRALNRVKKSEA |
| | HFVESYCHYVSRKVVDFALKHNAKYINMENLKGYDTSQFILRNWSYYKLQQYITYK |
| | ADRYGIVVRKINPCYTSQVCSVCGHWEPDQRKTQASFECANSECESHKKYKYGFNAD |
| | FNAARNIAMSTLFMEDGEVTEKKKEEAREYYGIKK |
| 194 | MPTITRKIELTLCTEGLSDEQRKEQWKLLYHINDNLYKAANNISSKLYLDEHVSSMVR |
| | MKHAEYLSLLKELARAEKQQTPDEGLIAELSRKLSAAEKEMADQELAICKYATEMST |
| | QTLSYNFAKEIETNIFGQILTCLRQGVYATFNSDAKDVKRGERAIRNYKKGMPIPFPW |
| | NNSLKIEADSGEFYLRWYNGLRFLLTFGKDRSNNRMIVKRCMKMDEDFEGEYKLCN |
| | SSIQLAKRDGKPKLFLLLVVNIPQEHVELNKKIVVGVDLGVNVPAYVATNITEERKAI |
| | GDREHFLNTRMAFQRRYKSLQRLKGTAGGKGRTKKLEPLERLRDAERNWVHTQNHL |
| | FSREVVNFAVQARAATIHMEDLSGFGKDKDGNADEKKEFVLRNWSFYELQNMIAYK |
| | AAKYGIKVVKIRPAYTSKTCSWCGQQGDRKSTTFICENPECKHYGESIHADYNAARNI |
| | ANSNDIVKENE |
| 195 | MDNTITITRKYALIPEFSDRKEWKKRVYDFTINDLEQKIDYRNKKKQDTSKLESQLEYI |
| | KNGGDFTRSMVNNYTYSLVSTAMEEEARRKNYILSWIFSEMRANRVDQMESLKDKF |
| | KFVSDTINYAYRKAGSNKGSLFDETEIRCILKSYGIAFSQELTKEIKELVKNGVLEGKV |
| | VIPTYKLDSPFTIAKSHFSFEHDYDSFEELCEHISDSDCKMYMNYGGDNKKDGINPASI |
| | ARFRISLGHGKNKDELRATLLKVYSGEYQYCGSSIQITKNKIILNLTMKIPKIETKLDKN |
| | TVVGVDLGIAVPAMCALNNNMYERLAIGSADEFLRVRTKYQAQRRRLQKSLKNSSA |
| | GHGRKKKLKALDRMDKAESHFVETYCHIVSKRVVEFAVKNRAKYINIENLNGYDTS |
| | QFILRNWSYYKLQQYITYKAERYGIAVRKINPCYTSQVCSVCGNWEDGQRKTQASFK |
| | CANQKCESHKKYKYGFNADFNAARNIAMSTLFMEDGEVTEKKKEEAREYYGIKKEN |
| | SKAV |
| 196 | MSYTEQFYIDKIAYYKNKLTKTKGKEEKQKIKDKLASLEEQENEFEESGILTQANVTD |
| | YTYDLVRNAMASEANRKNAIISYIILELLHNGGQTMDFNARNKLINDLVNYGLRVKG |
| | SSKGSLFDELDIENPLNAYGFAFKQDLKKKIRDMVNSKRVLDGKSSVITYKADSPFSIN |
| | KENMSFTHDYSSFEELSDHIRDNDTNLYFNFGSSGNPTIARFKINLGAGRHKKNKDELI |
| | ATLLKLYSGEYQFCGSRIGIEKNKIILNLVLSIPKKVRALDENTVVGVNLGVAVPAMC |
| | ALNNNEYERLAIGSADEFLRVRTKLQAQRRRLQKSLKDASGGHGRTKKLKALERVA |
| | KAESHFANTYCHMISKRIVDFALKNNAKYINLENLTGYDTNDFILRNWSYYKMQQYT |
| | TYKAEKYGIIVRKVNPCYNAQACSVCGNYAPGQRKSRAVFICANPACKSHKKNHGK |
| | LDAEFNNARNVAMSTLYMNDGQVTEKSFKEARDYFGIEEEIETI |
| 197 | MAKKQNKNTMQINRKITLIPVASDNDGWKKKMNTYLEKFYFDKKIKAKERQIKNTS |
| | KPERKEEYKQQLTEYKKQQEQFLNGELEDYTKQMVMSYTYDLVRDCMESEARQKN |
| | FIMSYMFSEMIREKVCYLKNKKEKEKWVNDNINVAFRVKGSPKGSIFDDVEIYSPLRA |
| | LSIQGYTQELKGRVKKFATDGGLDGKLAVDNYKLDSPFHMSKQGFDIVHEYEDLTEL |
| | KKNIGKSSCEIYVNLGNGGVPTFARFQLDFGHKGNREELISTITKVFTGEYKVCGSSIQI |
| | NKKNKIILNLGLEIPKRLTELDENTVVGVDLGLAVPAVCSLNNNQYKKEYIGDGEAFV |
| | KQRGKIQKEKQRLQKALKLSKGGHGRKRKMLALERFKEREANFVNTYCHRISKKVV |
| | EYALKNNAKYINIENLKGYDSSKFILRNWSFYQLQQDITYKAERYGIEVRKINPAFTSQ |
| | VCSFCGYWESGQRINQKTFKCGNPNCKSHNLKFFNADYNAARNISMSTLFTSDNYKF |
| | GKESLQKAADYYGIDLEIDSEDQEIA |
| 198 | MITVRKIKVRCEDKTFYDFMRKEQREQNKALNLSIGYIHTNSILKSVDSGAETLILNSIE |
| | KLNKKVDKLKKDLEKPKITDKKREQTEKAIQTNLKLIKDEQIKLEEGKQFRQGLDKQF |
| | SEIYINNNNLYHVLKSQTQVQYMRTLDLVTQKVKQDYSNNFVDIVTGKCSLMNYKS |
| | DFPLMIDKKCINIFKEEQNYKIRIMLGYELDIILGRRNNENVNELKSTLEKCISGEYKIC |
| | QSSISINKNDVIFNLTLDIPNTTNYEPVHGRVLGVDVGVKYPVYMCVSDNTYKRKHIG |
| | SAADFLKVRQQFQERKRKLQASLDITKGGKGRKKKTQALERFKDKERNFAKTYNHQ |
| | VSKKVVEFAKKNKCETIALEKITKEGIGDTILRNWSYYELQHMIEYKAKRECIKVEFV |
| | DPAYTSQTCSKCGHVSKDNRQTQEHFKCVNCGYELNADHNAAINIARRSIDYKPKEII |
| | EKTIEKTIQPITTDLVEETGEFKQLTFI |
| 199 | MQRSITLKIIRASDDKITWEELGYLLRGLSLKICRMSNFCMTHHLLHALKLETEMLNP |
| | KGNLYCYPHLAEEYPEVPAGIVCAAESRARKLFRRCGAKVLRSEMSLPSFRKDNSIPIP |
| | VAGYRILQDGDTNYAEIQLLSRQGAKTQKLPGRIRLTLADNWRDKAARPALQKLAA |
| | GKIRRGVASLYRAKNDWYLCIPYEVEAANTGGDFEPGLVMGVAFGVYNALVYGFNT |
| | LLKRGAISGEEILSHQAKVKARRKKIQEQYPWSGRKGQGREDTLKPLRHLHEAEKNY |
| | RELVNHRYAKWVVDIAVKNRCGEIHLDDGHAPPLGKNKILLSRWSLYDLKNKICRKA |
| | EEKGIRVTECSVPDLRTRCSHCGTEQVAGNHKRMFLCADCGYGSTEKNRGTGYISVD |
| | YNAARNLAMYDTGKNEPNMERDLIPKDDPAYDRNEGFREELSGSGQ |
| 200 | MATEYTCNTRKIEVHLHRHGEDEEAKQRLIDDYRVWDTINDNLYKAANRIVSHCFFN |
| | DAFEYRLKIHSPRFQEIEKLLKYPKRNKLTDEDIKQLKAERKQLFADFKKQRHTFLRG |
| | GVAEGANPEQNSTYKVISNEFLEVIPSEILTNLNQNISSTYKNYSLDVERGIRTIPNYKR |
| | GIPVPFSIKQRGELMLKRRDDGSIYVRFPLGLEWDLSFGRDRSNNREIVERVLSGQYD |
| | VGNSSIQESKNRKRFLLLVVKIPKENHNLNPDRIVGVDLGINIPLYAALNDNEYGGMGI |
| | GSREQFLNMRMRMVAKKRELQRNLRQSTNGGHGRAQKLQALERFEGKERNWVHLQ |
| | NHIFSKSIIEYAVKNNAGTIQMERLTGFGRDKNDEVDSDFKFILRYWSFFELQTMIEYK |
| | ANAAGIEVRYVDPYHTSQTCSFCGHYEKGQRLNQSTFVCKNPDCEKGKGKKLSNGT |
| | YQGINADWNAARNIALSDKIVDRKKK |
| 201 | MLITRKIELHISEADPELRKEHWKYLSFLNSEIYKAANLIVTNQLFNNFLENRVVDADG |
| | NVMDIGKRIRSLYRNKDKNADEIEKLKAAKLELRTEVKKFYLKSKQNTTYSITSHNFP |
| | EIDASIITTLNAQITGVLKKEWNEVERGSRSLRTYKKGMPIPFNLTSKSKKWFEKVDDE |
| | IFLTWFKGIKFKLFFGRDRSNNRAIVDRCLSGEYKYSDSSIQYKDRKIFLLLVVDIPEST |
| | KVLDENVSVGVDLGITVPAYCALSDGLKRLAIGSSEDLLRVRLQFQSRKRRLQRALKS |
| | SKGGQGRDKKLKALDNVADKEKRYVSTYNHMISSNIVKFAKDNNAGVIKLEMLEGF |
| | GEDEKNKFILRNWSYYQLQTMIQYKAKRENIKLVFIDPYHTSQTCSICNHYEAGQREK |
| | QSEFICKNPDCKNFDTPINADFNAALNIAVSKKVVTSKEDCEYFKKEN |
| 202 | MILTRKIKLVIVSENQKEGYSLIRNEIREQYKALNLAYNHLYFEHNAIQKLKQNDEDY |
| | KQKRSKLQELINKKYEEHQKVKNLEKKEALREAYNKKKQELYNFEKEYNEKARQAY |
| | QQVVRFTQQTRVRNLINRECNLMSDTKDGITSKVTQDYKNDCKAGLLIGKRSLRNYK |
| | KDNPLLVRGRSLKFYKEDGDYFIKWNKGTIFKCILHIRKKNVAELQSVLENVLLGAYK |
| | ICDSSIGFNNKDMILNLSLNIPDKETHDYIPGRVVGVDLGLKIPAYVSLSDKVYVRKGI |
| | GSIDDFLRVRTQMQKRRRRLQESLAAVKGGKGREKKLKALDHLKGKEANFAKTYNH |
| | FLSTQIVTFAVKNQAGQINMEFLEFDKMKNKSLLRNWSYYQLQMMVEYKAKREGIII |
| | KYVDAYLTSQTCSKCDHYEEGQRETQERFKCKSCGYEVNADYNASRNIAKSTRYISD |
| | STESEYHRKKQEALKEILGENDTINEQLSLFDKRDDIA |
| 203 | MQRSLKLKIVRPYDESITWEEIGYLLRGISYQICKMSNYCMTHHLLRALGMETENLNP |
| | QGNLYCYPRLAKEYPDVPTGIICAAEGRARKLFKRNAPGILRSETALPSFRKESSIPIPV |
| | AGYSLAKIGPDIYVADVQLLSRKAAKTGKLPGRIQFVLANNWRDKKAGSVLHQLAE |
| | GTIKRGIASIFRNNRDWYIGIPYSVDPAPSDGELDPDLVMGVAFGTHSALAYAFNNLL |
| | KRGELGGEEVLSHREKFKARRQHIREQYNWSGRKGHGRENALKPIRELYEKERNYRN |
| | LTNERYAKWVVEIAKKNHCGKINLDAGGDRRNGKQNILLAYWPQSELHMKIKNKAE |
| | AYGIQVQECTATDIRRRCSRCGAVQETSGDKRWFICSHCGYGKEEKKTSSGFVSVDY |
| | NAARNVSMWEGTT |
| 204 | MIAVRKLKVICKDKEFYDFFNMEQREQNKALNMAISLRHVNNTLKRIDSGAEVIIIKSI |
| | DKLNRKIETLEKELKNENITEAKKEKTLKDIETHKKIIKGEKKKLEEGRIYRKGLDKEF |
| | SQNYFDKTQLYHVLDGMTSIQHKRTIELVREKVKKDYENNFIDIVTGKQSLPNYKSDF |
| | PLMIDGSSIHIFEEDGIYKIKIMRGYELEVILGRRENENILELRKTLDRCSNGQYKVCQS |
| | EIQRDKNNNIIFNLTIDIPIENKKYTPVEGRVLGVDLGIKYPVYMCLNDDTYKRTSLGNI |
| | NNFLRVREQMQERRRAFQKDLTLTKGGKGKSKKTQLLNKLRENEKNFCKTYNHTMS |
| | KRVVEFAKKHNCEYIHIENLTKDGFSNSILRNWSYYELQKFIKYKADREGIVVKYIDP |
| | AYTSQMCSKCGYTDKENRQTQAEFKCISCGFKLNADHNASINIARSNKFIK |
| 205 | MPTITRKIELNLCTEGLADEEQKAQWNLLYHINDNLYKAANNISSKLYLDDHVSSMV |
| | MLKHAEYLTLVRALEKAQKQKTPDETVIEDLRRQVAAAEKDMTDQELAICNYATEM |
| | STQSLSYRFATEIETNIFAQILDCLKQGVYATFNSDAKDVKRGERAIRNYKKGMPIPFP |
| | WNKSLKIEHKDGEFYLLWYNGLRFHLNFGKDRSNNRLIVQRCMKMDKDYEGDYKM |
| | CNSSIQFVKREGKPKFFLLLVVNIPQEHVELDKKIVVGVDLGINSPAYVATNVTMERQ |
| | QIGRRETFLNGRMSFQRRYKSLQRLQTTAGGRGRKKKLEPLERLRNAEHNWVHTQN |
| | HLFSREVVQFAVKARAATIHMEDLSGFGKDNDGNADERKEFVLRNWSYYELQTMIS |
| | YKAQKYGIKVEKIRPAYTSRTCSWCGHEGFRKGEIFICENPACEKCGEKENADYNAA |
| | RNIANSKDIIKNHG |
| 206 | MGNNRMTICRKIKLFPVGDKDEINRVYDFIRNGQYAQYQACNLLMGQLMSEYYKYN |
| | RDIKNEEFKARQKEIMTNSNIILKDIDFATGVDTPSAVTQKVKQDFSTALKNGLAKGE |
| | RTVTNYKRTNPLITRGRNLTFYHEYETYHDFLDKINDSDLAVYVKWVNKIVFKVVFG |
| | NPHRSLELRSVIQNILEENYKVQGSSIEIDGKSIILNLSISIPKQLRELDENIVVGVDLGIA |
| | VPAMCALNNNLYERLAIGNADDFLRIRTKMQAQRKRLQKSLRNTSGGHGRAKKLKA |
| | LERLQKAEVHFVETYCHMISKRVVDFALKHNAKYINIENLTGYDTSDFILRNWSYYK |
| | LQDYITYKAAKYGIEVRKINPCYTSQICSVCGNWEFGQRKSQSVFECANENCDSHKK |
| | YEKTGFNADFNAARNIAMSTLWMGSGQVTEKSKQEAREYYDISEKYEQSKNGSENN |
| | KVA |
| 207 | MNIVKKIKLRIIDNDKELCKKQYLGFTEEQKKELIDKQYKFIRDSQYQQYLGFNRAMG |
| | FLMSGYYANNMDIKSDNFKEHQKKLTNSLYIFDDIKFGVGIDSKSLIVQRVKKDFSTA |
| | LKNGLAKGERSVTNYKRTYPLLTRHRSIKFLYAENELDIYLDWVNKIRFRCELGNHK |
| | NSLELQHTLRKVITGEYKISDSSLEFNKKNELILNLNLNIPETKATFIKDRTLGVDLGM |
| | AIPAYVSLSDTPYIRKGFGSYEEFAKVRNQFKDRRKRLLKQLSLVAGGKGRAKKLHS |
| | MEFLKNKEKQFAKTYNHSLSKKIIDFALKNNCEYINLEDIKSTSLEDRVLGQWGYYQL |
| | QEQIEYKAKLVGIKVRKVKAAYTSQTCSECGNIDKENRKNQSTFKCTNEDCKLNKKG |
| | INADWNASINIARSKEFIK |
| 208 | MATDYTVITRKIEVHLHRHGDSEEAIQRYKEEFRIWDEINDNLYKAANRIVSHCFFND |
| | AYEYRLKIHSPRFQEIEKLLRYSKRNKLTDEDIKQLKNERKQLFTDFKKQRLAFLRGG |
| | ATEGTNPEQNSTYKVVSNEFGDIIPSDILTCLNQNIASVYKAYSKEVEFGMRTIPNYKK |
| | GIPIPFTIRPNGVLNLQKREDGSIYIRMPKGLEWDLSFGRDRSNNCEIVERVLSGQYDV |
| | GNSSLQESKNKKRFLLLVVKIPKVNKALNQDRVVGVDLGINTPLYAALNDNEYGGM |
| | SIGSREQFLKMRMRMNAQKRELQHNLRHSTNGGHGRKQKLQALERLEGKERNWVH |
| | LQNHIFSKSIIEYALKNDAGVIQMERLTGFGRNKNDEVDEGYKFILRYWSFSELQNMIE |
| | YKANAAGIEVRYIDPYHTSQTCSFCGHYEKGQRISQSTFVCKNPECTKGKGKHKSDGS |
| | YEGINADWNAARNIALSNNIVGRKKK |
| 209 | MATEYTCITRKIEVHLHRHGDSEEALQRYKEEFRIWDEINDNLYKAANRIISHCFFNDA |
| | YEYRLKLHSPRFQEIEKLLKYAKRNKLTDDDIKALKAERKELFAEFKRQRQSFLGGSE |
| | QNSTYKVVTDEFLEVIPSHVLTCLNQNISSTYREYALDVEHGRRTIPNFKKGIPVPFPIK |
| | ATGELLLRKREDGSIYIRFPKGLEWDLNFGRDRSNNREIVERVLSGQYDVGNSSIQETK |
| | NRKRFLLLVVKIPKESRALNPDRVVGVDLGVAVPLYAALNDNPYGGMSIGSADQFLK |
| | VRMRMAAQKRELQRTLRNSTNGGHGRKHKLQALDRLEGKERNWVHLQNHIFSKEL |
| | VEFALRNEAGAIQMERLTGFGHDRNDEVDEGFKFILRYWSFFELQTMIEYKAKAAGIE |
| | VRYVDPYHTSQTCSFCGHYEKGQRVNQATFICKNPDCTKGKGKERSDGTFEGINADW |
| | NAARNIALSDKIVERKKK |
| 210 | MVEQGVTITLNKSDKNTYTIARKVRLAVDGDKEEVNRVYQFLRDGIYNQNKAYNIFI |
| | SSVYSAIINGASTEELNEIYKRGSRKPKEDDETYSLYKFGEIEFPVGVGTTASLKQMVK |
| | NDLKKAKNDGLFKGKISLPNRKLNAPLRIESACFSFIHNYNSYQEFLDHLYTDDCEIIM |
| | KFVNNIRFKVNLGQACKTHELRSVFQNIFEENYKVCGSSIEIDGTKIILNLSLTIPKKKH |
| | ELDESIVVGVDLGIAVPAVCSLNNNTYIKKFIGSRNDFLRERTKLKAQRRNVQKSLKF |
| | TSGGHGRKKKLRHLEVFTEHEKNWVKNYNHRVSKEIVDFALSNDAKYINIENLQGFD |
| | SSNFLLANWSYYQLQQQIEYKADMHGIIVRKVNPYHTSQRCSCCGFESPDNRPKDKK |
| | GQAYFKCLNCGTEMNADFNASQNIARSTDFSIGEVTLEEDKKKHNKSKKIPKEKQAA |
| 211 | YISSHMFFNEAYIERLKAHSHRYREIKKTLKKIDEFTPEEAKALNDEKRVLEKQFVEER |
| | LRFLKGGDANGSGSVMSSTRQAVVEAFPEIPFKVLDCLNREMSKTFSQYKRDIENGKR |
| | TLSNYKKYYPVPFSMEQGKQLRKREDGSIYVMFPGKLEWDLYFGKDPSNNREIVERIF |
| | NGEYQACDSSIKEIKGRKKQILHLVVKIPKKTIKRDSNRVVGIDLGVNTPLYAALNDSE |
| | RERFSIGSREAFLNVRMRFNAQRRELQRNLRHTTNGGHGRKQKLQALERLEGKERN |
| | WVHLQNHIFSKSLIEFAQKCEAGVIQMEELSGFGRDKFDSVDDGYKFILRYWSFFELQ |
| | NMIEYKANAAGIEVRYVNPAYTSQTCSYCKHYEKGQRISQSTFVCKKCTEGKGKQRK |
| | DGSYEGINADWNAARNIAMSDKIVDRKKK |
| 212 | MITTRKLKLTILGDEETRKLQYKLIRDEQYEQYKALNLCMSLLNTHNILSGYNTGAEN |
| | KLNSQIDKLNKKLEKAQFEIKKNDIKQSKLDKLNSDIELYQNELNKLKEQFTQSSKYR |
| | SDIDLKFKEMYIDDLYTIVQNQVTFKNKDLMSLVTQRAKKDYSIALKNGMARGERSL |
| | TNYKRDFPLMTRGERWLKFKYDENSDDILIDWISGIKFKVILGYRKNENSIELRHTLHK |
| | VINKEYKICDSSIQFDRNNNLILNLTLDIPNNSKSEFIENRTLGVDLGIKYPAYICLSDDT |
| | FKRESIGCAEDFIRVREQIRNRRKRLQQQLKMVQGGKGREKKLKALDRISDKERNFV |
| | KTYNHMISKNIVNFAKKHKCQYINLEKLTKDGFPNMILSKWSYYELQQMIEYKAERE |
| | NIKVRYIDPAYTSQTCSRCSHIEKDNRETQEKFKCLKCEFELNADHNAAINISRSVNFK |
| 213 | MISTRKIKVRCDDNTFYTFFRQEQREQNKALNIGMGIIHADAILHNIDSGAEKKLKKSI |
| | EGLQGKIDKLNKYLEKENITDKKKKEVLKAIETNKKILDGEKKAFKESEEYRKGIDEL |
| | FKSTYLKSNTLYHVLDSMVNIQYKRTLSLVTQRIKKDYSNDFVGVVTGQQSLRNYRN |
| | DNPLMISNQQLDLKYIEDTFYLDVMCGYRLEIILGRRDNQNVNELKSTLHRILSKEYK |
| | VCDSSMQFDKNNKDVILNLIIDIPNKSNMYEAIKERTLGIDLGVEVPIFMCLNDNTYIK |
| | KGIGDINNFLRVRQQIQIRRRKLQKDLTLTNGGKGRKKKLQLLEKLQENEKNFVKTYS |
| | HVLSKRIIEFAKKNKCEYINLEKLTKDGFDNIILRNWSYFDLQRMIEYKAKREGITVRY |
| | VNPSFTSQKCSKCGEIDKENRQTQADFKCTNCGFELNADHNAAINIARSIDFV |
| 214 | MQRSIKLKILRPFDGNITWEALGYLLQGLSFKVCRISNFCLTHHLLRALKLETENLNPK |
| | GHLYCYPRLAEEYPEVPAGIICAAEGRARKVFNQKGKSIMRSEMALPTFRKNCSIPIPT |
| | AGYNLRWEGKDTCIAEVQLLSRQGAKTGKLPGRISLVLADNRRDKTAGAVMRKLAE |
| | GTLRRGAATLFREKKDWYISIPYETEAIHTEKDFVPGLVMGVAFGIRCTLAYGFNRLL |
| | KRGEIKGDEVLAHQEKYLARRKKIQEQYNWSGRRGHGREHALKPLRHLYEKERNYR |
| | SLVNARYAKWIVEIAEKNRCGAIHLDGENRLQRGKYPALLARWPLEELRQKIREKAE |
| | LYGIKVSECTEAGIRERCSRCGAVQEDAADGYKFTCTACGYGAKGNNSSTGYISVDY |
| | NAARNLAVWEPETDS |
| 215 | MATEYTCITRKIEVHLHKHGDSEEALQRLNEECRIWDEINNNLYKAANRIISHCFENDT |
| | YEYRLKLQSPRLQEIEKLLSNPKRNKLSDEDIKQLKAERKQLFANFKKQRQVFLRGGV |
| | EEGANPEQNSTYRVVSNEFIDVIPSEVLTNLNQNISSTYREYSLDVERGSRTIPNYKKGI |
| | PVPFSIKRSGELMLKKREDGSIYVRFPKCLEWDLFFGRDRSNNREIVERVLNGQYDVG |
| | ISTIQETKNKKRFLLLVVKIPKESKKLNPNRVVGVDLGINIPLYAALNDNEYGGLGIGS |
| | REQFLKVRMRMVAQKRALQRNLRHTTNGGHGRAQKLQALDQLEGKERNWVHLQN |
| | HIFSKSIIEYALKNGAGVIQMERLAGFGRDKNEEVENEFKFILRYWSFFELQTMIEYKA |
| | NVAGIEVRYIDPYHTSQTCSFCGHYEKGQRINQSTFVCKNPDCVKGKGKQHADGSYD |
| | GINADWNAARNIALSTTVVD |
| 216 | MQRSIKLKILRPFDGNITWEALGYLLQGLSFKVCRISNFCLTHHLLRALKLETENLNPK |
| | GHLYCYPRLAEEYPEVPAGIICAAEGRARKVFNQKGKGILRSEMALPTFRKNCSIPIPT |
| | AGYNLRWEGKDTCIAEVQLLSRQGAKTGKLPGRISLVLADNRRDKTAGAVMRKLAE |
| | GTLRRGAATLFREKKDWYISIPYETEAIHTEKDFVPGLVMGVAFGIRCTLAYGFNRLL |
| | KRGEIKGDEVLAHQEKYLARRKKIQEQYNWSGRRGHGREHALKPLRHLYEKERNYR |
| | SLVNARYAKWIVEIAEKNRCGAIHLDGENRLQRGKYPALLARWPLEELRQKIREKAE |
| | LYGIKVSECTEAGIRERCSRCGAVQEDAADGYKFTCTACGYGAKGNNSSTGYISVDY |
| | NAARNLAVWRQETNS |
| 217 | MGNDRMTICRKIKLFPVGDKEEINRVYDFIRNGQYAQYQACNLLMGQLMSEYYKYN |
| | RDIKNKEFKARQKEIMTNSNIILKDIDFVTGVDTPSAVTQKVKQDFSTALKNGLAKGE |
| | RTVTNYKRTNPLITRGRNLTFYHEYETYQNFLDKINDSDLAVYIKWVNKILFKVVFGN |
| | PHRSLELRSVVQNILEENYKVQGSSIEIDGKSIILNLSISIPKQLRELDENIVVGVDLGIA |
| | VPAMCALNNNIYERLAIGNADDFLRIRTKMQAQRKRLQKSLRNTSGGHGRAKKLKA |
| | LERLQKAEVHFVETYCHMISKRVVDFALKHNAKYINIENLTGYDTSDFILRNWSYYK |
| | LQDYITYKAAKYGIEVRKINPCYTSQICSVCGSWEFGQRKSQSVFECANENCDSHTKY |
| | ERGFNADFNAARNIAKSTLWMESGQVTEKSKQEAREYYGISEKYEQSKNEVENNKV |
| | A |
| 218 | MQRSIILKIIRPYDESIKWDDVGYLWRGLSFKVCKISNYCMTHHLLRAMNLETENLNP |
| | QGRLYCYPHLAKEYPEIPAGIICAAEGRARKVFKQNAKGILYSETSLPSFRKDCSIPIPV |
| | SGYSLLKAGADTYVASIQFLSRQAAKTQKLPGRIQLVLASNWRDKSAGRILQQLAEG |
| | TLKRGIASLFRKKRDWYFSIPYEVEPSGADDSFEPELAMGVVFGFQCALAYGFNRLLK |
| | RGMLGGDELLAHREKMLARKKQILTQYRWSGRKGHGRESALKPLQALYEKERNYR |
| | NLTNERYAKWIAEIAKKNRCGKIYLDTGGSSGHGTQNILLAYWPKEALRKKIMNKAE |
| | AYGIEVVECTDAEIRNRCSRCGTLQEPLENKKWFVCNHCGYGKDDKKTGDGFISVDY |
| | NAARNLAVYDTGSKESV |
| 219 | MISKRKIKVRCDDNTFYTFFRQEQREQNKALNIGIGIIHSNAILHNIDSGAEKKLKKSIE |
| | SLQGKIDKFNKHLEKEKLTDKKKEEVLKAIETTKKILDGEKKAFKKSEEYRKGIDELF |
| | KSTYLKSNTLDHVLDSMVNIQYKRTLSLVTQRIKKDYSNDFVGIITGQKSLRNYRNDN |
| | PLMISNQQLDFKYIEDTFYLDVMCGYRLEIILGRRDNQNVNELKSTLHRILSKEYKVC |
| | DSSMQFDKNNRDVILNLGIDIPNKSNMYEAIKGRTLGIDLGVEVPIFMCLNDNTYIKK |
| | GIGDINNFLRVRQQIQVRRRKLQKDLTLTNGGKGRKKKLQLLDKLQENEKNFVKTYS |
| | HALSKRIIEFAKKNKCEYINLEKLTKDGFDNIILRNWSYFDLQRMIENKAKREGITVRY |
| | VNPSFTSQKCSKCGEIDKKNRQTQADFKCINCGFELNADHNAAINIARSLDFV |
| 220 | ITWEALGYLLQGLSFKVCRISNFCLTHHLLRALKLETENLNPKGHLYCYPRLAEEYPE |
| | VPAGIICAAEGRARKVFNQKGKGILRSEMALPTFRKNCSIPIPTAGYNLRWEGKDTCIA |
| | EVQLLSRQGAKTGKLPGRISLVLADNRRDKTAGAVMRKLAEGTLRRGAATLFREKK |
| | DWYISIPYETEAIHTEKDFVPGLVMGVAFGIRCTLAYGFNRLLKRGEIKGDEVLAHQE |
| | KYLARRKKIQEQYNWSGRRGHGREHALKPLRHLYEKERNYRSLVNARYAKWIVEIA |
| | EKNRCGAIHLDGENRLQRGKYPALLARWPLEELRQKIREKAELYGIKVSECTEAGIRE |
| | RCSRCGAVQEDAADGYKFTCTACGYGAKGNNSSTGYISVDYNAARNLAVWRQETN |
| | S |
| 221 | MQRSIILKIIRPYDESIKWDDVGYLWRGLSFKVCKISNYCMTHHLLRAMNLETENLNP |
| | QGRLYCYPHLAKEYPEIPAGIICAAEGRARKVFQQNAKGILYSETSLPSFRKDCSIPIPV |
| | SGYSLLKAGADTYVASIQFLSRQAAKTQKLPGRIQLVLASNWRDKSAGRILQQLAEG |
| | TLKRGIASLFRKKRDWYFSIPYEVEPSGADDSFEPELAMGVVFGFQCALAYGFNRLLK |
| | RGMLGGDELLAHREKMLARKKQILTQYRWSGRKGHGRESALKPLQALYEKERNYR |
| | NLTNERYAKWIAEIAKKNRCGKIYLDTGGSSGHGTQNILLAYWPKEALRKKIMNKAE |
| | AYGIEVVECTDAEIRNRCSRCGTLQEPLENKKWFVCNHCGYGKDDRKTGNGFISVDY |
| | NAARNLAVYDTGSKESV |
| 222 | MATEYTCITRKIEVHLHRHGDSEEAAQRLKEEYRIWDEINDNLYKAANRIISHCFFND |
| | AYEYRLKLHSPRFQEIEKLLKYAKRNKLTDDDIKALKAERKELFAEFKRQRQSFLGGS |
| | EQNSTYKVVTDEFLEVIPSDVLSCLNQNISSTYREYALDVEHGRRTIPNFKKGIPVPFAI |
| | KVHRELALRKREDGSIYIRFPKGLEWDLNFGRDRSNNREIVERVLSGQYDVGVSSIQE |
| | AKNGKRFLLLVVKIPKESRALNPDRVVGVDLGVNIPLYAALNDNTYGGLSIGSRDQFL |
| | NVRMRMAAQKRELQRNLRVATNGGHGRKQKLQALDRLEGKERRWVHLQNHIFSKS |
| | IIEYALRNEAGAIQMERLTGFGHDRNDEVDEGFKFILRYWSFFELQTMIEYKAKAAGI |
| | EVRYVDPYHTSQTCSFCGHYEKGQRVNQATFICKNPDCTKGKGKERSDGTFEGINAD |
| | WNAARNIALSDKIVERKKK |
| 223 | MPIITRKIELKLCTEGLSEETIKSQRMLLYHINDNLYRVANNISSKLYLDEHVSSLVRM |
| | KNGDYLSLEKQLVKAKKQKNPDTKTITELEERILTVKREMNSQEIAISKYATEMSTKT |
| | LAYNFAKELGSDIFGQILACLQQNVHSTFSEDAQEVRRGERAIRNYKKGMPIPFPWNR |
| | SIRIEALDGEFYLRWYNGIRFHLFFGKDRSNNRQIVKRALCLDPDYSGENYKLCNSSIQ |
| | LVKREHERKTFLLLVVDIPKEVRKLNKDIVVGVDLGINVPAYVATNSTEERKEIGDRE |
| | HFLNERMSFQRRYKSLQRLKCTAGGKGRKKKLEPLERLREAEHKWVHTQNHRFSRE |
| | VVDFALHAEAATIHMENLSGFGKDQEGNADEKKEFVLRNWSYYELQNMIAYKAAK |
| | YGIKVEYVKPAYTSKTCSWCGKEGFRQSTIFICENHECKMCGIKVHADYNAARNIANS |
| | NNIIKNE |
| 224 | MATEYTCITRKIEVHLHRHGDSEEAAQRLKEEYRIWDEINDNLYKAANRIISHCFFND |
| | AYEYRLKLHSPRFQEIEKLLKYAKRNKLTDDDIKALKAERKELFAEFKRQRQSFLGGS |
| | EQNSTYKVVTDEFLEVIPSDVLTCLNQNISSTYREYALDVESGRRTIPNFKKGIPVPFAI |
| | KVHRELALRKREDGSIYIRFPKGLEWDLNFGRDRSNNREIVERVLSGQYDVGVSSIQE |
| | AKNGKRFLLLVVKIPKESRALNPDRVVGVDLGVNIPLYAALNDNTYGGLSIGSRDQFL |
| | KVRMRMAAQKRELQRNLRVATNGGHGRKQKLQALDRLEGKERRWVHLQNHIFSKS |
| | IIEYALRNEAGAIQMERLTGFGHDRNDEVDEGFKFILRYWSFFELQTMIEYKAKAAGI |
| | EVRYVDPYHTSQTCSFCGHYEKGQRVNQATFICKNPDCTKGKGKERSDGTFEGINAD |
| | WNAARNIALSDKIVERKKK |
| 225 | MQRSITLKIIRPEDETISWEELGYLLRGLSFKVCRMCNFCMTHQLLHALKLETELLNPQ |
| | GNLYCYPRLAEEYPDVPTGIICAAETRARKLFRRSAEAVLHSETSLPRFRKDSSIPVPV |
| | AGYKILQDADHNVYADVQLLSRQGAKTQKLPGRIRLVLADNWRDQSAKAALQQIVA |
| | GKVKRGVASLFRVKNDWYFQIPYVTEAVNTEEIFQPDLVMGVAFGLQDALVYAFSTS |
| | MKRGAVSGEEVLAHQEKYVARRKKIQEQYNWSGRKGHGREDALKPLRHLYETERN |
| | YRNLVNSRYAKWVVDIAAKNRCGVIHLDSANYVSSGKNILLSRWPLYDLKEKIRRKA |
| | EEKGIQVTECSIPNLRTRCSRCGKEQEPEGEKRTFVCKDCGYGKADKNRRGGFITVDY |
| | NAARNLAVYESEDAKL |
| 226 | MATEYITITRKIEVYLHRHGDSDEAKQRYQQEWQIWHDINDNLYKAANRIVSHCFFN |
| | DAYEFRLRLHSPRFAEIEKALKHSKKNKLSDDEVKALKAERQELYKSFKQQRLAFLR |
| | GGATEGANPEQNSTYKVISDEFGDIIPSDILTCLNQNIASVYKAYSKEVQFGDRTIPNY |
| | KKGIPAPFSIKAGGALLLKKREDGSIYVRMPKGLEWDLVFGRDRSNNREIVERILSGQ |
| | YDAGNSSIQQAKNGKCFLLLIVKIPKSNIALNKDRVVGVDLGINIPLYVALNDNEYGG |
| | MGIGSREQFLKMRMRMSAQKRELQRNLRHSTNGGHGRKQKLQALERLEGKERNWV |
| | HLQNHIFSKSVIEFAQKHNAGVIQMERLKGYGKDANGEVREEAKFLTRYWSYFKLQT |
| | MIEYKANAAGIEIRYIDPYHTSQTCSFCGHYEKGQRISQSTFICQNPECKQGKGKQKSD |
| | GTFEGINADWNAARNIALSEQYVDKKKK |
| 227 | MATEYTCITRKIEVHLHRHGDSDEALQRYKEEYRIWDEINDNLYKAANRIISHCFFND |
| | AYEYRLKLHSPRFQEIEKLLKYAKRNKLTDDDIKALKAERKELFAEFKRQRQSFLGGS |
| | EQNSTDRVVSHEFLDVIPSDILTCLNQNISSTYREYALDVEHGRRTIPNFKKGIPVPFPIK |
| | ATGELLLRKREDGSIYIRFPKGLEWDLNFGRDRSNNREIVERVLSGQYDVGNSSIQETK |
| | NRKRFLLLVVKIPKESRALNPDRVVGVDLGVNIPLYAALNDNTYGGLSIGSRDQFLKV |
| | RMRMAAQKRELQRNLRVATNGGHGRKQKLQALDRLEGKERNWVHLQNHIFSKSIIE |
| | YALRNEAGAIQMERLTGFGHDRNDEVDEGFKFILRYWSFFELQTMIEYKAKAAGIEV |
| | RYVNPYHTSQTCSFCGHYEKGQRVNQATFICKNPDCTKGKGKERSDGTFEGINADWN |
| | AARNIALSDKIVERKKK |
| 228 | MATEYTCITRKIEVHLHRHGDSEEAAQRLKEEYRIWDEINDNLYKAANRIISHCFFND |
| | AYEYRLKLHSPRFQEIEKLLKYAKRNKLTDDDIKALKAERKELFAEFKRQRQSFLGGS |
| | EQNSTDRVVSHEFLDVIPSEVLTCLNQNISSTYREYALDVEHGRRTIPNFKKGIPAPFSI |
| | KANRELALRKREDGSIYIRFPKGLEWDLNFGRDRSNNREIVERVLSGQYDVGVSSIQE |
| | AKNGKRFLLLVVKIPKESRELNPDRVVGVDLGVAVPLYAALNDNQYGGMSIGSADQ |
| | FIKVRMRMAAQKREMQRTLRNSTNGGHGRKQKLQALDRLEGKERRWVHLQNHIFS |
| | KELVEYALRNEAGAIQMERLTGFGHDRNDEVDEGFKFILRYWSFFELQTMIEYKAKA |
| | AGIEVRYVDPYHTSQTCSFCGHYEKGQRVTQSKFVCKNPDCTKGKGKERSDGTFEGI |
| | NADWNAARNIALSDKIVERKKK |
| 229 | MPTITRKIELTLCTDGLSDEERKAQWGLLYHINDNLYKAANNISSKLYLDEHVSSMVR |
| | LKHAEYLSLQKELAKAERQKMPDVDVIEELRERLSAAEQEMSDQELAICKYATEMST |
| | NTLAYRFATEIETNIFGQILARLENNAQAVFLTDAPDVKRGERAIRNYKKGMPIPFPW |
| | NNSIKIECEGGEFYLRWYSGLRFHFNFGKDRSGNRLIVQRCLKLDKEYDGEYKLCNSS |
| | IQMVKRDGSTKFFLLMVVNIPQEYVELNKHIVVGVDLGINVPAYVATNITPERKAIGD |
| | REHFLNTRMAFQRRYKSLQRLKTTAGGKGRTKKLEPLERLRQAEHNWVHTQNHLFS |
| | REVVNFALQTHAATIHLEDLSGFGKDSDGNADERKEFVLRNWSYYELQNMITYKAA |
| | KYGIRVEKIRPAFTSRTCSCCGHEGFREGVTFICENPECQQFGEKVHADYNAARNIANS |
| | KDIIKKNE |
| 230 | MATEFTCITRKIEVHLHRHGDSEEAIARYKNEWDMWDEINNNLYKAANRIVSHCFFN |
| | DAYEYRLKMHSPRFQEIEKLLRYTKRNNLSDNDIKQLKEERKNLFADFKKQRLAFLQ |
| | GNSGIGSEQNSTYKVISNEFLDTIPSQILTNLNQNISSTYREYTQDIERGIRTIPNFKKGIP |
| | VPFSIKKGGDLMLKKRNDGSIYIRFPKGLEWDLNFGRDRSNNREIVERILNGQYDVGN |
| | SSIQETKNKKRFVLLVVKIPKENQKLDTNRIVGIDLGINTPLYAALNDNEYGGFSIGSR |
| | DQFLKMRMRMAAQKRELQRNLRHTTNGGHGRNQKLQAFKRLQDKEKNWVRLQNH |
| | IFSKSIIEYALKNKAGVIQMERLTGFGRDKNDEVNEDYKFILRYWSFFELQTMIEYKAN |
| | AAGIEVRYIDPYHTSQTCSFCGHYEKGQRINQQTFICKNPDCTKGKGKQEKDGTYKGI |
| 231 | MPTITRKIELTLCTEGLSDEQRKAQWGLLFHINDNLYKAANNVSSKLYLDEHVQSMV |
| | RMKHDEYLGLLKELARAEKQKMPDAAVIAELREKIAAAEKEMTDQELAICKYATEM |
| | PTQSLSYRLVTEIETNIFAQILDCLKQNVFATFNSDARDVKRGERAIRNYKKGMPIPFP |
| | WNKAIKIESQGDDFFLRWYSGIRFKFVFGKDRSNNRAIVMRCLKLDEDYDGEYKLCN |
| | SSIQMVKRDGGMKLFLLLVVSIPQEHVELNKKIAVGVDLGVNVPAYVATNITEERKAI |
| | GDREHFLNTRMQFQRRYKSLQRLKTTAGGKGRTKKLEPLERLRDAERNWVHTQNHL |
| | FSREVVNFAVQTHAATIHLEDLSGFGKDYDGNADERKEFVLRNWSYYELQNMITYK |
| | AAKYGIHVVKVRPAFTSRTCSCCGQQGFREGVTFICENPECKQYGEKVHADYNAARN |
| | IANSNDIIQDNHE |
| 232 | MITSRKIKLSIVSNNSTEAYNFIRKEMKVQNKALNVAMNHLYFNSIARQKILLADKAY |
| | QQKLKAAINSQEKSFNTLKELEQKQCIEEDSEKKQALKERLIKTKNAYEKAKEKVSNL |
| | RKSRNKDSFQEYKNIIGQVEQTHLRDIISSQFNLHSDTKDRLTMIAKQDFENDIAEVLS |
| | GDRSLRTYKKNNPLYIRGRNTVLYKEGNEFFIKWIKGIVFKCILGVKNQNKTELYKTL |
| | ECVLAEIHKICDSSMNFNQQDKLILNLTLDMPDKSEHRKVPERIAGVDLGLKIPAYFA |
| | VNDVPYIRKSIGKIEDFLKVRISIQSQKRSLQRALQSSKGGKGRKKKLKTLDQFKEKEK |
| | NYITTYNHFISKKIISLAVQYGVEQINLELLTLKETQKKLLLRNWSYYQLQQFIEYKAN |
| | REGISIKYVDPFHTSQTCSKCGHYEDGQREKQDTFCCKSCGFKDNADYNAARNIAAST |
| | RYITNKKESEYYQQNNNEIA |
| 233 | MVKLKHNEYASLEKKLQKAQKEKTSDDNLIADLEEKLAAAKREMTDQARAICKYAT |
| | EMSTETFAYKLATEIETNVFGQILSYLKKAVQSNFSNDAQAVRRGERSIRNYKKGMPI |
| | PFPLKDRIKIKDNDFYLHWYNNIRFKLHFGKDRSNNRQIVNRCFNSVNDYKLGSSASI |
| | QIVKRNGSPKFFLLLVINIPKEEVELNKKIVVGVDLGINVPAYVATNMTEERKAIGDRE |
| | HFLNNRMAFQRRFKSLQRLRGTAGGKGRTKKLEPLENLRKTERNWVHTQNHLFSRE |
| | VVKFAVQTHAATIHMEDLSNFGKDKDGNADEHKEFVLRNWSYYELQEMIRYKAKK |
| | YGIEVRNVRPAYTSQTCSWCGEQGFRQGATFICKNPECKQYSEKIHADYNAARNIAKS |
| | KDIIKKNE |
| 234 | LYKAANNISSKLYLDEHVSSLVRLKHKEYKDLMRELAKAKKQKNLDEASIIEMEGRL |
| | RSYEEEMTDQELAICKYADEMSSLSLAYGFATELELDIYAQILTQIQSKVHQDFQNDQ |
| | KDVREGKRSIRTYKKGMPIPFPWNNSIRIEISKKNKVEEQEKKTRRDSEDYDFYLNWY |
| | NGLRFRLHFGKDRSNNYQIIKRCFKLDEYCHDNYQLKASSIQLVIKNKKPELYLLLVV |
| | DIPQEKYSLNNKVVVGVDLGINTPAYVATNVTEDRKAISDREHFLNARMAIKRRYRS |
| | LQRLKGTAEGRGRKKKLEPLERLREAESNWVHTQNHLFSRDIIKFALRVKAATIQME |
| | KLEGFGKDEDGNVEEDKKFLLGEWSYYELQNMVKYKAAKVGIKVHFVKPAYTSQT |
| | CSWCGERGIRDGTAFYCQNPNCKQHGKKDINADYNAARNIAKSTEIVK |
| 235 | MRIIRPYYGDEIEKFITAGKEKSKADGADGALTKIFWDRLKASHPEIVSAGEFYGLLCA |
| | MRMEAVVYYNRAISKLYQSLIVDVDGAQVSTAKALSAGPYHEFRERFTSYISLGLRQ |
| | KLQSNFRRKELLRCQLALPTAKSDRFPIPISKQVDKQGKGGFKVSELQNGDFIIELPLM |
| | AYHKAKGKTEREYVELDAGPAILNIPVILSTQRRRANKTWFKDEGTDAEIRRVMSGE |
| | YKVSWLEILQRNRFGKAHGDWYVNFTIKYQPKDCGLDPKVKGGIDIGLSSPLVCAVT |
| | NSLDRLTIRDNDLVAFNKRALARRRTLLRRNRYKRAGHGSANKLEPITALTAKNELY |
| | RKAIMRRWAREAADFFRHNKAAAVNMEDLTGIKDREDYFSQMLRSYWNYSQMQTV |
| | LENKLKEYGIAVNYINPKDTSKTCHSCGHVNQHFDFSYRSANKFPMFKCVKCGVECG |
| | ADYNAARNIAVA |
| 236 | MIIARKIRLTVISDNRDEAYSFLRNEMYYYYKALNFAMNHVYFNYVAKEKIKMADD |
| | KYIEREQRYINAINNTHATLKKVRTESQKDLALKRIELNEQNLKKLRQSTSKEAREML |
| | SQAISSAERTNTSDAVQKAFPMLTRDSIDFAASKATTEFNNDLKLGLLSGERVLRTYK |
| | KTNPLQIRGRLLKFFKEDGDYCIKFAKGIIFKCVLGIKRKNNTELAHTLEKVIDGTYKV |
| | CDSSFEYNDSKLILNLALQIDITARQQQPKVQGRVVGVDLGLKIPAFCALNDSIYIRERI |
| | GDIEDFLKVRTQLQARRKRLQRALKSAKGGKGQGKKLKALERFREHEKNFVKTYNH |
| | YLSKKIVHFSVLYGAEQINLELLQMAESQNKSILRNWSYYQLQQMIEYKAAKEGIKIK |
| | YVDPYRTSQTCSKCGNYENGQRKTQATFECKSCKFKENADFNAARNIAKSTAYITDK |
| | FQSEYYKNYMDCKLGARASGFH |
| 237 | MISTRKIKVRCDDNTFYTFFRQEQREQNKALNIGIGIIHSNAILHNIDSGAEKKLKKSIE |
| | GLQGKIDKFNKHLEKEKLTDKKKEEVLKAIETTKKILDGEKKAFKKSEEYRKGIDELF |
| | KSTYLKSNTLDHVLDSMVNIQYKRTLSLVTQRIKKDYSNDFVEIITGQKSLRNYRNDN |
| | PLMISNQQLDFKYIEDTFYLDVMCGYRLEIILGRRDNQNVNELKSTLHRILSKEYKVC |
| | DSSMQFDKNNKDVILNLVIDIPNKSNMYEAIKERTLGIDLGMEVPIFMCLNDNTYIKK |
| | GIGDINNFLRVRQQIQVRRRKLQKDLTLTKGGKGRKKKLQLLDKLQENEKNFVKTYS |
| | HALSKRIIEFAKKNKCEYINLEKLTKDGFDNIILRNWSYFDLQRMIENKAKREGIVVRY |
| | VNPSFTSQKCSKCGEIDKKNRQTQADFKCINCGFELNADHNAAINIARSIDFV |
| 238 | MIITRKIAITIVSEEAQESYNYLRQQMYYYYKALNFGMNHIYFNYVAKEKIKLADSAY |
| | KEREEKYINAIHIAKEKLQKDLSVSQRAQAEKSLEVNTNNLDKLRKAISKDAKETFQK |
| | VMGAVERTNVTDAIKKEFPILQRDSIDFAASKVASDENNDLKLGLMTGSRTLRIYKRN |
| | QAYPFRSRRLKLYKENGDFFIKSSKSLLFKCLLGVKRQNSKELIQILEKILDNQYKICDS |
| | SLEFNKKKLILNLCIEVDENTHSENMKVPGVVKGRIVGVDLGIQIPAYCTLNDSPFKKK |
| | AIGSVDDLLRIRTQMQARRRRLSKNLISARGGKGRGKKLKALDRFEEYERNYVTTYN |
| | HFISKQIIQFTLQNQAEQINLELLQMEHTKSKSILRNWSYYQLQQMIEYKAKREGIVIK |
| | YVDPYHTSQVCSKCGHFEENQRMDQNTFRCKKCKYRTNADYNAAKNIANSTRYISSI |
| | QESEYYNIKNRNIVK |
| 239 | MELKRTARVKLAIPDDRRDDLKRTMLTFREVAQRFADRGWERDEDGYVITSRTRLQS |
| | LVYKQVREDTGLHSDLCIGAVNLAADSLRSAVERMKAGKNVGKPTFTVPTATYNTG |
| | AVSYFTDGDGTGYCTLAAYGGRVRAEFVYPPDEDCPQRQYLGGDEWEPKGATLHYE |
| | RDDGEYYLHVTVERDEPETELGEAENGTVLGVDLGVENIAVTSAGAFYSGGLFNHRR |
| | DEYERIRGSLQQTGTESAHRTIEKMGDRERRWNTDVLHRISKAIVQEAITHDCSHIAFE |
| | DLTDIRDRMPGAKKFHGWAFRQLYEYVEYKAAEFGIATTQVDPAYTSQRCSKCGTTL |
| | RENRTSQAAFCCQKCGYEVHADYNAAKNVATKLLRSGQKSPAGGATNQLALKSGTL |
| | NGNGDFTPASS |
| 240 | MKEQLNKTLTFGLGKPLGWDIRGKAFIDPTEDQRREIYISLRATSRISAQMVNMLNAR |
| | EYVRRIMKIPEAFVNEFKGSYIPIKQELKTLGLEEVDDISGATLSQTWALGVKPDFAGE |
| | HGKRLLMKGDRQLPTHRIDGTHPIYGRADGTKIILHEDRYFLIVQLFSSKWANKNEFP |
| | SGWIAFPVKIKPRDKTLAGQFKRIIDGEWKLKNSHILRNPRKRGNTWLGQVVVSYTPD |
| | PFKDIDPKIIMGIDLGVSVPACLHIRENGKAKKWAMQVGRGRDMLNTRGIIRSEIVRII |
| | RSLRSKDSPLDNESKRAAKAKLKNLRKREKRVMKTASQKIAASIADVARRNGAGTW |
| | KMELLSENIKDEDPWLRRNWAPRMVVDAVRWQAEQVGAKLEFVDPAYTSQRCSKC |
| | GHISRENRPKGKKGAAHFECVRCGYKDHADKNAARNISTPGIVDLIKEQISKSPNGEE |
| | R |
| 241 | MCRVLRRTVCLKLDVPDDRRDDLHETTDRFRQAAQIAVDRAFERNDDGYVITHKTK |
| | LHHLTYEQAREATDGLNANLVQAARNLAGDAAKGVVSRWENGKRASKPEFTAPTV |
| | VYNKKALTYREDGVSLATVNGRVECDFVLPPEGENPQTEYLRGDEWELRESTLHYRS |
| | ESGEYYLHTTVAKDEDVSEEAENGTVLGVDVGVENIAATSTGRFWSSGLLNHRREQY |
| | ESVRAGLQQTGTESAHRTIQQLGEREQRWVDDLLHRISKDIVSEAVEHDCSRIAFEDL |
| | TDIRERMPGAKKFHAWAFRRLFDLVSYKADERGIRTVQVDPAYTSQRCSKCGHTARN |
| | NRPSQAEFRCGRCGYENHADYNAAKNVAMKHVRAGQKSPRGRANRHLALKTGTLN |
| | GNGEFSPADA |
| 242 | MVITRKIELHLVHTGLSDEEYDQQWKFLHSINDNLYRAANRLVNQLYLNDEIDILLRY |
| | NNKEYMELRKQLAKKNLDKPVRAELKEKEKLVLEEIKAHRSAIFQRPYASVAYSMVT |
| | SQNEANIMTKILDVLKQDVLSHYSTNAKEVARGERSISNYRYGMPIPFAFDRTEKASIC |
| | IYEENKKYFLKWYNNLRFELSFGRDRSNNQLVVQRCLGISNDGAKYKACNSSIQMVR |
| | KNGSTRLFLLLCVDVPKEINKHIKGKVVGIDLGLNVPIYAAVNDGPERKSIGSREAFLD |
| | QRASFQRRFRSLQKLQMTKGGHGRLHKLEPLERVREAERNWVKNQNHLFSKEVVEF |
| | AKKVEAEIIQMERLKNFGRDDNEEIKEDKKYVVRNWSYFELQSMIEYKAKRAGIIVQ |
| | YVNPAYTSQTCSECGQKGIRDNIHFKCLNPECNCFGKDIHADYNGARNIAKSKEIVKD |
| 243 | MKIKRTIKLVVKPSEKEKHILFKTFEEYKFAYNFVAEIGWKSNIHNSVKLHNLTYIVVR |
| | EKTSLPSQLVISARNVASESLKSAFSRKKKGLAVSCPYSKNPAIRYDKRSYSVWFDRE |
| | EISIATIEGRLKLKIKIPEYFRQYFSWDIRSASLKYNKRLKKFFFNIVVEKEIEEIPENDTV |
| | VGVDLGLSKLATLSTADGKINKFFDGGHIRAVSERYFAIRKKLQSKGTPSAKRHLRKL |
| | SQKEKRFRTAINHKIAKEIVNLIPAGGTIVLEELKGIRERIRVYKRERRWVHSWNFAQL |
| | KQFIEYKAKSKGIKVVYINPKYTSQRCSKCGYVSKSNRKDQSHFKCSYCGYTVNADL |
| | NASRNIAINYLVSQKERLGHRVASLPVWAVVNQPNVRRLAVSSHS |
| 244 | MQRTIRIQLKPDIETDSVLSQTIEQYTWSFNAVCKHGWKNDLANGVELHKATYYDHR |
| | AITGLPSQLVCAARVKATEALKSAKSLKKKGKTVSCPISKRCPIRYDARSYTAWFDRS |
| | ELSILSINGRVKLSFEIAEYYRQYLAWKNTSADLLQDRKGCWWLHVVMEIETPQASV |
| | TDEVVGVDLGIASPAVDSRGSKYGSGHWKKIEDKTFELHRRLQSKGTKSAKGHLKKL |
| | SGRQRRFRKDCDHVLSKRLARSVESGATLVFEDLTNIRGRAKMRKAQRRRLHGWSF |
| | AQFQAFVTYKAEARGVNVGFVDPRYTSQKCSQCGHIERGNRPSQAEFRCKKCGYER |
| | HADYNAAINIRAEGIRMLKAEGSAVSAVGGEIRPKLGRKSKLRHSPVSTEADTVLGTP |
| | SQCG |
| 245 | MPTIRRKIELLLDRTGLSKEEVDARWHTLHQINNNLYRTANNLINKLYLTDEIDDILRL |
| | NNQEYIDLKKQLGKKGLDETTKAELEERMHKIYATMNQHRSEILQRPRQSFAYSAVT |
| | GGDDTEIFNAKILDTLKQNVLAHYNADMKEVRRGEKSISNYKKGMPIPFSFDKSVRLY |
| | ERNGQFFLKWYRDIQFILFFGHDASNNQLIVERCLGISEDGIAYKMCSSSLQMKGKKIF |
| | LLLVVDVPKEPFEQQKGMVVGIDLGLNVPIYATTNLTPERRAIGNRESFLNQRIAFQK |
| | RYKALQRLQLTKGGRGRSHKLEPLERLRETERNWVRTQNHLFSKEVIEFAKQVGASTI |
| | NMERLASFGKNMSGEVYEDKKFVLRNWSYYELQNLIEYKAKRANIKVRYVNPAFTS |
| | QTCSECGQTGERDSIHFKCTNPECKNFGKSIHADYNGAKNIAKSTNIIKE |
| 246 | MVITRKIELWIAEEDKTKRNETWDFLRMLDKEIFRAANVVVNNQYFNDFYEERIIQQD |
| | EKIGDVSKKIRLLYSKLRKANDEEQQSLQNEIEILKEQKKESQTRVRSEFYKTSKQNTT |
| | YQILTKQFPEIPSDILTCLNNQIYSVIGKEKKEVLQGKRSIRSYRKGMPIPFRFAQHPRLE |
| | FFENEYYLRWLNNISFVLRFGRDKSNNRAILEKIFSKEYKLCDSSIQIDDRKIFLLLVVD |
| | IPKSEHNLNKELSVGVDLGLNVPAYCALSEGFARLAIGHKDDFVRVRQQMQRRRKAL |
| | QKSLVLTSGGKGRTRKLKALDALGEKERHFVRTYNHTVAKRIVEFAEKYNAGVITME |
| | LLEGYGKDENGKSRKGDFVLRNWSYFELQTLLKDKAGRKGMDVVFIDPYHTSQTCA |
| | LCGHYEIGQRETQANFICKNPVCKNFDEKVNADYNAALNIARSKKFVAKKEECEYFK |
| | LHKETRS |
| 247 | MKRTVKIKLSMNERKALEETIKQFKRACQMTVEEGWNENGLNNYKKYKLQKRVYD |
| | EIRETTDLQANLVVRAIARGAQAVKGCTQLFENGFKASKPNFTSDSIAYDKRTLSVYP |
| | DEKRCTISTVNGRIDANFVLPEEKNDYYKEYLDGSWEITQSTIEKHEYEKENSFYLHL |
| | GLEKEDEEIEHEDPTVMGVDLGMNNLAVTSTGKFFKGNQLDHNRKRFEEMRGKLQQ |
| | KGTRSAHLTIQRMSERENRYACDTLHCISKELVEEAERNDVDIITFENLKYIRERMPKN |
| | KWYHVWAFNKLYQYVEYKAKERGIQVKQIDPRNTSRRCSKCGHTEKNNRNGNKFK |
| | CKQCGYELDADYNAAKNIGIKLLHRRQKSSAGAGNGQLALKSGTLKLNGEYSPTFLS |
| | ARLCLRYFGIQKEF |
| 248 | MNTIKNTYIRTLKFNLNLTPKFETEEDNKKYINDVYSYLRDAIWAQNRAMNIVLDRT |
| | KEAYTLGRGMNRVKEIYYSYSHQKPISNDKKESFLESLLQYAPIDDQFVKNEVKKLRK |
| | FYESKKKPPKEETVSKNCEALKNKYIKYVGKSKDDVKRELDLLENYCAYPEDIYEKF |
| | ANGLSTPAYIKQKVESYWKQDGIKTKVIYSMDENLRRIKDAPLFIPPNVFYNKKDELI |
| | GLVYDYTDYISFLEDLENKRNVNIYLSIPYKKGEDKLKFKLVLGNPHKSRDSRLSIKRI |
| | FEEEYRIKGSSIGFTKNKETGKNTNLTLYLTVEVPQNKDNTLDENVVVGVDVGIAIPC |
| | VCALNNDKYTRENIGSYDTLFAKRTQFKMQRSRLNSQLKLSKGGHGRKRKLKKLELL |
| | SGKEKNYVDTECRKYASDVIKFCLKHHAKYINLEHLKGYRENPKVLAGWSFYKIQTY |
| | IEQAAEKHGIIVRKINPCYTSQICSVCGNWHPENRPKGKLGQAYFNCHNIDCKTHNTD |
| | LYKYGINADFNAARNIAMSTLFITDSDEITKKHWKEAREYYGIDESDDKEEKLNKVA |
| 249 | MTKVVKLALINNVTDKNGNKVEYYDLNKCLWDLQKETRDLKNTVIRECWEWYGFS |
| | NDYYKLNEEYPAERDHLKREKANGTIKDYSLDGFIYSKCSKKYKLHSGNLCTTLRAA |
| | SGAFKTSLKDLLRGDKSVLSYKADQPLDVQKKCIVLEYDKDTNTYYITLTLLNKAGV |
| | KCYNISDFRFKITVKDNSTRTILERCYDEVYSISSSKLIWNKKKGQWFLNLCYSFDKTE |
| | TKELDKNKILGVNLGVYYPIYASISGEKDRLAISGDELIEFRNRIEARRNALKKQAAVC |
| | GDGRIGHGYKTRMKPVLNISDKIANFRDTFNHKASRKLIDFAVKNDCGIIQLENLKGV |
| | TKDTEGFLKNWSFYDLQSKIENKAKERGIKVVYIEPAYTSLRCSKCGYIHKDNHPTRE |
| | QFICQECGYRTLHDYNASQNIAVKDIDKIIKAELEKMGIKKNKDEEKPEK |
| 250 | MTKVVKLALINNVTDKNGNKVEYYDLNKCLWDLQKETRDLKNTVIRECWEWYGFS |
| | NDYYKLNEEYPAERDHLKREKANGTIKDYSLDGFIYSKYSKKYKLHSGNLCTTLRTA |
| | SGAFRTSLKDILRGDKSVLSYKADQPLDVQKKCIVLEYDKDTNTYYITLTLLNKTGVK |
| | FYDIGDFRFKVTVKDNSTRTILERCYDEIYSISASKLIWNKKKGQWFLNLCYSFDKTET |
| | KELDKNKILGVNLGVYYPIYASISGEKDRLAISGDELIEFRNRIEARRNALKKQAAVCG |
| | DGRIGHGYKTRMKPVLNISDKIANFRDTFNHKASRKLIDFAVKNDCGIIQLENLKGVT |
| | KDTEGFLKNWSFYDLQSKIENKAKERGIKVVYIEPAYTSLRCSKCGYIHKDNHPTREQ |
| | FICQECGYRTLHDYNASQNIAVKDIDKIIKAELEKMGIKKNKDEEKPEK |
| 251 | MTTKTYAIKLIKPVDDSWDFAGETLRNLEYIVKRFKNKAATDQYLSIVSKDKKSAAEI |
| | NTGVKQGLAELAYLNYAQECFNGAIKEAVDKVSKDFKGIVTGKSSLITYKDGQPIPVR |
| | SRQITLENDNGTYYASIGLLSREYATELGREGRQKARIKFVLSSKGNEKVVLDRILSGE |
| | YKLCDSSIQRKGNAWYLQLAHSFEAKAKTDLIANRVLGIDIGISKAVYMAVSDSPVN |
| | AFIDGGEIEQFRNKTEHRRNQMRNQLKWCSDNRKSHGRNTLLKPLEVLESKVSDFRK |
| | LTNHRYAKYVVDFAVKNQCSIIQMEDLSGINTRSAFLKRWSYFDLQTKIEDKAAAHGI |
| | KVVKVNPKYTSQRCYNCGVIAKDNRESQSVYKCECTRRTKKGVVAYKVNADLNAA |
| | RNLSVLGIDKEIKAQCKAQKIAY |
| 252 | MITVRKLKVRCEDKSFYDFLRLEQREQNKALNLAIGYIHTSNILKSNDSGAETKIIKSIS |
| | KLEDKIKKLNDELNKEKITDTKREKTLKAIETTTKILEGEKKILEEGKEFRIGLDKKFNE |
| | IYIDKNNMYHVLKTQTNVQYMRTLDLVKQKVSADYSNNFIDIVTGKISLMNYKQDFP |
| | LMIDNKNINLFKENDKYYIGIMLGYELEIVLGQRVNENILELKSILDKIIEDEEMAKQN |
| | KDHVKQYRFNQSSIQFDKNNNVIFNLTFTIPQDKTFKPVEGRVLGVDLGVKYPAYMC |
| | LSDDTYKREHIGSINDFLKVRTQMQNRRRELSKALSLTNSGKGRNKKTQALKRLSEK |
| | ERNFAKTYNHAISKRIVDFAKKHKCEYIHLEKLTKDGFNDRILRNWSYYELQRMVEY |
| | KADRIGIKVKYINPSYTSQKCSKCGHIDKENRQTQEKFVCTQCDFELNADHNAAINIA |
| | RATE |
| 253 | MITVRKLKLTIVGDEETRNQQYKLIRDEQYQQYRALNLCMSLLSTYNILNNWNSGAE |
| | NKLNSQIEKLNKKVEKNKNDLKKDNLKENRIKKINESIKTLAKEKEKLQQEYLSSSEY |
| | RSDIDKKVKEMYIDDLYTVVQSQVNFKSKDMMSLVTQRSKKDFTTALKNGMAKGER |
| | SLTNYKRDFPLMTRGERWLKFEYDEESDDIYINWLHGIRFKVVLGYKKNENSIELRHT |
| | LHKVINKEYKICDSSMQFDRNNNLILNLTLDIPLNAKNEHIEGRTLGVDLGIKYPAYVC |
| | LSDDTYKRKSIGCAEDFIKFREQIRSRRYRLQKQLSMVKGGKGRNKKLQALDRIKDKE |
| | RNFVKTYNHMISKNIVEFAKNHKCESINLEKLTKDGFPNMILSKWSYYELQNMIEYKA |
| | EREGISVKYVDPAYTSQTCSKCGYVDKENRTSQEKFKCIECGFELNADHNAAINIARS |
| | NNYVK |
| 254 | MANKNVNDSKIKTHILTKKVQLIVDTDRFDTDEEKKAEVDRVYKYLRDSMKCQSRE |
| | MNQYYMHLWMMSVARNLNDDRYSMKKYMDNIIDAVHPYLDKNNKEFNNKQKKIT |
| | RDFEKKIKNLCEEYQAETEILNNDTLRELNKCFNRKKDGAYDTNFVNEMPEGLGIVM |
| | GRTVEQDFQNDCKAGLLSGIRNPRSYKINYPLIIPKSFVAYGVGSGKAMQGRGIIFPDM |
| | EYSDFHNMLFSTKNPNITYNFVHDIDFKLVFGSMKRSHELRVIFDRIKMGEYSICGSTIE |
| | INNKKKIMLNLSYEAPIYEKPSLDENTVVGVDLGMAIPAVCSLNNDDRTYKYIGDSHE |
| | LEFIKKGIQAQRRSYQRNAVYNKGGHGRNRKLENLDRLKKRERNTTRTHNQRYAKQI |
| | VDFALANNAKYINLENLKGFSNNDKNKLVLRNWCYYELQQYIEIDASKYGIKVRYIY |
| | PMNTSRTCSVCGTLTTDEDVKNGVGRVSQDEFICKDPNCPSHTLYTKGPKGHKVPYF |
| | NADRNASRNIAMSEDFVKKNAKDNAFKKIDDLYEINNDIDAA |
| 255 | MITVRKLKLTIVGDEEIRKEQYKFIRDSQYAQYQGLNLAMGVLTSSYLLSGGDVKSD |
| | YFKDAQKSLKNSNKIFNEINFGKGIDSKSYITKQVKKDFSTSLKNGLAKGERGFTNYK |
| | RDFPLMTRGRDLKFYEEDKEFYIKWVNKIVFKILTGRKDKNKVELIHTLNKVLNKEY |
| | KVSQSSLQFNKNNNLILNLTIDVKSDVKVEVIKDRVCGVGVGINTPIYVALNDILYISQ |
| | SIGSNDELIKQKKQFEARKKRIRERVKNKKELNSLKEKERNWINTYNHMLSKRVVEF |
| | AKKNKCEYIYLEKINDNEFKNKVLKKWPYCELQKMIRYKAAGFGIEVKYIHSYHIFQ |
| | KCSRCGYEYNKSIAIQKRFKCLNCGLEVNSDYNIARNISKYDILKDKSRITGNLVSE |
| 256 | MLNNKFIKDEKKLQESLNKLYMEKKESETKIKRSKIDEEIKKKVNVLKKMRDKESKE |
| | ATKILQQAIKINLSNTTREIINQQFNLISDTKDRITQKVSQDFKADIKNGLLRGERVLRT |
| | YKKNSPLLIRGRTLQFYRKGNDILIKWYGGITFKCIIGKRKNNNHELYILLNKILENVC |
| | KVCDSSITIGRKLILNLSVALTGFEADAPTVKGRVLGVNFGIKVPIYMSLNDKSYVQKS |
| | VGNLNDLLKLRVQLYKRKKKLENLITNAIGDKVEKLKALNRLKEKEKNMLTTYNHY |
| | LSYNIVRFAKENQVGQINIEYLPFVKAKNKALKSWPYYQLQQFIEYKAKQKKIEVKYI |
| | NSYLINEKCSNCGKNITHQLNSTNVFNCKKCEYKAHLDFNISQNIALSTEYISIRK |
| 257 | MIITKKIKIIIIGENKDKYNKFIREEYYNQNKALNVAMNHLYFLHVAEEKIRMLNNKFI |
| | KDEKKLQESLNKLYMEKKESETKIKRSKIDEEIKKKVNVLKKMRDKESKEATKILQQ |
| | AIKINLSNTTREIINQQFNLISDTKDRITQKVSQDFKADIKNGLLRGERVLRTYKKNSPL |
| | LIRGRTLQFYRKGNDILIKWYGGITFKCIIGKRKNNNHELYILLNKILENVCKVCDSSITI |
| | GRKLILNLSVALTGFEADAPTVKGRVLGVNFGIKVPIYMSLNDKSYVQKSVGNLNDL |
| | LKLRVQLYKRKKKLENLITNAIGDKVEKLKALNRLKEKEKNMLTTYNHYLSYNIVRF |
| | AKENQVGQINIEYLPFVKAKNKALKSWPYYQLQQFIEYKAKQKKIEVKYINSYLINEK |
| | CSNCGKNITHQLNSTNVFNCKKCEYKAHLDFNISQNIALSTEYISIRK |
| 258 | MIITKKIKIIIIGENKDKHNKFIREEHYNQNKALNAAMNHLYFLHVAEEKIRMLNNKFI |
| | QDEKKLQESLNKLYTEKKESKTKIKRSEIDEKIKKKVNSLKKMRDKESKEAERILQQA |
| | IKINLSNTTREIINQQFNLISDTKDRITQKVSQDFKTDIKNGLLRGDRVLRTYKKTNPLLI |
| | RGRTLQFYRKGNDILIKWYGGVTFKCIIGQRKNNNHELYILLNKILENDSKVCDSSITI |
| | GRKLILNLSVALTGFEEDIPTVKGRVLGVNFGMKVPIYMSLNDKPHVQKSVGNLNDL |
| | LKLRVQLYKRKKKMKNLIIKSIGDKAEKLKVLNRFKEKEKNILTTYNHYLSYNIVQFA |
| | KENQVGQINIEYLPLVKTKNKALKSWPYYQLQQFIEYKAKRKKIEVKYINAYLLNKK |
| | CSNCGKDTTHQSNSNNIFNCKKCQYRAPLDSNISRNIALCTEYISIRKE |
| 259 | MKLKRTIKLVVKPSEEEKQILFKTLEEYKFAYNFVAEIGWKSKVSNSIKLHNLTYTTV |
| | REKTSLPSQLVISARMVASESLKSAFNRRKKGLKVSCPYSNNPAIRYDKRSYSVWFDR |
| | EEISIATVEGRLKLKIKIPEYFKQYLNWKIRSASLKYDKRLKKFFFNIVVEKEIEEIPEND |
| | TVIGVDLGLSKLAVISTADGKINKFFDGRHIRAVSERYFAIRKKLQSKGTPSAKRHLKK |
| | LSQKEKRFRTAINHKIAKEIVSLVPAGGTIVLEELKGIRERIKVSKKERRWIHSWNFAQ |
| | LQQFIEYKAQSKGIKVVYINPKYTSQRCNKCGHISKSNRKDQSHFKCSSCGYTINADL |
| | NASRNIAINYLVSQQERLGHRVASLPVWAVVNQPNVRGLANFSHS |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A system comprising:
(i) an anti-CRISPR (Acr) polypeptide comprising an amino acid sequence having about 80% or more sequence identity with an amino acid sequence set forth in any one of SEQ ID NOs: 1-6, or a nucleic acid encoding the Acr polypeptide; and
(ii) a CRISPR-associated protein or a nucleic acid encoding the CRISPR-associated protein.

2. The system of claim 1, wherein the CRISPR-associated protein is a Cas12f or μCas.

3. The system of claim 1 or 2, wherein the CRISPR-associated protein comprises an amino acid sequence set forth in any one of SEQ ID NOs: 7-9 or an amino acid sequence having about 80% or more sequence identity with any one of SEQ ID NOs: 7-9.

4. The system of claim 1 or 2, wherein the CRISPR-associated protein comprises an amino acid sequence set forth in any one of SEQ ID NOs: 10-259 or an amino acid sequence having about 80% or more sequence identity with any one of SEQ ID NOs: 10-259.

5. The system of any one of claims 1-4, wherein the Acr polypeptide is capable of modulating an activity of the CRISPR-associated protein.

6. The system of any one of claims 1-5, wherein the Acr polypeptide is capable of specifically modulating an activity of a Cas12f or a μCas CRISPR-associated protein, and wherein the Acr does not modulate the activity of a Cas9 or a Cas12a CRISPR-associated protein.

7. The system of claim 5 or 6, wherein the activity comprises one or more of: single-strand nucleic acid cleavage, double-strand nucleic acid cleavage, nucleic acid binding, and base editing.

8. The system of any of claims 5-7, wherein modulating comprises fully or partially inhibiting the activity of the CRISPR-associated protein.

9. The system of claim 8, wherein the Acr polypeptide is capable of decreasing the activity of the CRISPR-associated protein by at least 5%, at least 10%, at least 15%, at least 20%, at least 25%, at least 30%, at least 40%, at least 50%, at least 60%, at least 70%, at least 80%, at least 90%, or more compared to CRISPR-associated protein activity in the absence of the Acr polypeptide.

10. The system of any one of claims 1-9, wherein the ratio of ON target to OFF target activity of the CRISPR-associated protein in the presence of the Acr polypeptide is greater than the ratio of ON target to OFF target activity of the CRISPR-associated protein in the absence of the Acr polypeptide.

11. The system of claim 10, wherein the OFF target comprises a nucleic acid sequence having 1, 2, 3 or more amino acid differences compared to the ON target.

12. The system of any one of claims 1-11, wherein the system comprises the nucleic acid encoding the Acr polypeptide and/or the nucleic acid encoding the CRISPR-associated protein.

13. The system of claim 12, wherein the system comprises a first nucleic acid encoding the Acr polypeptide and a second nucleic acid encoding the CRISPR-associated protein.

14. The system of claim 12, wherein the Acr polypeptide and the nucleic CRISPR-associated protein are encoded by a single nucleic acid.

15. The system of any one of claims 12-14, wherein a translation control element is operably linked to the Acr polypeptide coding sequence and/or to the CRISPR-associated protein coding sequence.

16. The system of claim 15, wherein the translation control element is selected from the group consisting of an IRES sequence, a ribosome skip sequence, a noncanonical start codon or any combination thereof.

17. The system of any one of claims 12-14, wherein a first promoter is operably linked to the Acr polypeptide coding sequence and a second promoter is operably linked to the CRISPR-associated protein coding sequence.

18. The system of claim 17, wherein the first promoter is a stronger promoter than the second promoter.

19. The system of claim 17, wherein the second promoter is a stronger promoter than the first promoter.

20. The system of any one of claims 1-19, further comprising at least one guide RNA or a nucleic acid encoding the at least one guide RNA.

21. The system of any one of claims 1-20, wherein one or more of the nucleic acids encoding the Acr polypeptide, the CRISPR-associated protein or the guide RNA is comprised in a viral vector.

22. The system of claim 21, wherein the viral vector is an adeno-associated virus (AAV) vector.

23. The system of any one of claims 1-22, wherein one or more of the nucleic acids encoding the Acr polypeptide, the CRISPR-associated protein and/or the guide RNA is comprised in a lipid nanoparticle (LNP).

24. The system of any one of claims 1-12, wherein the system comprises the Acr polypeptide and/or the CRISPR-associated protein in protein form.

25. The system of claim 24, wherein the Acr polypeptide and/or the CRISPR-associated protein is comprised in a lipid nanoparticle (LNP).

26. A method of modifying a target nucleic acid comprising contacting the target nucleic acid with a system of any one of claims 1-25.

27. The method of claim 26, wherein the activity of the CRISPR-associated protein on the target nucleic acid is modulated by the Acr polypeptide.

28. The method of claim 27, wherein the activity comprises one or more of single-strand nucleic acid cleavage, double-strand nucleic acid cleavage, nucleic acid binding, and base editing.

29. The method of claim 27 or 28, wherein modulating comprises fully or partially inhibiting the activity of the CRISPR-associated protein.

30. The method of claim 29, wherein the Acr polypeptide is capable of decreasing the activity of the CRISPR-associated protein by at least 5%, at least 10%, at least 15%, at least 20%, at least 25%, at least 30%, at least 40%, at least 50%, at least 60%, at least 70%, at least 80%, at least 90%, or more compared to CRISPR-associated protein activity in the absence of the Acr polypeptide.

31. The method of claim 29 or 30, wherein the Acr polypeptide preferentially inhibits the activity of the CRISPR-associated protein on one or more OFF targets as compared to the target nucleic acid sequence.

32. The method of claim 31, wherein the OFF target comprises a nucleic acid sequence having 1, 2, 3 or more amino acid differences compared to the target nucleic acid sequence.

33. The method of any one of claims 26-32, wherein the contacting occurs in vivo.

34. The method of any one of claims 26-33, wherein the contacting occurs in a eukaryotic cell.

35. The method of claim 34, wherein the eukaryotic cell is an animal cell.

36. The method of claim 34 or 35, wherein the animal cell is a human cell.

37. The method of any of claims 34-36, wherein the eukaryotic cell is a stem cell.

38. The method of any of claims 26-37, wherein the target nucleic acid encodes a gene product.

39. The method of any one of claims 26-38, wherein the method further comprises measuring editing efficiency.

40. The method of any one of claims 26-39, wherein the method further comprises measuring editing efficiency at one or more OFF targets.


SEQUENCE LISTING (267 sequences)

SEQ ID NO: 1 - protein, 149 aa, synthetic construct
MTDNNYISEIQLKYKPKPISDSIKGSQDIYNLLISKVYDEDTIGYKETFKILLLNNANKVIGYTTIAEGGLTSTVVDLRVILQTALVANATSIIATHNHPSGVKGPSIQDDMLTAKIKEACKAVELRLLDHIIVTPEDGYYSYCDDGKL

SEQ ID NO: 2 - protein, 330 aa, synthetic construct
MSKVFLLGANKEIDRAKQVVEINQIIQMEGYSYDRYVVYEIRKNDWGIAYKLINQRTKEFYTTDIIRPLKEKFGIGYYYDSENPQFMDSFEVAILLQQAKDKKKSEDKKAEQEKIRVEQVKEIGRKRFAEIFPEDAQAVIVARLRQNESDSYTDYYSYSTQRTVIIGFSKHKRDLFSEMRKHASNFEETAYLAEFNEDYEHREKYSMGDGYYLGKSKYSGWIIEKEPVYNRERTIEDFAYTAGNEDNIHLSKTDTMPQSKPTEENTGGCTLVEYSAKAVAVFGDTKSIKDELKAMGGRFNSRLTFNGKKLAGWIFPKSQEQRLAYYFGLN

SEQ ID NO: 3 - protein, 72 aa, synthetic construct
MRMILSDIGKVLWKALSAVVTATSNIAFCAGGLVVGIIGEILFGMLSIMFGILISLFSVMAAIGFFIWLLTI

SEQ ID NO: 4 - protein, 85 aa, synthetic construct
MFWLGGLTGYVAGTLVTLLVTYFGYRIGEMNKVDEEWLAESVDKEIEQLRAIRDDNRDHNRESLKNVRSKFSEYMEKKDKERGNE

SEQ ID NO: 5 - protein, 198 aa, synthetic construct
MSKEKNLNDIITKELAETFDRYQLEAYVLECVKARGYCQLRARNSQVRFFNFTDEDPEGRLPGSFHACYLGVVMGLLHEWGFHTERIVATGTNAQIGECDVYVTLQGMVEPRELHRLFSSASPQEIGAATNPIHTVLRISQPVKMERMEVTNSIANAYNDPLLLTQTNPVGLRSLIIAVTLTAALVALVAMAVLQSIT

SEQ ID NO: 6 - protein, 256 aa, synthetic construct
MAEKGLSTGEAYKLMLRSALEKFESLIEWNKSNYDEYKILLRKLKISRGDIKATSNDETTDNTKDVGDALENLVNFIINKSFFFKVTSNIRTGSNEIDEVITLTKDGKAALEYFNIPRSLLVIEDNLFLGECKNYKTPLSVTYVGKFYSLMKQCDCNLGIMFTYKGLSGKETSWNDSHGLTKVLRLIEKHSSNNPNFYILEFKLEDYEAILEGKTFFNLISSKMIALQISANHNKFLEEPLEDDLQVLINYCKENK

SEQ ID NO: 7 - protein, 529 aa, unidentified
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 8 - protein, 422 aa, unidentified
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 9 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 10 - protein, 429 aa, unidentified
MTKVIKLALICQQSDSNGMPVDYKEVNKILWELQRQTREIKNKSIQYCWEYHNFSSDYYKRNGEYPKEKDVLLFTLGGYVNDKFKTGNDLYSANCSTTVRGVCGEFKNSKKDFISGKRSIISYKENQPLDLHNKSIRLEYSDHEFYVYLKLLNRQGFKKFNFADTQIMFKILVRDNSTKTILERCLDEVYSVSASKLIYDKKKKCWVLNLSYSFSGEITHNLDENRILGVDLGIHYPICASVYGEWKRFTIDGGEIEEYRRRVEARKKTLLKQGKNCGDGRIGHGVKTRNKPVYSIEDRISRFRDTANHKYSRALINYAIKNNCGVIQMENLEGVTAHSDKFLKNWSYYDLQTKIEYKAKEAGIKVVYINPRYTSQRCSKCGYTDTDNRPEQAKFICKKCGFSENADFNASQNIGIKNIEQIIKEEIQI

SEQ ID NO: 11 - protein, 438 aa, unidentified
MQKVLKVYLICEQLDPDGSPVDYKDIFKLLWDLQKQTREIKNKSIQYCWEFSNFSSDYYKEHHEYPKDKEILNYTLGGFVNDKFKTGNDLYSANCSTTVRTACAEFKNAKADFMRGEKSIISYKANQPLDLHNKSIRLEYQKGTFLFYLKLLNRSAVKKHGFQSSEIRFKAIVKDNSSQTILERCTAGVYDMAASKLLYDQKKKCWVLNLVYAFEPETPEALDPEKILGVDLGVHYPICASVYGDLKRFIIDGGEIESFRKRVEARKISMLKQGKNCGGGRIGHGIQTRNKPVYAIADKIARFRDTVNHKYSRALIDYAIKNNCGVIQMEKLTGVTADANRFMKNWTYFDLQTKIEYKAQEAGIQIVYIEPKYTSQRCSKCGYIDRENRPEQSKFICRKCGFSENADYNASQNIGIRNIEKLIEKQLQTKCESETDTT

SEQ ID NO: 12 - protein, 424 aa, unidentified
MKKTVRLQIVKPMDEDWEILGRVLHDIRYQTRQVLNKTIQLCWEYSNFSSEYKALHGDYPKNKEILHYTSMHGYAYNQLKEQYYYIQSGNCSQTVKRAVDKWKSDLKEILRGDRSIPSYKKDIPIDIVKDAVSLEHDEKNGNYIATLSLMSTAYRKEMERKSGQFRVLLHSGDNSKKTILKRLVQGEYQHTASQIVKKGKKWFLNLSYKFDVLQTPFQTERVMGVDMGVIYPIYMAFNYHDHLRYKIQGGEIERFRRQVESRKKALQDQGKYAGSGRVGHGTKTRIDPLEVIRDKIANFRETKNHHYSRYVVDMAEKHECATIQLEELKGIHQDDAFLKRWSYHDLQEKITYKAEEKGIQVIKVDPQKTSQRCHHCGNIDSNNRKEQASFLCTSCGMETNADFNAAKNISIPGIEQIIQTEMKS

SEQ ID NO: 13 - protein, 430 aa, unidentified
MCALTKIMKYELRYLDGFPDFSAMQNAVWPLQRQTREILNRTIQEAYRWDYFSATKKKETGEYPDLQKETGYKRLDGYIYHVLSPDYPDFSSSGVNATIQKAWKKYKSSKADVWKGEMSLPSYKSDQPIVLHAKQIKLSGDTRAAAATLSLFSNKFKKEHEISGNVQFAITLHDNTQRTIYQKLRNGEYKLSESQLVYDKKKWFLYLAYSFNPAEHALDPEKILGVDMGEKFALYASSFGEYGHFKIEGSEVTEYAKALERRRRSLQQQARYCGEGRIGHGTKTRVGVVYREEDRIANFRSTINHRYSKALIEYAVKNGYGTIQMENLTGIKENLQFPRRLQHWTYYDLQSKIEAKAKEHGIAVVKVNPKHTSQRCSRCGHIAAENRPKQEVFQCVKCGYACNADFNASQNISIKDIEKLIQETIGANPK

SEQ ID NO: 14 - protein, 427 aa, unidentified
MVLTRKLQLVPCAEGMSKEEAKKEVDRVYKILRDGIYAQNKAYNIFISRRYTAILLGASKEELAKLTLIGERNPKKDDPSYSLYEYGKINFIKGIPQASALGQHAISDLSKQKKDGLFKGKVALACRRLDAPMWIKQKYEFYHNYADNKELSENLYSEDLKIYMKLANICVFEVVLGNPHKSAAIRAELERVFDENYKKLDSSIQIVNNKIMLYLAINIPEKQIELKEDVVVGVDLGIAIPAVCALNNSRYIKKSIGSANEFLRIRTQLQSEKKRLQRKLRDTNGGHGRKKKLAPLDKLSKRERNFVQTYNHMISRRVVDFAVKNNAKYINVEDLSDYKNNGSEYILRNWSYYELQQQIEYKAEMYGIVVRKVNPYHTSQICSQCGHWEEGQRKSQSEFECKACGYTANADFNAARNIALSTDFVKK

SEQ ID NO: 15 - protein, 433 aa, unidentified
MSKGVVTKVMKYTLRYIGGCGDFHKMQEAVWKLQRQTREVLNKTVQLAFDWDHRSREAYRTSGEYLDIVKETGYKRLDGYIYNRLKTDYADFASSNLNATIQVAWKKYMASKSDILTGKMSFPSYKSNQPIVLHNSSIRFSTEKYGVPAAELTVFSNALKKENGLSTNPAFEILLMDGTQRSIFQRVISGEYKHGQCQINYEKRKWFLYLTYTFEAGKTPLDPDKILGVDIGETLAICASSTSEWGRFVIQGGEVTRYSKQIEERRRSQQRQATYCGEGRIGHGTKARVAPIYATEDRIANFRDTINHRYSKALIEYAVKHGFGTIQMEDLSGIKGEKDFPKFLRHWTYYDLQNKIEAKAKEQGINVVKVQPAYTSQRCSKCGCIDKENRKNQELFCCVKCGFKANADFNASQNISIKGIDKIIEKEYNANIE

SEQ ID NO: 16 - protein, 437 aa, unidentified
MNKVVKLALISKVKDKDGNDVKYGDVCKILWELQRQTREIKNKAVQFCWEWNGFSSEYNNLFGEYPKDKDYLKNKSEGKPIVLRSFVYDRLKSDYYLNSSNLSTTTSLAFKEFKQYLTDIRKGERSVLNYKNNQPLELHNECIWLESNNGKFITRFSFLNKAGKDFYSIDNFTFEVIVKDNSTKTILERCIDSIYGIRASKLIYNQKKKQWFLNLSYSFEAKEIATLDKDKILGVDLGIALPICASVYGDLDRFTIKGGEIEHFRKSIESRKRSLLQQGKVCGDGRIGHGIHTRNKPAYNIEDKIARFRDTANHKYSRALIDYAVRKGCGTIQMEMLKGITEEKDPFLKNWTYFDLQQKIEYKAKEKGIKVVYIAPKYTSRRCSKCGHIDKDNRLTQANFLCLNCGYKENADYNASQNIAIKDIDKIIIEETKGGES

SEQ ID NO: 17 - protein, 434 aa, unidentified
MNKVVKLSLICEQTDKDGNKIEHGEVYKILWELQRQTREIKNKTIQYCWEYSNFSSDYYKINHEYPNEKEILSFTLKGFVNDKFKKGNDLYSGNCSTTTGNVCSEFKNSKSDFLKGEKSIINYKAYQPLDIHNKCILIEHTNNEFYVRLKLLNRPAIKKYNFANSEFNFKIIVKDNSTRTILERCIDKIYDVAASKLIYDKKKKMWVLNLVYAFDNKSEYVLDKNRILGVDLGIHYPICASVYGEWNRFTIDGGEIEKFRKTVEARKKSMLRQGKNCGDGRIGHGISARNKPVYKIEDKIAKFRDTANHKYSHALIQYAIKNNCGVIQMEDLTGITNEADRFLKNWSYYDLQTKIKYKAKEVGIDIVYIKPKYTSQRCSKCGYIDKENRNKQASFVCLKCGFKANADYNASQNISIKDIDKLIEEMYNSSANTE

SEQ ID NO: 18 - protein, 432 aa, unidentified
MSKGSLAKVMKYELRYLDGAGSFEQMQERLWVLQRQTREILNRSTQISFHWDYTSREHFEQTGQYLDVFSETGYKRLDGYIYSRVKDSCGDMASGNINATLQKAWNKYGTSKLDVLRGQMSLPSYKKDQPLVIEKHNIRLSMDGQQALAEITLFSNKFKKENSLSSNVRFAFQLHDGTQRRILNSVLSGEYGLGQCQLVYDRPKWFLLLTYTFTPQNRQLDPDRILGVDLGECYALCASVFGEYGSLRIEGGEVTAYAKKLEARKRSLQKQAAVCGEGRKGHGTKTRVADAYQMQDRIANFRDTVNHRYSKALIDYALKNQCGTIQMEDLSGIRQDTGFPKFLQHWTYYDLQSKIENKAKEHDIRIVKINPRYTSQRCSKCGAIDSGSRTSQARFCCTKCGFTANADYNASQNISIKGIDLLIEKELGAKAE

SEQ ID NO: 19 - protein, 438 aa, unidentified
MGKGEISKVMKYELRYLDGSGSFEEMQQRVWALQRKTREIQNRTVQIAFHWDYINREHFIQTGNNLNVLQETGYKRLDGYIYDRLKGQSAEMSGANLNATIQTAWKKYNSAKPKVLSGTMSVPSFKRDQPLIINSNCVKFSRSESECLAELTLFSREYKKEHDLSSNVRFAIRLHDSTQRSILERVLSGAYRKGQCQLVYQRPKWFLFLTYSFFPMQHDLDPEKYLGVDLGECCALYASSVGEYGSLKLEGGEITAFAKQLEARKRSMQKQAAYCGEGRIGHGTKTRVADVYKMENRIANFRDTVNHRYSKALIDYAVKHQYGTIQMEDLSGIKNDTGFPKFLRHWTYFDLQEKIDAKAREHGIHVVKVNPQYTSQRCSKCGSIDSRNRKSQKEFCCLNCGYKVNADFNASQNLSIKGIDVIIQKYIGAKSKQTENNG

SEQ ID NO: 20 - protein, 430 aa, unidentified
MKEIAKVVKLELGWVFTDDGERAFPYSDLFEIQRQVALVKNKTIQLCWEWNNFGADFHAMSGAYPKTGDVTGYKTLDGYVYQRLKDDFSRMFKKNLNASVRSAQKAFETAKKSLHKGERSILSYRKDAPIELHNSAVSFSQEGRKYKAAVKVFSLSYAKEKGYAGTGVEFELSHLQGSPKEIVQRCMSGEYKIGESKLIWNEKKKKWFLYLTYKFTPAAVALDPEKIMGVDLGIACVAYMGFRFCEDRHVIPGHEVEHFRRRVEARKVELQRQGKFCGEGRIGHGRATRTKPVDQIGHAIARFRDTANHKYSRFIVDMAVKHGCGVIQMEDLHVHAEDKLLKDWTYFDLRTKLEYKAKEKGIEVRFVNPRYTSQRCSCCGYIEKENRKTQKEFICLECGFAANADYNAALNLATAGIEQIIDEYVSANHK

SEQ ID NO: 21 - protein, 432 aa, unidentified
MSKGTLAKVMKYELRYLDGFPSFYEMQEAVWGLQRQTREILNKTLQMAFHWDYTSREHFKETGSYLDVRTETGYKRFDGYVYECLKSDYSDIASKNLNATLQKAWKKYRNTRLDVLKGTMSLPSYRSDQPLTLDKNTVKLHSDGVDDWVELTLFSKAYKTQHGLSANVRFAIPMHDRTQRSIFQNLIDGVYALGECQLVYDKKKWFLLVTYVFTPEQHTLDPEKILGVDMGEAYAIYASSVYGYGTLKIEGGEVTDYAKKLERRKWSYQKQARYCGDGRIGHGTKTRIAEVYKAEDRIANYRDTINHRYSKAVVDYAVKNGYGTIQMEDLSGIKEDTGFPRRLQHWTYYDLQTKIENKAKEHGIRVVKIDPRCTSQRCSRCGHIDPKNRPSQSQFCCTACDFRANADFNASQNISTKGIDKIIAKTLRAKPE

SEQ ID NO: 22 - protein, 430 aa, unidentified
MGTVTKVMKYELRYLDGSGSFHDMQNYVWQLQRQTREILNRTIQEATLWDYRSREHFLEAEEYLDVYAETGYKTLDGYIYNRLKGSYGDFAGANLNATLRKAWKKYKTSKTEVLRGTMSLPSYKGDQPLVLHNGSVKLQGDSRDAVVELTLFSNTFKKREGIKGNPSFSLLVRDNTQRSIYQSLIDGVYKLGECQLVYQKKKWFLLLTYTFEARQHEVDPEKILGVDLGEAYAIYASSKDNFGSLKIEGGEVTDYAKGLERRKRALQQQARYCGEGRVGHGTKTRVTEAYKAEDRLANFRKTINHRYSKALIDYAVKNGYGTIQMEDLSGIKADTGFPKRLQHWTYYDLQSKIEAKAKEYGIHVVKVDPSYTSQRCSKCGHIDSQNRKTQERFLCVSCGFSCNADFNASQNLSIKGIEKIIKKTKGAKVE

SEQ ID NO: 23 - protein, 455 aa, unidentified
MAKKGNSQKKQIVKVMKYELKYEKGCADFNEMQNELWKLQRQTREVMNRTIQLCYHWSYVQAEYCKQHGCARRDVKPCDVYETNATSLDGYIYQLLKVEYPDFFMKNLNATLRKAHQKYDALLFDIQEGNSSIPSFKKDQPLIFEKKAICISKCLPDKRQITLSCFSDSYIDAHPTLDKITFTVRARSASEKSIFDHIISGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGTNPK

SEQ ID NO: 24 - protein, 441 aa, unidentified
MDLRNWLRRVRIHMAKGTVTKVMKYELRYLSGFSDFHAMQQAVWGLQRQSREILNKTIQMAFHWDYISRENFNANGVYLDVKAETGYKTYDGYIYNSLKSAYADMAAANLNAAIQKAWKKYKDAKMEVLRGTMSTPSYRSDQPVLINKNCVKLFDGGVRLTLFSDRFKRENNLNGNLEFAVQLHDGTQRSIFANLLNGTYALGQCQLVYDKRKWFLLVTYIFTPEKHELDPEKILGVDLGQTYALYASSVCARGTFRIEGGEAAECAHRLEQRKRSLQQQARFCGEGRVGHGTKTRVAAVYSAGDKIASYRDSINHRYSKALVEYAVKNGYGTIQMEDLTGIQNDLDHPKRLQHWTYYDLQTKIENKAKEHGVGVVKVNPRYTSQRCSRCGHIERENRPTQKVFCCKACGFEGNADYNASQNLSMRNIDKIIEKELSAKGE

SEQ ID NO: 25 - protein, 437 aa, unidentified
MSGSTIAKVMKFELFYREGGGEFHEMQKLLWELQRQTREVLNKSVQIYYQWKWKKQQHFEETGQSLDIYTELHYHRISSYAYNVLKEKYSSFYKANLSSTIKTACDKCESSEKDILCGTMSVPSYKRDQPLLLHDTSLSIRRNGTQWFADCKLFSAELVKNLGLKRGQSLVFSIKALDKTQINILERIEDGNYAIRQSQLTYEKKKWFLYLTYRFDKPKSELDPNRILGVDLGVSNAFCASVYGELDKLMIPGDEAIETIRRLEQIKYSKLRQARYCGEGRIGHGTSTRIAPAYSTRDKISNLQKTLNHRWSHAIVRYALRQGCGVIQMEDLSGIKEANDFPLRLQHWTYYDLQTKIKNKANEYGIEVRQVDPQFTSQRCSKCGCIQKENRPAQAKFCCIKCGYRTNADYNASQNLALPDIDRIIQEELKQIGANRK

SEQ ID NO: 26 - protein, 437 aa, unidentified
MQQVVRFEILKPVDNDWKILGRVFRELQYESRLVLNKTIQYNWEFSNCVIGFKEKFGIAPKISDISKYKDGKRGLEGYIYDKLKDIYTKNYSKNLGCLISKATSQWYAIKNDVYKGEKIPPEYKKSNTPIIVDKQAMTLFKENNLYYAKVALVSTNHIKEYGLNSCKFTILLNTKNNGNKVILDKVINGEFDYSQSQIIEKNKKWFLYLSYKIKEKTLPDDYNRVMGIDLGKNKAVVIAVHGTEIRDYILGGEIIDYKKKMYNMVWHRQKQSRYCGEGRIGHGRKTRLKDVYNIKEKIANFSDLTNHKYSKYIVELAKRHKCGIIQMEDLSGLSTDNKFLKQYPIYDLQQKIIYKAEREGIKVVKIKPHFTSQMCSNCHYISTQNRPKDDRGWEYFKCVNCGLEIDADLNAARNIANPQIELIIEEQLKIQEIDDKI

SEQ ID NO: 27 - protein, 437 aa, unidentified
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 28 - protein, 488 aa, unidentified
MANEFTCITRKIEVHLHKHGDSDEAIQRYKEEYRMWDDINNNLYKAANRIVSHCFFNDTYEYRLKLHSPRFQEIEKLLSNPKRNKLSDDDIKELKAERKLLFSDFKSQRQTFLRGGIETGTNPEQNSTYKVISNEFIDCIPSEVLTNLNQNISSTYREYTLDVERGIRTIPNFKKGIPVPFSIKQHGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRNLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQPTFICKNPDCTKGKGKQKSNGAYEGINADWNAARNIARSNEFVEKKKK

SEQ ID NO: 29 - protein, 486 aa, unidentified
MATEYTCITRKIEVHLHRHGDSEEDTQRLKDEYHIWDVINDNLYKAANRIVSHCFFNDAYEYRLKLHSPRFQEIEKLLRYSKRNKLTDEDIKQLKAERKELFSIFKKQRLEFLQGGSGKGSEQNSTYKVVSNEFGEIIPSHVLTCLNQNITSTYSAYSKEVEYGNRTIPNFKRGIPVPFPIKQQGTLQLKRREDGSIYIRFPLGLEWDLSFGRDRSNNREIVERVLNGQYDVGNSSIQETKNKKRFLLLIVKIPKQAVTLNPDRIVGVDLGINIPLYAALNDNEYGGMGIGSREQFLKMRMRMAAQKRELQRNLRHTTHGGHGRTQKLQALERLEGKERNWVHLQNHIFSKSIIEYAQRNDAGVIQMERLTGFGRDKHDEIDSDFKFILRYWSFFELQTMIEYKAKAAGIEVRYIDPYHTSQTCSFCGHYEKGQRISQSTFVCKNPDCEKGKGKKHSDGTYEGINADWNAARNIALSTKIVDRKKK

SEQ ID NO: 30 - protein, 488 aa, unidentified
MATEYTCITRKIEVPLHRHGEDEEAKQRLIDDYRVWDTINDNLYKAANRIVSHCFFNDAYEYRLKIHSLRFQEIEKLLKYSKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIFIRFPMGLEWDLSFGRDRSNNREIVERVLSGQYDVGNSSIQESKNRKRFLLLVVKIPKENHNLNPDRIVGVDLGINIPLYAALNDNEYGGMGIGSREQFLNMRMRMDAKKRELQRNLRQSTNGGHGRKQKLQALERLEGKERNWVHLQNHIFSKSIIEYAVKNNAGAIQMERLTGFGRDKNDEVDSDFKFILRYWSFFELQTMIEYKANAAGIEVRYIDPYHTSQTCSFCGHYEKGQRLNQSTFVCKNPDCEKGKGKKLSNGTYQGINADWNAARNIALSDKIVDRKKK

SEQ ID NO: 31 - protein, 488 aa, unidentified
MATEYTCITRKIEVHLHKHGDSEEAAQRFKEEYRIWDDINNNLYKAANRIISHCFFNDTYEYRLKLHSPRLQEIEKLLSNPKRNKLSDEEVKQLKAERKQLFADFKKQRHVFLRGGVEEGANPEQNSTYKVVSNEFIDFIPSEVLTNLNQNISSTYREYSLDVERGVRTIPNYKKGIPVPFSIKQKGEIVLKKREDGSMYVRFPKGLEWDLNFGRDRSNNREIVERVLSGQYDVGNSSIQETKNKKRFLLLVVKIPKQVASFDPSRIVGVDLGINVPLYVAINDNEYGGMGIGSREQFLKMRMRMAAQKRELQRNLRHTTNGGHGRTQKLQALNRLEGKERNWVHLQNHIFSKSIIEYAVRNNAGVIQMERLTGFGRDKNDEVGADFKFLLRYWSFFELQSMIEYKAKATGIEVRYINPYHTSQTCSFCGHYEKGQRINQATFVCKNPECTKGKGKQRTDGTFEGINADWNAARNISFSTDFVDKKKK

SEQ ID NO: 32 - protein, 481 aa, unidentified
MATEYTCITRKIEVHLHRHGDSEEAAQRLKEEYRIWDEINDNLYKAANRIISHCFFNDAYEYRLKLHSPRFKEIERLLKYAKRNKLTDDDIKALKAERKELFAEFKRQRQSFLGGSEQNSTDRVVSHEFLDVIPSEVLTCLNQNIASTYKEYARDVERGVRTISNFKKGIPVPVRVKRNGALLLRKREDGSIYLSFPKGLEWDLNFGRDRSNNREIVERVLSGQYDVGGSSIQEAKNGKRFLLLVVKIPKESRALNPDRVVGVDLGVNIPLYAALNDNTYGGLSIGSRDQFLKVRMRMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 33 - protein, 488 aa, unidentified
MATEYTCITRKIEVHLHKHGDSEEATQRLKNEYHIWDEINNNLYKAANRIVSHCFFNDTYEYRLKLHSPRFQEIEKLLNNSKRNNLSAEEIRQIKIERKLLLSEFKKQRYAFLRGGIEEGANPEQNSTYKVVSNEYIDKIPSDVLTNLNQNISSTYKEFSLDVEKGVRTIPNYKKGLPIPFSIKKNGDLLLKKRDDGTIYIRFPKGLEWDLSFGRDRSNNREIVERILSGQYDVGNSTIQETGNKKRFLLLVVKVPKKNIVSNPNRVVGVDLGINYPLYAALNDNEHGGISIGSRDQFLKMRMRMAAQKRELQRNLRHTTNGGHGRTQKLQALERLEGKERNWVHLQNHIFSKSIIEYAIKNNAGTIQMERLTGFGRNENNEVGSEYKFLLRYWSFFELQTMIEYKAKASGIDVRYINPYHTSQTCSFCGHYEKGQRLNQATFVCKNSACTKGKGKQKSDGTYEGINADWNAARNIALSTDFVDKKKK

SEQ ID NO: 34 - protein, 481 aa, unidentified
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGMSIGSRDQFLKVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 35 - protein, 488 aa, unidentified
MATDVTCITRKIEVHLHKHGDSEEGAQRLKEEYRIWDDINNNLYKAANRIISHCFFNDTYEYRLKLHSPRFQEIEKLLSKPKCNKLSADDIKQLKAERKVLFADFKKQRQVFLRGGLEEGTNREQTSTYRVASKEFIDTIPSEVLTNLNQSISSTYKKYALEVERGVRTIPNYKKGIPVPFAIKHKEELALKKRDDGSIYVRFPKGLEWDLSFGRDRSNNREIVERVLSGQYDVGNSSIQETKNKKRFLLLVVKIPKENRVLNKERVVGVDLGINTPLYAALNDNKYGGLSIGSRDQFLKVRMRMTAQKRELQRNLRHTTNGGHGRTQKLQALDRLEGKERNWVHLQNHIFSKSIIEYALQNDAGVIQMERLTGFGHDNNDEVDEKFKFILRYWSFFELQTMIEYKAKAAGIEVRYINPYHTSQTCNFCGHYEKGQRINQATFVCKNPDCIKGKGKQHSDGSFAGINADWNAARNIALSNDVVDKKKK

SEQ ID NO: 36 - protein, 488 aa, unidentified
MTAEYTCITRKIEVHLHKHGESEEATQRFKDEYRIWDDINNNLYKAANRIISHCFFNDAYEYRLKLQSPRFQEIEKLLGNTKRNKLSAEDIKVLKAERKLLFSDFKKQRQIFLRGGVEEGPNPEQNSTYKVVSQEFIDVIPSEVLTNLNQNISSIYREYALDVERGIRAIPNYKKGIPVPFSIKQKGEIVLKKREDGSIYVRFPKGLEWDLNFGRDRSNNREIVERVLNGQYDAGNSSIQETKNKKRFLLLVVKIPKESRSLNKERIVGVDLGINVPLCAALNDNEYGGISIGSRDQFLKVRMRMAAQKRELQHNLRHTTTGGHGRTQKLQALDRLEGRERNWVHLQNHIFSKTIIEYALKNNAGVIQMERLTGFGRDGKEEVQNEYKFILRYWSFFELQTMIEYKAKAVGIEVRYINPYHTSQTCSFCGHYEKEQRISQTTFVCKNPKCTKGKGKLKSDGTFEGINADWNASRNIAKSTEFVDKKKK

SEQ ID NO: 37 - protein, 487 aa, unidentified
MATEYTCITRKIEVHLHKHGDSEEATQRFKDEFRIWDDINNNLYKAANRIITHCFFNDAYEYRLKLHSPRLQEIEKLLSNSKRNKLSDEEVKQLKAERKQLFADFKKQRQVFLRGGVEAGANPEQNSTYKVVSNEFIDTIPSEVLTNLNQNISSTYREYSLDVERGIRTIPNYKKGIPVPFSIKQKGEIVLKKREDGSIYVRFPKGLEWDLSFGRDRSNNREIVERVLSGQYDVGNSSIQETNNKKFLLLVVKIPKQMASVDPNRIVGVDLGINVPLYAALNDNEYGGMGIGSRDQFLKVRMRMAAQRRELQRNLRYTTNGGHGRTQKLQALDRLEGKERNWVHLQNHIFSKSIIEYAVRNNAGIIQMERLTGFGRDENDEVGTDFKFLLRYWSFFELQSMIEYKAKAANIDVRYINPYHTSQTCSFCGHYEKGQRINQSTFVCKNPECAKGKGKQRADGTFEGINADWNAARNIAFSTEVVDKKKK

SEQ ID NO: 38 - protein, 488 aa, unidentified
MATEYTCITRKIEVHLHRHGDSDEAIQRYKDEFHIWDEINNNLYKVANRIISHCFFNDTYDYRLKLHSPRFQEIEKLLRNPKRNKLSGEDVKRLKAERKALDADFKKQRQAFLRGGVEEGTNKEQTSTYIVVSHEFIDIIPSEILTNLNKNIFSTYKKYRLDVEKGARTIPNYKKGIPVPISIKRSGELMLKKREDGSIYVRFPKGLEWDLFFGRDRSNNREIVERVLNGQYDVGISTIQETKNKKRFLLLVVKIPKESKNLNPNRVVGVDLGINIPLYAALNDNEYGGLGIGSREQFLKVRMRMAAQKRELQRNLRHTTNGGHGRAQKLQALDRLEGKERNWVHLQNHIFSKSIIEYALRNGAGVIQMERLAGFGRDKNEEVENEFKFILRYWSFFELQTMIEYKANAAGIEVRYIDPYHTSQTCSFCGHYEKGQRINQSTFVCKNPDCVKGKGKQHADGSYDGINADWNAARNIALSTTVVDKKKK

SEQ ID NO: 39 - protein, 488 aa, unidentified
MATEYITKTRKIEVYLHRHGDSDEAKQRYQQEWQIWHDINDNLYKVANRIMTHHFLNDEFVSRLRSTNPRYVEIEKILKHCKRNKLSQEEINSLQQENRALDALFTEKKNEFLGTTQEHNTILRIVRKEFGDVIPNDVYDCVIAERVKYTDKQKHLQIINGESSVPNYRKGMPVPFRIKIGANHNTLGILRRNDNPNHIYVKFPKGLEWDLVFGKDPSNNRKIVERILSGQYDAGNSSIQQAKNGKCFLLLVVKIPKSNIQLNKDRVVGVDLGINIPLYAALNDNIHSRLSIGSREQFLKMRMRMYAQKRELQRNLRHSTNGGHGRKQKLQALERLEGKERNWVHLQNHIFSKSVIEFAQKHNAGVIQMERLTGYGKDANGEMREEAKFLTRYWSYFELQTMIEYKANAAGIEIRYIDPYHTSQTCSFCGHYEKGQRVSQSTFICQNPECKQGKGKQKSDGTFEGINADWNAARNIALSTQYVDKKKK

SEQ ID NO: 40 - protein, 484 aa, unidentified
MATEYTCITRKIEVHLHRHGEDEDAVQRYKNEFQIWNEINNNLYKVANFISSHLFFNDAFVDRLRVQSNEYRDLLDLISKTTDAKEIKALENRKKALDAEFKRQQKIFLKGGSEDEKGSEKTAIRRIAVETFPNIPYSIINSLNDQISKTYNSSRFDVSIGKRTVPNYKKGIPVPFLMANGSGKIALREREDGSPYVLFPRGLEWDLHFGKDSSNNREIVKRVFNGEYKACDSSLQQAKNKKIFLSLVVKIPKKNHNLNPDRIVGVDLGINIPLYAALNDNDYGGMGIGSREQFLKVRMRMSAQKRELQRNLRQSTNGGHGRAQKLQALERLEGKERNWVHLQNHIFSKSIIEYALKNNAGAIQMERLTGFGRDKNDEVDSNFKFILRYWSFYELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 41 - protein, 497 aa, unidentified
MNDSPVIKNKRHVKVLRLRILKPVSGTWQDLAKLLRDTRYRVYRLANLAVSEAYLGFHMWRTGRAETYELDTPGALNRRLRRMLDEEGVRADELDRFSKTGALPDTVVGALSQYKIRAATGKSKWQEVIRGKSSLPTYRLDMAIPLRCDKRNHARLARVENGDVTLDLMLCLRPYPRVVIQTGNIGGGAQAVLDRLLANPSQNPDGYRQRLFEIKHDDRDNKWWLYITYDFPAADPPRSSADRIVGVDIGVSCPIYVAINDGHARLGRRQFSSLGARIRSLQNQIVARRRSMQAGGKVALSGQTSRSGHGRKRKLRPIQKLEGRISHAYTTLNHQLSSSVIDFALSHGARVIQMEDLASLKDALRGTFIGARWRYHQLQQFLEYKAKESGLTLRKINPQFTSRRCSRCGFIHVEFDRARRDASRRDGYVARFVCPAPKCGFEADPDYNAARNIATPDIEKLISDQCKIQSIPTRSLTDQSEAADKDTLAQGQSRSGG

SEQ ID NO: 42 - protein, 492 aa, unidentified
MSEKRHNKVAKFQILKPAAGTTWPELANLLFAVRYRVFRLANLCISEHYLHYHLWRMGKTEEIPKLKISELNKKLREMIIEENDKKEKQNKINQDAINKKGALTSYVVDTLSQNKLGAVTSKSKWKEVILGKASLPTFRLNMAIPVRCDKPEQCRLKINANGDVELELMICERPRPRIILKTGGLSGSMKSVLDRLLENSAQSMEGYRQRNYEIIQDRNDGKKWYLHVSYDFPATERKPNSEIIVGVDVGFALPLYAALGNGHARLGWKQFHSLAKRIRSLQNQVVSRRRKMLRGGKDSLTQDTARSGHGRNRLLQPIEKLAGRIEKAYTTLNHQLSRSVVDFAKNHGAGIIQMEDLEGMKDAINGTFLGERWRYFELRQFIEYKAKEAGIEVRLANPKYTSRRCSACGYINMAFTREYRDSHRKNGKSAEFFCPECDKLPADDEQPQKPYPTDADYNAAKNLAALDIEKIIRRQCEKQGISYDKSPENNDL

SEQ ID NO: 43 - protein, 489 aa, unidentified
MANTEFTCITRKIEVHLHRHGDSDEAALRLKNEYHFWDEINDNLYKAANRIISHCFFNDTYEYRLKLHSPRFQEIEKLLKNAKRNKLTDEEIKELKAERKLLFSDFKKQRYTFLRGGIEDGANPEQNSTYRVVSNEFIDTIPSEVLTNLNQNISSTYKNYTIDVERGLRTIPNYKRGMPVPFSIKKHGVLALRKREDGSIYVAFPKGLEWDLSFGRDRSNNREIVERVLSGTYDVGNSSIQEAKNGKRFLLLVVKIPKEVKILDTSRVVGVDLGVAVPLYAAISDNEYGGMSIGSYDQFIKVRMRMNAQKREMQHNLRHTTNGGHGRKQKLHALERLEGKERNWVHLQNHIFSKSIIEYALQNNAGAIQMERLTGFGHDKNNEVDEGYKFILRYWSFFELQTMIEYKAKAAGIEVRYIDPYHTSQTCSFCGHYEKGQRLNQSKFVCKNPDCVKGKGKQRSDGSFEGINADWNAARNIALSTKIVDRKKK

SEQ ID NO: 44 - protein, 522 aa, unidentified
MENNITITRKYALIPEFSDRKEWKKRVYDFMINDLEQKIDYRNKKKQDTSELESQLEYIKNGGDFTRSMVNNYTYSLVRKAMEEETRRKNYILSWIFSEMRANRIDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHINDSDCKMYMNYGGDNRKDGINPASIARFRISLGHGKNKDELKSTLLKVYSGEYQYCGSSIQITKNKIILNLTMKIPKIETKLDENTVVGVDLGIAVPAMCALNNNMYERLAIGSADDFLRTRTKLQSQRRRLQKSLKNSNGGHGRNKKLKVLERLGKSETHFAETYCHMVSKRIVDFALKNNAKYINIENLNGYNTSSFILRNWSFYKLQQYITYKAERYGIVVRKINPCYTSQVCSVCGNWEDGQRKTQASFECANPKCESHKKYKYGFNADFNAARNIAMSTLFMEDGEVTEKKKEEAREYYGIKKENSEAV

SEQ ID NO: 45 - protein, 490 aa, unidentified
MATEYTCITRKIEVHLHKHGDGEEAEKRRAEEFRMWNEINDNLYKAANRIVSHCFFNDAYEYRLKIQSPRYKEIQRKLRYSKSNKLTDDEIKSLKAERKELDNEFRKQYRAFMLGGSSEGFKSTTEQNSTERIVNNEFGDIIPSNVLSCLNQNVFQTYKQYRTDVEFGKRTISNFKKGMPVPFSIKAHKSLMLKKREDGSIFVYFPKGLEWDLSFGRDRSNNREIVERILSGQYDAGTSSLQEGKNGKIFLLLVVKIPKQSNALDPNRVVGVDLGINIPLYAALNDNEYGGMSIGSREQFLKMRMRMVAQKRELQRNLRHSTNGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFVCKNPECEKGKGKKRSDGTYTGINADWNAARNIALSDKFVDKKKK

SEQ ID NO: 46 - protein, 490 aa, unidentified
MANDEICITRKIEVHLHLHGDDEEGRARRKKDFETWNTINDNLFKVANLIATHQFFNDAYEFRAKIHSPQYTKIEKDLNNSQKLKLTQEQVRELEKEKSKLDKEIEEQRKTFLQCSRQNSTFRVASKLFLDVIPSNVLTCLNQKICSTYTSYKSEVESGKRTLPNFKKGLPVPFQMNLNKKLQLRRRTDGSIFVLFPKGLEWDLFFGKDKSNNREIVNRVLSGEYGVGESSIQQNKKGKTFLLIVVKIPKKAICLDSKRVVGVDLGINVPLCAALNDNESAKMYIGSREQFMKVREQLYVRKRELQRSLTTSTRGGRGRKQKLQALERHEGKERNWTHLQNHIFSKSVIEFAQKQNAGVIQMENLSGFGRDKNDEVDEGYKYILRYWSYFELQQMIEYKAKASNIEVRYVDPKYTSQTCSYCGHYEKGQRISQSTFVCKNPECEKGKGKKTKDGKYEGINADWNAARNIALSGKVEERKKKKKRKIQSNQ

SEQ ID NO: 47 - protein, 576 aa, unidentified
MNTINNTYIRTLKFNLNLTPKFETEEDNKKYINDVYSYLRDAMWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDIKRELDLLENYCAYPEDIYEKFANGLSTPAYIKQKVESYWKQDGIKTKVIYSMDENLRRIKDAPLFIPPNIFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYADTECRKYASDVIKFCLKNHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 48 - protein, 497 aa, unidentified
MIIARKLKITVIGSDEERKEKYRWIRDEQYNQYRGLNMGMTYLATGEILRMNESGLEIRLEKQKTELESKVEKAKLNIEKIKIKIEKIKTSKKINEEKILENQNKIDDEKANIIKYKNNIIKIEQALKVAKIKRMDIQMEFKEKYIDDLYQVLDKVPFQHLDNKSLITQRVKNDIKADKTSGLLKGERSIRNYKRTFPLLTRGRDLKFYYDDKDIKIKWIEGIEFKVVLGNRIKNSLELRHTLNKVVNEEYKICDSSLQFDKNNNLILNLTLDIPENNKNEKVEGRVVGVDLGMKIPAYVVLNDVEYIKKSIGSIDDFLKVRTQMQSRRRKFQKQLQSANGGRGRNKKLQSLSRFEEKEKKFAKTYNHFISSNIIKFAVDNKASQINLEFLSLKETQEKSVLRNWSYYQLQQFIEYKAKREGIGIKYVDPYHTSQTCSVCGNYEEGQREIQEKFICKNPKCKCELNADYNAARNIAKSTKYIKSKEESEFYKLNKKE

SEQ ID NO: 49 - protein, 427 aa, unidentified
MTKVVKLALICEQNDKDNNPIDYKEIYKILFELQRQTREIKNKSIQYCWEYSNFSSDYYKLNHEYPKEKEILSYTLDGFVNDKFKNGNDLYSGNCSTTVRSACGEFKNSRSDFLKGTKSVINYKGNQPLDLHNKAIRFDCIGKEYYVYLKLLNRPAFKKHNYANTEIRFKVLLYDNSSKTIVERCIDKIYKISASKLIYNEKKKCWMLNLSYSFSNDSETELDKDKILGVDLGIHYPICASINGERKFFKIDGGEIEHTRRKIEARKKSLLKQGISCGEGRIGHGIKTRNKPVYDIEDKISRFRDTANHKYSRALINYAVNNNCGIIQMEDLTGITSDSNRFLKNWSYFDLQTKIEYKAQEVGIKVVYIDPHYTSQRCSKCGYISKDNRTEQALFWCQKCGYKTNADYNASQNIAIKDIDKIINAKK

SEQ ID NO: 50 - protein, 482 aa, unidentified
MLKIKIECLNREVKNIITTRALKLTIVGDEETRNKQYKYIRNEQYEQYKALNLCMSLLNTHYVLNSYNTGAENKLKNQLEKLQNKIDKNNLELEKEDIKNSKKEKLTKQNMQFKGELIKLQEEYNKASKYRSDVDLAMKDMYIDDLYMAIQNQVTFKNKDFMSLVTQRAKKDFKNCLINGLARGERSLTNYKRNFPLMTRGERWLKFRYKEESDDILIDWIQGITFKVILGSRKNENTTELRHTLHKVITGEYKICDSEMKFDKSNNLMLNLTMDIPVKENTNYIDGRVLGVDLGIKYPAYVCLSDDTYKRMAIGSAQDFIRVREQIRTRRFRLQEQLKMVKGGKGREKKLGALERIKDKERGFVKTYNHMVSKNIVEFAYKNKCEYIHVEDLNKNGFDNAILSKWSYYELKTMIEYKAERKGIKVRYVNPEYTSQKCSKCGHTDKENRQSQEKFKCLNCGFELNADHNASINIARSNDIKK

SEQ ID NO: 51 - protein, 474 aa, unidentified
MADFVITRRIEVHLHHDPVADPEKVEYNRQWEYWRTINNNLYLAANRISSHLFFTDEYEHRLRIQHPRYRDIERTLASVSKVKRMSKEEIAALRVERRTIEQELRAQTAAFLQTSRQNVTYRIASDEFGDLIPSDVLTNLNQNITSTYNEFKKQVVRGERTLSNYKKGIPVPFSMKKGEGLRRRDDGSYYVLFPGGLEWDLAFGRDRSNNRAIVERAFNGDYEVGNSSLQEKNRKVFLLLVVKIPAQELALDPNRIVGVDLGLNIPLYAALNDNEYGGLAIGSREQFMKVRERMSARRRELQRALRHSTQGGRGREHKLQALERFQAKERNWVHTQNHIFSRAVVEYAKQNSAGVIQMESLKSFGRDKEDHIEAGFRYVVRYWSYFELQTLIAEKAKREGIEVRMIDPYHTSQTCSFCGHYEKGQRISQGVFICKNPECAKGKGRQLKDGTYSGINADWNAARNIALSTEVVKK

SEQ ID NO: 52 - protein, 472 aa, unidentified
MITARKIQVTIVSNNRDEDYRFIRNEMREQNKALNVGMNHLYFNYIARQKLRLADLAYQEKESKLVSQIDKIYDDIKKAKTDEKREQLKEKLEKQKKKLEKMRKQKNNDLFQQYQQIIGTSEQTSVRDAISEQFNLMSDTKDRLSQKVTQDFKNDIKAGLLTGERVLRTYKKDNPLYIRGRSLNLCKEEDTFYFKWIKGIVFQCVLGIKGQNKTELYKTLERVLDGNYKICDSALQFNKNNKLILILTLDIPDAVRESKIEGRVVGVDLGLKIPAYCSLNDDKYPRLAIGDIKDFLKVRTSLQRQYRSLQRALKSSKGGKGRYKKLKALDRFREKEKNYVTTYNHFLSREIVKFARKYRAEQINLELLSMAESTNKSVLRNWSYYQLQQFIEYKAAREGIKVKYVDPYRTSKTCSECGHFEEGQRTDQAHFTCKQCGFEANADYNAARNVAKSTKFITTKEQSEYYQKDSVS

SEQ ID NO: 53 - protein, 458 aa, unidentified
MNKVIKIYASLDKAQQCALPIYGKDSLSVQLLKVQSEVRSLKNRAMRMSYDYDQAQYEYFKYMERCKQEYGLNSYPSAQPSDFSKYKTFDGYLYDALAKDYPLMNKRISATVTRKVWGEYKKDKGEILSGKKSLRTYREGQPIPIRAKDTKLLYEDNFDYTMTVSVFSKDAAKTLGMKPGGCRFILHEQTDSEKAILDRLLSGEYKLCETLLAYDDRKNHDTKMARGWYFCIGYSFEKDTSNPSLDKDKILGVDIGVANVIYLGWSKDDHFKKYIPGSEIRKFQATEERRKKDILRCSVARGDGNVGHGRKCATRKAEKHEHHIHNFKETKNWHYAHFVVDTAVENGFGTIQMEDLSGINKSETFDRTWTFYSLQQKIEQLAAENGIVVKKVKPQYTSQMCSKCGYISSRSRRSQSEFRCVSCDYRRNADHNAAMNISKSKIEALVEEQLLRQGGEAD

SEQ ID NO: 54 - protein, 482 aa, unidentified
MYVEFIRIKGGEILITARKIKLTIAENREEGYSFIRKELQEQNKALNMAINHLYFNYVAREKIKLADETYKVKLEEGECYLERKYIELKEAKTDKQKENIKKSIEATKKKLETLRKVENKEVSNNFKEIIATSEQINLRDLISNNFNLKSDTKDKLTQKVVQDFYNDIVGVLRGERTLRRYKKDNPLYIRGRSLTLYREGEDYYIKWMNGIVFKCVLGVKKQNSLELQKTLDKVIFEEYKLCDSSIGFKDNKLILNLTLDIPVSNTNKFEKVIGRIVGVDLGMKIPAYCALNDSEDVRKAIGSIDDFLKVRTQMRSRRRKLQRALKSTNGGQGKNKKLSALNSFEAKEKNFAKTYNHFLSSNIIKFATDNKAEQINMEFLSLSETQNKSVLSNWSYYQLQQMIEYKAERIGIKVKYVDPYLTSQTCSECGHYEDGQREVQSEFQCKKCGCKTNADYNASRNIAKSDKYITKKEESEYYKNKI

SEQ ID NO: 55 - protein, 391 aa, unidentified
MRDKLKASFAGEKLKKIKNAQISSPSATSDRFPIPMWQQTGFRVETNNEDLVIDIPFPLYKYREEVDKFKPWEKLEFIDTSKKKHIQLILSTNSRKRNVGWVKDWSTEAEIKRVMSKKLVINKIEITRGKRINERNYWFVNFVIAYEKPVRKLDSKITGGIDVGVSNPVVCAINSGLNRLTIRDNDVVDFTRAELARLRSQRRGDRFRRGGHGIKHKFKPSETIQKKYEQRRKKKMEEWASRITRFFLNNGVGLVYMENIDKKSISDGEDYFKVQLRVTWPVKEMQKLFERKLKENGIEVKSIDPKYSSQLCSKCGKWNTHFNFRYRQLNGYPPFECKYCDYGKDKEKEIIYADYNAARNLANPNFDKRRKVAIPSEVLKGNIKEEVVAEN

SEQ ID NO: 56 - protein, 467 aa, unidentified
MITVRKLKLTIVGDEETRNQQYKLIRDEQYQQYRALNLCMSLLSTYNILNNWNSGAENKLNSQIEKLNKKVEKNKNDLKKDNLKENRIKKINESIQTLTKEKEKLQQEYLSSSEYRSDIDKKVKEMYIDDLYTVVQSQVNFKSKDMMSLVTQRAKKDFTTALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRTLGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKYGFPNMILSKWSYYELQNMIEYKAEREGISVKYVDPAYTSQTCSKCGHVDKENRTSQEKFKCIECGFELNADHNAAINIARSNNYVK

SEQ ID NO: 57 - protein, 441 aa, unidentified
MPQGKKLVTAQIKLYLHRPVEPDNISWAQAGQILRDVSYETVLGLNHAITEWHLYERERARHYRETKENLPAEERKKTWNRIYSEVRGLMKTASSGMASMVTRTAITRYQNSLKDIRSLKQSVPSYRLGHPILLREGGGETKLWRDNGNYMFRATLRNRSNEPTRLTFLLDTFKLEKSKKAVLDRVISGEYKLGACQVAQDRRKRWFTRIAYSFPRPELKKDTSICVGVDLGLACPFYCAVNNGHDRLSCNEALIVERFRWQIRRRRRAFQNSLKFSNRGGHGRNKALAPLEKLAEKEINFRDTKYHQYTSRIIEFALKQNAGVIQIEGLEGFRASQQGILRDWAIADFHSKLKYKAEHAGIEVREIDARYTSQRCSECGNINEANRQSQSDFLCTVCGYKTHADYNAARNISIVGIEKIIAEEIARKGLAEQEAQDVPQG

SEQ ID NO: 58 - protein, 411 aa, unidentified
MVKRGEHMNTVRKIKIIINNENNELRKKQYKFIRDSQYAQYQGLNRCMGYLMSGFYVNNMEIKSEEFKTWQKGVTNSANFFQEISFGKGIDSKSSITQKVKKDFSTALKNGLAKGERNINNYKRTFPLMTRGRDLKFKYDDNELDILINWVNKIQFKCVLGEHKNSLELQHTLHKVINNEYKIGQSSLYFNKKNELILILTIDIPTAKSSYEPIKDRILGVDLGMAVPVYMSINDNSYIKKSLGSYSEFAKVRKQFKERRNRLYKQLEACKGGRGRKDKLKAMNQFKEKEKNFAKTYNHFLSKNVVEFALKNKCEFIHLEKIESKGLENSVLENWTYYDLQEKIIYKAKREGIEIKFVNSSYTSQTCSKCNYVDKENRKTQVKFICKNCGFKANADYNASQNISKSKEFIK

SEQ ID NO: 59 - protein, 402 aa, unidentified
MITVRKLKLTIINDDETKRNEQYKFIRDSQYAQYQGLNLAMSVLTNAYLSANRDIKSDLFKETQKNLKNSSSIFNDIPFGKGIDSKSSITQKVKQDFSIAIKNGLAGGERNITNYKRTFPLMTRGRDLKFSYKDDCSDEIIIKWVNKIVFKVVIGRKDKNYLELMHTLNKVINGEYKVGQSSIYFDKSNKLILNLTLYIPEKKDDNSIKGRTLGVDLGIKYPAYVCLNDDTFIRQHIGESLELSKQREQFRNRRKRLQQQLKNVKGGKGREKKLAALDKVAVCERNFVKTYNHTISKRIIDFAKKNKCEFINLEQLTKDGFDNIILSNWSYYELQNMIKYKADREGIKVRYVNPAYTSQKCSKCGYIDKENRPTQEKFKCIKCGFELNADHNAAINISRLEE

SEQ ID NO: 60 - protein, 404 aa, unidentified
MNTVRKIKIIINNGDDEIRKSQYQFIRNAMYAQYQGLNRCMGYLMSGYYANNMDIKSQGFKDHQKTITNSLYIFNDIEFGKGIDSKSSITQKVKKDFSTALKNGLAKGERTVTNYKRSFPLMTRGRDLKFSYGDNDEILINWVNKIQFKAITGNSKNSIELEHTLHKIINGDYKVGQSSLTFNKKNELILILTIAIPEVKGDEYKPVANRTLGVDLGLAFPVYMALNDITYIRKSLGSYNEFAKQKLQYKARRERLYKQLDSVKGGKGRKDKLKALDQFKEKEKNFAKTYNHFLSKKIVLFAIKNQCEYINLEKIDSSGLENRVLGLWTYYDLQKDIEYKAKLAGIKVRYVKAAYTSQRCSRCGDIEKENRQEQSKFVCKKCGLDINADYNASINIAQSTEFIK

SEQ ID NO: 61 - protein, 428 aa, unidentified
MGNINIGGENMKEFRTVKKLKLTIVADTKEEREEKYKFIRDSQYAQYQGLNLAMGILVSGFLKGNRKLDSEAFKQAQKEMMAIREQTFEDINFGKGITSSSLITQKVKADFKTALKNGLAKGERNVTNYKRTFPLMIKGNADSCYDQGKRKPLDFYYDNDDIYIRWCNGIIFKVVLGTRINENTTELKHTLHKIIKGEYRVSQSSLQFDKNNNLIMNVNIRFKKELNTDFIEGRTLGVDLGLKYPAYVCLSDNTYIRKGLGSAEEFLKTRQQMKKRRTTLQHQLKLVKGGKGRNKKLKALEQFQNKERNFAKTYNHQLSCNIVKFAKENKCEFINLEKLTKEGFDNNILSSWSYYELQNMIEYKAERENIKVRYINPAYTSQKCSKCGYIDKENRKTQSEFNCLECGLKLNADHNAAINIANSTEYIK

SEQ ID NO: 62 - protein, 400 aa, unidentified
MITVRKVKLIVNSEEAEEINRTYKFIRDSMYAQYQGLNRCMGYLLSGYYANGMDIKSDGFKNHMKTIKNSLNIFDDINFGIGIDSKSAITQKVKKDFSTSLKNGLAKGERGATNYKRNFPLMTRGRDVKISYLEDTNTFVIKWVNKIEFKVILGQKDNIELSHTLHKIINKEYTLGQCTFEFDKNNKLLLALNINIPDNLISKNKEIIPGRVLGVDLGVKVPAMICLNDNTFIKKSIGSYNEFFKVRSQFKARRERLYKQLESSNGGKGRKHKLKATMQFRDKEKNFARTYNHFLSKNIIEFAQKYTCETINLEELNKKGFDNNLLGKWGYYQLQSMIEYKAERVGIKVKYVDPAFTSQTCSKCGYVDEENRITQDKFECQKCGFTLNADHNAAINIARK

SEQ ID NO: 63 - protein, 442 aa, unidentified
MPNITRKYQLKVVGDKEEIDRVYKYIREGTEAQNKALNEAMSALYAANLLDMSKDDKKELSKLFSRVTNGKNESGFTDDICFATGLGTTSSIKQKVKQDFNNACKKGLMYGRVSLPSYKADNPLLVSKSYVQLLSESDKNFGIYNTYETPMDLVDALEKETNPEVYLKFANNILFKFVFGNPWKGREQRKVFERIFSGEYKICGSSIGIDGKKIILNLCMDIPKQKHNLDESIIVGVDLGLAIPAMCALNNDDYKRLSIGSIDDLLRVRIQLQNERRRIQGNLKNSKGGHGRQKKLKALENLKDRERNFVQTYNHMVSKRVVDFAVKNNARYINIEDLSGFWKTRYGKSKSEDEKVLRNWSYYELQNYITYKAQLHGITVRKVRAEYTSQTCSYCGNKGIRKEQKKFVCVNPDCKCHKIYDGYINADFNAARNIAMSNDFAE

SEQ ID NO: 64 - protein, 428 aa, unidentified
MKLVKTMRYQIIKPLSCDWDTLGTVLRELQRDTHSVLNKTIQLCWEWQGYSSEYKAANGTYPTPKDTLGRSLEGYVYDRLKVQFPKMYTPNLSQTIQRAMLKWSADSKAIFKGEVSIPSYKKDVPLDLRKDSIHIERRGHDYILSLGLVSRAYKKELGLPECQIEVLIGTPDKTQRVILARLLTGEYTVSGSQIVWDKRNRKWFVNLAYHFEARPEQLDKTKILGVDLGVVFPVYMAVADGHFRAGIPGGEIEEFRRRVEARRRQLLRQGKYCGDGRIGHGRATRTRPLDKIADKIARFRDTINHKYSRYVVETARKLGCGVIQMEDLTGIREENLFLANWPYHDLQRKIEYKAREYGIEVRYVRPQYTSQRCSDCGYIHPDNRPEQAKFRCLACGFETNADYNAARNIATEGIEELIAAALNKASVV

SEQ ID NO: 65 - protein, 447 aa, unidentified
MVKTMKFQIIKPINMLWKDFENILRQLQQDTRNIKNKTIQLCWEYQGFSSEYKEKHGEYPKHSDILNYKTITGYIYDRLNNEYYRLNTGNLSDTIKSATDKWRNDIVDILKGEVSIPSYKRDAIIHVKNTNYKIVPEDGRYYLRLSLLSNVYRKELDLEKGQIDVAIRVADKNQKVTLERIFSGEYKQSSSQLMKKKNKWFFYMAFKFDPKTAKDLDPNNVMGIDMGITHPIYFAFNNSLKRGKIEGGEVESFRRRVEQRRRELLAQGKYCGKGRRGRGYETRVKPIKRIGDKIERFKDTANHKYSRYIVDVAVKNNCGIIQMEDLKGISKNNIFLKNWPYFDLQTKIEYKAKEKGIVVKKIAPRYTSQRCSKCGYINKENRVSQDTFKCVKCDFGHKFYVNADYNAARNIATPGIEEIIKKQIEKQKEMDEWENETIKLPLVGSDK

SEQ ID NO: 66 - protein, 439 aa, unidentified
MFMTKVTKVYLISEQIDKDGNKIDFKKISELLWNLQRQTRDIKNKCVQLCWEWLNFSSDYYKKSEEYPKEKDTLGYTLSGFVYDRIKNGSDLYSSNLSTSSRDTCTAFSNYKKEMLNGERSVLSFKANQPLDIHNKAIKLSYENGNFFVALKMLNRAGKEKYGINDDLRFRMQVRDKSVRTILERLMNDEYKVSASKLMYDKKKKLWKLNLCYSFDNHVISTLDPEKIMGVDLGVVYPIMASVNGDYARFSIKGGEIEAFRNRVEARRRSLLNQSRYCGDGRIGHGRKKRTEPAAQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPEVPTE

SEQ ID NO: 67 - protein, 439 aa, unidentified
MKPMNKVVRLALICEHSDKDGNPVDYSDVYKLLWQLQAQTREIKNKTIQYCWEYSNFSSDYYKENHEYPKEKDVLHYALDGFVNDKFKVGNDLYSSNCSYMTRKVCAEFKKSKSDFLKGTRSIISYKSNQPLDLHNKSIRIEYKDNDFFAFLKLLKRPAFNRLGYKNSEIGFKVIVRDKSTRTILERCVDQIYGISASKLIYNKKKKQWFLNLVYAFEPDNANNLDPSKILGVDLGIHYPICASVYGDLQRFTIHGGEIEEFRRRVESRKLSLLKQGKNCGDGRIGHGGKTRNKPVYSIEDRIARFRDTVNHKYSRALIDYAVKKECGTIQMEDLSGITAESDRFLKNWSYYDLQSKIEYKAKEKGIKIVYIDPKYSSQRCSKCGHIDKENRKTQSSFVCLKCGFEENADYNASQNIGIKDIDKIIENDLSSKCETDVN

SEQ ID NO: 68 - protein, 433 aa, unidentified
MKITKTMKYEIDKSIDVPWKTFLSVLRDVQYAVWKTGNIAVKMTWDFQQEAWSYRQRFGEQLKFSDLGTGNKSQSTDIYQRACSEYPNVASSVLDATIRMAQDRYKTDAPDIYDGLKTIPYFKRELPIPIRAQQTKLTRKGTKRYVSFALLSKEGAKKSELPTRYNVQIRTGKGAREIFDRLVDGEYKLCDSKILRKKGKWYLALSYSFEAENPQELDPKRVMGVDLGIVKTAYMAFNFDEYLRYEIEGGEISAFRGRIESRRKSLLKQARYCGEGRRGHGRKTRMKPLEKLRDKVANFRRTKNHHYSKYIVEMAAKHGCGTIQMEDLSGINKRDKFLANWSYYELQSFVKYKAKERGIKVVLVDPNYTSQRCSCCGYIAEGNRKTQETFKCVICGYKTNADFNAARNIAEPRIKALIDAELKRQEKERKEAM

SEQ ID NO: 69 - protein, 427 aa, unidentified
MSKVMKYELKYLGEEDFYEMQKMLWSLQEDTREILNKTIQIFFHWDYTNKESLETTGKALNLVEETGYKDISGYVYDKLKSRYPDMSRGNLSATIRAASKKYRSSKVDILKGTMSIPSYKKDQPIILRPDGIRLHEREMIYTVELSLFSGDFKKKKAWKSNVLFQIKACDKAQKAIMQRLLSREYKLGESKLVYKKKKWFIYITYSFKKTDAKLDKNKILGVDLGVTYAIYACSIGEYGSFSIKGEEALEYAKRLEARTISKQKQARYCGEGRIGHGIKTRLSTVYSTRNKLANHRDTLNHRYSKAVVDYAVKNRYGTIQMENLSCIKKNTGFPKRLQHWTYYDLQSKIEYKAAEQGIQVIKINPKFTSLRCSQCGCIHKDNRKTQESFQCVECGYKDNADHNAALNISIPQIDLIIKEEMTSAKEK

SEQ ID NO: 70 - protein, 462 aa, unidentified
MPIKALRVQIIKPFNTDYDSQPITWDELGRTLRDLRYAASKMANYVIQQNYMWEFFRQQYKQEHGSYPSVSEHKDKLYCYPRLTAMFPLAAGQMVNQIERHAKTVWSARKSEVLKLHQSVPSFKLNFPIIVHHDSYRISEVPEDGKSSTHVFLLQANLLSREAATRTRYSFLINAGEKSKQTIVERIISGEYRQGALQIVGDRKNKWYCIIPYEFKTEENNTLDPQCIMGIDLGISKAVYWAIIGSHKRGWIDGHEIEEFRRRVQARRKSIQEQGKYCGDGRIGHGRKRRLLPIEVLENRESNFKNTTNHRYSRFIIEAAIKNQCGVIQMEDLSGINERSTFLRNWTYYDLQMKIKAKAEEVGIEVRIVNPQYTSQRCSQCGHIDRDNRSNQATFVCTHCGYGGLYHCFACGKSQVEAGVCHLCGGETKIMKINADYNAARNLAICGIDQIIVQTLEGEGVR

SEQ ID NO: 71 - protein, 427 aa, unidentified
MTQHIRVMQYQIVKPCNGDWSTLAKVLSYLQNATRQVLNKTIQLCWEWQGFGSDYKRKFGENAVDRELLGYSLFGFCYHQLRQEFPLIHSSNISQSIQRAVLRWNSDAPEILNGVKSVPCYKRGVGVDLHKDTVRLKRGKNNEFVVSMNLLSLIGRKEFGFKSAALDTVIKVADGRQRQIFSRVLSGEYGLGACQIVCHKRKWFLQMRYKLAVKERQLNSNHILAVRMGIERPLYIAFSHTSSRVTLNGDNVTAFRKKFMARYAGMIRQKTMQGGGNIGRGKAKRFERIESMRTKISGFQKSLNHKYSRIIVEQAVLNRCGTIQIESPNVIRRQTAFLGNWAYFDLKRKVEYKAQQLGIKVVCLKTRDYEQRCSQCGHLNAVRETSKVLSGAYARSFFCESCGLLTTVDENAVQNLTLPNKPVMSQD

SEQ ID NO: 72 - protein, 444 aa, unidentified
MALVKSVRIQISRCEELDYKRMSSIFRDIRYKTCRASNEAMRLFLLNAFQSIRYKELHDIYPDIKSLTGKSLNTYIYNSMKEIMDICQTGNVAQTQQFVKNRFNTDIKDLLVNEVSYTNFKKDMPLFLHNKSYTICKDEDSGKYKVECSLFNRQYQKENNIKRVTFVLGKMDGNQKATLDKIIAGEYKQGAGHIKQDKKKKWYFTISYSFEPQIRKINTNRILGVDLGMVNTAVLQIWDIDKQKWDWLEWKECMLDGGKVYNFNQRVEGMKRSLQRSRKVSSVTSPYSKGKKGRGVQARIKALNRLNHKISNFKDTINHQYSKYIIDFALKHNCGIIQMEDLSKIKDKAEEKFLAHWTYYDLQSKIGYKAKAHGIKVVKIKPAYTSLRCSKCGHIDKENRKEQAKFKCVKCKYKLHADVNAARNIAIPEIERLIEIEKEEVNAI

SEQ ID NO: 73 - protein, 447 aa, unidentified
MTKNKMTTKTMKYEIRFEKALYNLLSDIQFEVFMLKNKATSIAYDWQNFSFSYHSRFGEYPKIKELSGVTLTNDILGELREVQAKFVSSATVASSVKEAVEKFTSDKSKILKGEISIMRYKRDGSFPIRSQQINHLTKINSKTYTCKLSLLSREGAKEREMKNGQMDVELRTGKGAYEILDRIIEGSYKLCDSRITKNKNKFYLLITYSFESDKVETLDENRIMGIDLGINIPAMLAISDNKYYREAVGDVTEISNFQKQVESRKRKLQKQRKWCGEGSIGHGTKTRIKPLEVLSGKIKRFKDTKNHNWSRYIVDQAVKHNCGIIQMEDLSGIAEENTFLKTWTYYDLQQKIKYKAEEKGINVVFIKPNYTSQRCSCCGHISKENRDVEKNGQDKFICVNCGYGSKFYVNADWNAAKNIATKDIENIIKEQLESQEKELKHNMKYAM

SEQ ID NO: 74 - protein, 444 aa, unidentified
MDVGTVVKMTRCRIEQDDELYKVLDDLRYMIYRIKNKATSMAWDWEQFSFGYHERFGEYPVAKEVVGKNVSRDAYQHVKGLGEEFSSSFVDTAVEEASKHFKNHRSAILRGKEGIPVYRSDTSFTIRHTQIKGLKKEQRDKYSGLFTLLSPKGSKVREQASTRYLFKIRSGGSSSAILDRILEGTYSLCDSKIVYEKKKRHYFLLVTYKFEAQKIEMNPERVMGVDVGFAVPATIAITDNPYACHFVGDAREVLAFEQRVLGTRRALYRSRSRAGDGSRGHGRKTLMKPTEVINSKVANFKATKNHEWSKFIVDYAVRHGVSTIQLENLEKIAEESPFLKRWTYYDLQQKIAYKAKEYGIKVIRVSPDYTSARCNKCGAIHRKQERTLWRPKQSQFNCLHCHHRDHADRNAARNLAIPNIDQIIKAEKVEWTSYWTNVYKNPPA

SEQ ID NO: 75 - protein, 450 aa, unidentified
MESEKEKYVVQSYGIKLIKPVGVDEGDWDFAGKVLRDLDYACFRVKNKAATRTYMNVIEKLEYENIHGKGTYDKYFKTRYGKTFSAYNSECAREDKELNKGDLYREYFNLMAREGEKVVKYNMKKILNGNASNITFKRNQPVPITSRMIFITKESGKYYAELTLLSPEKAKELGRKGKNGTRIKFLLSSKGQEKVILDRLTSGEYDLRDSHIHVKKKGNKLTNYLIIAYRHKVKDDNDLIPNKVLGVDLGVSKAAYMAVSESPVSEYINGGEIEQFRNGVEARRNGMRNQLKYHSSNRSGHGRATKLKPLEKLREKVSNFRKLTNHRYAKFIVDTALKNNCSIIQMEELKGISKNDTFLKRWSYFDLQEKIENKAIAVGIEVKKVSPKFTSQRCNKCGYIDKESRKSQEKFECVNCGHKTNADLNAARNLSMLNVEKEIKAQCKAQKIKY

SEQ ID NO: 76 - protein, 426 aa, unidentified
MLQVTKAVRFQIIKPLNFSWDEFGRILNDLSYHTTLMCNAAVQMYWEHNVMRNRYKAEHGRYPQDKEIYGQSFRNVVYHRLREMYPLMASSNVSQTNQFALKRWQTDLREVMRLQKSVPSFRLGTPVQVANQNYSLYIAKGEPPEYCAEITLLGKDAACRRFTVLLDAGDAPKKAVFRRIVEGKYKQGVMQIIKHPRKKKWFCIVSYTITKDPAPGLDQERVMGVNLATGEAVYWAFSFSPKRGSIPAGEIEAAEKKIRAITARRREMQRTAGVTGHGRKRRLKATRVLAGKTANIRDAINHKYSRRIVRIAAANRCGKIRLADMSALGMSGALKAWPWSDLVQKIGYKAAEQGIDVEIVEKPGDRAKAWHTCSECGYSAPENVGDNTEFLACKECGARISLEYNAALNIAVLARDSIPEQQTSAS

SEQ ID NO: 77 - protein, 431 aa, unidentified
MEITKATRYQLIKPLDVSWEDFGQILRDLSYHTTKMCNAAVQLYWEYHNQRLAYKQEHGKYPEDKVMYGMSFRNVVYHRLRGIYPLMASSNTSQTNQFALNRWKNDVPDVMRLQKSIPSFRLGAPIQVANANYRLYVAEGEKPEFRADVTLLGKDAAQGRFSLLLDGGDAPKKAIFRHIVDGTYKQGVMQIVRHPRKKKWFCIISFTFTSEEKSLDESRAMGVNFGSGEALCWAFNFGPKRGTIPAAEIEAAEAKIAAITARRRGMLRTAEARGHGRTRRLKPTESLQGQAANIRDAINHKYSRKIVSVAVGNRCGIIRLADTSGLELDGAFKHWPWAGLAEKIRYKAEEAGIVVETADAKKAFYTCSKCGYSHPENTDGNKEFLTCKNPDCEAQVNLQYNTAKNIAVNVPGPEEKKSPKKEKRKKEIAKQ

SEQ ID NO: 78 - protein, 411 aa, unidentified
MTKAKDRELSKVFRVEVLKPLNLTWDEFGGLLRRTQYHAAHLANEVITTQYLLAKGKLERNGSFCALVAHACHDCSLHADVKCTVCKWARDKFKADARRILRADISLPSYKNNLCMIKNRSVKLRETPDGWAARLAILPKADGKNQVQPEVLLRTEEMKRRSPGAYQVLERIASKEYKQGTTQVKRDTRTGKIYLLISYSFRPERESGLEASRVMGVDLGVSTPAYCAFNDSLKRKSLLIEGRKLLKTKWQIEGRRRDIRRHNDQRDLRRGHGKEAKFRPMEAVEQHWVDFRQSWNHVLARRIIEYALSNHAGAIHLEDLSPGTNSKFLGRNWPVAELLDYIEYKAKERGIAVKRVNPFKTSQTCSDCGAIKESFTFGDRKKAGFPDFVCDACGFRTHADYNAARNIAKAP

SEQ ID NO: 79 - protein, 495 aa, unidentified
MNKCIKVILNKCINIDIKEAKKIIKNMSYLSCKASNKAIDMWKQHSLNIMELKSNDKNFNQKEYEQATYGKNYKNVIEGYMKEIMNICNTSNVSTLHQQQVQNDWKRLRKDVLNYRANLPTYKLDTPCYLKNNNYKLRNHNGYFVDISLFSMKGLEQIGQKKGYQLQFEIDKMDGNKKSTINKLINNGYKQGSAQLKISDKGKIELIMSFSFEAKESNLDENRILGIDLGIVNVATMAIWDGNTQEWDWVNYKHNILNGQELIRFRQKLFNMGMSEFEMQNEVYKQNQKIHQKQLNKHNIGAIDGLELVKYRDTIDKKKREMSIASKWVGEGRVGHGYKNRMKPLEKIRNKASNFADTFNHKYSKYIVEFAIRGNCGVIQMEDLSGATKNTHGKFLKDWSYYDLQTKIEYKAREVGIDVIYVKPQYTSKRCSKCGNIHTDNRDCKTNQAKFKCMNVTCGHEENADINASKNISIPYIDKIIEEYIKENDIYKNKK

SEQ ID NO: 80 - protein, 433 aa, unidentified
MIQNRRQNRLILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 81 - protein, 471 aa, unidentified
MATKEKRIVKVMRLRILKPAGEMSWSQLGKLLRDTRYRVFRLANLAVSEAYLNFHLWRTGRSQEFKADDMGKLSRRLRQMLADEGVAADELDRFSPTGAVPDAVSGPLFQYKIRAITNKNKWREVIRGTASLPTFRLDMAIPVRCDKARMRRLERMENGDVQVELTICRKPYPRVVLQTGDIGGGQEAILARLLDNTGNDLTGYRQRVFEIKQERQTSKWWLYITYDLPAPQTGKADPDVVVGVDVGYAVPLYVAINNGHARLGWRQFDALGRRIRKLQTQVLARRRSIQRGGRVNISHATARSGHGVKRKLLPTEILQRRIDKAYQTLNHQLSASVIDFARDHGAGVIQVEDLEGLKEELTGTYIGARWRYHQLHQFLKYKAEENGIEFRAVNPRFTSRRCSKCGHINVAFDRAYRDAHRENGKTARFICPQCGFEADADYNAARNLATLDIEALIEAQCARQGLTKDAL

SEQ ID NO: 82 - protein, 408 aa, unidentified
MITVRKLKLSIRESDEESRKVKYQFIRDSQYAQYRALNLAVGILSSAYLKSNKDTKQESYKNAIKSLTNSNPIFSEIEFGRGVDTLSLVTQRAKKDFKNSIKNGLARGERNLTSYKRTYPLMTRGRDLKFRYEGDDIVIRWVNKIEFNVITGSNKIKENTVELKHTLHKVINKEYNVKESSLMFDKNNNLILNLTLDIPNELSYQPIEGRTLGVDLGIATPAYVCLSDDTYVRKGIGSIEDFLKVRTQFQKRRRVLNQSLVMAKGGKGRKKKLKALESFKEKERNFAKTYNHQLSHEIVKFAKNHKCESINLEKLTKEGFNNRILRNWSYYELQSMIEYKAEREGIKVRYVNPAYTSQKCSKCGHVDKANRQTQAQFKCVECDFELHADHNASINIARSEEFVQGAGS

SEQ ID NO: 83 - protein, 411 aa, unidentified
MVKRGEHMNTVRKIKIIINNENNELRKEQYKFIRDSQYAQYQGLNRCMGYLMSGFYVNNMDIKSEEFKTWQKGVTNSANFFQEISFGKGIDSKSSITQKVKKDFSIALKNGLAKGERNINNYKRIAPLMTRGRNLKFKYDDNELDILINWVNKIQFKCVLGEHKNSLELQHTLHKVINNEYKIGQSSLYFNKKNELILILTIDIPTAKSSYEPIKDRILGVDLGMAVPVYMSINDNSYIKKSLGSYSEFAKVRKQFKERRNRLYKQLEACKGGRGRKDKLKAMNQFKEKEKNFAKTYNHFLSKNIVEFALKNKCEFIHLEKIESKGLENSVLANWTYYDLQEKIIYKAKREGIGIKFVNSSYTSQTCSKCNYVDKENRKTQAKFICKNCGFKANADYNASQNISKSKEFIK

SEQ ID NO: 84 - protein, 481 aa, unidentified
MITVRKIKLTIMGDEETRNRQYKWIKDEQYNQYKALNIGMSYLATHLFLKMSESGLEQKTEKNIKTIEKQISKIENNITKEESKKKVNEEKLNNLINELDTLNASLSKLKEELEEISNNRSNVDDTFKRMYVDDLYNALSKVPFQHSDMKSLVSRKVKLDFNTDMKDLMSGNRSVRNYKRNHPLLVRGRDLRFRYDGSNIKIKWIQGIEFKAILGKISKTIELRHILNKVIDGEYKVCDSSLEFNKNNHLILNLAIDFPYTNKIEFIEGRVVGVDLGIAVPAYVALNDIAYVKKSIGDIDDFLRVKTQMKKRRNLQINLTSVKGGKGRSKKLKALDRLSEKESNFVRTYNHFLSKSIVKFAIDNKAGQINLELLSENALSDKIIKNWSYYQLQQFIKYKAERYGIKVKYVDPYRTSQTCSVCGHYEEGQRSKQDIFTCKNEKCKMFEKEVNADYNAARNIAISTKYIDDIKESEYYYKKSF

SEQ ID NO: 85 - protein, 475 aa, unidentified
MSIPLTRKIKLSVYVPNTITEEKERHEYKTFVWQLLRKVNQQNYEFHNLLVKKLIELDSVLEGRLTEDDNFIEIRNRYYSDRKDKKNQREFFDYIRMKKDELIKEFGGKNLHGYIYTYLKNYIKSLPEEQQFMASYTYGSISKNVVDKYTNDQFDVFRGVKTIATYKNTQPIPINIIGHVKKNEKGELYKNTGKEWFNKYDDIYTFKFKSIKKHEIELQLNFGKDRSNNRIIVDRIYNNDPNYKICDSKIQVVGTEIFLLLVFKQFVTKRELGLDVRKVIGVDLGVKNVATIATNFSDHIEIIGEGEFVLMRTKLQIEKQKRSIQRNSKYSRGGHGRNRKLQKLNEYRNYERNFRSTFNHKISKDVIEIAIKHGAGQINIEDLSSIPLKEKNNRILRFWSYYDLIQKIIYKAKREGIIVNLINPSYTSQKCHQCGQIGNRPKQDTFLCTNPTCKAFNEPINADVNAAKNIAKISL

SEQ ID NO: 86 - protein, 419 aa, unidentified
MQRSITLKILRPRDEKISWEEMGYLLGGLSMKVCRMSNFCMTHHLLHALKLETELLNPRGDLYCYPVLAEEYPEVPSGIICAAETRARKLFKRSAAKVLRSETSLPSFRKDSSIPIPVAGYRILQDGDGNYCAEIQLISRQGAKTQKLPGRICLVLADNWRDKSAKSALQKVAAGKVRRGVATLFRAKKDWYLCIPYVTEPADIGENFEPGLVMGVAFGMFDVLAYGFNTLLKRGAISGEEVLSHQDKFMARRKKIQEQYAWSGRKGQGREDALKPLRHLYEVEKNYRDLVNNRYAKWVVDIAVKNRCGEIHLDSGNSTSKGNKEILLSHWSLYDLKDKICRKAEEKGIRVTECNVPNLRTRCSHCGTEQAVENRKRMFLCKNCGYGTTDKNKSNGYISADYNAARNLAVYDTGDTEPV

SEQ ID NO: 87 - protein, 481 aa, unidentified
MIITRKIAITIVSEEAQESYNYLRQQIYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPMLQRDSIDFAASKVASDFNNDLKLGLMTGSRTLRIYKRNQAYPFRSRRLKFYKENGDFFINSSKSLLFKCLLGVKRQNSKELIQILEKILDNQYKICDSSLEFNKKKLILNLCIEVDENTHSENMKVPGVVKGRIVGVDLGIQIPAYCTLNDSPFKKKAIGSVDDLLRIRTQMQARRRRLSKNLISARGGKGRGKKLKALDRFEEYERNYVRTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIAK

SEQ ID NO: 88 - protein, 481 aa, unidentified
MIITRKIAITIVSEEAQESYNYLRQQIYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPMLQRDSIDFAASKVASDFNNDLKLGLMTGSRTLRIYKRNQAYPFRSRRLKFYKENGDFFINSSKSLLFKCLLGVKRQNSKELIQILEKILDNQYKICDSSLEFNKKKLILNLCIEVDENTHSENMKVPGVVKGRIVGVDLGIKIPAYCTLNDSPFKKKAIGSVDDLLRIRTQMQARRRRLSKNLISARGGKGRGKKLKALDRFEEYERNYVRTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIAK

SEQ ID NO: 89 - protein, 531 aa, unidentified
MNDRSIITRKLTLIPAFSDRPKWEEKVMSYTEQFYIDKIAYYKNKLTKTKGKEEKQKIKDKLASLEEQENEFEESGILTQANVTDYTYDLVRNAMASEANRKNAIISYIILELLHNGGQTMDFNARNKLINDLVNYGLRVKGSSKGSLFDELDIENPLNAYGFAFKQDLKKKIRDMVNSKRVLDGKSSVITYKADSPFSINKENMSFTHDYSSFEELSDHIRDNDTNLYFNFGSSGNPTIARFKINLGAGRHKKNKDELIATLLKLYSGEYQFCGSRIGIEKNKIILNLVLSIPKKVRALDENTVVGVNLGVAVPAMCALNNNEYERLAIGSADEFLRVRTKLQAQRRRLQKSLKDASGGHGRTKKLKALERVAKAESHFANTYCHMISKRIVDFALKNNAKYINLENLTGYDTNDFILRNWSYYKMQQYTTYKAEKYGIIVRKVNPCYNAQACSVCGNYAPGQRKSRAVFICANPACKSHKKNHGKLDAEFNNARNVAMSTLYMNDGQVTEKSFKEARDYFGIEEEIETI

SEQ ID NO: 90 - protein, 471 aa, unidentified
MITARKVKLTITENREDGYNFIHNELREQNQALNMAMNHLYFNYVAREKIKLADETHKIKLAEDQGYLDQKYTELKEVKTDKKKQNIRKSIQAAKKRLETLRKAENKQVAEKFKEIIAASEKTNLRDFITDNFNLTSDTKDRLTQKVSADFKNDIVDVLRGERTLRRYKKGNPLYIRGRNLTFYIKDEEYYIKWMKSIVFKCVLGVKKQNSLELQKTLDKVIEGKYKVCDSSIEFKQNSLILNLTLNIPVCNSFDKVEGRVVGVDLGMKIPAYVTLNDSDYIRRAIGSIDDFLKVRTQMQSRRRNLQRALKSTKGGKGREKKLKALNQFEVKEKNFAKTYNNFISSNIVKFASDNKAKQINMEFLSLSETQNKSVLRNWSYYQLQQMIEYKANRVGIKVKYVDPYHTSQICSKCGHYEEGQREKQEVFICKNPECKNFNIEVNADYNASRNIAKSNKYITKKEESEYYKIN

SEQ ID NO: 91 - protein, 496 aa, unidentified
MILTRKIKLVIVSENREEGYNLIRTEIREQHKALNLAYNHLYFEHNAIQKLKQNDEDYKQKRNKLQELINKKYEEHQKAKNLEKKEALREAYNNKKQELYNFEKEYNEKARQTYQQVVGFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVVELQSVLENVLLGAYKVCDSSIGFNNKDMILNLSLNIPDKETQGYIPGRVVGVDLGLKIPAYLSLSDKVYVRKGIGSIDDFLRVRTQMQKRRRRLQKSLAAVKGGKGREKKLKALDHLKGKEANFAKTYNHFLSTQIVTFAVKNQAGQINMEFLEFDKMKNKSLLRNWSYYQLQIMVEYKAKREGIIIKYVDAYLTSQTCSKCDHYEDGQREKQENFMCKNCGLEVNADYNASQNIAKSTSYISDSTESEYHKKKQQVLKEILGENDIMNEQLSLFNNCDDIA

SEQ ID NO: 92 - protein, 453 aa, unidentified
MSKITRKIKIIPDIDGITHEESNKKCYNTFYKFDRKLYKVANLLVSQLYGLDSLLSLMRLQNDEYVKCLSKLSFKSITDATKEEIKKRMKEIDAELISIKNDIAPKHPQTYSYRAVTSSEYAKDIPSDILNNLKQDVYQHFNENKKEQIRGERSLATYKKGMPIPFNLKKKHGIISVGDNYYLPWFEDTRFRLNFGRDRSNNRAIIDNCIKTKKYKLCAAAKIQLKERKLFLLITVDIPKAESVPVKGKVMGVDLGVVNPAYVAVNDGPERSRIGNGEAFQKQRDVFRRRFRELQRSQLTQGGHGRKHKTKATEILRGKERNWVQTENHRISREIVNLASRWKVETIQMESLKGFGKNQEGEVEYNHKRLLGRWSYFELQKDIEYKAAMAGIAVQYVNPAYTSQTCHVCGQRGNRIERDTFICTNPECTCYNQAQDADMNAAINIAKSKDVIK

SEQ ID NO: 93 - protein, 415 aa, unidentified
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVDLGMKIPAYVTLNDSEYIGKPIGDINDFLKVRKQFKERKERLQKQLAINKGGRGITNKMQLMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNADRNASFNIARSTKYISSKEESDFYKQLK

SEQ ID NO: 94 - protein, 417 aa, unidentified
MQRSVTLKIIRPEDETISWEELGYLLRGLSFKVCRMCNFCMTHQLLHALKLETELLNPQGNLYCYPRLAEEYPDVPTGIICAAETRARKVFRRSAEAVLHSETSLPRFRKDSSIPVPVAGYKILQDSDHNVYADVQLLSRQGAKTQKLPGRIRLVLADNWRDQSAKAALRQIADGKVKRGVASLFRVKNDWYFQIPYVTEAVNTGEGFEPDLVMGVAFGLQDALVYAFNTSLKRGAVSGEEVLAHQEKYAARRKKIQEQYNWSGRKGHGREDALKPLRHLYETERNYRSLVNSRYAKWVVDIAMKNRCGMIHLDSANYVSSGKKILLSRWPLYDLKEKIRRKAEEKGIQVTECSIPNLRTRCSLCGKEQEPEGEKHTFVCKDCGYGKADKNRRSGSITVDYNAARNLAAYKSEDTKL

SEQ ID NO: 95 - protein, 400 aa, unidentified
MITVKLQLYKPTKCKEQRLFSIIREFTSCANWYLDKLQESRTTSRVKIHNGYYETARKSFRLLSANVQLALDKAIETQRAFLNKKGKKSVPKFKKQFACFRQDTFKIFDRYVQFNMPGRERVNIPFKVCNPNHKQFIKQQPKRSQLINKKGKWFLYVSYETDKPAIDGNINIIGVDLGVKKIVTVSNPEASVNVFFSGNKAIYTRNKYQRYRKQIQRAKDTGKAKRGYRALKRISGKEKNWIKDTNHKISKQIVNIAKQNKADIAIENLKGIRERIKATKKVRRMLHSWSFRQLISFLQYKSAMAGVRIVSVDPRHTSQRCPRCGHISKDNRKSQSAFKCSQCHYSVNADLVGSRNIALTALNLYGEGKRPSERAVMLLPMAEGKTLMASCLEAPSVRAG

SEQ ID NO: 96 - protein, 400 aa, unidentified
MPTITLRLELHNPTKVKQDMYERMTEVNTAFTNWLLNHPKLNQATSKIFKEFSPQRFPSAVVNQTIREVKSQKKNQKTKKFQTLWCCFNNQNVKVEKKGSFYTVSFPTLEKRIGVPVVTRPYQEAWLNRLLDGTAKQGAAKLYKKRKKWYLAIAITFDVKPRHETKVMGVDVGLRYIAVASVGTKSLFFKGSQCAFIRRRYAALRRTLGKAKKLQMIRKIGRKESRWMKDQNHKISRQIVNVALANGVGVIRMEALTGIRKRAKSAKEAGRSLHAWAFHQLQTMIAYKAEMAGIRVEWVDPTYTSQTCKCGHREKANRNGIRFRCQRCGYTLHADLNGAINIAKAISGFAAEPSALVTGAPPIGVHRNPMGRGDDTPLKLLRCPNQKWMRTQTTQESHAL

SEQ ID NO: 97 - protein, 412 aa, unidentified
MWHVELKRTARVKLAIPDDRRDDLKRTMLTFREVAQRFADRGWERDEDGYVITSRTRLQSLVYKQVREDTGLHSDLCIGAVNLAADSLRSAVERMKAGKNVGKPTFTVPTATYNTGAVSYFTDGDGTGYCTLAAYGGRVRAEFVYPPDEDCPQRQYLGGDEWEPKGATLHYERDDGEYYLHVTVERDEPETELGEAENGTVLGVDLGVENIAVTSAGAFYSGGLFNHRRDEYERIRGSLQQTGTESAHRTIEKMGDRERRWNTDVLHRISKAIVQEAITHDCSHIAFEDLTDIRDRMPGAKKFHGWAFRQLYEYVEYKAAEFGIATTQVDPAYTSQRCSKCGTTLRENRTSQAAFCCQKCGYEVHADYNAAKNVATKLLRSGQKSPAGGATNQLALKSGTLNGNGDFTPASS

SEQ ID NO: 98 - protein, 437 aa, unidentified
MPTITLRLELYNPTKVKQDMYERMTEVNTAFANWLLNHPELNQATSKIFKEFSPQRFPSAVVNQTIREVKSQKKNQKTKKFQTLWCCFNNQNVKVEKKGSFYTVSFPTLEKRIGVPVVTRPYQEAWLNRLLDGTAKQGAAKLYKKRKKWYLAIAITFDVKPRHETKVMGVDVGLRYLAVASVGTKSLFFKGSQCAFIRRRYAALRRTLGKAKKLQMIRKIGRKESRWMKEQNHKISRQIVNVALANGVGVIRMEALTGIRKRAKSAKEAGRSLHAWAFHQLQTMIAYKAEMAGIRVEWVDPTYTSQTCKCGHREKANRNGIRFRCQRCGYTLHADLNGAINIAKAISGFAAEPSALVTGAPPIGVHRNPMGRGDDTPLKLLRCPNQKWMRTQTTQESHAFRRAECQIISSQTRVPRPDGSAGSTMSRRGRRWRISCT

SEQ ID NO: 99 - protein, 414 aa, unidentified
MKASRFLILNDYEKNGILSSPQGGESMPTITLRLELYNPTKVKQDMYKRMTEVNTAFANWLLNHPELNQATSKLFKEFSSQRFPSAVVNQTIREVKSQKKNQKTKKFRTLWCCFNNQNVKVEKKGEFYTVSFPTLEKRIGVPVVTRSYQEAWLNRLINGTAKQGAAKLYKKRKKWYFAIAITVEVQQREETKVMGIDLGLRYIAVASVGTKSLFFKGSQCAFIRRRYAALRRTLGKAKKLHMIRKIGRKESRWMKDRNHKISRQIVRFALANGVGVIRMEELTGIRKRETSAKEAGRSLHSWSFHQLQTMIAYKAEMAGIRVEWVKPTYTSQTCRCGHREKANRNGIHFQCKKCGYTIHADLNGAINIAKAISGFAAEPSALVTGAPPIGVHLNPMGRGDDTPLNLGVVRTRNG

SEQ ID NO: 100 - protein, 557 aa, unidentified
MANKNVNDSKIKTHILTKKVQLIVDTDRFDTDEEKKAEVDRVYKYLRDSMRCQSREMNQYYMHLWMMSVARNLNDDRYSMKKYMNNIIDAVHPYLDKNNKEFNNKQKKITRDFEKKIKNLCEEYQAETEILNNDTLRELNKCFNRKKDGAYDTNFVNEMPEGLGIVMGRTVEQDFQNDCKAGLLSGIRNPRSYKINYPLIIPKSFVAYGVGSGKAMQGRGIIFPDMEYSDFHNMLFSTKNPNITYNFVHDIDFKLVFGSMKRSHELRVIFDRIKMGEYSICGSTIEINNKKKIMLNLSYEAPIYEKPNLDENTVVGVDLGMAIPAVCSLNNDDRTYKYIGDSHELEFIKKGIQAQRRSYQRNAVYNKGGHGRNRKLENLDRLKKRERNTTRTHNQRYAKQIVDFALANNAKYINLEKLKGFSNNEKNKLVLRNWCYYELQQYIEIDASKYGIKVRYIYPMNTSRTCSVCGTLTTDEDVKNGVGRVSQDEFICKDPNCPSHTLYTRGPKGHKVPYFNADRNASRNIAMSEDFVKKNAKDNAFKKIDDLYEINNDIDAA

SEQ ID NO: 101 - protein, 498 aa, unidentified
MQLNTITLTVKLQIRPLGDKEAQNEVWRNLRNINRDVFKAANLLMSHLHFVEAFEHQFMQTDRILEKEMLDRENKKKNKKLNAQQIETIAQEIAEIKQKRKELKIEVAEKVKIFYNGKNESFAYNFIRHEYPQIPSYSVSILVKTVQNKFKEEWKEVKKGEKSISNFKKTIPIPIDDKQIANTDKGKKLCYIKKEGEKFVWELNSLEAKFEIVTGKYKQSKYMGEDKNKRTTLERVMNFEYKVADSSLKIEENKIFLLLVCKIPQQQATSLPNVSIGVDLGIRTPACIACNDGLRVISLGSKQDFLAIRQRFYEHRKRLQKSLAMTKGGKGREKKLKALEKLRKAERNWVRTYNHTLSKKIIHFAIQCRASRIQIELLEGFGRNENKVDEKGNYLLGKWSYYELQNMIKQKAEQYKLVVTTIDPYHTSKTCHVCGELGYRKGADFFCQNERCKEYQKRQNADHNAAFNIAKSTTFVAKKEDCTYFKLEQEKKSKKEEE

SEQ ID NO: 102 - protein, 399 aa, unidentified
MRRTVKIKLSLNQNQQKLLQETIKQFKQACQRVVDYGWNRNGLKTYQKNKLHKATYSEIREKTDLPANLVIRARDRASETIKACVKKIKNGEKASKPTFKSDSIVYDKRTLTVWLEEERCSIATTNGRIKADFVLPNEYNDYYDKYLNNGWKITQSTIEKHSYEDDEPFYLHLGLEKEIQKNQSVNPTIMGIDLGIENLAVTSTGRFYSGTELFSRRERYEEVRGKLQAKGTRSAHLTIKQMSGRENRFACDTLHRISKKIVEEAISKDVDVIAMEELEKIRQKISNNKKFQTWAFKKLQEYIEYKANERGIEVKFVDPKYTSQRCSRCGTTLKQNRINQHFECKDCGYRVDSDYNAAKNIGFKTILDGQMSQSRMGNGQLALKSGVLKPNGNYYSYPN

SEQ ID NO: 103 - protein, 409 aa, unidentified
MIVTRKIQIRPINKEHYKILEDYLRTCRLIANKSQTLFYVYWQEIMANNLKGKNIDAYFKERYQHSFKQTIYHILRPKHLEIPSRITDDTLNIAYKDFCNDLKNGLLKGERSLRTYNNGWIPVRSQTTKITKQNNKYILEWLKGIKLEVYFGRDKSGNQVVVDRILNGQSKFCDSKFIKKEKKWFLLLCVNEPEKENNLFDDISIGVDCGINIPAVCAVNKGYGRAYIGSSVLLTRFRMQKDQRQRQKNYISSSGMHGRQKVDRAIFKAQDFEHRYMHTFHHKISKEVIKFALKNRASKIIMEDLKRFGQNQEESEEKKKIRRFWGYQQLQSMIEYKAKLENIAVKYIPPAYTSQTCSQCGYTDANNRKQSKFVCLNPKKKCGFEANADYNAALNIAKGGIKKVNNKDV

SEQ ID NO: 104 - protein, 460 aa, unidentified
MPTITLRLELHNPTKVKQGMYERMTEVNTAFANWLLXLELHNPTKVKQGMYERMTEVNTAFANWLLNHPELNQATSKIFKEFSSQRFPSAVVNQTIREVKAQKKKQKAKKFRTFWCCFNNQNVKVEKKGVFYTVSFPTLEKRIGVPVVTRSYQEAWLNRLLNGTVKQGAAKLYKKRKKWYLAVAITVEVQQREETKVMGVDLGLRYIAVASVGTKSLXAKLYKKRKKWYLAVAITVEVQQREETKVMGVDLGLRYIAVASVGTKSLFFKGNQCAFVRRRYAALRRRLGKAKKLHMIRKIGRKESRWMKDQNHKISRQIVAKKLHMIRKIGRKESRWMKDQNHKISRQIVRFAVANGVGVIRMEALTGIRKRATSAKEAGRSLHAWAFHQLQTMIAYKAEMAGIRVEWVNPTYTSQTCKCGHREKANRNGIRFRCQRCGYTLHADLNGAINIAKAISGFAS

SEQ ID NO: 105 - protein, 496 aa, unidentified
MILTRKIKLVIVSENREEGYNLIRTEIREQHKALNLAYNHLYFEHNAIQKLKQNDEDYKQKRNKLQELINKKYEEHQKAKNLEKKEALREAYNNKKQELYNFEKEYNEKARQTYQQVVGFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVVELQSVLENVLLGAYKVCDSSIGFNNKDMILNLSLNIPDKETQDYIPGRVVGVDLGLKIPAYLSLSDKVYVRKGIGSIDDFLRVRTQMQKRRRRLQKSLAAVKGGKGREKKLKALDHLKGKEANFAKTYNHFLSTQIVTFAVKNQAGQINMEFLEFDKMKNKSLLRNWSYYQLQIMVEYKAKREGIIIKYVDAYLTSQTCSKCDHYEDGQREKQENFMCKNCGLEVNADYNASQNIAKSTSYISDSTESEYHKKKQQVLKEILGENDIMNEQLSLFNNCDDIA

SEQ ID NO: 106 - protein, 471 aa, unidentified
VITARKVKLTITENREDGYNFIHNELREQNQALNMAMNHLYFNYVAREKIKLADETHKIKLAEDQGYLDQKYTELKEVKTDKKKQNIRKSIQAAKKRLETLRKAENKQVAEKFKEIIAASEKTNLRDFITDNFNLTSDTKDRLTQKVSADFKNDIVDVLRGERTLRRYKKGNPLYIRGRNLTFYIKDEEYYIKWMKSIVFKCVLGVKKQNSLELQKTLDKVIEGKYKVCDSSIEFKQNSLILNLTLNIPVCNSFDKVEGRVVGVDLGMKIPAYVTLNDSDYIRRAIGSIDDFLKVRTQMQSRRRNLQRALKSTKGGKGREKKLKALNQFEVKEKNFAKTYNNFISSNIVKFASDNKAKQINMEFLSLSETQNKSVLRNWSYYQLQQMIEYKANRVGIKVKYVDPYHTSQICSKCGHYEEGQREKQEVFICKNPECKNFNIEVNADYNASRNIAKSNKYITKKEESEYYKIN

SEQ ID NO: 107 - protein, 422 aa, unidentified
MMTKSVKIKLGTPIDSNWNTVRKILADLRYNSSKMLNFSIQQCYQWMIYRNEYREKYGKYPRAKDIYGYSHRNHIYRQAAEIFPIFNRGNISQTVGMATSRWSSDQKDVMSLRKSIPSYRLQAPIYIANQSYTISRTENGLVVDCSLVSKKYAKEETNDKTRYRISLVVKDNSTETILDRIVSGEYSQGYGQIVKDRRKEKWYLLVAYRFEPKKTKETGRILGIDLGIVYPLYMALNDSHHRYRIDGGEIEHFRRNIEKRKNQLLDQGKYCGDGRRGHGIRTRIAPIEFAREKIKNFRNTTNHKYSKFVVDIAQKHDVETIQLEKLDGISEDSTFLKNWSYYDLQQKIQYKAQEKGIKVAYIDPRYTSQRCSRCGNISRENRQDQQRFRCTKCGFSANADYNAAKNISTQNIEKIIEEELKK

SEQ ID NO: 108 - protein, 480 aa, unidentified
MTTKVMRYQLIEMVDNEKRFMYKMLDDLRYEVFKISNRAIQMFWDIDNTSYAFKQKFQENLDLKELTGVKSLAYISRALKEEYQKLNNTSVEQVSRKVEKEWKKNKSNMITADTSMIRYKRKNANIKLKNTQFKIEPLDNNFYRISARLLSKSYAKDLHENGFEFAQKFKEKGKKKEKTIKEFIKKNDDNMWVHFKIKAHDGSQKSIIERVVNKEYKVGGSDIIYCDRKRKYYLNLSYTFEAEQAKVDENKILGIDVGVNTPATLAISDDKWYKEFIGDKQEIENYRNQVESRRRRLQKNAALYSGEGSTGHGRKTRLKSVDKIRDKIARFKDYKNHIWSRAIVNEAIKHGCGTIQMEDLTGIAANTNEKFLKTWSYFDLQTKIQYKAEEVGIKVVKVKPAHTSARCNNCGHIHSKENKDKWRPKEFHHEKFICQNCNHTAHADLNAAKNIAMKDIEKIIKDQLESQEKYYKNQMKYILD

SEQ ID NO: 109 - protein, 512 aa, unidentified
MITTRKFKLAIVSDNRNEAYNFIRSEIRNQNKALNVAYNHLYFEHIATEKLKHSDEEYQQHLTKYQEVASNKYQDYLKVKEKAKASKDDEKLQKRVDKAREGYNKAQEKVYKIEKEFNKKSKETYQKVVGLSKQTRIGKLVKSQFTLHYDTEDRITSTVISHFNNDMKTGLLRGDRSLRTYKNTHPLLVRARSMKFYEENGDYYIKWIKGIVFKIIISAGSKQKANIGELKSVLINILDGHYKVCDSSISLNRDLILNLSLNIPVSKENVFVPGRVVGVDLGLKIPAYVSVNDTPYIKRGIGNIDDFLKVRTQLQSQRKRLQKALKSTSGGKGRSKKLKGLDRLKAKEKNFVNTYNHFLSKNIIQFAVKNNASVIHMEELHFDKLKHKSLLRNWSYYQLQTMIEYKAEREGIEVKYVDASYTSQTCSKCGHCEEGQRVLQNAFICKNKECKGYGHKVNADFNASQNIAKSTDIIRGTEIAKTNDTAKNTKSIKGNQQSDDEVERKQLELELN

SEQ ID NO: 110 - protein, 398 aa, unidentified
MSDTSPYKHQTKNIGLLVHPEVSKATSKIFKEFSHGKFPSAVVNQTIREVKSKKKKQNAKSFKKLWCCFNNQNLKMEKVGDFYTVSFPTLEKRIGVPVVARPYQQAWLERILNGTVKQGASELYRKKKEWYIAIPITFEVEQRETKVMGVDLGLRYIAVASVGTKSLFFKGNQVAFVRRLFAARRRKLGKLKKLSAIKKSKDKESRWMKDQNHKISRQIVDFALTNGVGIIRMEDLTEINRAKSKKEAGRNLHSWAFYQLQKMIKYKAEMVGICFELVKPDYTSQTCKCGHREKANRIGIQFRCKKCGYTCHADLNGAINIAKAHSGLVAPSVLVTGTPPMWVHSNHMGRGDDTPLNLGVVQNGNGLRTLTTQESHGFSRVECQYGRIICWTWTKTVY

SEQ ID NO: 111 - protein, 481 aa, unidentified
VITVRKIKLTIMGDEETRNRQYKWIKDEQYNQYKALNIGMSYLATHLFLKMSESGLEQKTEKNIKTIEKQISKIENNITKEESKKKVNEEKLNNLINELDTLNASLSKLKEELEEISNNRSNVDDTFKRMYVDDLYNALSKVPFQHSDMKSLVSRKVKLDFNTDMKDLMSGNRSVRNYKRNHPLLVRGRDLRFRYDGSNIKIKWIQGIEFKAILGKISKTIELRHILNKVIDGEYKVCDSSLEFNKNNHLILNLAIDFPYTNKIEFIEGRVVGVDLGIAVPAYVALNDIAYVKKSIGDIDDFLRVKTQMKKRRNLQINLTSVKGGKGRSKKLKALDRLSEKESNFVRTYNHFLSKSIVKFAIDNKAGQINLELLSENALSDKIIKNWSYYQLQQFIKYKAERYGIKVKYVDPYRTSQTCSVCGHYEEGQRSKQDIFTCKNEKCKMFEKEVNADYNAARNIAISTKYIDDIKESEYYYKKSF

SEQ ID NO: 112 - protein, 428 aa, unidentified
VKLVKTMRYQIIKPLSCDWDTLGTVLRELQRDTHSVLNKTIQLCWEWQGYSSEYKAANGTYPTPKDTLGRSLEGYVYDRLKVQFPKMYTPNLSQTIQRAMLKWSADSKAIFKGEVSIPSYKKDVPLDLRKDSIHIERRGHDYILSLGLVSRAYKKELGLPECQIEVLIGTPDKTQRVILARLLTGEYTVSGSQIVWDKRNRKWFVNLAYHFEARPEQLDKTKILGVDLGVVFPVYMAVADGHFRAGIPGGEIEEFRRRVEARRRQLLRQGKYCGDGRIGHGRATRTRPLDKIADKIARFRDTINHKYSRYVVETARKLGCGVIQMEDLTGIREENLFLANWPYHDLQRKIEYKAREYGIEVRYVRPQYTSQRCSDCGYIHPDNRPEQAKFRCLACGFETNADYNAARNIATEGIEELIAAALNKASVV

SEQ ID NO: 113 - protein, 450 aa, unidentified
VEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKILGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 114 - protein, 462 aa, unidentified
VPIKALRVQIIKPFNTDYDSQPITWDELGRTLRDLRYAASKMANYVIQQNYMWEFFRQQYKQEHGSYPSVSEHKDKLYCYPRLTAMFPLAAGQMVNQIERHAKTVWSARKSEVLKLHQSVPSFKLNFPIIVHHDSYRISEVPEDGKSSTHVFLLQANLLSREAATRTRYSFLINAGEKSKQTIVERIISGEYRQGALQIVGDRKNKWYCIIPYEFKTEENNTLDPQCIMGIDLGISKAVYWAIIGSHKRGWIDGHEIEEFRRRVQARRKSIQEQGKYCGDGRIGHGRKRRLLPIEVLENRESNFKNTTNHRYSRFIIEAAIKNQCGVIQMEDLSGINERSTFLRNWTYYDLQMKIKAKAEEVGIEVRIVNPQYTSQRCSQCGHIDRDNRSNQATFVCTHCGYGGLYHCFACGKSQVEAGVCHLCGGETKIMKINADYNAARNLAICGIDQIIVQTLEGEGVR

SEQ ID NO: 115 - protein, 438 aa, unidentified
MQKVLKVQLICEHFDQEGNAVDYKTICKLLWELQKQTREIKNKSIQYCWEYSNFSSDYYKEHHEYPKEKDILSYTLGGYVNDKLKAGNDLYSANCSTTIRTACAEFKNAKSDFLRGDKSIISYKANQPLDLHNKSIRLEYQNGTFYFWLKLLNKSAVKANGFRNTEIRFKALVKDNSTRTILERCAASVYDIAASKLLYDRKKKCWYLNLVYAFEPQLAKALDPEKILGVDLGIHYPICASVFGDLKRFTIDGGEIEAFRRRVEARKISMLKQGKNCGEGRIGHGIRARNKPVYAISDKIARFRDTMNHKYSRALIDYAVKNGCGVIQMEKLTGVTADANRFMKNWTYFDLQTKIEYKAKEAGIQIIFIDPHYTSQRCSKCGYIDRENRPVQSRFSCQKCGFTENADYNASQNISIRNIEKLIEAQLQTKCESQADNS

SEQ ID NO: 116 - protein, 442 aa, unidentified
LATKCIKLAGEYAKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 117 - protein, 413 aa, unidentified
MKITKQTKIRFNFQSPNAETNRAVYEKLNGCIYLTWKAFNRAYNEFIFQHINKQKKGENYTAEENKKFRNSGYRSILDIDIHSSIKASISQKAYKQFLNDTKKGGVLSGSRTWSSYKLPSPIPFQIRMLSIFKDTDGYFMRIPYFSKDFPFNLMIERNEQKVIVDRILSKEYELNDSSIQKDKIHNKWYINLCYSFDKEPDSSIDKSIVVGVDLGIAIPVMCAINSSEYIRGSFGNRQEIDNFRARIKSKRWQILKQNNSFYDLRTGHGKSGKLKPLIPLEDRIKKFMNTYNHKLSHAIVAFALQNKAGIINFENLENLSEVKQKNMYLRDWNNADIITKTEYKAKEQGIEVHFINPAYTSQRCSKCGHIEKENRESQSEFKCTKCGYEANADFNAARNISQMNDKSSLKIDT

SEQ ID NO: 118 - protein, 417 aa, unidentified
MQRSVTLKIIRPEDEKISWEELGYLLRGLSFKVCRMCNFCMTHQLLHALKLETELLNPQGNLYCYPRLAEEYPDVPTGIICAAETRARKLFRRSAEAVLHSETSLPRFRKDSSIPVPVAGYKILQDADHNVYADVQLLSRQGAKTQKRPGRIRLVLADNWRDQSAKAALQQIAAGKVKRGVASLFRVKNDWYFQIPYVTEVVNTGEGFEPDLVMGVAFGLQNALVYAFNTSLKRGAISGEEVLAHQEKYAVRRKKIQEQYNWSGRKGHGREDALKPLRHLYETERNYRSLVNSRYAKWVVDIAVKNRCGMIHLDSANYVSSGKKILLSRWPLYDLKEKIRRKAEEKGIQVTECSIPNLRTRCSRCGKEQEPEGEKRTFVCKDCGYGKADKNRRGGFISVDYNAARNLAVYKSEEKEL

SEQ ID NO: 119 - protein, 431 aa, unidentified
MAKGTLSKVMKYELSYLDGCGDFQNMQKELWALQRQTREILNRTIQIAYHWDYTDREKFKKTGQHLDVKSETGYKRLDGYIYDELKEDVKNFASVNVNATIQKAWSKYKSSKTDVLRGDMSLPSYKSDQPLVLHGQSMKLSEGEDGVVMQATLFSNTYKKEQEYSNVRFAVRLHDTTQRTIMKNILSGDYGLGQSQIVYKRPKWFLYLTYNFSPKQHEADPDKILGVDLGETIAIYASSIGEYGGLRIEGGEVRAFAKQLEARKRALQKQATYCGEGRVGHGTKTRVADVYKAEDKIANFRNTVNHRYSKKLIDYAIQHQYGTIQMEDLTGVKKDTGFPKFLQHWTYYDLQQKIEAKAKEHGIRIIKVNPAFTSQRCSKCGNIDSGNRPSQAVFCCTKCGFKANADFNASQNISIPGIDKIIKESYGANME

SEQ ID NO: 120 - protein, 436 aa, unidentified
MNKVVRIYLISEHTDKKGDPVNYQDINKLLWELQKQTRTIKNKTIQYCWEYQNFSSDYYKEHHAYPSEKEILSYTLDGYVNDKLKNSSDLYSVNRSSTIRNAIKEFKNAKADMIKGVKSVISYKSDQPLSLHNQSVRIEYIGGQYFANIKLVNSPYAKEHDFASTMIRFKFWIRDKSAETIIQRCLSNEYKISESEMFYDRKKKQWYINLCYSFSASKNDSLDIHKILGVDLGIAYPMCASVYGDYARFTIHGGEIEKFRRTVEARKLSMLKQGKNCGEGRKGHGIKCRNKPAYNISDKIARFRDTINHKYSKALIDYALKNNCGVIQMEELTGITADADRFLKNWTYYDLQTKIKYKAEENGIKFKLIKPKYTSQRCSKCGYIDKENRKTQAHFLCLKCGFECNADYNASQNISIENIDMIIEQELKSDANIGCT

SEQ ID NO: 121 - protein, 440 aa, unidentified
MTKVVRVYLIIEQKDKNGDIVEYTKINKLLWDLQKQTRIIKNKAIQYYWEYKNFSRDYCEKNGNYPSVEEILTYKTVDGYINNRLKIDNDLCAINRSSTIKHAIAEYKNAESDIKDGTRSIINYKSDQPLDLHNTSIHIERVKEEYYLFANMVSREYAKANNFANARICFKLMLKNNKSAQTIIDRCLNGDYKISESKIIYDRKKKQWCINLAYSFSPSNVQQLDYNKILGVDLGITYPLCASVYGEYDRLAIHKGEIENFRNKVEARRYSMLRQGKNCGDGRIGHGIKCRNKPAYNIGDKIARFRDTTNHKYSRALIEYAIKNNCGTIQMENLAGITDKAERFLKNWTYYDLQTKIQYKAKECGIKIQIINPQYTSQRCSRCGYIHSDNRKTQENFLCLKCGFAANADYNASQNISIKDIDKIIKKNWKICEPDIYRIT

SEQ ID NO: 122 - protein, 468 aa, unidentified
MPTITRKIELTLCTEGLSEEQRKEQWGLLYHINDNLYKAANNISSKLYLDDHVSSMVRMKHAEYLSLLKELARAEKQKTPDADAIAELRKKVAAAETEMTDQEHAICKYATEMSTETLAYKFATEIETHVFGQILTCLKQAAQSNFNSDAKDVKRGERAIRNYKKGMPIPFPWNRSLKIEADGGNFYLRWFNGLRFLLNFGKDRSNNRLIVKRCMKMDADYEGEYKLCNSSIQIAKREGKTKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKAHAATIHMEDLSGFGKDNDGNADEKKEFVLRNWSYYELQNMIAYKAAKYGIKVEKIRPAYTSKTCSWCGQQGFREGVTFICENPACKQCGEKVHADYNAARNIANSKAIIKKNE

SEQ ID NO: 123 - protein, 468 aa, unidentified
MPTITRKIELTLCTEGLSEEQRKEQWGLLYHINDNLYKAANNISSKLYLDDHVSSMVRMKHAEYLSLLKELARAEKQKTPDADAIAELRKNVAAAEKEMTDQERAICKYATEMSTQSLSYRFATELETNIFAKILDCLKQGVFATFNSDARDVKRGERAIRNYKKGMPIPFAWNDSLRIEKDNKDFYLRWYNGLRFLFNFGKDRSNNRLIVERCLKMDADYDGEYKLCNSSIQIAKREGKVKLFLLLVVSIPQEHVELNKKIVVGVDLGINVPAYVATNITEERKAIGDREHFLNTRMAFQRRYKSLQRLKGTAGGKGRTKKLEPLERLRKAEHNWVHTQNHLFSREVVDFAVKTHAATIHMEDLSGFGKDNDGNADERKEFVLRNWSYYELQNMIAYKAAKYGIKVEKIRPAYTSKTCSCCGQQGFREGVTFICENPECKQYGEKVHADYNAARNIANSKEIIKKNE

SEQ ID NO: 124 - protein, 445 aa, unidentified
MPTKCIKVALEFIKKDNNISEKQINKELKDIQFKTHLACNRAMTYMYSNDSETIIQRDIGIPKEDDKFLYGKSFGSWIENRMNEIMDGVLSNNVAQTRAFVINTYNQDKKNGLFKGNVTLSQFKRDMPIILHNKAFKIIETSKGLGVEIGLFNLKKQKELGIKRIVFLTPRLGESEKSIFKRLMDKSYKLGTAQISYNQRKRKWMIAISYTFNKEKDTIYLDNNKVMGIDLGIVNVVAMSIYDNAKEQYIKMSWKDRLISGTELISYRQKLESRRKNLAIASKWASSNRCGHGYKNRMRAVNKQGDKFNRFKDTFNHKISRYIVNMALKYHAGIIQMEDLSGFSNEQSESLLKNWSYYDLQEKIIYKAKENGIEVILIDPKYTSKRCSECGNIDDKNRDCKKDQEHFKCTACNYTDNADINASKNIAILKIDSIIENYLKNKNKG

SEQ ID NO: 125 - protein, 439 aa, unidentified
MIKIVKTMKYEIKYDKELYNLLSDIQHAVWLIKNRATTAAYDWQQFSFAYNERFGEYPKEKDVIGKTLAPDVYGFLKEIGSFVSSSIVDSAVQEAITKFKNDKVKILKGEQSIQIYRRNGSFPIRASQLKGLTKLDNKTYNAKLSLLSNEGAKERDCKGQFLVTLVTGNGAYEILDRVINGEYKMCDSRIYKRKNKFYLLLTYKFEKETDKVLDENRIMGVDIGVAVPAVLAINEDKFYRQYVGDAKEVSDFVAQINDRKKRLQRSRKWAGEGSRGSGRKKLMKPVDAISNKIHNYRETKNHTWSRFIVNEALKNECGTIQIEDLSGISKDNAFLKEWTFYSLQQKIIDKAKEHGIQVVKVKPNYTSQRCNKCGFIHKDVNKEIWRPTQSSFKCLNCGHETNADLNAARNIAMKDIEKIIVDQLNVQEQHQKHAEKYLV

SEQ ID NO: 126 - protein, 469 aa, unidentified
MGAKIVKLQLIYKSDEVMPYKDYCKELFALMREMSIIKNKIATYLYLDKRFSVALPIEKDTKVKSASGLASAFASELLVNYKTGEVEVLKNHSSNRSAAGQDVQAKFKKFLKESMGAYAVNPPSFKDNGTLCLHDRCIQIYYDEDNKDYGAKLFLLSHSYAKELGIKSSNGFDYKLLVGDDSSRAIIERIIAGEYKISASNLKWDKRKKKWYLLLCYSFTEKRTTSYDPKEYENNVMGINFGVICPMYMSFNHCKNRYNIEANEIEAFRKQVEARRIALRRQRKYCGDGSIGHGKNKRNSPAMKIDDKIARFRDTCNHKYARYAVDMAIKHQCGIIVIEDLTSIAEKEERLFLKTWSYYDLQNKIEYKAKEAGIKVIKINPQYVSRRCSKCGYISFEKEENIKAPDYRKFHCVECGFQSHVDYNASQNNATIGIEAIIAEQIKEHNSENVKNQPAAKAAKSKSKIKETA

SEQ ID NO: 127 - protein, 476 aa, unidentified
MITSRKIKLAIVSDNKDTAYSFIREETRNQNRALNVAYTHLYFEYVAQEKLKQSDKEYQQHLEKYKNAAAKKYQEFLTIKEKSKSDENLQPKMDKVRETYNKAMEKVYKIEKDYSKKAREIYQQSVGLAKQTRLGKLIKSEFDLHYDTVDRIGSNAMSDFSNDRKSGILSGERSLRNYKKTNPLMVRARSMKLYEEDNNFYIKWINDIVFKIIISAGSKQRMNIAELKSVFIKLLSGQCKMCDSSISLDKGLILNLSIDMPITKENVFIPNRVLGVDLGLKIPAYVSLNDTHYIKGAIGNIDDFLKVRTGLQSQRRRLQKSLQSTGGGKGRRKKLQALERLKTKEKNFVNTYNHFLSKNIVQFAVKNNAGAIHMEELKFDKMKNKSLLRNWSYYQLQTMVEYKAKSEGIEVYYVDASYTSQTCSKCGNLEEGQREARDTFVCKKCGYNVHADYNASQNIAKSTKAINKTIEITNIV

SEQ ID NO: 128 - protein, 437 aa, unidentified
MTKVTKVYLISEQIDKDGNKIDFKKISELLWNLQRQTRDIKNKCVQLCWEWLNFSSDYYKKSEEYPKEKDTLGYTLSGFVYDRIKNGSDLYSSNLSTSSRDTCTAFSNYKKEMLNGERSVLSFKANQPLDIHNKAIKLSYENGNFFVALKMLNRAGKEKYGINDDLRFRMQVRDKSVRTILERLMNDEYKVSASKLMYDKKKKLWKLNLCYSFDNHVISTLDPEKIMGVDLGVVYPIMASVNGDYARFSIKGGEIEAFRNRVEARRRSLLNQSRYCGDGRIGHGRKKRTEPAAQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPEVPTE

SEQ ID NO: 129 - protein, 439 aa, unidentified
MIKVVKTMKYEIMYDKELYDLLSEIQYAIWLIKNRATPAVYDWQQFSFSYNERFGEYPKEKDVLGKTLAPDIYGFLKELGSFVSSQIIDTAVQEAIKKFKNDKMKILKGEQSIQIYRRNGSFPIRASQLKDLTKINNKTYEAKLSLLSNAGAKERDCKGQFLVKLVTGNGAYEILDRIINGEYKMSDSRIYKKKNKFYLLLTYKFEKETEKVLDENRIMGVDIGVAVPAVLAINEDKYYRQYVGDAKEVSDFVAQINDRKKRLQRSRKWAGEGSRGNGRKKLMKPVDAISNKIHNYRETKNHTWSRFIVNEALKNECGTIQIEDLSGITTDNAFLKDWTFFSLQQKITDKAKEHGIKVVKVKPNYTSQRCNKCGFIHKNVGKEIWRPTQSSFKCLNCNHVTNADLNAARNIAMKDIEKIIEVQLKAQEQNAKHEEKYLV

SEQ ID NO: 130 - protein, 431 aa, unidentified
MPKQISKVMKYELKYLGEEDFYEMQKMLWSLQEDTREILNKTIQIFFHWDYTNKESLETTGKALNLVEETGYKDISGYVYDKLKSRYPDMSRGNLSATIRAASKKYRSSKVDILKGTMSIPSYKKDQPIILRPDGIRLHEREMIYTVELSLFSGDFKKKKAWKSNVLFQIKACDKAQKAIMQRLLSREYKLGESKLVYKKKKWFIYITYSFKKTDAKLDKNKILGVDLGVTYAIYACSIGEYGSFSIKGEEALEYAKRLEARTISKQKQARYCGEGRIGHGIKTRLSTVYSTRNKLANHRDTLNHRYSKAVVDYAVKNRYGTIQMENLSCIKKNTGFPKRLQHWTYYDLQSKIEYKAAEQGIQVIKINPKFTSLRCSQCGCIHKDNRKTQESFQCVECGYKDNADHNAALNISIPQIDLIIKEEMTSAKEK

SEQ ID NO: 131 - protein, 488 aa, unidentified
MAEHTVITRKIEVHLHRSGDSEEAKELLREKYHMWDTINDNLYKAANLIISHCFFNDAYEDRLRIQSPRFKQIQDSLRNAKRNKLGESEIKELKSEREQLFDEFKKQRYTFLRGGVAEGPNPEQNSTYRVASDNFLDTIPSDILTCLNKNITTTYKSYRKEIEFGNRTIPNFKKGIPVPFPIKKDKKLRISKRDDGSIFIKFPGRLEWDLDFGRDRSNNREIVERVLNGMYDVGDSSIQETRSGKRFLLLVVKIPKVKNVSLDSNRVVGIDLGINTPLFAALNDNDYERVSIGSRDQFLNVRNRMNAQKREMQKNLRSSTTGGRGRRHKLQALERLEGKERNWVHLQNHIFSKGAIEFAIKNNAGVIQMERLTGFGRNANDEVENDRKFLLRNWSYFELQQLIEYKADAAGIEVRYIDPYHTSQTCSFCGHYEAGQRVDQAHFICKNPECEKGKGKKNDDGTYAGINADWNAARNIARSNKIVDRKKK

SEQ ID NO: 132 - protein, 424 aa, unidentified
LILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 133 - protein, 468 aa, unidentified
MKTKKHTKVMRYEIIKPLDSTWEVFGQVLRQVQYETRHALNKSIQLSWEWQGFSAEYKRQSDHYPNLKEITNYTNLQGYAYNTLKHEFPSLYRGNFSQTVKRATDKWNSDRGEILRGERSIPNFKKDIPIDVVAAAFKEGKEQNLRIRKILSSEEYNNETGKRIQGYTVKVNLISDSYKKELGRNSTAFEMLIKVGDNTQKAIIERLIEGKYNIAASQILYQKGKGSGKKGKWFLNLSYSFEKEDVTLNPDLIMGIDMGIVHPIYIAFSDSYARYHINKGEINRFRNQIEKRKKELLHQGTYCGEGRKGHGIQARIKPIEVISDKIANFRKLCNHRYSKFVVDIAVKHGCGTIQMEDLQGISKDDVFYKNWSYFDLQEKIAYKAKESGIKVIKVNPRYTSQRCSQCGNIDSENRVNQADFLCTACGFKALADYNAARNISTRNIEKIIDKAVGKEIIDVVDLEYGNIS

SEQ ID NO: 134 - protein, 429 aa, unidentified
MVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 135 - protein, 424 aa, unidentified
MPTRVMKYQIVKPMNCDWKLLNRHLLDLQQEAHQILNKTIQLCWEWQGFCSEYKAKYDIYPVDKDIFHLTLAGYVNRMLRLQFPKSNSNNLGTSRIKAINRWHTDLKDVKSGQKSIASFKADVPIDLHYLSIKLHKEKDCYYADLSLISNIYKKELGRESGKLLVLLKSGDEVSRDILRNCLDGIYKIRASDISHKKNKWFLNLHYNYEETNKSLDKDRILGIDMGIAYPVYMSVYNTQITSYIEGGEIERFHKQVEKRSAEFQRQGKYCGNGRIGHGVKTRLKPLSFATDKIANFRETTNHKYSKYIVDFAVKNNCGNIQMEDLTGIKNERIFLRNWTYFDLQKKIRYKAEENGINVLLIKPHYTSQRCSQCGDIDKKSRITQENFTCISCGYQTNADHNASINISTPNIENIINDFMSLKID

SEQ ID NO: 136 - protein, 439 aa, unidentified
MFMTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 137 - protein, 425 aa, unidentified
MNKVMRYQIIKPIDIDWKTFGDILNKMRQEVRFTKNKTIALYNDWLTYCFQYKNEHNEYPKLINYCGYKVFSGYAYDKFKTDVVFSNTANYTTSVREACSAYDTHKTDILKGNCSIPSMGANQPIDLHNKSLSVDVNESGDYIATISLLSNRGKKEFGLKSGQIKVVLKAGDKSSKDILQRCVSKEYKICGSKIIYKGKKTFINLCYGFEPEASDLDKSKVMGIDLGVSVPAYMAFNFDKYKRDSIKDNRIMTTKWMMDQQLSIAKQSCKYLSDGNSGHGRKKKMICYDKYSNKSRNLSQTINHGWSKYIVDVAFRNGCGTIQMEDLSGVTSEKDKFLKNWTFYDLQQKIEYKAKEKGIDVVKINPRYTSQRCSECGCICKRNRPDQKTFKCISCGNSSNADFNAAKNIATIGIEDIIANTEVIE

SEQ ID NO: 138 - protein, 450 aa, unidentified
MSIAVKVMKYQIVCPVNVEWKVFETYLRTLSYQSRTIGNRTIQKIWEFDNLSLNHFKETGEYPSAQHLYGCTQKTISGYIYDQLKEEYQDINKANMSTTIQKTLKNWNSRKKEIWRGEMSIPSFRNNLPIDIHGNSIQIIKEKSGDYIASVSLFSSKFIKENDLPNGKILVKLSTRKQNSMKVILDKIVNSIYAKGACMLHKHKNKWYLSITYKATIKEEHKFDEELIMGIDMGKINVLYFAYNKGLVRGAISGEEIEAFRKKIEYRRISLLRQGKYCSENRIGKGRKKRIKPIDVLNDKVAKFRNATNHKYANYIVQQCLKYNCGTIQLEDLQGISKEQTFLKNWTYFDLQEKIKNQANQYGIKVVKIDPSYTSQRCSECGYIHKKNRQDQSTFECQQCSFKIHADYNAAKNISVYNIEKVIQKQLELQEKLNLTKYKEQYIEQMENIN

SEQ ID NO: 139 - protein, 453 aa, unidentified
MTYLSIAVKVMKYQIVCPVNVEWKVFETYLRTLSYQSRTIGNRTIQKIWEFDNLSLNHFKETGEYPSAQHLYGCTQKTISGYIYDQLKEEYQDINKANMSTTIQKTLKNWNSRKKEIWRGEMSIPSFRNNLPIDIHGNSIQIIKEKSGDYIASVSLFSSKFIKENDLPNGKILVKLSTRKQNSMKVILDKIVNSIYAKGACMLHKHKNKWYLSITYKATIKEEHKFDEELIMGIDMGKINVLYFAYNKGLVRGAISGEEIEAFRKKIEYRRISLLRQGKYCSENRIGKGRKKRIKPIDVLNDKVAKFRNATNHKYANYIVQQCLKYNCGTIQLEDLQGISKEQTFLKNWTYFDLQEKIKNQANQYGIKVVKIDPSYTSQRCSECGYIHKKNRQDQSTFECQQCSFKIHADYNAAKNISVYNIEKVIQKQLELQEKLNLTKYKEQYIEQMENIN

SEQ ID NO: 140 - protein, 436 aa, unidentified
MNKVVRLALICEHSDKDGNPVDYSDVYKLLWQLQAQTREIKNKTIQYCWEYSNFSSDYYKENHEYPKEKDVLHYALDGFVNDKFKVGNDLYSSNCSYMTRKVCAEFKKSKSDFLKGTRSIISYKSNQPLDLHNKSIRIEYKDNDFFAFLKLLKRPAFNRLGYKNSEIGFKVIVRDKSTRTILERCVDQIYGISASKLIYNKKKKQWFLNLVYAFEPDNANNLDPSKILGVDLGIHYPICASVYGDLQRFTIHGGEIEEFRRRVESRKLSLLKQGKNCGDGRIGHGGKTRNKPVYSIEDRIARFRDTVNHKYSRALIDYAVKKECGTIQMEDLSGITAESDRFLKNWSYYDLQSKIEYKAKEKGIKIVYIDPKYSSQRCSKCGHIDKENRKTQSSFVCLKCGFEENADYNASQNIGIKDIDKIIENDLSSKCETDVN

SEQ ID NO: 141 - protein, 433 aa, unidentified
MGKGVLAKVMKYELRYLDGCGDFSNMQEQVWALQRQTREILNRSIQIAFQWDCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 142 - protein, 459 aa, unidentified
MRIKIIAKKKGINMNKIMKYQILKPTNISWEDFGNILYNLRSEVRKIKNRTIALYHEWTNYTLECHDRTGEWPKPKEVYNYGTMGGYIYDRLKGEVKYSNSVNFNSSVRDAMSKYDTHKKDILAGKASVPSMGDGQPIDIYNKNIVLHHLDNEKKDYAATLSLLNNGAKAELGLPSGRVDVILTIKNETQTAILDRCLSGEYRICGSQLIYEAAGKEKKGKKDKPKVWLYLCYGFEPEAPELDDSRIMGIDLGMKLPAVMAFNFNDKKYEVIDDNRILDRKIRLDKMLSMSKHQCQWRCDGNSGHGRKKKVGVYENYANKSHNMSMTINHQWSKYIVDTAVKNKCGVIQMEDLSGIKASRQNFLGNWTYYDLQQKITYKAEEKGIKVIKVDPQYTSQMCPICGYINKRNRATQADFECLECGHIANADYNAARNIATPDIANIIKNRLTQQKKEGKSID

SEQ ID NO: 143 - protein, 434 aa, unidentified
MNENMCALTKIMKYELRYLDGFPDFSAMQNAVWPLQRQTREILNRTIQEAYHWDYFSATKKKETGEYPDLLKETGYKRLDGYIYHVLAPDYPDFSSSGVNATIQKAWKKYKSSRADVWKGEMSLPSYKSDQPIVLHAKQIKLSGDNRAAAVTLSLFSNKFKKEHAISGNVQFAITLHDNTQRTIYQKLRNGEYKLSESQLVYDKKKWFLYLAYSFNPAEHALDPEKILGVDMGEKFALYASSFGEYGHFKIEGSEVTEYAKALERRRRSLQQQARYCGEGRIGHGTKTRVGAVYREEDRIANFRSTINHRYSKALIEYAVKNGYGTIQMENLTGIKENLQFPRRLQHWTYYDLQSKIEAKAKEHGIAVVKVNPKHTSQRCSRCGHIAAENRPKQEVFQCVKCGYACNADFNASQNISIKDIEKLIQETIGANPK

SEQ ID NO: 144 - protein, 449 aa, unidentified
MQQVVRFEIIKPIDNDWKVLGKVLRDLQYESRQVLNKTIQYNWEFSNCAIGFKEKFGINPKISDISKYKGSHALQNYIYNELKNVYTKISTSNLTCLIEKATSQWKTFQKDVYKGERNPPEYKKSNTPIIIHKQCINILKENGKYYADIALVSKKHLEEYKLNSCRFTLLLNTKNNGNKAILDMILNGELDYSQSQIIEKNKKWFLYLSYKVAEKVLPDDYNRIMGIDLGVNKAVVIAIHGTEIRDYIPGGEIIAYKNKMYNMVWHRQKQARYCGEGRIGHGRKTRLKNIYKIKEKIANFSDLTNHRYSKYIVELAKKHKCGVIQMEDLSGLSTNNKFLKKYPIYDLQQKIIYKAEREGIKVVKIKPNYTSQMCSNCHFISEENRPKDERGWEYFKCVNCGLEIDADLNAARNMANSQIENIIKEQLKIQGIKNKNNGTDEESRKAVNS

SEQ ID NO: 145 - protein, 422 aa, unidentified
MKYELTYLDGCGDFHNMQNELWALQRQTRELLNRTVQIAYHWDYKGSEHYRETGQPMDVYTETGYKRLDGYIYSCLKDDADSFSAANVNATIQKAWKKYKSSKSDIMQGKMSLPSYKRDQPLILYARNVKISNEQRNPAVQFTLFSKSYKEEKGYSDVQFVMHLNDATQKAIFQKIASKEYGLGECQLVYSKPKWFLLLTYNFTPEDRRLDPDRILGVDMGETFALYASSKDEYGSLKIEGGEVREFAKRMEARKRSMQRQSAHCGEGRIGHGTKTRVSDVYKAEDKIANFRSTVNHRYSKKLIEYAVQHQYGTIQMEDLSGIRESTGFPKFLRHWTFYDLQQKIEAKAREQGIRVVKVDPSYTSQRCSKCGNIDKANRPTQAQFCCSKCGYKTNADFNASQNLSIRGIDRIIKETLGANPK

SEQ ID NO: 146 - protein, 460 aa, unidentified
MSIKAIRLEIVKPYNESDAENVVTWNELGEALREVRYACSKAQNYVITERYLWERFKIDYKNQNGVYPDPKAFKERTNLYSQLTKMFPHVASNIINQTDRLATKKWSNEKKDVLSLKRSLTSFKLDVPIPVHYEGYKIFKVNDSEREKYIIRVTLLSKKSDKQMAYNLLLKVKDNSSKTILDRLISEELDSKIIQIVSTNKKKWFCIIPYDFTERNTEVVDGRIMGIDLGIAKAVYYAFNDSYKRGSIDGGEIERFRKSVRARRITIQNQGKYCGDGRIGHGVKRRLKPVEILREKEKNFRNLINHRYSRHLVEIAVKNKCAVIQMEDLTGITKDNAFLKDWPYYDLQQKIKEKAAEYGILFKTINPYKTSQRCSRCGYIDRENRPEQSVFVCQNCGYGSLYLCENCNKEQNHAGICDTCGGKTNLITVNADYNAAKNIATENIEEIIKKEMGKEYNPPK

SEQ ID NO: 147 - protein, 431 aa, unidentified
MSVKIASFSLIYKEDYNELNYDDLYNVLIQLQNESAKIANRAVQIYWENSNYCRQVKSMTGRFPTKDELLKHYGCSEQNYVYRLLTKEFYKNSTGNISTIIQFVGKRYKRYYPDYLSGKRSIESYKSSFPIYLCKNNIRVFKEDGKYYIKLGLISALYKSELSIHSGSVIFELGFGKSSSYKTLLDNIINQLFSLTSSKIIFLKKKIIIQLGYNTNSNIILDNSSCRVMGIDIGVAKPFVYAFNDITDFNFVDGNEIKNFQKQMLSRRQSLGRQTKNCADSKIGHGIHKRIEGIEKLGQKESNFRNRINHQYSRMIVDAAIKYKCTTIQIEDLSGISSENKFLKSWPYYDLQSKIEYKAKECGIDVVKINPKFTSQRCSKCGYISKENRKTQAGFKCKNCGFEENADLNAARNIAIPKIDSIIQESLRATT

SEQ ID NO: 148 - protein, 414 aa, unidentified
MVKTTKIYLCMDSKDTYKLLWKLQDNTRMLKNKAVQMLWEWNNFSQEYKKEHEEYPKPKDILNYTVGGYMYDKLKSESLLASSNLSSTLQLVEKQFKTNAKDFLRGDKSIICFKKDQPLDIHNKSIRLSHENGTFYADIVLMNKATANEHNGGGCALPFRLWIKDKSTRTIVERCYDGVYSVSGSKIKYDEKKKMWYINLSYGFDKYSTAELDKEQVLGVYLAQETPFTASVIGDHDRLQVSVNEIEHYRNAVESRRRSILKQSAVCGEGRIGHGYKKRVEPMEKLSHKVADTRDTINHKYSKAIVEYAVKKGCGTIRMEKLTGISETDRYLRNWPYFDLQTKIEYKAKERGIDIVYVDSADIQRCCRCGHVNEEETTGSRFKCTECGFEHDVAYNASQNLSAGGKDIKVNGRK

SEQ ID NO: 149 - protein, 469 aa, unidentified
MRLYSIKCNKEKKLNEVKRMESEKEKYVVQSYGIKLIKPVGVDEGDWDFAGKVLRDLDYACFRVKNKAATRTYMNVIEKLEYENIHGKGTYDKYFKTRYGKTFSAYNSECAREDKELNKGDLYREYFNLMAREGEKVVKYNMKKILNGNASNITFKRNQPVPITSRMIFITKESGKYYAELTLLSPEKAKELGRKGKNGTRIKFLLSSKGQEKVILDRLTSGEYDLRDSHIHVKKKGNKLTNYLIIAYRHKVKDDNDLIPNKVLGVDLGVSKAAYMAVSESPVSEYINGGEIEQFRNGVEARRNGMRNQLKYHSSNRSGHGRATKLKPLEKLREKVSNFRKLTNHRYAKFIVDTALKNNCSIIQMEELKGISKNDTFLKRWSYFDLQEKIENKAIAVGIEVKKVSPKFTSQRCNKCGYIDKESRKSQEKFECVNCGHKTNADLNAARNLSMLNVEKEIKAQCKAQKIKY

SEQ ID NO: 150 - protein, 421 aa, unidentified
MDKTLRDIQYVTCKASNKAMQMYYMWEYEKLEYKNKYGEYPNEKEMYGKTYRNVVEAEVKAIMNTINTSNTGQTNAFVMKKWNTDKKDIMNYRKSVASYKLNMPIYLKNSSYKILQGESGYEIDCAIFNKSQGLKHLTFTIDKLDGTKKATLNKLIESKNLSELINGAYKQGAMQIVKKKNKWCMLISFGFEAVKRELDTNRIVGVDVGIVNALTFQVWDNTTQKWDRLSWRDCLLDGKELIHYRQKIMARRIALLKNSKLSDENKGKAGHGRVKRIGPINTISDKVKAFRDTLNHKYSKYVIDFAIKNNCGCIQMEDLSGYSESVSETFLKNWSYFDLQSKIKYKAEENGLAINFIKPYHTSLRCSLCGNIQKENRDCRNNQSRFKCTVCGYEENADINAAKNISLPNIEQLIKEQLKIK

SEQ ID NO: 151 - protein, 450 aa, unidentified
MEGEKEKYVVQSYGIKIIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 152 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKILGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 153 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECMNCGHKTNADLNAARNLSMLDIEKVIKAQCKAQKIKH

SEQ ID NO: 154 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 155 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVEKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 156 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKTQCKAQKIKH

SEQ ID NO: 157 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 158 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYANFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 159 - protein, 450 aa, unidentified
MEGEKEKYVVQSYGIKLIKPVGVDEGDWDFAGKVLRDLDYICFRVKNKTATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 160 - protein, 442 aa, unidentified
MATKCIKLAGEYVKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 161 - protein, 442 aa, unidentified
MATKCIKLAGEYAKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 162 - protein, 441 aa, unidentified
MATKCIKLAGEYAKENSLEKDKFFKELRDIQYKTWLACNRAITYFYSNDMQNLIQKDVGIPKEDDKTIFGKSFVAWVENRMNEIMEGISSANVAQTRQFVNNRYSQDKKNGLLKGSVSLSQFKRDLPIIIHNKAYNVIETSKGLGVEISFFNKEKQKELNVKRIKLLFPKLDNSSKQILIRLMDKTYKQGSIQVTYNRRKKKWMFAISYTFENKLEKVLDDNLVMGIDLGITKVATMSIYDIEKHEYKKMYFKEQTIDGTELIHYRQRIEARRKSLSIASKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQSKFECVVCGHKENADINASKNIAIPYIDKIIKEYIKDTK

SEQ ID NO: 163 - protein, 442 aa, unidentified
MATKCIKLAGEYAKENSLEKDKFFKELRDIQYKTWLACNRAITYFYSNDMQNLIQKDVGIPKEDDKLLYGKAFKSWVANRISEILGDGISSYATDCITQFVSNRYKNDKKAGLLKGNVALSQFKRDIPVMLRERAYSIIDTPKGLGVEISFFSKTKQQELGIKRILFTFPKIDGSSKSILTRIMDKTYKQGSIQITYNKRKKKWMFAISYTFENKLEKVLDDNLVMGIDLGITKVATMSIYDIEKHEYKKMYFKNQTIDGTELIHYRQRIEARRKSLSIASKWASDNATGHGYKRRMKKVNNIGDKYNRFKDTYNHKVSRYIVDLAYKYGVKTIQMEELRGFSEHQSVSLLKNWSYYDLQNKIKYKAEEKGINTIFINPQYTSKRCSKCGNIHEENRDCKNNQAKFECVICGHKENADINASKNIAIPYIDEIIKEYIKDTI

SEQ ID NO: 164 - protein, 450 aa, unidentified
MKINKCIKVTLIKCLNYDYKEIKQIIRDFNYTACKASNKAMRMWLFHTQDMIDKRNEDKLFNQIQYEKDTYGKSYRNVIEGEMKKLMPLANTSNVGTLHQQLVQNDWGRLKKDILSCKANIPNYKINTPYFIKNDNFKLRNHNGYFVGIAFFNKEGLQQYGYKIGHKFEFQIDKLDGNKKATINKIINGEYKQGSAQISISKKGKIELIISYGFEKEEIPVLDNNRILGIDLGITNVATMSAYDSIKDEYDYFSWKTNVISGKELIAFRQKYYNLRRDLSIASKTAGKGRCGHGYKTKMKPVDKIRNRIANFADTYNHKISKYIVEFAVKNRCGVIQMEDLSGATSEVHNKMLKDWSYYDLQQKIEYKAREQGIEVKKVNPKYTSKRCNKCGCIHEDNRDCKNNQAKFECKVCGHSENADINASRNIAIPEIDKIIDKTEILHSENRQAS

SEQ ID NO: 165 - protein, 436 aa, unidentified
MKFNKCIKVTLIRCLNYDYRETKQIVRDFQYKYSKAYNMATNYLYLWDTNSMNLKNLYDTKIVDKELLGKSKCAWIENRMNEIIQDSNTSNVAQARQDVVNKYNKCKKDGLFKGKVSLPTYKLDSKVIIHNNSYKLRNHNGYFIDIGLLNKDKQKELNVGRFEFQIDKLDGNKKATINKIINGEYKQGSAQISISKKGKIELIISYSFDKEEVPVLDKNRVLGIDLGITNVATMSVYDSIKDEYDYFSWKTNVISGKELIAFRQKYYNLRRDISIASKVAGKGRCGHGYKTKMKPVDKIRNKIANFSDTYNHKISKYIVEFAVKNNCGVIQMEDLSGTTADTNNKMLKDWSYYGLQQKIEYKAKEQGIEVKKVNPKYTSKRCNKCGCIHEDNRDCKNSQAKFECKVCGHKDNADINASKNIAIPDIDIIIKETEIL

SEQ ID NO: 166 - protein, 493 aa, unidentified
MNSIKINKCIKVTLKKCLNLDIKEVKNIIKDMNYLACKASNKAIKMWQLHTEEMMERKFEDKNFNISQYEKDTYKKSYRNVIEGKMKDIMDICNTSNVGTLHQQLVQNDWGRLKKDVLNYRANVPTYKLDTPYFIKNHNYKLYNNGGWFVDIAFFNKQGLIQYGYKVGHKFEFEIDKIDNNKKSTITKIINGEYKQGSAQISLSKKGKIELIISFSFEKELKNNLDKNRILGIDLGIVNTATMSIWDNNKQAWDWVDYKENRIDGKELIKFRQKLFSMGMSNSEIEKEIFKANSKIKENQLRKRELGVIDGLELAKYRDTVYKKRREMSIASKYAGKGRSGHGRKTKMKPVDKIRNKVYNFADTYNHKYSKYIVDFAVKHNCGIIQMEDLSNATANKKEKFLKDWSYFDLQTKIEYKAKEYGIEVIKINPKYTSKRCSKCGGIHIDNRDCQHNQANFECKICGHKENADINASRNISIPYIDKIIAKTKVQAD

SEQ ID NO: 167 - protein, 490 aa, unidentified
MVTKVVECKIIGFDSLLFEKEDVTENAKKDNKKAKEAWKRNIEMIDQAVRDFVAAKNRAITYLFTEYIKVRDAMATSGESSFSSVWKKMHDEKGRSKAMYHYLVKALPGYLTGNVSEGSRRLEKRFDQAVKDGLLYGRVSLPSFRDNAALDFRGDSVKIKQEIIKGEEKLYVELNLKRGIKLHCNIGFSGNKGAKAVAERIGKGEYKAGTASIVKRKSRYFIQICYSFDSDIYMRSNGMVGLAEGLDPKRTLGVDLGLARPVAMQPYDSDSRKYLSGGKNDEFVNTERYTNRFTISGDRIAAFRKQVEKKKSVLQRDLRDVSPGHTGKGRIKRIKPVEKFKDRIANFRKTTNFVYAKRIVDTAIKYHCGTIRIEDLTIDVSSRKSERFLKTDWTYYDLQQKIESRAANFGIVVQKVAPQYTSQKCSKCGHVSADNRKDQSHFKCVSCGFEMNADLNAARNIAALSPNDKIKKEKNKEAKDGQMQLEFILN

SEQ ID NO: 168 - protein, 483 aa, unidentified
MVTKVVKCKVVGFDSLPFEKKDAAGNAKINNKKAKEAWNRNIEMIDQAVKDFVAAKNRGITYLFTESIKIQDAMDASGESSFYDAWKKMHGGTQLRTTMDHFLVKELPGYLAENVCESSGRLVTRFNQAKKDGLFKGCVSLPSFRDNAALDFRGSSVKIKQETINGEEKLYVELRFKRGIKLHCNIDFSGNNGAKAIAKRVCNGEYKSGTASIVKRKGKYFIYICYSFEPETYMRLNGMDGLAEKLDTKRILGVDLGLAKPVAMQPYDSISQKYLSGGKNDEFVNTEEYTNRFTISGDQITAFRKQVEKRKRALQRDLRDVSPGHTGKGRIKRIKPVETFKDRIANFRNTTNFIYAKRIVDTAIKYHCGIIRIEALTIDVSSRKSERFLKTDWTYYDLQQKIESRAANFGIVVQKVKPNYTSQKCSKCGHVSADNRKDQSHFKCVSCGFEMNADLNAARNIATLSPTDKINKKKKREVKDDQV

SEQ ID NO: 169 - protein, 419 aa, unidentified
MTKTKMITKATRFQIIKPLDTGWDELGQVLRDLSYHTTKLCNMAVQLYWQHHNYRLAHKEETGKYPAAAEDKERYGCSFRNHVYRKMREMYPDMASSNTSQTNQFAMSRWQNDVREIMRLQKSIPSFRLGTPAQVANANYTLSVEEQDNGRSSFIATITLLSLAAGKQTRYSILLDGGDRSKKAIFRRIMEGEYKQGAMQITYNKRKKKWFCIVSFSFIPEKQNRELDPDRAMGLMFGTGNYTVFAAYSVGQKRYTLPAGEVIATEKKIHAIAERRKEIQRHAGYRGHGIKRKLQGTEVLAGQASAIRDTLNHKYSRRVVDVAMANRCGTIKILDKLVTPGASVSELVNYPWAGVVEKIKYKAEEKGIAVKVVSLDGTRCHKCDAEIEIDSDNESPLTCPSCGAKIDKDYNTAKRIAKS

SEQ ID NO: 170 - protein, 411 aa, unidentified
MITNTCKLEVSFIDTKFSDLFAMINATTQVKRYATDRITEWKSFSSRYHEENGEYPKLEDIYGFKSIEGLIYHELSKKKTGLSSRSLIATIHSCFLSYKQASKKHSHPNWSDSQPIIMQGKYLNLHHESGNYIVDYPLFSKQYRVEHEITHILRMRLNTKENHHKNILDRILSGEYHLCQSQLKYVKKQNKSHWYFLLAYSHKSEGNPNIDPTRVLGVFMSENVALYASSKRLPMVFKIDGGEIPTFASRQEAIIRSKQRQAACCGDGRIGHGYKTRTKSIYPNKQTLAHFRETINSRYAKALIDFAKTNGFGEVRLEDLKGIKADREFPRFLIHWTYFDLQEKIKNAARKCGIKVTLVSSETIGITCSSCGHIDPNNRDRGVKNRFVCTKCNIVKQTDWNASQVLSSLEI

SEQ ID NO: 171 - protein, 439 aa, unidentified
MLTKCIKIPIEYSKDNILKSEEFYKELRDIQYKSWRACNRALTYFYMHDMENIMLKESGKDIKSDKELYGKTYGSWIENRMNEIMKGVLSNNVAQTRQYISNRYGQDRKNGLLKGNVSLSEIKRNMPIIIHAKAFKIIDIIKGLGVEVSFFNLNKQRELGVKKIKFLFPKIHSSEKSILKRLVDKSYKQGIAQLSFNERKKKWLLTISYSFEKRIEELDEDLVMGIDLGISKITTFSILNAKTKEYIQMNFKDKFVDGKELIHYRQKLEARRRELSIASKYTSKNNLGHGYKTKMQSVNMAGDKYNRFKETYNHKVSRFIIEIALRYRVKNIQMEDLSGFSEHQTESLLGNWSYYDLQNKIAYKAEELGINIIFIDPKYTSQRCNKCGNINSKNRNCKENQEKFECIRCGYKENADINASMNIAIPNIENIINNTIKIK

SEQ ID NO: 172 - protein, 475 aa, unidentified
MKEKYFVKVAKFEILKPANGMTWKEFRQLLMSVRYRVFRLANLCVSENFLQFHLWRKKEIDKIPTLKISELNRQLREMLLEEKKTSDAEQTRICKRGALPASIVDALSQYKIRALTAKSKWRDVIRGNASLPSFRNDAAIPICCHKPSHRRLEKTSNGNVELELMICMKPYPRIILKTEKISGNMKATLERLLANRGNSDTGYQQRFFEVVQDRQEGRRWYLHVTYKFPASLLRLNPQVIVGIDLGFSCPLYAAINNGLARLGWRHYESLGKRIRNLQNQVFARRRSMQSGGNASLTMDTARGGHGRKRILRPIEKLAGRVNNAYSTLNHQLSRSVIEFAKNHGAGVIQMENLEGLKEQLTGTFLGSRWRYHELQQFIEYKAKEVGIEVRKINPQYTSRRCSECGYINIKFDRAFRDANRKDGKTAKFICPKCKWEGDPDYNAARNLATLDIENLIRQQLINQGIPSEREETPVL

SEQ ID NO: 173 - protein, 422 aa, unidentified
MQECKYVQRSIKLKIVCPLDENITWEEFGFLLRGLSFKVARASNFCMLHHLLYAMKLETVHLNRKGGLYCYPYLAEEYPEVPAGILCAAETRARKLFKQHAVEILRSDRALPNFRKDVGIPVPAASYKIMQDGKGDFLMEIQLLSRQAAKTGKLSGRICLALATNWRDKTAVAALQKIAEGSLKTGVGTLFREKKNWYFVVPYQREKGLSKEATENDECVMGVKLGVQNALVYAFDRSLKRGSLSGEEVKARQEQFYARKQNILEQYKWSGRKGHGRERALKPIQELYEKERNFRNTANLRYANWIVEIAVKNRCGEIHLESGKGVGFGQNSIMLHFWPVWDLKNRIKQKAEEHGIKVVECNVPDIWGACSECGAKSGNSVEKQSFSCPSCGYGKQEEKYGVGYVSAEYNAARNLALWRKEE

SEQ ID NO: 174 - protein, 438 aa, unidentified
MGKVHTRTIKLKLIVSTEDKNVAWKRIRQISNDAWRAANWIASGQLFNDQLVRRIYARRKINPREDSEAVEQIEKEFEAFFGTKRQATTEWDIKEAFPDLPPYVTNPLNHVVVASYKKEKPDMLSGNRSLRTYKRGMPIQTAKAAINFSKNETGQHFVKWTLGRKEFIDFEIYYGQDRANNRLTIDRIINGQIDYSAPMIQLKDKNLFLLLPVKEPEIAADLDPDIALGVDLGVATPAYMALSKGPARRAVGDKEDFLKVRLQMQSRKRRLQRSIKSAHGGKGRQKKLKALNQIGEKERQFARTYNHYISREIVNFAIRHNAGTIKLEMLEGFGQEEQQAFVLRNWSYFELQTFIEYKAKKVGIKILKIDPYRTSQTCSSCGHFEEGQRKDQSTFECKNCGEKLHADYNAALNIARSKKIVTKKEQCEYYLNRNQNTI

SEQ ID NO: 175 - protein, 467 aa, unidentified
MADQGKTLFKVMPLRILKPVGDTRWDALGQMLRDTRYRVYRLANLAISEAYLGFHLFRTGKAEQFKTDTIGQLSRRLRQMLLDEKVPRDNLDRSSMTGAVPDTVASGLHQYKIRSVTNVNKWQQVVRGKSSLPTFRADMAVPIRCDKAGQRRLERNTQGDVELDLMICRRPYPRVVLATGTLGPGQQTILDRLLENEDNAKSGYRQRLFEVKEDSQTHQWWLYVTYEFPAPVLPVAHGDIVVGIDLGVSVPLYAAINNGHARLGRRQFQALGYRIRTLQNQVIMRRRAIQRGGRVGVSQPTARSGHGVHRKLLPTEKLRRRIDKSYTTLNHQLSAAVIDFAKNHGAGVIQIEDLSTLKEQLVGTFLGGRWRYHQFQQFLAYKAKENGITLREVNPKYTSRRCSECGFIHVDFDRAFRDRHHTEGMVTKFVCPQCKYEADPDYNAARNIATLDIEQRIKVQCQVQKLM

SEQ ID NO: 176 - protein, 543 aa, unidentified
MAKKQDDNTMTIARKITLIPVASERKEWKKRIDAFLEKDFPMQIEIKKKQIKNTSKPERVDGYKQQLAELEKQYEEFKENGIKEYTHKMASDYTYDIVRRAMESEARRKNYILSYIYTKMIQDEVANLPTLTEKNKWVSANVKECYRKAGNKNGSIFTNVDIDNPLAGYGSDFGQAFTRKIKKLIKDGILEGNVSVPNYKLDSPFALSNQNFGIFTDCENITELKKNIGKPTYPVYVCLGKHGLPTIAKFKINFGHKQNKNKAELISTIIKILTGEYSVGGSTFGIDDDKIEMNLSITMQKQKMDLDENTVVGVDLGLAVPAVCALNNNEYDKQYIGSGNDLVTRRTKFQNEYTQLQKALKLAKGGHGRKRKLLALERLKEKEKNFVDTYCHRVSKKVVDYAIKHRAKYINIENLKGYDSSEFVLRNWSFYKLQQYITYKAEQCGIEVRKINPSFTSQVCSFCGHWEEGQRKDQATFKCKNPNCKSHKLYTVNADYNAARNIAMSTQFTDDKFKCSKKTIQDAADYYGIVLEDDKNNDNKKAA

SEQ ID NO: 177 - protein, 487 aa, unidentified
MLLIQIEEVNQMITSRKIKLAIVSDNKDTAYSFIREETRNQNRALNVAYTHLYFEYVAQEKLKQSDKEYQQHLEKYKNAAAKKYQEFLTIKEKSKSDENLQPKMDKVRETYNKAMEKVYKIEKDYSKKAREIYQQSVGLAKQTRLGKLIKSEFDLHYDTVDRIGSNAMSDFSNDRKSGILSGERSLRNYKKTNPLMVRARSMKLYEEDNNFYIKWINDIVFKIIISAGSKQRMNIAELKSVFIKLLSGQCKMCDSSISLDKGLILNLSIDMPITKENVFIPNRVLGVDLGLKIPAYVSLNDTHYIKGAIGNIDDFLKVRTGLQSQRRRLQKSLQSTGGGKGRRKKLQALERLKTKEKNFVNTYNHFLSKNIVQFAVKNNAGAIHMEELKFDKMKNKSLLRNWSYYQLQTMVEYKAKSEGIEVYYVDASYTSQTCSKCGNLEEGQREARDTFVCKKCGYNVHADYNASQNIAKSTKAINKTIEITNIV

SEQ ID NO: 178 - protein, 417 aa, unidentified
MPKQEEEGMIITRKIEIACRNKDQYPLLQDYLKQCRLMANKAMTNYYILWQEMLETKPKNQTQKDYFLKKHGCSFQNTGYRYLRRCFGGMPSYLRRDVANRVYQDFRADLKNGLLKGERSLRNYRTGHLPLHNKDIQLCKEETSSDYLLRWRMGIEFVLLFGRDRSGNRIIADRLIEGTYRVSGSQIYKDWRKKKWFVLLCVKIPQQDNNLDETAVVGVDLGLSTPAVCALSNGMARAYIGSSNLLKRFEMQKKQRSRQREFIPATNKHGRQKVVKAIYAMKDKERRFVHSFNHAISKKIIQFALKYKAAVIRMEDLSGYNGDETVLRNWSYYELQSMIEYKAEKEKIRVEKIPACYTSRACSQCGFIDKENRKTQAQFECVKCGFKENADYNAALNIARGGVKIPEENQEDFGENE

SEQ ID NO: 179 - protein, 424 aa, unidentified
MILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 180 - protein, 416 aa, unidentified
MQRSIILKILRPYDESIKWEELGYLLRGLSYKVCKISNYCMTHHLLRALNLETENLNPQGHLYCYPRLAQDYPDVPAGILCAAEGRARKVFLKCGKEVLRSETALPNFRKDCSIPIPVAGYSLLKAGEDTYVANVQLLSRQAAKTQKLPGRIQLVLAKNWRDRSAGPVLQQLTEGTLKRGIASLFRRKRDWYISIPYEAEAQKTEDGSFAPGLVMGVILGTQCALAYAFNRSPKRGAIGGEEILAHKEKFRARKKHIREQYNWSGRKGHGRGSALKPLQALYEKERNYRNLTNERYAKWVIEIAKKNRCGKIHLDGGSAGGTGTPHVMLAYWPQGELRKKIRYKAEACGIEVVECDGRDVWNRCSKCGAVQEVSGERRWFTCSHCGYGKDDKKTSSSFVTVDYNAARNMAIMESKP

SEQ ID NO: 181 - protein, 488 aa, unidentified
MATEYTCITRKIEVHLHKHGDTEEAAQRLKDEYHIWDNINDNLYKAANRIVSHCFFNDAYEYRLKIHSPEFRQIEKSLQHAKKNKLSEDDIKKLKAERKSLCADFREQRLAFLRGGATEGANPEQNSTYQVVSHEFLDVIPSDILTCLNQNIASVYKQYALEVEMGKRTIPNFKKGIPVPFAIKQNKQLRLRKRNDGSVYVLLPRELEWDLSFGRDRSNNREIVERVLSGQYDVGNSSIQETRSGKRFLLLVVKIPKASRTLDTKKVVGVDLGIATPLYAALNDNEYGGLSIGSQEQFLKIRMRMTAQKRELQRNLRYTTNGGHGRTQKLQALERLEGKERNWVHLQNHIFSKSIIEYALKNNAGVIQMERLTGFGHDKNDEVGTEYKFILRYWSFFELQSMIEYKAKAAGIEVRYINPYHTSQTCSFCGKYEKGQRINQATFVCKNPDCVKGKGRQKSDGTYEGINADWNAARNIALSEEFVDKKKK

SEQ ID NO: 182 - protein, 482 aa, unidentified
MNTVRKIKLTILGDIETRNKQYKWIRDEQYKQYRALNLCMTYMVTNLMLRNSESGLENRKEKEILKIQNKIKKDEEKLEKELKKEKSKEEKIQDIKFNIEELKLQKEKLENELRNIKEYRSNIDEEFKKMYVDDLYNVLNKVPFQHEDMKSLVTQRIKKDFNNDVKEIMRGDRSVRNYKRNFPILTRGRDLKFQYIEKSEDIEIRWIEGIKFKCILGKTSKSLELKHTLHKVINKEYKVCDSSLQFDKNNNLILNLTLDIPQDNKYEKITNRVVGVDLGLKIPAYVALNDTKYIRKAIGSIDDFLKVRTQMQSRVRKLQKSLQTARGGKGRNKKMKALDRFREKERNFARNYNHFLSYNIVKFALDNKAEQINLELLEMKETQNKSILRNWSYYQLQSFIKYKAERIGIKVEYIDPYHTSQICSECGNYEEGQRVEQATFVCKRCGHKINADYNAARNIAMSKEYISKKEESKYYKNNKNMV

SEQ ID NO: 183 - protein, 450 aa, unidentified
MSKNTYTITRKIQLIPVGDKEEVNRVYTYLRDGMKAQNMALNQYISALYFAMQNDATKDSRKELRNLFSRISTSKKGSAYDDTIEFAKGLPSCMMTRKVESDFKNAMKKGLKYGKLSLPTYRDTNPLLIHIDYVRLRSTNPHLDNGLYHNYKNHTEFLEHLYDENLELFIKFANKITFKIILGNPHKSSELRSVFKNIFEDCYHIQGSSIEIEKNKIILNMSMSIPVKKIELDENIVVGVDLGMAIPAVCALNTNEYIHKSLGNYNDFIRERTKIQAQKKRMQKSLTYTNGGHGRKKKLKPLNRFKERERNWVKTYNHKISKQIIDFAIKNKAKYINLEDLSGIAKEDKDKFVLRNWSYFELQQFITYKAEKYGIIVRKIKPEYTSQKCSCCGHLEENQRINQSEFICKNPNCKNFGKIVNADYNGARNIAMSTNFVEEKETKSKPKKVA

SEQ ID NO: 184 - protein, 416 aa, unidentified
MQRIIILKILRPYDESIKWEDLGYLLHGLSYKVCKISNYCMTHHLLRALNLETENLNPQGHLYCYPRLAQDYPDVPAGILCTAEGRARKVFQKCGKEVLRSETVLPNFRKDCSIPIPVAGYSLLKAGEDTYLANIQFLSRQAAKTQKLPGRIQLVLANNWRDRSAGKVLQQLTEGTLKRGIASLFRRKRDWYISIPYEVEAQKTEDGSFAPGLVMGVILGTQCALAYAFNRSPKRGAIGGEEVLAHKEKFRARKKHIREQYNWSGRKGHGRGSALKPLQALYEKERNYRNLTNERYAKWVIEIAKKNRCGKIHLDGGSAGGTGTPHVMLAYWPQGELRKKIRYKAEACGIEVVECDGRDVWNRCSKCGAVQEVSGERRWFTCSHCGYGKDDKKTSSSFVTVDYNAARNMAIMESKP

SEQ ID NO: 185 - protein, 472 aa, unidentified
MATITRKIELYIDKSNLTDDEYKAQWQYIRQIDNTLFLAANRISSHCLLNDELEIRLKLQMPEYCEIEKSLRNSKKNKLSKEEISELKLRRKELDAVVKRQKEEFLKTSQNSTYQLVAYEFTNIPTEILTNLNNDIVGKYGKARLDIIKGIKSPSTYKKGIPIPFSVNKKSPFVFIDGDLEWFKGKTKETTGKILRFKLHFGKDKSNNRAIVERLVESAKLGKKKGEAYIVNNSSIQLVEKENTTKIFLLLSLDIPTTKRALDSNLVMGIDLGINYPIYYATNGNAYIKGHIGDRDSFLNERMVFQRRFRELQRLQCTQGGRGRLKKLAPLEKLREKERNWVRTKNHIFSREIIKCALKIGAGTIHLEKLKNIGKDKDGNIEDSKKYILRNWSYHELQEMIEYKAAMEGITVKYVNPAYTSQTCSCCGNRGERISQSVFKCLCPECSEYGKEVNADYNAARNIAKSEIFVKK

SEQ ID NO: 186 - protein, 543 aa, unidentified
MDNTITITRKYTLIPTFSDTKEWTKKVMEYTKTSYIEKIKYYEEKIKKTKKKDKEEREKYENRLSQLKEQQLDFEENGTLLQTNVNDYTYDLVREAMASESDRKNMIISYVCGELINRDAKDMDFKERNKLISELCNYGYRVKGSKKGSLFDHLDIDNPLGGYGVSFCQDLTKKIKELVNNKRWLDGKVSTLHYKDDSPFSIAKATMGFAHDYDSFEELCEHIREKDCNLYFNFGNNGKPTIARFKINLGANRKNKDELISTIIRVYSGEYQYCGSSIGIEGTKIILNLSMKIPKQEKELDENTVVGVDLGIAVPAVCALNNNVYARKFVGNKDDFFKVRKQLNAQYKRVQSALKRASGGHGRKKKLKALERLRKKEAHFVETYCHMVSKAVVDFALKYNAKYINLENLTGYDTDDIVLRNWSYYKLQQYITYKASKYGIEVRKINPCYTSQICSECGNYHPENRPKGDKGQAYFNCHNKECITHGKKSPYQYGINADFNAARNIAKSTLWMGKGKVTEESKKKAREYYGIEEEYEELNKEVA

SEQ ID NO: 187 - protein, 469 aa, unidentified
MITARKIKLTIAENREEGYSFIRKELQEQNKALNMAINHLYFNYVAREKIKLADETYKVKLEEGECYLERKYIELKEAKTDKQKENIKKSIEATKKKLETLRKVENKEVSNNFKEIIATSEQINLRDLISNNFNLKSDTKDKLTQKVVQDFYNDIVGVLRGERTLRRYKKDNPLYIRGRSLTLYREGEDYYIKWMNGIVFKCVLGVKKQNSLELQKTLDKVIFEEYKLCDSSIGFKDNKLILNLTLDIPVSNTNKFEKVIGRIVGVDLGMKIPAYCALNDSEDVRKAIGSIDDFLKVRTQMRSRRRKLQRALKSTNGGQGKNKKLSALNSFEAKEKNFAKTYNHFLSSNIIKFATDNKAEQINMEFLSLSETQNKSVLSNWSYYQLQQMIEYKAERIGIKVKYVDPYLTSQTCSECGHYEDGQREVQSEFQCKKCGCKTNADYNASRNIAKSDKYITKKEESEYYKNKI

SEQ ID NO: 188 - protein, 478 aa, unidentified
MPILTRTIELIPIGDKEERDRCYKWIRDFMEEQSKMMNQYMSALYIAAVEEVSKDDRKELNNLYNRIATSKKGSAFSKEECNLPKGLGANYGQRVRSDFDTACENGLLHGRVSLPTYKKNFPIILAPIYVNLQKNNIEEKGKSAGFYHNYASYNELYDALKEENKPEIIWNFVQKMQYQIKFGNPYKSAFLRDEILHFLEGEYKAVGSQLSINSRGKIILNLSLDVPQKKVKLDENIVVGIDIGLAVPVMCAINNDYYKRLAVGDFEAFTRMREKLYSQKCKLQRQLKYTSGGHGRKKKLASLNAIRDREHRFVHTMNHKYSSEVINFALKNNAKYINMEDLTGFGKDNKGNAIDDYQFVLRNWSYFELQKMIQDKAQKYGIVVRKVESAYTSQLCSCCGEMGERVSQSVFRCLNPNCISHNKYEKQRKSGVGNYHFNADFNAARNISMSTNYTKKKRKTKAEKVEERKKNAIEKTAG

SEQ ID NO: 189 - protein, 425 aa, unidentified
MVKMKKETKQKRNTLCKKITLYPLGSKEEVTRVYNFIRDGQYMQYKGLNLIMGQLASQFYKCNSELDDPQYKEWVEQEIRNTNSLLQAMEFPKGIDSRSLIIQRVKQDFSSALKNGLARGERSITNYKRTLPLMTRGRDLKFSCNYSELEELEYKCFSKDFKVYVSWVNKIKFKVVLGSVKRSLALRKELCQILRSVYSIQGSSIEIKDKKIILNLSMGIPLMEKVLDENTVVGVDLGIAVPAVCGLNTNKNTRRFIGSKDDLLKIRTKVQSQRKSLNKKLRECTGGHGRSKKLQALDRLSESETNYVKTYLHMVSKEIIRFAVAHNAKYINIEKLEGYDASSFILRNWSYYQLQSFIEYKAKIKGIIVRKVNPYHTSQICSYCGHWEEGQRLSQDKFKCKCCGVELNADFNASRNIALSTEFVE

SEQ ID NO: 190 - protein, 437 aa, unidentified
MAKDTFTITRKIQLVPVGDKTEVNRVYNYIREGMKAQNLAMNQYISALYLGMQNDVSKDDRKELNNLFSRISTSKKGSAYDESIQFAKGLPIGSMTRKVKSDFDTAMKKGLKYGKLSLPTYKDSNPLLVHVDYVRLRSTNPHQDSGLYHNYTNHTEFLEHLYKSDFELFIKFANYITFKIILGNPHKSAEIRDVFKNIFEECYAIQGSSIGIYNNKIICNLSISIPKKQLCLDENIVVGVDLGLAVPAVCALNTVPYIHKSLGNYDDFVRERTKMQSQRKRLQKSLNYANGGHGRKKKLQSLERLKKRERNWVQTYNHKISKQIVDFAIKNKAQYINIEDLSGFDSSQFVLRNWSYFELQQFIEYKANKYGIIVRKINPYHTSQTCSFCGHWEEGQRISQSEFICKNPECVNHNKSINADYNAARNIAMSTDFVKNN

SEQ ID NO: 191 - protein, 517 aa, unidentified
MDNTITITRKYALIPEFSDRKEWKKRVYDFTINDLEQKIDYRNKKKQDASELESQLEYIKNGGDFTRNMVNNYTYSLVRTAMEEEARRKNYILSWIFSEMRANRVDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHISDSDCKMYMNYGGDNRKDGINPASIAKFKISIGHGKNKDELKSTLLKVYSGEYQYCGSSIQITKNKIILNLTMKIPKIETKLDENTVVGVDLGIAIPAMCALNNNMYERLAIGSADDFLRTRTKLQSQRRRLQKSLKNSNGGHGRNKKLKVLERLGKSETHFVETYCHMVSKRVVEFAVKNRAKYINIENLNGYDTSQFILRNWSYYKLQQYITYKAERYGILVRKINPCYTSQVCSVCGNWEEGQRKTQSSFECANPECKSHEKYKYGFNADFNAARNIAMSTLFMETGNVTEKSKEEARKYYGIEKD

SEQ ID NO: 192 - protein, 517 aa, unidentified
MDNTITITRKYALIPEFSDRKEWKKRVYDFTINDLEQKIDYRNKKKQDASELESQLEYIKNGGDFTRNMVNNYTYSLVRTAMEEEARRKNYILSWIFSEMRANRVDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHISDSDCKMYMNYGGDNRKDGINPASIAKFKISIGHGKNKDELKSTLLKVYSGEYQYCGSSIQIAKNKIILNLTMKIPKIETKLDENTVVGVDLGIAIPAMCALNNNMYERLAIGSADDFLRTRTKLQSQRRRLQKSLKNSNGGHGRNKKLKVLERLGKSETHFVETYCHMVSKRVVEFAVKNRAKYINIENLNGYDTSQFILRNWSYYKLQQYITYKAERYGILVRKINPCYTSQVCSVCGNWEEGQRKTQSSFECANPECKSHEKYKYGFNADFNAARNIAMSTLFMETGNVTEKSKEEARKYYGIEKD

SEQ ID NO: 193 - protein, 551 aa, unidentified
MNLHGYKTDAKGFSSFLQSGNPVNLHGCKTRKEIKMESTTVITRKYTLIPEFSECKEWKKRVYDFTVKDIEQRIEYKKKKKQDISDLEIQLECIKNNSEFTRSMVNNYTYNLVRTAMEEEARRKNYILSWIFSEMTANRVDQMESLKDKFKFVSSTINYAYRKAGSNKGSLFDETEIHCMLKSYGIAFSQELTKKIKELVKNGVLEGKVVIPTYKLDSPFTIAKAHFSFDHDYDSFEELCEHINDSDCKMYMNYGGNNTKTGTNPASIARFRINLGHGKNRDELKATLLKVYSGEYRYCGSSIQISKNKITLNLSMKIPKVEMKLDENTVVGVDLGIAIPAMCALNNNLYERESIGKVNDFTRVRKKHQAQVKRLQKSLKSATGSHGRSKKMRALNRVKKSEAHFVESYCHYVSRKVVDFALKHNAKYINMENLKGYDTSQFILRNWSYYKLQQYITYKADRYGIVVRKINPCYTSQVCSVCGHWEPDQRKTQASFECANSECESHKKYKYGFNADFNAARNIAMSTLFMEDGEVTEKKKEEAREYYGIKK

SEQ ID NO: 194 - protein, 468 aa, unidentified
MPTITRKIELTLCTEGLSDEQRKEQWKLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIEADSGEFYLRWYNGLRFLLTFGKDRSNNRMIVKRCMKMDEDFEGEYKLCNSSIQLAKRDGKPKLFLLLVVNIPQEHVELNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKAAKYGIKVVKIRPAYTSKTCSWCGQQGDRKSTTFICENPECKHYGESIHADYNAARNIANSNDIVKENE

SEQ ID NO: 195 - protein, 522 aa, unidentified
MDNTITITRKYALIPEFSDRKEWKKRVYDFTINDLEQKIDYRNKKKQDTSKLESQLEYIKNGGDFTRSMVNNYTYSLVSTAMEEEARRKNYILSWIFSEMRANRVDQMESLKDKFKFVSDTINYAYRKAGSNKGSLFDETEIRCILKSYGIAFSQELTKEIKELVKNGVLEGKVVIPTYKLDSPFTIAKSHFSFEHDYDSFEELCEHISDSDCKMYMNYGGDNKKDGINPASIARFRISLGHGKNKDELRATLLKVYSGEYQYCGSSIQITKNKIILNLTMKIPKIETKLDKNTVVGVDLGIAVPAMCALNNNMYERLAIGSADEFLRVRTKYQAQRRRLQKSLKNSSAGHGRKKKLKALDRMDKAESHFVETYCHIVSKRVVEFAVKNRAKYINIENLNGYDTSQFILRNWSYYKLQQYITYKAERYGIAVRKINPCYTSQVCSVCGNWEDGQRKTQASFKCANQKCESHKKYKYGFNADFNAARNIAMSTLFMEDGEVTEKKKEEAREYYGIKKENSKAV

SEQ ID NO: 196 - protein, 504 aa, unidentified
MSYTEQFYIDKIAYYKNKLTKTKGKEEKQKIKDKLASLEEQENEFEESGILTQANVTDYTYDLVRNAMASEANRKNAIISYIILELLHNGGQTMDFNARNKLINDLVNYGLRVKGSSKGSLFDELDIENPLNAYGFAFKQDLKKKIRDMVNSKRVLDGKSSVITYKADSPFSINKENMSFTHDYSSFEELSDHIRDNDTNLYFNFGSSGNPTIARFKINLGAGRHKKNKDELIATLLKLYSGEYQFCGSRIGIEKNKIILNLVLSIPKKVRALDENTVVGVNLGVAVPAMCALNNNEYERLAIGSADEFLRVRTKLQAQRRRLQKSLKDASGGHGRTKKLKALERVAKAESHFANTYCHMISKRIVDFALKNNAKYINLENLTGYDTNDFILRNWSYYKMQQYTTYKAEKYGIIVRKVNPCYNAQACSVCGNYAPGQRKSRAVFICANPACKSHKKNHGKLDAEFNNARNVAMSTLYMNDGQVTEKSFKEARDYFGIEEEIETI

SEQ ID NO: 197 - protein, 542 aa, unidentified
MAKKQNKNTMQINRKITLIPVASDNDGWKKKMNTYLEKFYFDKKIKAKERQIKNTSKPERKEEYKQQLTEYKKQQEQFLNGELEDYTKQMVMSYTYDLVRDCMESEARQKNFIMSYMFSEMIREKVCYLKNKKEKEKWVNDNINVAFRVKGSPKGSIFDDVEIYSPLRALSIQGYTQELKGRVKKFATDGGLDGKLAVDNYKLDSPFHMSKQGFDIVHEYEDLTELKKNIGKSSCEIYVNLGNGGVPTFARFQLDFGHKGNREELISTITKVFTGEYKVCGSSIQINKKNKIILNLGLEIPKRLTELDENTVVGVDLGLAVPAVCSLNNNQYKKEYIGDGEAFVKQRGKIQKEKQRLQKALKLSKGGHGRKRKMLALERFKEREANFVNTYCHRISKKVVEYALKNNAKYINIENLKGYDSSKFILRNWSFYQLQQDITYKAERYGIEVRKINPAFTSQVCSFCGYWESGQRINQKTFKCGNPNCKSHNLKFFNADYNAARNISMSTLFTSDNYKFGKESLQKAADYYGIDLEIDSEDQEIA

SEQ ID NO: 198 - protein, 492 aa, unidentified
MITVRKIKVRCEDKTFYDFMRKEQREQNKALNLSIGYIHTNSILKSVDSGAETLILNSIEKLNKKVDKLKKDLEKPKITDKKREQTEKAIQTNLKLIKDEQIKLEEGKQFRQGLDKQFSEIYINNNNLYHVLKSQTQVQYMRTLDLVTQKVKQDYSNNFVDIVTGKCSLMNYKSDFPLMIDKKCINIFKEEQNYKIRIMLGYELDIILGRRNNENVNELKSTLEKCISGEYKICQSSISINKNDVIFNLTLDIPNTTNYEPVHGRVLGVDVGVKYPVYMCVSDNTYKRKHIGSAADFLKVRQQFQERKRKLQASLDITKGGKGRKKKTQALERFKDKERNFAKTYNHQVSKKVVEFAKKNKCETIALEKITKEGIGDTILRNWSYYELQHMIEYKAKRECIKVEFVDPAYTSQTCSKCGHVSKDNRQTQEHFKCVNCGYELNADHNAAINIARRSIDYKPKEIIEKTIEKTIQPITTDLVEETGEFKQLTFI

SEQ ID NO: 199 - protein, 446 aa, unidentified
MQRSITLKIIRASDDKITWEELGYLLRGLSLKICRMSNFCMTHHLLHALKLETEMLNPKGNLYCYPHLAEEYPEVPAGIVCAAESRARKLFRRCGAKVLRSEMSLPSFRKDNSIPIPVAGYRILQDGDTNYAEIQLLSRQGAKTQKLPGRIRLTLADNWRDKAARPALQKLAAGKIRRGVASLYRAKNDWYLCIPYEVEAANTGGDFEPGLVMGVAFGVYNALVYGFNTLLKRGAISGEEILSHQAKVKARRKKIQEQYPWSGRKGQGREDTLKPLRHLHEAEKNYRELVNHRYAKWVVDIAVKNRCGEIHLDDGHAPPLGKNKILLSRWSLYDLKNKICRKAEEKGIRVTECSVPDLRTRCSHCGTEQVAGNHKRMFLCADCGYGSTEKNRGTGYISVDYNAARNLAMYDTGKNEPNMERDLIPKDDPAYDRNEGFREELSGSGQ

SEQ ID NO: 200 - protein, 488 aa, unidentified
MATEYTCNTRKIEVHLHRHGEDEEAKQRLIDDYRVWDTINDNLYKAANRIVSHCFFNDAFEYRLKIHSPRFQEIEKLLKYPKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIYVRFPLGLEWDLSFGRDRSNNREIVERVLSGQYDVGNSSIQESKNRKRFLLLVVKIPKENHNLNPDRIVGVDLGINIPLYAALNDNEYGGMGIGSREQFLNMRMRMVAKKRELQRNLRQSTNGGHGRAQKLQALERFEGKERNWVHLQNHIFSKSIIEYAVKNNAGTIQMERLTGFGRDKNDEVDSDFKFILRYWSFFELQTMIEYKANAAGIEVRYVDPYHTSQTCSFCGHYEKGQRLNQSTFVCKNPDCEKGKGKKLSNGTYQGINADWNAARNIALSDKIVDRKKK

SEQ ID NO: 201 - protein, 456 aa, unidentified
MLITRKIELHISEADPELRKEHWKYLSFLNSEIYKAANLIVTNQLFNNFLENRVVDADGNVMDIGKRIRSLYRNKDKNADEIEKLKAAKLELRTEVKKFYLKSKQNTTYSITSHNFPEIDASIITTLNAQITGVLKKEWNEVERGSRSLRTYKKGMPIPFNLTSKSKKWFEKVDDEIFLTWFKGIKFKLFFGRDRSNNRAIVDRCLSGEYKYSDSSIQYKDRKIFLLLVVDIPESTKVLDENVSVGVDLGITVPAYCALSDGLKRLAIGSSEDLLRVRLQFQSRKRRLQRALKSSKGGQGRDKKLKALDNVADKEKRYVSTYNHMISSNIVKFAKDNNAGVIKLEMLEGFGEDEKNKFILRNWSYYQLQTMIQYKAKRENIKLVFIDPYHTSQTCSICNHYEAGQREKQSEFICKNPDCKNFDTPINADFNAALNIAVSKKVVTSKEDCEYFKKEN

SEQ ID NO: 202 - protein, 496 aa, unidentified
MILTRKIKLVIVSENQKEGYSLIRNEIREQYKALNLAYNHLYFEHNAIQKLKQNDEDYKQKRSKLQELINKKYEEHQKVKNLEKKEALREAYNKKKQELYNFEKEYNEKARQAYQQVVRFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVAELQSVLENVLLGAYKICDSSIGFNNKDMILNLSLNIPDKETHDYIPGRVVGVDLGLKIPAYVSLSDKVYVRKGIGSIDDFLRVRTQMQKRRRRLQESLAAVKGGKGREKKLKALDHLKGKEANFAKTYNHFLSTQIVTFAVKNQAGQINMEFLEFDKMKNKSLLRNWSYYQLQMMVEYKAKREGIIIKYVDAYLTSQTCSKCDHYEEGQRETQERFKCKSCGYEVNADYNASRNIAKSTRYISDSTESEYHRKKQEALKEILGENDTINEQLSLFDKRDDIA

SEQ ID NO: 203 - protein, 415 aa, unidentified
MQRSLKLKIVRPYDESITWEEIGYLLRGISYQICKMSNYCMTHHLLRALGMETENLNPQGNLYCYPRLAKEYPDVPTGIICAAEGRARKLFKRNAPGILRSETALPSFRKESSIPIPVAGYSLAKIGPDIYVADVQLLSRKAAKTGKLPGRIQFVLANNWRDKKAGSVLHQLAEGTIKRGIASIFRNNRDWYIGIPYSVDPAPSDGELDPDLVMGVAFGTHSALAYAFNNLLKRGELGGEEVLSHREKFKARRQHIREQYNWSGRKGHGRENALKPIRELYEKERNYRNLTNERYAKWVVEIAKKNHCGKINLDAGGDRRNGKQNILLAYWPQSELHMKIKNKAEAYGIQVQECTATDIRRRCSRCGAVQETSGDKRWFICSHCGYGKEEKKTSSGFVSVDYNAARNVSMWEGTT

SEQ ID NO: 204 - protein, 461 aa, unidentified
MIAVRKLKVICKDKEFYDFFNMEQREQNKALNMAISLRHVNNTLKRIDSGAEVIIIKSIDKLNRKIETLEKELKNENITEAKKEKTLKDIETHKKIIKGEKKKLEEGRIYRKGLDKEFSQNYFDKTQLYHVLDGMTSIQHKRTIELVREKVKKDYENNFIDIVTGKQSLPNYKSDFPLMIDGSSIHIFEEDGIYKIKIMRGYELEVILGRRENENILELRKTLDRCSNGQYKVCQSEIQRDKNNNIIFNLTIDIPIENKKYTPVEGRVLGVDLGIKYPVYMCLNDDTYKRTSLGNINNFLRVREQMQERRRAFQKDLTLTKGGKGKSKKTQLLNKLRENEKNFCKTYNHTMSKRVVEFAKKHNCEYIHIENLTKDGFSNSILRNWSYYELQKFIKYKADREGIVVKYIDPAYTSQMCSKCGYTDKENRQTQAEFKCISCGFKLNADHNASINIARSNKFIK

SEQ ID NO: 205 - protein, 468 aa, unidentified
MPTITRKIELNLCTEGLADEEQKAQWNLLYHINDNLYKAANNISSKLYLDDHVSSMVMLKHAEYLTLVRALEKAQKQKTPDETVIEDLRRQVAAAEKDMTDQELAICNYATEMSTQSLSYRFATEIETNIFAQILDCLKQGVYATFNSDAKDVKRGERAIRNYKKGMPIPFPWNKSLKIEHKDGEFYLLWYNGLRFHLNFGKDRSNNRLIVQRCMKMDKDYEGDYKMCNSSIQFVKREGKPKFFLLLVVNIPQEHVELDKKIVVGVDLGINSPAYVATNVTMERQQIGRRETFLNGRMSFQRRYKSLQRLQTTAGGRGRKKKLEPLERLRNAEHNWVHTQNHLFSREVVQFAVKARAATIHMEDLSGFGKDNDGNADERKEFVLRNWSYYELQTMISYKAQKYGIKVEKIRPAYTSRTCSWCGHEGFRKGEIFICENPACEKCGEKENADYNAARNIANSKDIIKNHG

SEQ ID NO: 206 - protein, 462 aa, unidentified
MGNNRMTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYHDFLDKINDSDLAVYVKWVNKIVFKVVFGNPHRSLELRSVIQNILEENYKVQGSSIEIDGKSIILNLSISIPKQLRELDENIVVGVDLGIAVPAMCALNNNLYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKTGFNADFNAARNIAMSTLWMGSGQVTEKSKQEAREYYDISEKYEQSKNGSENNKVA

SEQ ID NO: 207 - protein, 423 aa, unidentified
MNIVKKIKLRIIDNDKELCKKQYLGFTEEQKKELIDKQYKFIRDSQYQQYLGFNRAMGFLMSGYYANNMDIKSDNFKEHQKKLTNSLYIFDDIKFGVGIDSKSLIVQRVKKDFSTALKNGLAKGERSVTNYKRTYPLLTRHRSIKFLYAENELDIYLDWVNKIRFRCELGNHKNSLELQHTLRKVITGEYKISDSSLEFNKKNELILNLNLNIPETKATFIKDRTLGVDLGMAIPAYVSLSDTPYIRKGFGSYEEFAKVRNQFKDRRKRLLKQLSLVAGGKGRAKKLHSMEFLKNKEKQFAKTYNHSLSKKIIDFALKNNCEYINLEDIKSTSLEDRVLGQWGYYQLQEQIEYKAKLVGIKVRKVKAAYTSQTCSECGNIDKENRKNQSTFKCTNEDCKLNKKGINADWNASINIARSKEFIK

SEQ ID NO: 208 - protein, 488 aa, unidentified
MATDYTVITRKIEVHLHRHGDSEEAIQRYKEEFRIWDEINDNLYKAANRIVSHCFFNDAYEYRLKIHSPRFQEIEKLLRYSKRNKLTDEDIKQLKNERKQLFTDFKKQRLAFLRGGATEGTNPEQNSTYKVVSNEFGDIIPSDILTCLNQNIASVYKAYSKEVEFGMRTIPNYKKGIPIPFTIRPNGVLNLQKREDGSIYIRMPKGLEWDLSFGRDRSNNCEIVERVLSGQYDVGNSSLQESKNKKRFLLLVVKIPKVNKALNQDRVVGVDLGINTPLYAALNDNEYGGMSIGSREQFLKMRMRMNAQKRELQHNLRHSTNGGHGRKQKLQALERLEGKERNWVHLQNHIFSKSIIEYALKNDAGVIQMERLTGFGRNKNDEVDEGYKFILRYWSFSELQNMIEYKANAAGIEVRYIDPYHTSQTCSFCGHYEKGQRISQSTFVCKNPECTKGKGKHKSDGSYEGINADWNAARNIALSNNIVGRKKK

SEQ ID NO: 209 - protein, 481 aa, unidentified
MATEYTCITRKIEVHLHRHGDSEEALQRYKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVAVPLYAALNDNPYGGMSIGSADQFLKVRMRMAAQKRELQRTLRNSTNGGHGRKHKLQALDRLEGKERNWVHLQNHIFSKELVEFALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 210 - protein, 464 aa, unidentified
MVEQGVTITLNKSDKNTYTIARKVRLAVDGDKEEVNRVYQFLRDGIYNQNKAYNIFISSVYSAIINGASTEELNEIYKRGSRKPKEDDETYSLYKFGEIEFPVGVGTTASLKQMVKNDLKKAKNDGLFKGKISLPNRKLNAPLRIESACFSFIHNYNSYQEFLDHLYTDDCEIIMKFVNNIRFKVNLGQACKTHELRSVFQNIFEENYKVCGSSIEIDGTKIILNLSLTIPKKKHELDESIVVGVDLGIAVPAVCSLNNNTYIKKFIGSRNDFLRERTKLKAQRRNVQKSLKFTSGGHGRKKKLRHLEVFTEHEKNWVKNYNHRVSKEIVDFALSNDAKYINIENLQGFDSSNFLLANWSYYQLQQQIEYKADMHGIIVRKVNPYHTSQRCSCCGFESPDNRPKDKKGQAYFKCLNCGTEMNADFNASQNIARSTDFSIGEVTLEEDKKKHNKSKKIPKEKQAA

SEQ ID NO: 211 - protein, 434 aa, unidentified
YISSHMFFNEAYIERLKAHSHRYREIKKTLKKIDEFTPEEAKALNDEKRVLEKQFVEERLRFLKGGDANGSGSVMSSTRQAVVEAFPEIPFKVLDCLNREMSKTFSQYKRDIENGKRTLSNYKKYYPVPFSMEQGKQLRKREDGSIYVMFPGKLEWDLYFGKDPSNNREIVERIFNGEYQACDSSIKEIKGRKKQILHLVVKIPKKTIKRDSNRVVGIDLGVNTPLYAALNDSERERFSIGSREAFLNVRMRFNAQRRELQRNLRHTTNGGHGRKQKLQALERLEGKERNWVHLQNHIFSKSLIEFAQKCEAGVIQMEELSGFGRDKFDSVDDGYKFILRYWSFFELQNMIEYKANAAGIEVRYVNPAYTSQTCSYCKHYEKGQRISQSTFVCKKCTEGKGKQRKDGSYEGINADWNAARNIAMSDKIVDRKKK

SEQ ID NO: 212 - protein, 466 aa, unidentified
MITTRKLKLTILGDEETRKLQYKLIRDEQYEQYKALNLCMSLLNTHNILSGYNTGAENKLNSQIDKLNKKLEKAQFEIKKNDIKQSKLDKLNSDIELYQNELNKLKEQFTQSSKYRSDIDLKFKEMYIDDLYTIVQNQVTFKNKDLMSLVTQRAKKDYSIALKNGMARGERSLTNYKRDFPLMTRGERWLKFKYDENSDDILIDWISGIKFKVILGYRKNENSIELRHTLHKVINKEYKICDSSIQFDRNNNLILNLTLDIPNNSKSEFIENRTLGVDLGIKYPAYICLSDDTFKRESIGCAEDFIRVREQIRNRRKRLQQQLKMVQGGKGREKKLKALDRISDKERNFVKTYNHMISKNIVNFAKKHKCQYINLEKLTKDGFPNMILSKWSYYELQQMIEYKAERENIKVRYIDPAYTSQTCSRCSHIEKDNRETQEKFKCLKCEFELNADHNAAINISRSVNFK

SEQ ID NO: 213 - protein, 461 aa, unidentified
MISTRKIKVRCDDNTFYTFFRQEQREQNKALNIGMGIIHADAILHNIDSGAEKKLKKSIEGLQGKIDKLNKYLEKENITDKKKKEVLKAIETNKKILDGEKKAFKESEEYRKGIDELFKSTYLKSNTLYHVLDSMVNIQYKRTLSLVTQRIKKDYSNDFVGVVTGQQSLRNYRNDNPLMISNQQLDLKYIEDTFYLDVMCGYRLEIILGRRDNQNVNELKSTLHRILSKEYKVCDSSMQFDKNNKDVILNLIIDIPNKSNMYEAIKERTLGIDLGVEVPIFMCLNDNTYIKKGIGDINNFLRVRQQIQIRRRKLQKDLTLTNGGKGRKKKLQLLEKLQENEKNFVKTYSHVLSKRIIEFAKKNKCEYINLEKLTKDGFDNIILRNWSYFDLQRMIEYKAKREGITVRYVNPSFTSQKCSKCGEIDKENRQTQADFKCTNCGFELNADHNAAINIARSIDFV

SEQ ID NO: 214 - protein, 417 aa, unidentified
MQRSIKLKILRPFDGNITWEALGYLLQGLSFKVCRISNFCLTHHLLRALKLETENLNPKGHLYCYPRLAEEYPEVPAGIICAAEGRARKVFNQKGKSIMRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWEPETDS

SEQ ID NO: 215 - protein, 484 aa, unidentified
MATEYTCITRKIEVHLHKHGDSEEALQRLNEECRIWDEINNNLYKAANRIISHCFFNDTYEYRLKLQSPRLQEIEKLLSNPKRNKLSDEDIKQLKAERKQLFANFKKQRQVFLRGGVEEGANPEQNSTYRVVSNEFIDVIPSEVLTNLNQNISSTYREYSLDVERGSRTIPNYKKGIPVPFSIKRSGELMLKKREDGSIYVRFPKCLEWDLFFGRDRSNNREIVERVLNGQYDVGISTIQETKNKKRFLLLVVKIPKESKKLNPNRVVGVDLGINIPLYAALNDNEYGGLGIGSREQFLKVRMRMVAQKRALQRNLRHTTNGGHGRAQKLQALDQLEGKERNWVHLQNHIFSKSIIEYALKNGAGVIQMERLAGFGRDKNEEVENEFKFILRYWSFFELQTMIEYKANVAGIEVRYIDPYHTSQTCSFCGHYEKGQRINQSTFVCKNPDCVKGKGKQHADGSYDGINADWNAARNIALSTTVVD

SEQ ID NO: 216 - protein, 417 aa, unidentified
MQRSIKLKILRPFDGNITWEALGYLLQGLSFKVCRISNFCLTHHLLRALKLETENLNPKGHLYCYPRLAEEYPEVPAGIICAAEGRARKVFNQKGKGILRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWRQETNS

SEQ ID NO: 217 - protein, 461 aa, unidentified
MGNDRMTICRKIKLFPVGDKEEINRVYDFIRNGQYAQYQACNLLMGQLMSEYYKYNRDIKNKEFKARQKEIMTNSNIILKDIDFVTGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENIVVGVDLGIAVPAMCALNNNIYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGSWEFGQRKSQSVFECANENCDSHTKYERGFNADFNAARNIAKSTLWMESGQVTEKSKQEAREYYGISEKYEQSKNEVENNKVA

SEQ ID NO: 218 - protein, 419 aa, unidentified
MQRSIILKIIRPYDESIKWDDVGYLWRGLSFKVCKISNYCMTHHLLRAMNLETENLNPQGRLYCYPHLAKEYPEIPAGIICAAEGRARKVFKQNAKGILYSETSLPSFRKDCSIPIPVSGYSLLKAGADTYVASIQFLSRQAAKTQKLPGRIQLVLASNWRDKSAGRILQQLAEGTLKRGIASLFRKKRDWYFSIPYEVEPSGADDSFEPELAMGVVFGFQCALAYGFNRLLKRGMLGGDELLAHREKMLARKKQILTQYRWSGRKGHGRESALKPLQALYEKERNYRNLTNERYAKWIAEIAKKNRCGKIYLDTGGSSGHGTQNILLAYWPKEALRKKIMNKAEAYGIEVVECTDAEIRNRCSRCGTLQEPLENKKWFVCNHCGYGKDDKKTGDGFISVDYNAARNLAVYDTGSKESV

SEQ ID NO: 219 - protein, 461 aa, unidentified
MISKRKIKVRCDDNTFYTFFRQEQREQNKALNIGIGIIHSNAILHNIDSGAEKKLKKSIESLQGKIDKFNKHLEKEKLTDKKKEEVLKAIETTKKILDGEKKAFKKSEEYRKGIDELFKSTYLKSNTLDHVLDSMVNIQYKRTLSLVTQRIKKDYSNDFVGIITGQKSLRNYRNDNPLMISNQQLDFKYIEDTFYLDVMCGYRLEIILGRRDNQNVNELKSTLHRILSKEYKVCDSSMQFDKNNRDVILNLGIDIPNKSNMYEAIKGRTLGIDLGVEVPIFMCLNDNTYIKKGIGDINNFLRVRQQIQVRRRKLQKDLTLTNGGKGRKKKLQLLDKLQENEKNFVKTYSHALSKRIIEFAKKNKCEYINLEKLTKDGFDNIILRNWSYFDLQRMIENKAKREGITVRYVNPSFTSQKCSKCGEIDKKNRQTQADFKCINCGFELNADHNAAINIARSLDFV

SEQ ID NO: 220 - protein, 401 aa, unidentified
ITWEALGYLLQGLSFKVCRISNFCLTHHLLRALKLETENLNPKGHLYCYPRLAEEYPEVPAGIICAAEGRARKVFNQKGKGILRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWRQETNS

SEQ ID NO: 221 - protein, 419 aa, unidentified
MQRSIILKIIRPYDESIKWDDVGYLWRGLSFKVCKISNYCMTHHLLRAMNLETENLNPQGRLYCYPHLAKEYPEIPAGIICAAEGRARKVFQQNAKGILYSETSLPSFRKDCSIPIPVSGYSLLKAGADTYVASIQFLSRQAAKTQKLPGRIQLVLASNWRDKSAGRILQQLAEGTLKRGIASLFRKKRDWYFSIPYEVEPSGADDSFEPELAMGVVFGFQCALAYGFNRLLKRGMLGGDELLAHREKMLARKKQILTQYRWSGRKGHGRESALKPLQALYEKERNYRNLTNERYAKWIAEIAKKNRCGKIYLDTGGSSGHGTQNILLAYWPKEALRKKIMNKAEAYGIEVVECTDAEIRNRCSRCGTLQEPLENKKWFVCNHCGYGKDDRKTGNGFISVDYNAARNLAVYDTGSKESV

SEQ ID NO: 222 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLSCLNQNISSTYREYALDVEHGRRTIPNFKKGIPVPFAIKVHRELALRKREDGSIYIRFPKGLEWDLNFGRDRSNNREIVERVLSGQYDVGVSSIQEAKNGKRFLLLVVKIPKESRALNPDRVVGVDLGVNIPLYAALNDNTYGGLSIGSRDQFLNVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 223 - protein, 468 aa, unidentified
MPIITRKIELKLCTEGLSEETIKSQRMLLYHINDNLYRVANNISSKLYLDEHVSSLVRMKNGDYLSLEKQLVKAKKQKNPDTKTITELEERILTVKREMNSQEIAISKYATEMSTKTLAYNFAKELGSDIFGQILACLQQNVHSTFSEDAQEVRRGERAIRNYKKGMPIPFPWNRSIRIEALDGEFYLRWYNGIRFHLFFGKDRSNNRQIVKRALCLDPDYSGENYKLCNSSIQLVKREHERKTFLLLVVDIPKEVRKLNKDIVVGVDLGINVPAYVATNSTEERKEIGDREHFLNERMSFQRRYKSLQRLKCTAGGKGRKKKLEPLERLREAEHKWVHTQNHRFSREVVDFALHAEAATIHMENLSGFGKDQEGNADEKKEFVLRNWSYYELQNMIAYKAAKYGIKVEYVKPAYTSKTCSWCGKEGFRQSTIFICENHECKMCGIKVHADYNAARNIANSNNIIKNE

SEQ ID NO: 224 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGVSSIQEAKNGKRFLLLVVKIPKESRALNPDRVVGVDLGVNIPLYAALNDNTYGGLSIGSRDQFLKVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 225 - protein, 417 aa, unidentified
MQRSITLKIIRPEDETISWEELGYLLRGLSFKVCRMCNFCMTHQLLHALKLETELLNPQGNLYCYPRLAEEYPDVPTGIICAAETRARKLFRRSAEAVLHSETSLPRFRKDSSIPVPVAGYKILQDADHNVYADVQLLSRQGAKTQKLPGRIRLVLADNWRDQSAKAALQQIVAGKVKRGVASLFRVKNDWYFQIPYVTEAVNTEEIFQPDLVMGVAFGLQDALVYAFSTSMKRGAVSGEEVLAHQEKYVARRKKIQEQYNWSGRKGHGREDALKPLRHLYETERNYRNLVNSRYAKWVVDIAAKNRCGVIHLDSANYVSSGKNILLSRWPLYDLKEKIRRKAEEKGIQVTECSIPNLRTRCSRCGKEQEPEGEKRTFVCKDCGYGKADKNRRGGFITVDYNAARNLAVYESEDAKL

SEQ ID NO: 226 - protein, 488 aa, unidentified
MATEYITITRKIEVYLHRHGDSDEAKQRYQQEWQIWHDINDNLYKAANRIVSHCFFNDAYEFRLRLHSPRFAEIEKALKHSKKNKLSDDEVKALKAERQELYKSFKQQRLAFLRGGATEGANPEQNSTYKVISDEFGDIIPSDILTCLNQNIASVYKAYSKEVQFGDRTIPNYKKGIPAPFSIKAGGALLLKKREDGSIYVRMPKGLEWDLVFGRDRSNNREIVERILSGQYDAGNSSIQQAKNGKCFLLLIVKIPKSNIALNKDRVVGVDLGINIPLYVALNDNEYGGMGIGSREQFLKMRMRMSAQKRELQRNLRHSTNGGHGRKQKLQALERLEGKERNWVHLQNHIFSKSVIEFAQKHNAGVIQMERLKGYGKDANGEVREEAKFLTRYWSYFKLQTMIEYKANAAGIEIRYIDPYHTSQTCSFCGHYEKGQRISQSTFICQNPECKQGKGKQKSDGTFEGINADWNAARNIALSEQYVDKKKK

SEQ ID NO: 227 - protein, 481 aa, unidentified
MATEYTCITRKIEVHLHRHGDSDEALQRYKEEYRIWDEINDNLYKAANRIISHCFFNDAYEYRLKLHSPRFQEIEKLLKYAKRNKLTDDDIKALKAERKELFAEFKRQRQSFLGGSEQNSTDRVVSHEFLDVIPSDILTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGLSIGSRDQFLKVRMRMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVNPYHTSQTCSFCGHYEKGQRVNQATFICKNPDCTKGKGKERSDGTFEGINADWNAARNIALSDKIVERKKK

SEQ ID NO: 228 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTDRVVSHEFLDVIPSEVLTCLNQNISSTYREYALDVEHGRRTIPNFKKGIPAPFSIKANRELALRKREDGSIYIRFPKGLEWDLNFGRDRSNNREIVERVLSGQYDVGVSSIQEAKNGKRFLLLVVKIPKESRELNPDRVVGVDLGVAVPLYAALNDNQYGGMSIGSADQFIKVRMRMAAQKREMQRTLRNSTNGGHGRKQKLQALDRLEGKERRWVHLQNHIFSKELVEYALRNEAGAIQMERLTGFGHDRNDEVDEGFKFILRYWSFFELQTMIEYKAKAAGIEVRYVDPYHTSQTCSFCGHYEKGQRVTQSKFVCKNPDCTKGKGKERSDGTFEGINADWNAARNIALSDKIVERKKK

SEQ ID NO: 229 - protein, 468 aa, unidentified
MPTITRKIELTLCTDGLSDEERKAQWGLLYHINDNLYKAANNISSKLYLDEHVSSMVRLKHAEYLSLQKELAKAERQKMPDVDVIEELRERLSAAEQEMSDQELAICKYATEMSTNTLAYRFATEIETNIFGQILARLENNAQAVFLTDAPDVKRGERAIRNYKKGMPIPFPWNNSIKIECEGGEFYLRWYSGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDIIKKNE

SEQ ID NO: 230 - protein, 464 aa, unidentified
MATEFTCITRKIEVHLHRHGDSEEAIARYKNEWDMWDEINNNLYKAANRIVSHCFFNDAYEYRLKMHSPRFQEIEKLLRYTKRNNLSDNDIKQLKEERKNLFADFKKQRLAFLQGNSGIGSEQNSTYKVISNEFLDTIPSQILTNLNQNISSTYREYTQDIERGIRTIPNFKKGIPVPFSIKKGGDLMLKKRNDGSIYIRFPKGLEWDLNFGRDRSNNREIVERILNGQYDVGNSSIQETKNKKRFVLLVVKIPKENQKLDTNRIVGIDLGINTPLYAALNDNEYGGFSIGSRDQFLKMRMRMAAQKRELQRNLRHTTNGGHGRNQKLQAFKRLQDKEKNWVRLQNHIFSKSIIEYALKNKAGVIQMERLTGFGRDKNDEVNEDYKFILRYWSFFELQTMIEYKANAAGIEVRYIDPYHTSQTCSFCGHYEKGQRINQQTFICKNPDCTKGKGKQEKDGTYKGI

SEQ ID NO: 231 - protein, 469 aa, unidentified
MPTITRKIELTLCTEGLSDEQRKAQWGLLFHINDNLYKAANNVSSKLYLDEHVQSMVRMKHDEYLGLLKELARAEKQKMPDAAVIAELREKIAAAEKEMTDQELAICKYATEMPTQSLSYRLVTEIETNIFAQILDCLKQNVFATFNSDARDVKRGERAIRNYKKGMPIPFPWNKAIKIESQGDDFFLRWYSGIRFKFVFGKDRSNNRAIVMRCLKLDEDYDGEYKLCNSSIQMVKRDGGMKLFLLLVVSIPQEHVELNKKIAVGVDLGVNVPAYVATNITEERKAIGDREHFLNTRMQFQRRYKSLQRLKTTAGGKGRTKKLEPLERLRDAERNWVHTQNHLFSREVVNFAVQTHAATIHLEDLSGFGKDYDGNADERKEFVLRNWSYYELQNMITYKAAKYGIHVVKVRPAFTSRTCSCCGQQGFREGVTFICENPECKQYGEKVHADYNAARNIANSNDIIQDNHE

SEQ ID NO: 232 - protein, 487 aa, unidentified
MITSRKIKLSIVSNNSTEAYNFIRKEMKVQNKALNVAMNHLYFNSIARQKILLADKAYQQKLKAAINSQEKSFNTLKELEQKQCIEEDSEKKQALKERLIKTKNAYEKAKEKVSNLRKSRNKDSFQEYKNIIGQVEQTHLRDIISSQFNLHSDTKDRLTMIAKQDFENDIAEVLSGDRSLRTYKKNNPLYIRGRNTVLYKEGNEFFIKWIKGIVFKCILGVKNQNKTELYKTLECVLAEIHKICDSSMNFNQQDKLILNLTLDMPDKSEHRKVPERIAGVDLGLKIPAYFAVNDVPYIRKSIGKIEDFLKVRISIQSQKRSLQRALQSSKGGKGRKKKLKTLDQFKEKEKNYITTYNHFISKKIISLAVQYGVEQINLELLTLKETQKKLLLRNWSYYQLQQFIEYKANREGISIKYVDPFHTSQTCSKCGHYEDGQREKQDTFCCKSCGFKDNADYNAARNIAASTRYITNKKESEYYQQNNNEIA

SEQ ID NO: 233 - protein, 408 aa, unidentified
MVKLKHNEYASLEKKLQKAQKEKTSDDNLIADLEEKLAAAKREMTDQARAICKYATEMSTETFAYKLATEIETNVFGQILSYLKKAVQSNFSNDAQAVRRGERSIRNYKKGMPIPFPLKDRIKIKDNDFYLHWYNNIRFKLHFGKDRSNNRQIVNRCFNSVNDYKLGSSASIQIVKRNGSPKFFLLLVINIPKEEVELNKKIVVGVDLGINVPAYVATNMTEERKAIGDREHFLNNRMAFQRRFKSLQRLRGTAGGKGRTKKLEPLENLRKTERNWVHTQNHLFSREVVKFAVQTHAATIHMEDLSNFGKDKDGNADEHKEFVLRNWSYYELQEMIRYKAKKYGIEVRNVRPAYTSQTCSWCGEQGFRQGATFICKNPECKQYSEKIHADYNAARNIAKSKDIIKKNE

SEQ ID NO: 234 - protein, 448 aa, unidentified
LYKAANNISSKLYLDEHVSSLVRLKHKEYKDLMRELAKAKKQKNLDEASIIEMEGRLRSYEEEMTDQELAICKYADEMSSLSLAYGFATELELDIYAQILTQIQSKVHQDFQNDQKDVREGKRSIRTYKKGMPIPFPWNNSIRIEISKKNKVEEQEKKTRRDSEDYDFYLNWYNGLRFRLHFGKDRSNNYQIIKRCFKLDEYCHDNYQLKASSIQLVIKNKKPELYLLLVVDIPQEKYSLNNKVVVGVDLGINTPAYVATNVTEDRKAISDREHFLNARMAIKRRYRSLQRLKGTAEGRGRKKKLEPLERLREAESNWVHTQNHLFSRDIIKFALRVKAATIQMEKLEGFGKDEDGNVEEDKKFLLGEWSYYELQNMVKYKAAKVGIKVHFVKPAYTSQTCSWCGERGIRDGTAFYCQNPNCKQHGKKDINADYNAARNIAKSTEIVK

SEQ ID NO: 235 - protein, 470 aa, unidentified
MRIIRPYYGDEIEKFITAGKEKSKADGADGALTKIFWDRLKASHPEIVSAGEFYGLLCAMRMEAVVYYNRAISKLYQSLIVDVDGAQVSTAKALSAGPYHEFRERFTSYISLGLRQKLQSNFRRKELLRCQLALPTAKSDRFPIPISKQVDKQGKGGFKVSELQNGDFIIELPLMAYHKAKGKTEREYVELDAGPAILNIPVILSTQRRRANKTWFKDEGTDAEIRRVMSGEYKVSWLEILQRNRFGKAHGDWYVNFTIKYQPKDCGLDPKVKGGIDIGLSSPLVCAVTNSLDRLTIRDNDLVAFNKRALARRRTLLRRNRYKRAGHGSANKLEPITALTAKNELYRKAIMRRWAREAADFFRHNKAAAVNMEDLTGIKDREDYFSQMLRSYWNYSQMQTVLENKLKEYGIAVNYINPKDTSKTCHSCGHVNQHFDFSYRSANKFPMFKCVKCGVECGADYNAARNIAVA

SEQ ID NO: 236 - protein, 484 aa, unidentified
MIIARKIRLTVISDNRDEAYSFLRNEMYYYYKALNFAMNHVYFNYVAKEKIKMADDKYIEREQRYINAINNTHATLKKVRTESQKDLALKRIELNEQNLKKLRQSTSKEAREMLSQAISSAERTNTSDAVQKAFPMLTRDSIDFAASKATTEFNNDLKLGLLSGERVLRTYKKTNPLQIRGRLLKFFKEDGDYCIKFAKGIIFKCVLGIKRKNNTELAHTLEKVIDGTYKVCDSSFEYNDSKLILNLALQIDITARQQQPKVQGRVVGVDLGLKIPAFCALNDSIYIRERIGDIEDFLKVRTQLQARRKRLQRALKSAKGGKGQGKKLKALERFREHEKNFVKTYNHYLSKKIVHFSVLYGAEQINLELLQMAESQNKSILRNWSYYQLQQMIEYKAAKEGIKIKYVDPYRTSQTCSKCGNYENGQRKTQATFECKSCKFKENADFNAARNIAKSTAYITDKFQSEYYKNYMDCKLGARASGFH

SEQ ID NO: 237 - protein, 461 aa, unidentified
MISTRKIKVRCDDNTFYTFFRQEQREQNKALNIGIGIIHSNAILHNIDSGAEKKLKKSIEGLQGKIDKFNKHLEKEKLTDKKKEEVLKAIETTKKILDGEKKAFKKSEEYRKGIDELFKSTYLKSNTLDHVLDSMVNIQYKRTLSLVTQRIKKDYSNDFVEIITGQKSLRNYRNDNPLMISNQQLDFKYIEDTFYLDVMCGYRLEIILGRRDNQNVNELKSTLHRILSKEYKVCDSSMQFDKNNKDVILNLVIDIPNKSNMYEAIKERTLGIDLGMEVPIFMCLNDNTYIKKGIGDINNFLRVRQQIQVRRRKLQKDLTLTKGGKGRKKKLQLLDKLQENEKNFVKTYSHALSKRIIEFAKKNKCEYINLEKLTKDGFDNIILRNWSYFDLQRMIENKAKREGIVVRYVNPSFTSQKCSKCGEIDKKNRQTQADFKCINCGFELNADHNAAINIARSIDFV

SEQ ID NO: 238 - protein, 481 aa, unidentified
MIITRKIAITIVSEEAQESYNYLRQQMYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPILQRDSIDFAASKVASDFNNDLKLGLMTGSRTLRIYKRNQAYPFRSRRLKLYKENGDFFIKSSKSLLFKCLLGVKRQNSKELIQILEKILDNQYKICDSSLEFNKKKLILNLCIEVDENTHSENMKVPGVVKGRIVGVDLGIQIPAYCTLNDSPFKKKAIGSVDDLLRIRTQMQARRRRLSKNLISARGGKGRGKKLKALDRFEEYERNYVTTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIVK

SEQ ID NO: 239 - protein, 409 aa, unidentified
MELKRTARVKLAIPDDRRDDLKRTMLTFREVAQRFADRGWERDEDGYVITSRTRLQSLVYKQVREDTGLHSDLCIGAVNLAADSLRSAVERMKAGKNVGKPTFTVPTATYNTGAVSYFTDGDGTGYCTLAAYGGRVRAEFVYPPDEDCPQRQYLGGDEWEPKGATLHYERDDGEYYLHVTVERDEPETELGEAENGTVLGVDLGVENIAVTSAGAFYSGGLFNHRRDEYERIRGSLQQTGTESAHRTIEKMGDRERRWNTDVLHRISKAIVQEAITHDCSHIAFEDLTDIRDRMPGAKKFHGWAFRQLYEYVEYKAAEFGIATTQVDPAYTSQRCSKCGTTLRENRTSQAAFCCQKCGYEVHADYNAAKNVATKLLRSGQKSPAGGATNQLALKSGTLNGNGDFTPASS

SEQ ID NO: 240 - protein, 462 aa, unidentified
MKEQLNKTLTFGLGKPLGWDIRGKAFIDPTEDQRREIYISLRATSRISAQMVNMLNAREYVRRIMKIPEAFVNEFKGSYIPIKQELKTLGLEEVDDISGATLSQTWALGVKPDFAGEHGKRLLMKGDRQLPTHRIDGTHPIYGRADGTKIILHEDRYFLIVQLFSSKWANKNEFPSGWIAFPVKIKPRDKTLAGQFKRIIDGEWKLKNSHILRNPRKRGNTWLGQVVVSYTPDPFKDIDPKIIMGIDLGVSVPACLHIRENGKAKKWAMQVGRGRDMLNTRGIIRSEIVRIIRSLRSKDSPLDNESKRAAKAKLKNLRKREKRVMKTASQKIAASIADVARRNGAGTWKMELLSENIKDEDPWLRRNWAPRMVVDAVRWQAEQVGAKLEFVDPAYTSQRCSKCGHISRENRPKGKKGAAHFECVRCGYKDHADKNAARNISTPGIVDLIKEQISKSPNGEER

SEQ ID NO: 241 - protein, 405 aa, unidentified
MCRVLRRTVCLKLDVPDDRRDDLHETTDRFRQAAQIAVDRAFERNDDGYVITHKTKLHHLTYEQAREATDGLNANLVQAARNLAGDAAKGVVSRWENGKRASKPEFTAPTVVYNKKALTYREDGVSLATVNGRVECDFVLPPEGENPQTEYLRGDEWELRESTLHYRSESGEYYLHTTVAKDEDVSEEAENGTVLGVDVGVENIAATSTGRFWSSGLLNHRREQYESVRAGLQQTGTESAHRTIQQLGEREQRWVDDLLHRISKDIVSEAVEHDCSRIAFEDLTDIRERMPGAKKFHAWAFRRLFDLVSYKADERGIRTVQVDPAYTSQRCSKCGHTARNNRPSQAEFRCGRCGYENHADYNAAKNVAMKHVRAGQKSPRGRANRHLALKTGTLNGNGEFSPADA

SEQ ID NO: 242 - protein, 461 aa, unidentified
MVITRKIELHLVHTGLSDEEYDQQWKFLHSINDNLYRAANRLVNQLYLNDEIDILLRYNNKEYMELRKQLAKKNLDKPVRAELKEKEKLVLEEIKAHRSAIFQRPYASVAYSMVTSQNEANIMTKILDVLKQDVLSHYSTNAKEVARGERSISNYRYGMPIPFAFDRTEKASICIYEENKKYFLKWYNNLRFELSFGRDRSNNQLVVQRCLGISNDGAKYKACNSSIQMVRKNGSTRLFLLLCVDVPKEINKHIKGKVVGIDLGLNVPIYAAVNDGPERKSIGSREAFLDQRASFQRRFRSLQKLQMTKGGHGRLHKLEPLERVREAERNWVKNQNHLFSKEVVEFAKKVEAEIIQMERLKNFGRDDNEEIKEDKKYVVRNWSYFELQSMIEYKAKRAGIIVQYVNPAYTSQTCSECGQKGIRDNIHFKCLNPECNCFGKDIHADYNGARNIAKSKEIVKD

SEQ ID NO: 243 - protein, 398 aa, unidentified
MKIKRTIKLVVKPSEKEKHILFKTFEEYKFAYNFVAEIGWKSNIHNSVKLHNLTYIVVREKTSLPSQLVISARNVASESLKSAFSRKKKGLAVSCPYSKNPAIRYDKRSYSVWFDREEISIATIEGRLKLKIKIPEYFRQYFSWDIRSASLKYNKRLKKFFFNIVVEKEIEEIPENDTVVGVDLGLSKLATLSTADGKINKFFDGGHIRAVSERYFAIRKKLQSKGTPSAKRHLRKLSQKEKRFRTAINHKIAKEIVNLIPAGGTIVLEELKGIRERIRVYKRERRWVHSWNFAQLKQFIEYKAKSKGIKVVYINPKYTSQRCSKCGYVSKSNRKDQSHFKCSYCGYTVNADLNASRNIAINYLVSQKERLGHRVASLPVWAVVNQPNVRRLAVSSHS

SEQ ID NO: 244 - protein, 403 aa, unidentified
MQRTIRIQLKPDIETDSVLSQTIEQYTWSFNAVCKHGWKNDLANGVELHKATYYDHRAITGLPSQLVCAARVKATEALKSAKSLKKKGKTVSCPISKRCPIRYDARSYTAWFDRSELSILSINGRVKLSFEIAEYYRQYLAWKNTSADLLQDRKGCWWLHVVMEIETPQASVTDEVVGVDLGIASPAVDSRGSKYGSGHWKKIEDKTFELHRRLQSKGTKSAKGHLKKLSGRQRRFRKDCDHVLSKRLARSVESGATLVFEDLTNIRGRAKMRKAQRRRLHGWSFAQFQAFVTYKAEARGVNVGFVDPRYTSQKCSQCGHIERGNRPSQAEFRCKKCGYERHADYNAAINIRAEGIRMLKAEGSAVSAVGGEIRPKLGRKSKLRHSPVSTEADTVLGTPSQCG

SEQ ID NO: 245 - protein, 455 aa, unidentified
MPTIRRKIELLLDRTGLSKEEVDARWHTLHQINNNLYRTANNLINKLYLTDEIDDILRLNNQEYIDLKKQLGKKGLDETTKAELEERMHKIYATMNQHRSEILQRPRQSFAYSAVTGGDDTEIFNAKILDTLKQNVLAHYNADMKEVRRGEKSISNYKKGMPIPFSFDKSVRLYERNGQFFLKWYRDIQFILFFGHDASNNQLIVERCLGISEDGIAYKMCSSSLQMKGKKIFLLLVVDVPKEPFEQQKGMVVGIDLGLNVPIYATTNLTPERRAIGNRESFLNQRIAFQKRYKALQRLQLTKGGRGRSHKLEPLERLRETERNWVRTQNHLFSKEVIEFAKQVGASTINMERLASFGKNMSGEVYEDKKFVLRNWSYYELQNLIEYKAKRANIKVRYVNPAFTSQTCSECGQTGERDSIHFKCTNPECKNFGKSIHADYNGAKNIAKSTNIIKE

SEQ ID NO: 246 - protein, 472 aa, unidentified
MVITRKIELWIAEEDKTKRNETWDFLRMLDKEIFRAANVVVNNQYFNDFYEERIIQQDEKIGDVSKKIRLLYSKLRKANDEEQQSLQNEIEILKEQKKESQTRVRSEFYKTSKQNTTYQILTKQFPEIPSDILTCLNNQIYSVIGKEKKEVLQGKRSIRSYRKGMPIPFRFAQHPRLEFFENEYYLRWLNNISFVLRFGRDKSNNRAILEKIFSKEYKLCDSSIQIDDRKIFLLLVVDIPKSEHNLNKELSVGVDLGLNVPAYCALSEGFARLAIGHKDDFVRVRQQMQRRRKALQKSLVLTSGGKGRTRKLKALDALGEKERHFVRTYNHTVAKRIVEFAEKYNAGVITMELLEGYGKDENGKSRKGDFVLRNWSYFELQTLLKDKAGRKGMDVVFIDPYHTSQTCALCGHYEIGQRETQANFICKNPVCKNFDEKVNADYNAALNIARSKKFVAKKEECEYFKLHKETRS

SEQ ID NO: 247 - protein, 412 aa, unidentified
MKRTVKIKLSMNERKALEETIKQFKRACQMTVEEGWNENGLNNYKKYKLQKRVYDEIRETTDLQANLVVRAIARGAQAVKGCTQLFENGFKASKPNFTSDSIAYDKRTLSVYPDEKRCTISTVNGRIDANFVLPEEKNDYYKEYLDGSWEITQSTIEKHEYEKENSFYLHLGLEKEDEEIEHEDPTVMGVDLGMNNLAVTSTGKFFKGNQLDHNRKRFEEMRGKLQQKGTRSAHLTIQRMSERENRYACDTLHCISKELVEEAERNDVDIITFENLKYIRERMPKNKWYHVWAFNKLYQYVEYKAKERGIQVKQIDPRNTSRRCSKCGHTEKNNRNGNKFKCKQCGYELDADYNAAKNIGIKLLHRRQKSSAGAGNGQLALKSGTLKLNGEYSPTFLSARLCLRYFGIQKEF

SEQ ID NO: 248 - protein, 576 aa, unidentified
MNTIKNTYIRTLKFNLNLTPKFETEEDNKKYINDVYSYLRDAIWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDVKRELDLLENYCAYPEDIYEKFANGLSTPAYIKQKVESYWKQDGIKTKVIYSMDENLRRIKDAPLFIPPNVFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYVDTECRKYASDVIKFCLKHHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 249 - protein, 453 aa, unidentified
MTKVVKLALINNVTDKNGNKVEYYDLNKCLWDLQKETRDLKNTVIRECWEWYGFSNDYYKLNEEYPAERDHLKREKANGTIKDYSLDGFIYSKCSKKYKLHSGNLCTTLRAASGAFKTSLKDLLRGDKSVLSYKADQPLDVQKKCIVLEYDKDTNTYYITLTLLNKAGVKCYNISDFRFKITVKDNSTRTILERCYDEVYSISSSKLIWNKKKGQWFLNLCYSFDKTETKELDKNKILGVNLGVYYPIYASISGEKDRLAISGDELIEFRNRIEARRNALKKQAAVCGDGRIGHGYKTRMKPVLNISDKIANFRDTFNHKASRKLIDFAVKNDCGIIQLENLKGVTKDTEGFLKNWSFYDLQSKIENKAKERGIKVVYIEPAYTSLRCSKCGYIHKDNHPTREQFICQECGYRTLHDYNASQNIAVKDIDKIIKAELEKMGIKKNKDEEKPEK

SEQ ID NO: 250 - protein, 453 aa, unidentified
MTKVVKLALINNVTDKNGNKVEYYDLNKCLWDLQKETRDLKNTVIRECWEWYGFSNDYYKLNEEYPAERDHLKREKANGTIKDYSLDGFIYSKYSKKYKLHSGNLCTTLRTASGAFRTSLKDILRGDKSVLSYKADQPLDVQKKCIVLEYDKDTNTYYITLTLLNKTGVKFYDIGDFRFKVTVKDNSTRTILERCYDEIYSISASKLIWNKKKGQWFLNLCYSFDKTETKELDKNKILGVNLGVYYPIYASISGEKDRLAISGDELIEFRNRIEARRNALKKQAAVCGDGRIGHGYKTRMKPVLNISDKIANFRDTFNHKASRKLIDFAVKNDCGIIQLENLKGVTKDTEGFLKNWSFYDLQSKIENKAKERGIKVVYIEPAYTSLRCSKCGYIHKDNHPTREQFICQECGYRTLHDYNASQNIAVKDIDKIIKAELEKMGIKKNKDEEKPEK

SEQ ID NO: 251 - protein, 425 aa, unidentified
MTTKTYAIKLIKPVDDSWDFAGETLRNLEYIVKRFKNKAATDQYLSIVSKDKKSAAEINTGVKQGLAELAYLNYAQECFNGAIKEAVDKVSKDFKGIVTGKSSLITYKDGQPIPVRSRQITLENDNGTYYASIGLLSREYATELGREGRQKARIKFVLSSKGNEKVVLDRILSGEYKLCDSSIQRKGNAWYLQLAHSFEAKAKTDLIANRVLGIDIGISKAVYMAVSDSPVNAFIDGGEIEQFRNKTEHRRNQMRNQLKWCSDNRKSHGRNTLLKPLEVLESKVSDFRKLTNHRYAKYVVDFAVKNQCSIIQMEDLSGINTRSAFLKRWSYFDLQTKIEDKAAAHGIKVVKVNPKYTSQRCYNCGVIAKDNRESQSVYKCECTRRTKKGVVAYKVNADLNAARNLSVLGIDKEIKAQCKAQKIAY

SEQ ID NO: 252 - protein, 469 aa, unidentified
MITVRKLKVRCEDKSFYDFLRLEQREQNKALNLAIGYIHTSNILKSNDSGAETKIIKSISKLEDKIKKLNDELNKEKITDTKREKTLKAIETTTKILEGEKKILEEGKEFRIGLDKKFNEIYIDKNNMYHVLKTQTNVQYMRTLDLVKQKVSADYSNNFIDIVTGKISLMNYKQDFPLMIDNKNINLFKENDKYYIGIMLGYELEIVLGQRVNENILELKSILDKIIEDEEMAKQNKDHVKQYRFNQSSIQFDKNNNVIFNLTFTIPQDKTFKPVEGRVLGVDLGVKYPAYMCLSDDTYKREHIGSINDFLKVRTQMQNRRRELSKALSLTNSGKGRNKKTQALKRLSEKERNFAKTYNHAISKRIVDFAKKHKCEYIHLEKLTKDGFNDRILRNWSYYELQRMVEYKADRIGIKVKYINPSYTSQKCSKCGHIDKENRQTQEKFVCTQCDFELNADHNAAINIARATE

SEQ ID NO: 253 - protein, 467 aa, unidentified
MITVRKLKLTIVGDEETRNQQYKLIRDEQYQQYRALNLCMSLLSTYNILNNWNSGAENKLNSQIEKLNKKVEKNKNDLKKDNLKENRIKKINESIKTLAKEKEKLQQEYLSSSEYRSDIDKKVKEMYIDDLYTVVQSQVNFKSKDMMSLVTQRSKKDFTTALKNGMAKGERSLTNYKRDFPLMTRGERWLKFEYDEESDDIYINWLHGIRFKVVLGYKKNENSIELRHTLHKVINKEYKICDSSMQFDRNNNLILNLTLDIPLNAKNEHIEGRTLGVDLGIKYPAYVCLSDDTYKRKSIGCAEDFIKFREQIRSRRYRLQKQLSMVKGGKGRNKKLQALDRIKDKERNFVKTYNHMISKNIVEFAKNHKCESINLEKLTKDGFPNMILSKWSYYELQNMIEYKAEREGISVKYVDPAYTSQTCSKCGYVDKENRTSQEKFKCIECGFELNADHNAAINIARSNNYVK

SEQ ID NO: 254 - protein, 557 aa, unidentified
MANKNVNDSKIKTHILTKKVQLIVDTDRFDTDEEKKAEVDRVYKYLRDSMKCQSREMNQYYMHLWMMSVARNLNDDRYSMKKYMDNIIDAVHPYLDKNNKEFNNKQKKITRDFEKKIKNLCEEYQAETEILNNDTLRELNKCFNRKKDGAYDTNFVNEMPEGLGIVMGRTVEQDFQNDCKAGLLSGIRNPRSYKINYPLIIPKSFVAYGVGSGKAMQGRGIIFPDMEYSDFHNMLFSTKNPNITYNFVHDIDFKLVFGSMKRSHELRVIFDRIKMGEYSICGSTIEINNKKKIMLNLSYEAPIYEKPSLDENTVVGVDLGMAIPAVCSLNNDDRTYKYIGDSHELEFIKKGIQAQRRSYQRNAVYNKGGHGRNRKLENLDRLKKRERNTTRTHNQRYAKQIVDFALANNAKYINLENLKGFSNNDKNKLVLRNWCYYELQQYIEIDASKYGIKVRYIYPMNTSRTCSVCGTLTTDEDVKNGVGRVSQDEFICKDPNCPSHTLYTKGPKGHKVPYFNADRNASRNIAMSEDFVKKNAKDNAFKKIDDLYEINNDIDAA

SEQ ID NO: 255 - protein, 402 aa, unidentified
MITVRKLKLTIVGDEEIRKEQYKFIRDSQYAQYQGLNLAMGVLTSSYLLSGGDVKSDYFKDAQKSLKNSNKIFNEINFGKGIDSKSYITKQVKKDFSTSLKNGLAKGERGFTNYKRDFPLMTRGRDLKFYEEDKEFYIKWVNKIVFKILTGRKDKNKVELIHTLNKVLNKEYKVSQSSLQFNKNNNLILNLTIDVKSDVKVEVIKDRVCGVGVGINTPIYVALNDILYISQSIGSNDELIKQKKQFEARKKRIRERVKNKKELNSLKEKERNWINTYNHMLSKRVVEFAKKNKCEYIYLEKINDNEFKNKVLKKWPYCELQKMIRYKAAGFGIEVKYIHSYHIFQKCSRCGYEYNKSIAIQKRFKCLNCGLEVNSDYNIARNISKYDILKDKSRITGNLVSE

SEQ ID NO: 256 - protein, 405 aa, unidentified
MLNNKFIKDEKKLQESLNKLYMEKKESETKIKRSKIDEEIKKKVNVLKKMRDKESKEATKILQQAIKINLSNTTREIINQQFNLISDTKDRITQKVSQDFKADIKNGLLRGERVLRTYKKNSPLLIRGRTLQFYRKGNDILIKWYGGITFKCIIGKRKNNNHELYILLNKILENVCKVCDSSITIGRKLILNLSVALTGFEADAPTVKGRVLGVNFGIKVPIYMSLNDKSYVQKSVGNLNDLLKLRVQLYKRKKKLENLITNAIGDKVEKLKALNRLKEKEKNMLTTYNHYLSYNIVRFAKENQVGQINIEYLPFVKAKNKALKSWPYYQLQQFIEYKAKQKKIEVKYINSYLINEKCSNCGKNITHQLNSTNVFNCKKCEYKAHLDFNISQNIALSTEYISIRK

SEQ ID NO: 257 - protein, 457 aa, unidentified
MIITKKIKIIIIGENKDKYNKFIREEYYNQNKALNVAMNHLYFLHVAEEKIRMLNNKFIKDEKKLQESLNKLYMEKKESETKIKRSKIDEEIKKKVNVLKKMRDKESKEATKILQQAIKINLSNTTREIINQQFNLISDTKDRITQKVSQDFKADIKNGLLRGERVLRTYKKNSPLLIRGRTLQFYRKGNDILIKWYGGITFKCIIGKRKNNNHELYILLNKILENVCKVCDSSITIGRKLILNLSVALTGFEADAPTVKGRVLGVNFGIKVPIYMSLNDKSYVQKSVGNLNDLLKLRVQLYKRKKKLENLITNAIGDKVEKLKALNRLKEKEKNMLTTYNHYLSYNIVRFAKENQVGQINIEYLPFVKAKNKALKSWPYYQLQQFIEYKAKQKKIEVKYINSYLINEKCSNCGKNITHQLNSTNVFNCKKCEYKAHLDFNISQNIALSTEYISIRK

SEQ ID NO: 258 - protein, 458 aa, unidentified
MIITKKIKIIIIGENKDKHNKFIREEHYNQNKALNAAMNHLYFLHVAEEKIRMLNNKFIQDEKKLQESLNKLYTEKKESKTKIKRSEIDEKIKKKVNSLKKMRDKESKEAERILQQAIKINLSNTTREIINQQFNLISDTKDRITQKVSQDFKTDIKNGLLRGDRVLRTYKKTNPLLIRGRTLQFYRKGNDILIKWYGGVTFKCIIGQRKNNNHELYILLNKILENDSKVCDSSITIGRKLILNLSVALTGFEEDIPTVKGRVLGVNFGMKVPIYMSLNDKPHVQKSVGNLNDLLKLRVQLYKRKKKMKNLIIKSIGDKAEKLKVLNRFKEKEKNILTTYNHYLSYNIVQFAKENQVGQINIEYLPLVKTKNKALKSWPYYQLQQFIEYKAKRKKIEVKYINAYLLNKKCSNCGKDTTHQSNSNNIFNCKKCQYRAPLDSNISRNIALCTEYISIRKE

SEQ ID NO: 259 - protein, 760 aa, unidentified
MKLKRTIKLVVKPSEEEKQILFKTLEEYKFAYNFVAEIGWKSKVSNSIKLHNLTYTTVREKTSLPSQLVISARMVASESLKSAFNRRKKGLKVSCPYSNNPAIRYDKRSYSVWFDREEISIATVEGRLKLKIKIPEYFKQYLNWKIRSASLKYDKRLKKFFFNIVVEKEIEEIPENDTVIGVDLGLSKLAVISTADGKINKFFDGRHIRAVSERYFAIRKKLQSKGTPSAKRHLKKLSQKEKRFRTAINHKIAKEIVSLVPAGGTIVLEELKGIRERIKVSKKERRWIHSWNFAQLQQFIEYKAQSKGIKVVYINPKYTSQRCNKCGHISKSNRKDQSHFKCSSCGYTINADLNASRNIAINYLVSQQERLGHRVASLPVWAVVNQPNVRGLANFSHSAASYNTHETICCONSTRUCTPKKKRKVAASYNTHETICCONSTRUCTGIHGVPAAAASYNTHETICCONSTRUCTKRPAATKKAGQAKKKKDNASYNTHETICCONSTRUCTACCGCTTCACCAAAAGCTGTCCCTTAGGGGATTAGAACTTGAGTGAAGGTGGGCTGCTTGCATCAGCCTAATGTCGAGAAGTGCTTTCTTCGGAAAGTAACCCTCGAAACAAAGAAAGGAATGCAACDNASYNTHETICCONSTRUCTCACACACACAGTGGGCTACCDNASYNTHETICCONSTRUCTCTGAGTCCCAGTCCATCACGDNASYNTHETICCONSTRUCTAGGGTTTTGGTGGCAATGGA

SEQ ID NO: 260 - protein, 7 aa, synthetic construct
PKKKRKV

SEQ ID NO: 261 - protein, 8 aa, synthetic construct
GIHGVPAA

SEQ ID NO: 262 - protein, 16 aa, synthetic construct
KRPAATKKAGQAKKKK

SEQ ID NO: 263 - DNA, 127 bp, synthetic construct
  source 1..127: mol_type = other DNA
accgcttcaccaaaagctgtcccttaggggattagaacttgagtgaaggtgggctgcttgcatcagcctaatgtcgagaagtgctttcttcggaaagtaaccctcgaaacaaagaaaggaatgcaac

SEQ ID NO: 264 - DNA, 20 bp, synthetic construct
  source 1..20: mol_type = other DNA
cacacacacagtgggctacc

SEQ ID NO: 265 - DNA, 20 bp, synthetic construct
  source 1..20: mol_type = other DNA
ctgagtcccagtccatcacg

SEQ ID NO: 266 - DNA, 20 bp, synthetic construct
  source 1..20: mol_type = other DNA
agggttttggtggcaatgga

SEQ ID NO: 267 - protein, 7 aa, synthetic construct
DXEXNPG